(19) United States
(12) Patent Application Publication
(10) Pub. No.: US 2009/0099174 A1
(43) Pub. Date: Apr. 16, 2009

(54) COMBINATION 059

(75) Inventor: Paul David SMITH, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/252,081

(22) Filed: Oct. 15, 2008

Related U.S. Application Data
(60) Provisional application No. 60/979,898, filed on Oct. 15, 2007.

(51) Int. Cl. A61K 31/5377; A61P 35/00

(52) U.S. Cl. 514/234.2

(57) ABSTRACT

This invention relates to a combination product, as defined herein, comprising a MEK inhibitor and a mTOR-selective inhibitor, and to methods for the production of an anti-cancer effect in a patient, which is accordingly useful in the treatment of cancer in a patient. More specifically the present invention relates to; a combination product, as defined herein, comprising a MEK inhibitor and a mTOR-selective inhibitor; a combination product, as defined herein, comprising a kit of parts comprising a MEK inhibitor and a mTOR-selective inhibitor; use of the combination product, as defined herein, in the treatment of cancer; a method of treating cancer comprising administering the combination product, as defined herein, to a patient. The combination product, as defined herein, and methods of the invention are also useful in the treatment of other diseases associated with the activity of MEK, and/or mTOR.

This application claims the benefit under 35 U.S.C. § 119(e) of Application No. US60/979,898 filed on 15 Oct. 2007, which is incorporated herein by reference in its entirety.

This invention relates to a combination product, as defined herein, comprising a MEK inhibitor and a mTOR-selective inhibitor, and to methods for the production of an anti-cancer effect in a patient, which is accordingly useful in the treatment of cancer in a patient. More specifically the present invention relates to; a combination product, as defined herein, comprising a MEK inhibitor and a mTOR-selective inhibitor; a combination product, as defined herein, comprising a kit of parts comprising a MEK inhibitor and a mTOR-selective inhibitor; use of the combination product, as defined herein, in the treatment of cancer; a method of treating cancer comprising administering the combination product, as defined herein, to a patient. The combination product, as defined herein, and methods of the invention are also useful in the treatment of other diseases associated with the activity of MEK, and/or mTOR.

The Ras, Raf, MAP protein kinase/extracellular signal-regulated kinase (MEK), extracellular signal-regulated kinase (ERK) pathway plays a central role in the regulation of a variety of cellular functions dependent upon cellular context, including cellular proliferation, differentiation, survival, immortalization, invasion and angiogenesis (reviewed in Peyssonnaux and Eychene, Biology of the Cell, 2001, 93, 3-62). Indeed, the ras-dependent raf-MEK-MAPK cascade is one of the key signalling pathways responsible for conveying both mitogenic and invasive signals from the cell surface to the nucleus resulting in changes in gene expression and cell fate.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 Apr.). In fact, ERK phosphorylation is enhanced in approximately 30% of all human tumours (Hoshino et al., Oncogene, 1999, 18, 813-822). This may be a result of overexpression and/or mutation of key members of the pathway, including RAS and BRAF genes.

mTOR (mammalian target of Rapamycin) is a key cell cycle and growth control regulator. mTOR is a mammalian serine/threonine kinase of approximately 289 kD in size, and in addition to a catalytic domain in the C-terminus, contains a FKBP12/Rapamycin complex binding domain (FRB). Because of the interaction of mTOR with the FK-506-binding protein FKBP12, and SEP (sirolimus effector protein), mTOR is alternatively referred to as FRAP (FKBP12 and Rapamycin associated protein), RAFT1 (Rapamycin and FKBP12 target 1), RAPT1 (Rapamycin target 1)). The mTOR protein is a member of the PI3-kinase like kinase (PIKK) family of proteins due to its C-terminal homology (catalytic domain) with PI3-kinase and the other family members, e.g. DNA-PKcs (DNA dependent protein kinase), ATM (Ataxia-telangiectasia mutated). Growth factor or mitogenic activation of the phosphatidylinositol 3-kinase (PI3K)/AKT signalling pathway ultimately leads to mTOR. (Brown, et al., Nature, 369, 756-758 (1994); Chiu, et al., Proc Natl Acad Sci, 91, 12574-12578 (1994); Sabatini, et al., Cell, 78, 35-43, (1994); Sabers, et al., J Biol Chem, 270, 825-822 (1995)).

mTOR is a key regulator of cell growth and has been shown to regulate a wide range of cellular functions including translation, transcription, mRNA turnover, protein stability, actin cytoskeleton reorganisation and autophagy (Jacinto and Hall, Nature Reviews Molecular and Cell Biology, 2005, 4, 117-126). mTOR dependant phosphorylation of S6-kinase (S6K1) allows translation of ribosomal proteins involved in cell cycle progression (Burnett, et al., Proc Natl Acad Sci, 95, 1432-1437 (1998); Terada, et al., Proc Natl Acad Sci, 91, 11477-11481 (1994); Jeffries, et al., EMBO J, 16, 3693-3704 (1997)). Cap-dependant translation is regulated by the phosphorylation of the eukaryotic translation initiation factor 4E (eIF4E)-binding protein 1 (4E-BP1 (PHAS-1)). This modification prevents PHAS-1 binding eIF4E, thereby permitting formation of an active eIF4F translation complex (Bjornsti and Houghton, Nat Rev Cancer, 4, 335-348 (2004); Gingras, et al., Genes Dev, 13, 1422-1437 (1999); Gingras, et al., Genes Dev, 15, 807-826 (2001)). Activation of these signalling elements is dependant on insulin, other growth factors and nutrients suggesting a gatekeeper role for mTOR in the control of cell cycle progression only under favourable environmental conditions. The PI3K/AKT signalling cascade lies upstream of mTOR and this has been shown to be deregulated in certain cancers and results in growth factor independent activation in, for example, PTEN deficient cells. mTOR lies at the axis of control for this pathway and inhibitors of this kinase (e.g. sirolimus (Rapamycin or Rapamune™) and everolimus (RAD001 or Certican™)) are already approved for immunosuppression and drug eluting stents (reviewed in Neuhaus, et al., Liver Transplantation, 7, 473-484 (2001); Woods and Marks, Ann Rev Med, 55, 169-178 (2004)), and are now receiving particular interest as novel agents for cancer treatment.

In addition to the evidence linking mTOR with cell cycle regulation (from G1 to S-phase) and that inhibition of mTOR results in inhibition of these regulatory events it has been shown that down regulation of mTOR activity results in cell growth inhibition (Reviewed in refs. Burnett et al; Huang and Houghton, Curr Opin Pharmacol, 3, 371-377 (2003); Sawyers, Cancer Cell, 4, 343-348 (2003)). The known inhibitor of mTOR, Rapamycin, potently inhibits proliferation or growth of cells derived from a range of tissue types such as smooth muscle, T-cells as well as cells derived from a diverse range of tumour types including rhabdomyosarcoma, neuroblastoma, glioblastoma and medulloblastoma, small cell lung cancer, osteosarcoma, pancreatic carcinoma and breast and prostate carcinoma (Huang and Horton). Rapamycin has been approved and is in clinical use as an immunosuppressant, its prevention of organ rejection being successful and with fewer side effects than previous therapies (Huang and Houghton, Curr Opin in Invest Drugs, 3, 295-304 (2002); Brunn, et al., EMBO J, 15, 5256-5267 (1996)). Inhibition of mTOR by Rapamycin and its analogues (RAD001, CCI-779) is brought about by the prior interaction of the drug with the FK506 binding protein, FKBP12. Subsequently, the complex of FKBP12/Rapamycin then binds to the FRB domain of mTOR and inhibits the downstream signalling from mTOR.

There is also evidence that endothelial cell proliferation may also be dependent upon mTOR signalling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signalling is believed to partially control VEGF synthesis through effects on the expression of hypoxia-inducible factor-1α (HIF-1α) (Hudson et al., Molecular and Cellular Biology, 2002, 22, 7004-7014). Therefore, tumour angiogenesis may depend on mTOR kinase signalling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of cancer comprising solid tumours such as carcinomas and sarcomas and the leukaemias and lymphoid malignancies. In particular, inhibitors of mTOR kinase should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

Recently mTOR has been shown to exist in two complexes; one with raptor (TORC1), which is Rapamycin sensitive, and one with rictor (TORC2) which is a Rapamycin insensitive complex. TORC1-dependent phosphorylation of 4E-BP1 and p70S6 kinase results in translation of proteins involved in cell cycle progression. The TORC2 complex has been shown to effect targets of the cytoskeleton such as phosphorylation of paxillin. Furthermore, TORC2 directly phosphorylates and activates the upstream kinase Akt. Inhibition of TORC1 alone can stimulate Akt phosphorylation by inhibiting the negative feedback loop between p70S6 kinase and IRS1, and this is a mechanism that has been clinically shown for the Rapamycin analogues. This finding supports that there is a therapeutic advantage for inhibitors of mTOR that can inhibit both TORC1 and TORC2 complexes.

The vast majority of mTOR pharmacology to date has focused on inhibition of mTOR via Rapamycin or its analogues. However, the potent but non-specific inhibitors of PI3K, LY294002 and wortmannin, have also been shown to inhibit the kinase function of mTOR, acting through targeting the catalytic domain of the protein (Brunn et al.). In addition to Rapamycin's ability to induce growth inhibition (cytostasis) in its own right, Rapamycin and its derivatives have been shown to potentiate the cytotoxicity of a number of chemotherapies including cisplatin, camptothecin and doxorubicin (Huang and Horton). Potentiation of ionising radiation induced cell killing has also been observed following inhibition of mTOR (Eshleman, et al., Cancer Res, 62, 7291-7297 (2002)). Experimental and clinical evidence has shown that Rapamycin analogues are showing evidence of efficacy in treating cancer, either alone or in combination with other therapies (Bjornsti and Houghton; Huang and Houghton; Huang and Houghton).

Accordingly, it has been recognised that an inhibitor of a protein of the MAPK kinase pathway should be of value both as an anti-proliferative, pro-apoptotic and anti-invasive agent for use in the containment and/or treatment of proliferative or invasive disease. Furthermore it has been recognised that an inhibitor of mTOR should be of value both for inhibiting proliferation and cell growth in the containment and/or treatment of proliferative disease. Thus, inhibition of a protein in the MAPK kinase pathway and inhibition of mTOR should be particularly useful, as both pathways are essential for cellular growth and survival.

International publication number WO2006044453 describes certain compounds that are 17-hydroxywortmannin analogues. The application describes certain 17-hydroxywortmannin analogues that may be used in combination with other compounds, such as MEK inhibitors. While the Applicants assert that the 17-hydroxywortmannin analogues are TOR (mTOR) inhibitors, by the Applicants own admission, these compounds are predominantly inhibitors of PI3K.

Surprisingly, we have found that concurrent inhibition of both MEK and mTOR, using a selective inhibitor of mTOR, yields synergistic or additive inhibition of tumour cell line growth or viability, in comparison with inhibition of MEK alone or selective inhibition of mTOR alone. We have found that concurrent inhibition of MEK and mTOR, using the mTOR inhibitor Rapamycin, yields synergistic or additive inhibition of tumour cell line growth or viability. However, we have found that in several of the cell lines tested, the maximal inhibition of cell growth achieved by the combination of a MEK inhibitor and Rapamycin was less than that achieved by the combination of a MEK inhibitor and a mTOR-selective inhibitor. In vivo results show that concurrent inhibition of MEK and mTOR, with a selective inhibitor of mTOR, yields synergistic inhibition of HCT-116 tumour xenografts and additive inhibition of LoVo, Calu-6 and A549a tumour xenografts. Concurrent inhibition of MEK and mTOR, using the mTOR inhibitor Rapamycin, yields additive inhibition of LoVo and Calu-6 tumour xenografts, while in A549a tumour xenografts the combination is antagonistic (i.e. the combination did not have any effect over Rapamycin used alone). However, we have found that in the tumour xenografts tested, a greater degree of tumour growth inhibition was achieved by the combination of a MEK inhibitor and a mTOR-selective inhibitor than that achieved by the combination of a MEK inhibitor and Rapamycin. It is expected that inhibiting two key components of the growth factor signal transduction pathways known to be involved in cancer, will lead to greater inhibition of tumour growth or viability than that which would be achieved by the inhibition of either MEK or mTOR alone.

The present invention provides a combination product comprising a MEK inhibitor and a mTOR-selective inhibitor. The combination product of the invention is useful in a method for the production of an anti-cancer effect in a patient, which is accordingly useful in the treatment of cancer in a patient.

According to a first aspect of the present invention there is provided a combination product comprising

a MEK inhibitor, or a pharmaceutically acceptable salt thereof, and

a mTOR-selective inhibitor, or a pharmaceutically acceptable salt thereof, in association with a pharmaceutically acceptable adjuvant, diluent or carrier.

The combination product of the present invention provides for the administration of a MEK inhibitor in conjunction with a mTOR-selective inhibitor. The combination product, as defined herein, may be in the form of a combined preparation of a MEK inhibitor and a mTOR-selective inhibitor. The combination product, as defined herein, may comprise a kit of parts comprising separate formulations of a MEK inhibitor and a mTOR-selective inhibitor. The separate formulations of a MEK inhibitor and a mTOR-selective inhibitor may be administered sequentially, separately and/or simultaneously. In one embodiment the separate formulations of a MEK inhibitor and a mTOR-selective inhibitor of the combination product, as defined herein, are administered simultaneously (optionally repeatedly). In one embodiment the separate formulations of a MEK inhibitor and a mTOR-selective inhibitor of the combination product, as defined herein, are administered sequentially (optionally repeatedly). In one embodiment the separate formulations of a MEK inhibitor and a mTOR-selective inhibitor of the combination product, as defined herein, are administered separately (optionally repeatedly). The skilled person will understand that where the separate formulations of a MEK inhibitor and a mTOR-selective inhibitor of the combination product, as defined herein, are administered sequentially or serially that this could be administration of a MEK inhibitor followed by a mTOR-selective inhibitor, or a mTOR-selective inhibitor followed by a MEK inhibitor. In one embodiment the separate formulations of a MEK inhibitor and a mTOR-selective inhibitor of the combination product, as defined herein, may be administered in alternative dosing patterns. Where the administration of the separate formulations of a MEK inhibitor and a mTOR-selective inhibitor of the combination product, as defined herein, is sequential or separate, the delay in administering the second formulation should not be such as to lose the beneficial effect of the combination therapy. Thus, the present invention provides a combination product, as defined herein, comprising a MEK inhibitor, or a pharmaceutically-acceptable salt thereof, and a mTOR-selective inhibitor, or a pharmaceutically-acceptable salt thereof, for use sequentially, separately and/or simultaneously in the treatment of cancer.

In another aspect there is provided a combination product, as defined herein, which comprises a kit of parts comprising the following components:
a MEK inhibitor, or a pharmaceutically acceptable salt thereof, in association with a pharmaceutically acceptable adjuvant, diluent or carrier; and
a mTOR-selective inhibitor, or a pharmaceutically acceptable salt thereof, in association with a pharmaceutically acceptable adjuvant, diluent or carrier,
   wherein the components are provided in a form which is suitable for sequential, separate and/or simultaneous administration.

In one embodiment the kit of parts comprises
a first container comprising a MEK inhibitor, or a pharmaceutically acceptable salt thereof in association with a pharmaceutically acceptable adjuvant, diluent or carrier; and
a second container comprising a mTOR-selective inhibitor, or a pharmaceutically acceptable salt thereof, in association with a pharmaceutically acceptable adjuvant, diluent or carrier, and
   a container means for containing said first and second containers.

In one embodiment the kit of parts further comprises instructions to administer the components sequentially, separately and/or simultaneously. In one embodiment the kit of parts further comprises instructions indicating that the combination product, as defined herein, can be used in the treatment of cancer.

In another aspect there is provided a combination product, as defined herein, comprising a pharmaceutical composition which comprises a MEK inhibitor, or a pharmaceutically-acceptable salt thereof, and a mTOR-selective inhibitor, or a pharmaceutically-acceptable salt thereof.

In another aspect there is provided a pharmaceutical composition which comprises a MEK inhibitor, or a pharmaceutically-acceptable salt thereof, and a mTOR-selective inhibitor, or a pharmaceutically-acceptable salt thereof.

In one embodiment the MEK inhibitor is a small molecular weight compound. In one embodiment the MEK inhibitor is selected from any one of an ATP-competitive MEK inhibitor, a non-ATP competitive MEK inhibitor, or an ATP-uncompetitive MEK inhibitor. In one embodiment the MEK inhibitor is selected from any one of AZD6244 as described in International Patent Publication Number WO03/077914, 2-(2-fluoro-4-iodophenylamino)-N-(2-hydroxyethoxy)-1,5-dimethyl-6-oxo-1,6-dihydropyridine-3-carboxamide, 4-(4-Bromo-2-fluorophenylamino)-N-(2-hydroxyethoxy)-1,5-dimethyl-6-oxo-1,6-dihydropyridazine-3-carboxamide, PD-0325901 (Pfizer), PD-184352 (Pfizer), XL-518 (Exelixis), AR-119 (Ardea Biosciences, Valeant Pharmaceuticals), AS-701173 (Merck Serono), AS-701255 (Merck Serono), 360770-54-3 (Wyeth). In one embodiment the MEK inhibitor is selected from AZD6244, 2-(2-fluoro-4-iodophenylamino)-N-(2-hydroxyethoxy)-1,5-dimethyl-6-oxo-1,6-dihydropyridine-3-carboxamide or 4-(4-Bromo-2-fluorophenylamino)-N-(2-hydroxyethoxy)-1,5-dimethyl-6-oxo-1,6-dihydropyridazine-3-carboxamide as described below. In one embodiment the MEK inhibitor is selected from AZD6244 or 2-(2-fluoro-4-iodophenylamino)-N-(2-hydroxyethoxy)-1,5-dimethyl-6-oxo-1,6-dihydropyridine-3-carboxamide, as described below. In one embodiment the MEK inhibitor is AZD6244 hydrogen sulphate salt. AZD6244 hydrogen sulphate salt may be synthesised according to the process described in International Patent Publication Number WO07/076,245.

In another embodiment the MEK inhibitor may inhibit gene expression, for example by interfering with mRNA stability or translation. In one embodiment the MEK inhibitor is selected from small interfering RNA (siRNA), which is sometimes known as short interfering RNA or silencing RNA, or short hairpin RNA (shRNA), which is sometimes known as small hairpin RNA.

In one embodiment the mTOR-selective inhibitor is selective for mTOR over PI3K. In one embodiment the mTOR-selective inhibitor is greater than 2 fold selective for mTOR over PI3K. In one embodiment the mTOR-selective inhibitor is greater than 10 fold selective for mTOR over PI3K. In one embodiment the mTOR-selective inhibitor is greater than 100 fold selective for mTOR over PI3K. In one embodiment the mTOR-selective inhibitor inhibits TORC2. In one embodiment the mTOR-selective inhibitor inhibits TORC1 and TORC2. In one embodiment the mTOR-selective inhibitor is a small molecular weight compound. In one embodiment the mTOR-selective inhibitor is selected from any one of an ATP-competitive mTOR-selective inhibitor, a non-ATP competitive mTOR-selective inhibitor, or an ATP-uncompetitive mTOR-selective inhibitor. In one embodiment the mTOR-selective inhibitor is selected from any one of the small molecular weight compounds disclosed in International Patent Publication Number WO2006/090167, WO2006/090169, WO2007/080382, WO2007/060404 or International Patent Application Number PCT/GB2007/003179, or a pharmaceutically acceptable salt thereof. In one embodiment the mTOR-selective inhibitor is OSI-027 (OSI Pharmaceuticals).

In one embodiment the mTOR-selective inhibitor is a compound of formula I:

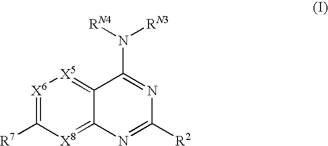

wherein:
one or two of X⁵, X⁶ and X⁸ is N, and the others are CH;
R⁷ is selected from halo, OR^{O1}, SR^{S1}, NR^{N1}R^{N2}, NR^{N7a}C(═O)R^{C1}, NR^{N7b}SO₂R^{S2a}, an optionally substituted C₅₋₂₀ heteroaryl group, or an optionally substituted C₅₋₂₀ aryl group,
where R^{O1} and R^{s1} are selected from H, an optionally substituted C₅₋₂₀ aryl group, an optionally substituted C₅₋₂₀ heteroaryl group, or an optionally substituted C₁₋₇ alkyl group; R^{N1} and R^{N2} are independently selected from H, an optionally substituted C₁₋₇ alkyl group, an optionally substituted C₅₋₂₀ heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted C₅₋₂₀ heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted C₅₋₂₀ heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted C₅₋₂₀heteroaryl group, or an optionally substituted C₁₋₇ alkyl group;
R^{N7a} and R^{N7b} are selected from H and a C₁₋₄ alkyl group;
R^{N3} and R^{N4}, together with the nitrogen to which they are bound, form a heterocyclic ring containing between 3 and 8 ring atoms;
R² is selected from H, halo, OR^{O2}, SR^{S2b}, NR^{N5}R^{N6}, an optionally substituted C₅₋₂₀ heteroaryl group, and an optionally substituted C₅₋₂₀ aryl group,
wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group;
R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms,
or a pharmaceutically acceptable salt thereof,
with the proviso that when R² is unsubstituted morpholino, R^{N3} and R^{N4} together with the nitrogen atom to which they are attached form an unsubstituted morpholino and R⁷ is unsubstituted phenyl, and X⁵ is CH, then X⁶ is not N and X⁸ is not CH, or X⁶ is not CH and X⁸ is not N, and
when R² is unsubstituted piperidinyl, R^{N3} and R^{N4} together with the nitrogen atom to which they are attached form an unsubstituted piperidinyl and R⁷ is unsubstituted phenyl, and X⁵ is CH, then X⁶ is not CH and X⁸ is not N.

In one embodiment of the first aspect, when R² is unsubstituted morpholino, R^{N3} and R^{N4} together with the nitrogen atom to which they are attached form an unsubstituted morpholino, R⁷ is unsubstituted morpholino or di-methylamino, and X⁶ is CH, then X⁵ is not N and X⁸ is not CH, or X⁵ is not CH and X⁸ is not N.

In another embodiment of the first aspect, when R² is unsubstituted piperazino or N-formyl piperazino, R^{N3} and R^{N4} together with the nitrogen atom to which they are attached form an unsubstituted morpholino, unsubstituted piperidinyl or unsubstituted oxidothiomorpholino, R⁷ is unsubstituted morpholino or benzylamino, and X⁶ is CH, then X⁵ is not N and X⁸ is not CH, or X⁵ is not CH and X⁸ is not N.

In another embodiment of the first aspect, when R² is unsubstituted morpholino, unsubstituted piperidino, unsubstituted pyrrolidino, R^{N3} and R^{N4} together with the nitrogen atom to which they are attached form a morpholino, piperazinyl, unsubstituted piperidinyl or unsubstituted pyrrolidinyl, R⁷ is unsubstituted morpholino, unsubstituted piperidinyl, unsubstituted pyrrolidinyl, and X⁵ is CH, then X⁶ is not N and X⁸ is not CH, or X⁶ is not CH and X⁸ is not N.

According to another aspect of the present invention there is provided a compound of formula I(A):

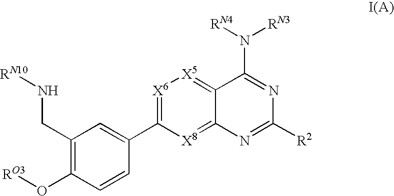

wherein:
one or two of X⁵, X⁶ and X⁸ is N, and the others are CH;
R^{N3} and R^{N4}, together with the nitrogen to which they are bound, form a heterocyclic ring containing between 3 and 8 ring atoms;
R² is selected from H, halo, OR^{O2}, SR^{S2b}, NR^{N5}R^{N6}, an optionally substituted C₅₋₂₀ heteroaryl group, and an optionally substituted C₅₋₂₀ aryl group,
wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group; R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms;
R^{O3} is selected from hydrogen or an optionally substituted C₁₋₆ alkyl group; and
R^{N10} is selected from C(═O)R^{C2}, C(═S)R^{C3}, SO₂R^{S3}, an optionally substituted C₅₋₂₀ heteroaryl group, an optionally substituted C₅₋₂₀ aryl group, or an optionally substituted C₁₋₁₀ alkyl group where R^{C2} and R^{C3} are selected from H, an optionally substituted C₅₋₂₀ aryl group, an optionally substituted C₅₋₂₀ heteroaryl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heteroaryl group, an optionally substituted C₅₋₂₀ aryl group or R^{N11} and R^{N12} together with the nitrogen to which they are bound form a heterocyclic ring containing between 3 and 8 ring atoms; and R^{S3} is selected from H, an optionally substituted C₅₋₂₀ aryl group, an optionally substituted C₅₋₂₀ heteroaryl group, or an optionally substituted C₁₋₇ alkyl group,
or a pharmaceutically acceptable salt thereof.

Accordingly, another aspect of the present invention provides a compound of formula I(B):

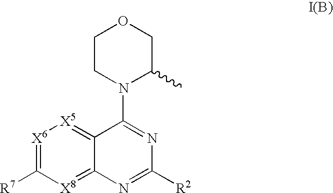

, or a pharmaceutically acceptable salt thereof, wherein:
one or two of X⁵, X⁶ and X⁸ is N, and the others are CH;
R⁷ is selected from halo, OR^{O1}, SR^{S1}, NR^{N1}R^{N2}, NR^{N7a}C(O)R^{C1}, NR^{N7b}SO₂R^{S2a}, an optionally substituted 5- to 20-membered heteroaryl group, or an optionally substituted C₅₋₂₀ aryl group, where R^{O1} and R^{S1} are selected from H, an optionally substituted C₅₋₂₀ aryl group, an optionally substituted 5- to 20-membered heteroaryl group, or an optionally substituted C₁₋₇ alkyl group; R^{N1} and R^{N2} are independently selected from H, an optionally substituted C₁₋₇ alkyl group, an optionally substituted 5- to 20-membered heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted 5- to 20-membered heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted 5- to 20-membered heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted 5- to 20-membered heteroaryl group, or an optionally substituted C₁₋₇ alkyl group;
R^{N7a} and R^{N7b} are selected from H and a C₁₋₄ alkyl group;
R² is selected from H, halo, OR^{O2}, SR^{S2b}, NR^{N5}R^{N6}, an optionally substituted 5- to 20-membered heteroaryl group, and an optionally substituted C₅₋₂₀ aryl group,
wherein R^{O2} and R^{S2b} are selected from H, an optionally substituted C₅₋₂₀ aryl group, an optionally substituted 5- to 20-membered heteroaryl group, or an optionally substituted C₁₋₇ alkyl group; R^{N5} and R^{N6} are independently selected from H, an optionally substituted C₁₋₇ alkyl group, an optionally substituted 5- to 20-membered heteroaryl group, and an optionally substituted C₅₋₂₀ aryl group, or R^{N5} and R^{N6} together with the nitrogen to which they are bound form a heterocyclic ring containing between 3 and 8 ring atoms.

According to another aspect of the present invention there is provided a compound of formula I(B)i or I(B)ii:

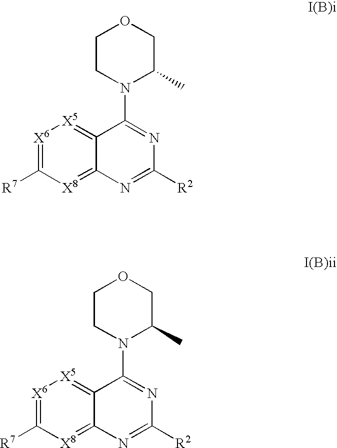

or a pharmaceutically acceptable salt thereof, wherein:
one or two of X⁵, X⁶ and X⁸ is N, and the others are CH;
R⁷ is selected from halo, OR^{O1}, SR^{S1}, NR^{N1}R^{N2}, NR^{N7a}C(O)R^{C1}, NR^{N7b}SO₂R^{S2a}, an optionally substituted 5- to 20-membered heteroaryl group, or an optionally substituted C₅₋₂₀ aryl group, where R^{O1} and R^{S1} are selected from H, an optionally substituted C₅₋₂₀ aryl group, an optionally substituted 5- to 20-membered heteroaryl group, or an optionally substituted C₁₋₇ alkyl group; R^{N1} and R^{N2} are independently selected from H, an optionally substituted C₁₋₇ alkyl group, an optionally substituted 5- to 20-membered heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted 5- to 20-membered heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted 5- to 20-membered heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted 5- to 20-membered heteroaryl group, or an optionally substituted C₁₋₇ alkyl group;
R^{N7a} and R^{N7b} are selected from H and a C₁₋₄ alkyl group;
R² is selected from H, halo, OR^{O2}, SR^{S2b}, NR^{N5}R^{N6}, an optionally substituted 5- to 20-membered heteroaryl group, and an optionally substituted C₅₋₂₀ aryl group,
wherein R^{O2} and R^{S2b} are selected from H, an optionally substituted C₅₋₂₀ aryl group, an optionally substituted 5- to 20-membered heteroaryl group, or an optionally substituted C₁₋₇ alkyl group; R^{N5} and R^{N6} are independently selected from H, an optionally substituted C₁₋₇ alkyl group, an optionally substituted 5- to 20-membered heteroaryl group, and an optionally substituted C₅₋₂₀ aryl group, or R^{N5} and R^{N6} together with the nitrogen to which they are bound form a heterocyclic ring containing between 3 and 8 ring atoms.

According to another aspect of the present invention there is provided a compound of formula I(B)i:

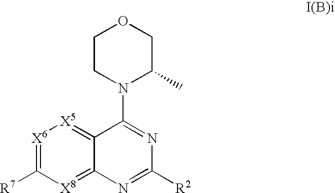

, or a pharmaceutically acceptable salt thereof, wherein:
one or two of X⁵, X⁶ and X⁸ is N, and the others are CH;
R⁷ is selected from halo, OR^{O1}, SR^{S1}, NR^{N1}R^{N2}, NR^{N7a}C(O)R^{C1}, NR^{N7b}SO₂R^{S2a}, an optionally substituted 5- to 20-membered heteroaryl group, or an optionally substituted C₅₋₂₀ aryl group, where R^{O1} and R^{S1} are selected from H, an optionally substituted C₅₋₂₀ aryl group, an optionally substituted 5- to 20-membered heteroaryl group, or an optionally substituted C₁₋₇ alkyl group; R^{N1} and R^{N2} are independently selected from H, an optionally substituted C₁₋₇ alkyl group, an optionally substituted 5- to 20-membered heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted 5- to 20-membered heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted 5- to 20-membered heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted 5- to 20-membered heteroaryl group, or an optionally substituted C₁₋₇ alkyl group;
R^{N7a} and R^{N7b} are selected from H and a C₁₋₄ alkyl group;
R² is selected from H, halo, OR^{O2}, SR^{S2b}, NR^{N5}R^{N6}, an optionally substituted 5- to 20-membered heteroaryl group, and an optionally substituted C₅₋₂₀ aryl group,
wherein R^{O2} and R^{S2b} are selected from H, an optionally substituted C₅₋₂₀ aryl group, an optionally substituted 5- to 20-membered heteroaryl group, or an optionally substituted C₁₋₇ alkyl group; R^{N5} and R^{N6} are independently selected from H, an optionally substituted C₁₋₇ alkyl group, an optionally substituted 5- to 20-membered heteroaryl group, and an optionally substituted C₅₋₂₀ aryl group, or R^{N5} and R^{N6} together with the nitrogen to which they are bound form a heterocyclic ring containing between 3 and 8 ring atoms.

### DEFINITIONS

The term “aromatic ring” is used herein in the conventional sense to refer to a cyclic aromatic structure, that is, a structure having delocalised π-electron orbitals.

Nitrogen-containing heterocyclic ring having from 3 to 8 ring atoms: The term “Nitrogen-containing heterocyclic ring having from 3 to 8 ring atoms” as used herein refers to a 3 to 8 membered heterocylic ring containing at least one nitrogen ring atom. The term “together with the nitrogen to which they are bound, form a heterocyclic ring containing between 3 and 8 ring atoms” as used herein refers to a 3 to 8 membered heterocylic ring containing at least one nitrogen ring atom. Examples of these groups include, but are not limited to:
N₁: aziridine (C₃ ie 3 membered), azetidine (C₄ ie 4 membered), pyrrolidine (tetrahydropyrrole) (C₅ ie 5 membered), pyrroline (e.g., 3-pyrroline, 2,5-dihydropyrrole) (C₅ ie 5 membered), 2H-pyrrole or 3H-pyrrole (isopyrrole, isoazole) (C₅ ie 5 membered), piperidine (C₆ ie 6 membered), dihydropyridine (C₆ ie 6 membered), tetrahydropyridine (C₆ ie 6 membered), azepine (C₇ ie 7 membered);
N₂: imidazolidine (C₅ ie 5 membered), pyrazolidine (diazolidine) (C₅ ie 5 membered), imidazoline (C₅ ie 5 membered), pyrazoline (dihydropyrazole) (C₅ ie 5 membered), piperazine (C₆ ie 6 membered);
N₁O₁: tetrahydrooxazole (C₅ ie 5 membered), dihydrooxazole (C₅ ie 5 membered), tetrahydroisoxazole (C₅ ie 5 membered), dihydroisoxazole (C₅ ie 5 membered), morpholine (C₆ ie 6 membered), tetrahydrooxazine (C₆ ie 6 membered), dihydrooxazine (C₆ ie 6 membered), oxazine (C₆ ie 6 membered);
N₁S₁: thiazoline (C₅ ie 5 membered), thiazolidine (C₅ ie 5 membered), thiomorpholine (C₆ ie 6 membered);
N₂O₁: oxadiazine (C₆ ie 6 membered);
N₁O₁S₁: oxathiazine (C₆ ie 6 membered).

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes saturated alkyl, alkenyl, alkynyl, saturated cycloalkyl, cycloalkyenyl, cylcoalkynyl, etc., discussed below. Unless otherwise specified, preferable “alkyl” groups are saturated alkyl or saturated cycloalkyl groups, more preferably saturated alkyl groups.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

The term saturated alkyl group includes saturated linear alkyl and saturated branched alkyl.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C═CH) and 2-propynyl (propargyl, —CH₂—C═CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:
saturated monocyclic hydrocarbon compounds: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);
unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);
saturated polycyclic hydrocarbon compounds: thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);
unsaturated polycyclic hydrocarbon compounds: camphene (C₁₀), limonene (C₁₀), pinene (C₁₀);
polycyclic hydrocarbon compounds having an aromatic ring: indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms. Preferably the ring heteroatoms are selected from O, N and S. The heterocyclic ring may, unless otherwise specified, be carbon or nitrogen linked, and wherein a —CH₂— group can optionally be replaced by a —C(O)—, and a ring sulphur atom may be optionally oxidised to form the S-oxides.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl” or “5 to 6 membered heterocyclyl”, as used herein, pertains to a heterocyclyl group having 5 or 6 ring atoms. Examples of groups of heterocyclyl groups include C₃₋₂₀ heterocyclyl (ie 3 to 20 membered heterocyclyl), C₅₋₂₀ heterocyclyl (ie 5 to 20 membered heterocyclyl), C₃₋₁₅ heterocyclyl (ie 3 to 15 membered heterocyclyl), C₅₋₁₅ heterocyclyl (ie 5 to 15 membered heterocyclyl), C₃₋₁₂ heterocyclyl (ie 3 to 12 membered heterocyclyl), C₅₋₁₂ heterocyclyl (ie 5 to 12 membered heterocyclyl), C₃₋₁₀ heterocyclyl (ie 3 to 10 membered heterocyclyl), C₅₋₁₀ heterocyclyl (ie 5 to 10 membered heterocyclyl), C₃₋₇ heterocyclyl (ie 3 to 7 membered heterocyclyl), C₅₋₇ heterocyclyl (ie 5 to 7 membered heterocyclyl), and C₅₋₆ heterocyclyl (ie 5 to 6 membered heterocyclyl).

Examples of monocyclic heterocyclyl groups include, but are not limited to, those derived from:
N₁: aziridine (C₃ ie 3 membered), azetidine (C₄ ie 4 membered), pyrrolidine (tetrahydropyrrole) (C₅ ie 5 membered), pyrroline (e.g., 3-pyrroline, 2,5-dihydropyrrole) (C₅ ie 5 membered), 2H-pyrrole or 3H-pyrrole (isopyrrole, isoazole) (C₅ ie 5 membered), piperidine (C₆ ie 6 membered), dihydropyridine (C₆ ie 6 membered), tetrahydropyridine (C₆ ie 6 membered), azepine (C₇ ie 7 membered);
O₁: oxirane (C₃ ie 3 membered), oxetane (C₄ ie 4 membered), oxolane (tetrahydrofuran) (C₅ ie 5 membered), oxole (dihydrofuran) (C₅ ie 5 membered), oxane (tetrahydropyran) (C₆ ie 6 membered), dihydropyran (C₆ ie 6 membered), pyran (C₆ ie 6 membered), oxepin (C₇ ie 7 membered);
S₁: thiirane (C₃ ie 3 membered), thietane (C₄ ie 4 membered), thiolane (tetrahydrothiophene) (C₅ ie 5 membered), thiane (tetrahydrothiopyran) (C₆ ie 6 membered), thiepane (C₇ ie 7 membered);
O₂: dioxolane (C₅ ie 5 membered), dioxane (C₆ ie 6 membered), and dioxepane (C₇ ie 7 membered);
O₃: trioxane (C₆ ie 6 membered);
N₂: imidazolidine (C₅ ie 5 membered), pyrazolidine (diazolidine) (C₅ ie 5 membered), imidazoline (C₅ ie 5 membered), pyrazoline (dihydropyrazole) (C₅ ie 5 membered), piperazine (C₆ ie 6 membered);
N₁O₁: tetrahydrooxazole (C₅ ie 5 membered), dihydrooxazole (C₅ ie 5 membered), tetrahydroisoxazole (C₅ ie 5 membered), dihydroisoxazole (C₅ ie 5 membered), morpholine (C₆ ie 6 membered), tetrahydrooxazine (C₆ ie 6 membered), dihydrooxazine (C₆ ie 6 membered), oxazine (C₆ ie 6 membered);
N₁S₁: thiazoline (C₅ ie 5 membered), thiazolidine (C₅ ie 5 membered), thiomorpholine (C₆ ie 6 membered);
N₂O₁: oxadiazine (C₆ ie 6 membered);
O₁S₁: oxathiole (C₅ ie 5 membered) and oxathiane (thioxane) (C₆ ie 6 membered); and,
N₁O₁S₁: oxathiazine (C₆ ie 6 membered).

Examples of substituted (non-aromatic) monocyclic heterocyclyl groups include those derived from saccharides, in cyclic form, for example, furanoses (C₅ ie 5 membered), such as arabinofuranose, lyxofuranose, ribofuranose, and xylofuranse, and pyranoses (C₆ ie 6 membered), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring (3 to 7 membered) joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms. Commonly, heteroatoms are selected from oxygen, nitrogen or sulphur.

Examples of C₅₋₂₀ heteroaryl groups include, but are not limited to, C₅ heteroaryl groups (5 membered heteroaryl groups) derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups (6 membered heteroaryl groups) derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups (9 membered heteroaryl groups) derived from benzofuran, isobenzofuran, benzothiophene, indole, isoindole; C₁₀ heteroaryl groups (10 membered heteroaryl groups) derived from quinoline, isoquinoline, benzodiazine, pyridopyridine; C₁₋₄ heteroaryl groups (14 membered heteroaryl groups) derived from acridine and xanthene.

The above defined groups eg alkyl, heterocyclyl, aryl etc, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cylic amino groups may be substituted on their ring by any of the substituents defined here, for example carboxy, carboxylate and amido.

Aminosulfonyl —S(═O)₂NR¹R², wherein R¹ and R² each independently is an amino substituent, as defined for amino groups. Examples of aminosulfonyl groups include, but are not limited to, —S(═O)₂NH₂, —S(═O)₂NHCH₃, —S(═O)₂NHCH₂CH₃ and —S(═O)₂N(CH₃)₂.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

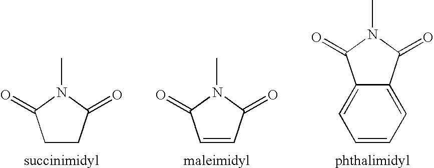

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

In addition, two or more adjacent substituents may be linked such that together with the atoms to which they are attached from a C₃₋₇ cycloalkyl, C₃₋₂₀ heterocyclyl or C₅₋₂₀ aryl ring.

As mentioned above, the groups that form the above listed substituent groups, e.g. C₁₋₇ alkyl, C₃₋₂₀ heterocyclyl, and C₅₋₂₀ aryl, may themselves be substituted. Thus, the above definitions cover substituent groups which are substituted.

Accordingly, a further aspect of the present invention provides a compound of formula I:

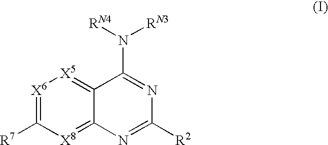

wherein:
one or two of X⁵, X⁶ and X⁸ is N, and the others are CH;
R⁷ is halo, OR^{O1}, SR^{S1}, NR^{N1}R^{N2}, NR^{N7a}C(═O)R^{C1}, NR^{N7b}SO₂R^{S2a}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
where R^{O1} and R^{S1} are H, a C₅₋₂₀ aryl group, a C₅₋₂₀ heteroaryl group, or a C₁₋₇ alkyl group where each C₁₋₇alkyl, C₅₋₂₀heteroaryl, or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N1} and R^{N2} are independently H, a C₁₋₇alkyl group, a C₅₋₂₀heteroaryl group, a C₅₋₂₀ aryl group or R^{N1} and R^{N2} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{C1} is H, a C₅₋₂₀ aryl group, a C₅₋₂₀ heteroaryl group, a C₁₋₇ alkyl group or NR^{N8}R^{N9} where R^{N8} and R^{N9} are independently selected from H, a C₁₋₇ alkyl group, a C₅₋₂₀ heteroaryl group, a C₅₋₂₀ aryl group or R^{N8} and R^{N9} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{S2a} is H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N7a} and R^{N7b} are H or a C₁₋₄ alkyl group;
R^{N3} and R^{N4}, together with the nitrogen to which they are bound, form a heterocyclic ring containing between 3 and 8 ring atoms optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R² is H, halo, OR^{O2}, SR^{S2b}, NR^{N5}R^{N6}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
wherein R^{O2} and R^{S2b} are H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N5} and R^{N6} are independently H, a C₁₋₇ alkyl group, a C₅₋₂₀ heteroaryl group, a C₅₋₂₀ aryl group, or R^{N5} and R^{N6} together with the nitrogen to which they are bound form a heterocyclic ring containing between 3 and 8 ring atoms where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
or a pharmaceutically acceptable salt thereof,
with the proviso that when R² is unsubstituted morpholino, R^{N3} and R^{N4} together with the nitrogen atom to which they are attached form an unsubstituted morpholino and R⁷ is unsubstituted phenyl, and X⁵ is CH, then X⁶ is not N and X⁸ is not CH, or X⁶ is not CH and X⁸ is not N, and
when R² is unsubstituted piperidinyl, R^{N3} and R^{N4} together with the nitrogen atom to which they are attached form an unsubstituted piperidinyl and R⁷ is unsubstituted phenyl, and X⁵ is CH, then X⁶ is not CH and X⁸ is not N.

According to a further aspect of the present invention there is provided a compound of formula I(A):

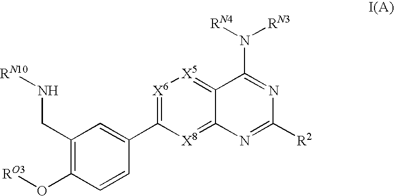

wherein:
one or two of X⁵, X⁶ and X⁸ is N, and the others are CH;
R^{N3} and R^{N4}, together with the nitrogen to which they are bound, form a heterocyclic ring containing between 3 and 8 ring atoms optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R² is H, halo, OR^{O2}, SR^{S2b}, NR^{N5}R^{N6}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
wherein R^{O2} and R^{S2b} are H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N5} and R^{N6} are independently H, a C₁₋₇ alkyl group, a C₅₋₂₀ heteroaryl group, a C₅₋₂₀ aryl group, or R^{N5} and R^{N6} together with the nitrogen to which they are bound form a heterocyclic ring containing between 3 and 8 ring atoms where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{O3} is hydrogen or a C₁₋₆ alkyl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino); and
R^{N10} is C(═O)R^{C2}, C(═S)R^{C3}, SO₂R^{S3}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₁₋₁₀ alkyl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
where R^{C2} and R^{C3} are H, a C₅₋₂₀ aryl group, a C₅₋₂₀ heteroaryl group, a C₁₋₇ alkyl group or NR^{N11}R^{N12}, where R^{N11} and R^{N12} are independently H, a C₁₋₇ alkyl group, a C₅₋₂₀ heteroaryl group, a C₅₋₂₀ aryl group or R^{N11} and R^{N12} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), and R^{S3} is H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
or a pharmaceutically acceptable salt thereof.

According to a further aspect of the present invention there is provided a compound of formula I(B):

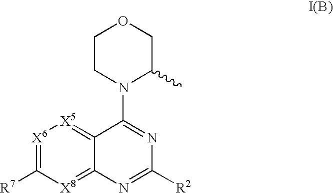

, or a pharmaceutically acceptable salt thereof, wherein:
one or two of X⁵, X⁶ and X⁸ is N, and the others are CH;
R⁷ is halo, OR^{O1}, SR^{S1}, NR^{N1}R^{N2}, NR^{N7a}C(═O)R^{C1}, NR^{N7b}SO₂R^{S2a}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, is C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
where R^{O1} and R^{S1} are H, a C₅₋₂₀ aryl group, a C₅₋₂₀ heteroaryl group, or a C₁₋₇ alkyl group where each C₁₋₇alkyl, C₅₋₂₀heteroaryl, or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N1} and R^{N2} are independently H, a C₁₋₇alkyl group, a C₅₋₂₀heteroaryl group, a C₅₋₂₀ aryl group or R^{N1} and R^{N2} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{C1} is H, a C₅₋₂₀ aryl group, a C₅₋₂₀ heteroaryl group, a C₁₋₇ alkyl group or NR^{N8}R^{N9} where R^{N8} and R^{N9} are independently selected from H, a C₁₋₇ alkyl group, a C₅₋₂₀ heteroaryl group, a C₅₋₂₀ aryl group or R^{N8} and R^{N9} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{S2a} is H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N7a} and R^{N7b} are H or a C₁₋₄ alkyl group;
R² is H, halo, OR^{O2}, SR^{S2b}, NR^{N5}R^{N6}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
wherein R^{O2} and R^{S2b} are H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N5} and R^{N6} are independently H, a C₁₋₇ alkyl group, a C₅₋₂₀ heteroaryl group, a C₅₋₂₀ aryl group, or R^{N5} and R^{N6} together with the nitrogen to which they are bound form a heterocyclic ring containing between 3 and 8 ring atoms where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino).

According to a further aspect of the present invention there is provided a compound of formula I(B)i or I(B)ii:

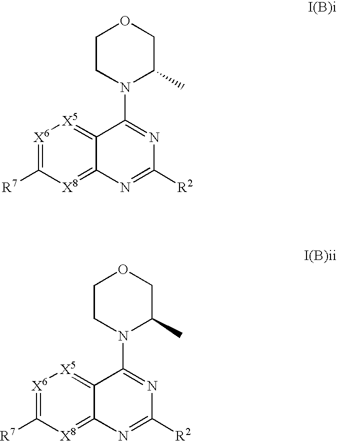

or a pharmaceutically acceptable salt thereof, wherein:
one or two of X⁵, X⁶ and X⁸ is N, and the others are CH;
R⁷ is halo, OR^{O1}, SR^{S1}, NR^{N1}R^{N2}, NR^{N7a}C(═O)R^{C1}, NR^{N7b}SO₂R^{S2a}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
where R^{O1} and R^{S1} are H, a C₅₋₂₀ aryl group, a C₅₋₂₀ heteroaryl group, or a C₁₋₇ alkyl group where each C₁₋₇alkyl, C₅₋₂₀heteroaryl, or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N1} and R^{N2} are independently H, a C₁₋₇alkyl group, a C₅₋₂₀heteroaryl group, a C₅₋₂₀ aryl group or R^{N1} and R^{N2} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{C1} is H, a C₅₋₂₀ aryl group, a C₅₋₂₀ heteroaryl group, a C₁₋₇ alkyl group or NR^{N8}R^{N9} where R^{N8} and R^{N9} are independently selected from H, a C₁₋₇ alkyl group, a C₅₋₂₀ heteroaryl group, a C₅₋₂₀ aryl group or R^{N8} and R^{N9} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{S2a} is H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N7a} and R^{N7b} are H or a C₁₋₄ alkyl group;
R² is H, halo, OR^{O2}, SR^{S2b}, NR^{N5}R^{N6}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
wherein R^{O2} and R^{S2b} are H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N5} and R^{N6} are independently H, a C₁₋₇ alkyl group, a C₅₋₂₀ heteroaryl group, a C₅₋₂₀ aryl group, or R^{N5} and R^{N6} together with the nitrogen to which they are bound form a heterocyclic ring containing between 3 and 8 ring atoms where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino).

According to a further aspect of the present invention there is provided a compound of formula I(B)i:

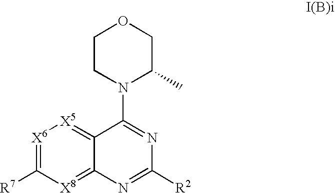

or a pharmaceutically acceptable salt thereof, wherein:
one or two of X⁵, X⁶ and X⁸ is N, and the others are CH;
R⁷ is halo, OR^{O1}, SR^{S1}, NR^{N1}R^{N2}, NR^{N7a}C(═O)R^{C1}, NR^{N7b}SO₂R^{S2a}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
where R^{O1} and R^{S1} are H, a C₅₋₂₀ aryl group, a C₅₋₂₀ heteroaryl group, or a C₁₋₇ alkyl group where each C₁₋₇alkyl, C₅₋₂₀heteroaryl, or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N1} and R^{N2} are independently H, a C₁₋₇alkyl group, a C₅₋₂₀heteroaryl group, a C₅₋₂₀ aryl group or R^{N1} and R^{N2} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{C1} is H, a C₅₋₂₀ aryl group, a C₅₋₂₀ heteroaryl group, a C₁₋₇ alkyl group or NR^{N8}R^{N9} where R^{N8} and R^{N9} are independently selected from H, a C₁₋₇ alkyl group, a C₅₋₂₀ heteroaryl group, a C₅₋₂₀ aryl group or R^{N8} and R^{N9} together with the nitrogen to which they are bound form a heterocyclic ring containing between 3 and 8 ring atoms, where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{S2a} is H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N7a} and R^{N7b} are H or a C₁₋₄ alkyl group;
R² is H, halo, OR^{O2}, SR^{S2b}, NR^{N5}R^{N6}, a C₅₋₂₀ heteroaryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino), or a C₅₋₂₀ aryl group optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino),
wherein R^{O2} and R^{S2b} are H, a C₅₋₂₀ aryl group, a C₅₋₂₀ heteroaryl group, or a C₁₋₇ alkyl group where each C₁₋₇alkyl, C₅₋₂₀heteroaryl or C₅₋₂₀aryl is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino);
R^{N5} and R^{N6} are independently H, a C₁₋₇ alkyl group, a C₅₋₂₀ heteroaryl group, a C₅₋₂₀ aryl group, or R^{N5} and R^{N6} together with the nitrogen to which they are bound form a heterocyclic ring containing between 3 and 8 ring atoms where each C₁₋₇alkyl, C₅₋₂₀heteroaryl, C₅₋₂₀aryl or heterocyclic ring is optionally substituted by one or more groups selected from halo, hydroxyl, nitro, cyano, carboxy, and thiol, or C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino (each optionally substituted with one or more groups selected from halo, hydroxyl, nitro, cyano, carboxy, thiol, C₁₋₇alkyl, C₂₋₇alkenyl, C₂₋₇alkynyl, C₃₋₇cycloalkyl, C₃₋₇cycloalkenyl, C₃₋₂₀heterocyclyl, C₅₋₂₀aryl, C₅₋₂₀heteroaryl, ether, acyl, ester, amido, amino, acylamido, ureido, acyloxy, thioether, sulfoxide, sulfonyl, thioamido and sulfonamino).

### (i) Further Preferences:

The following preferences can apply to each aspect of the present invention, where applicable. The preferences for each group may be combined with those for any or all of the other groups, as appropriate.

### X⁵, X⁶, and X⁸

When two of X⁵, X⁶ and X⁸ are N, preferably X⁵ and X⁸ are N.

It is preferred that only one of X⁵, X⁶ and X⁸ is N. More preferably one of X⁵ and X⁸ is N, and most preferably X⁸ is N.

### R⁷

R⁷ is preferably selected from an optionally substituted C₅₋₂₀ aryl group, OR^{O1}, SR^{S1}, NR^{N1}R^{N2}, NR^{N7a}C(═O)R^{C1} and NR^{N7b}SO₂R^{S2a}, where R^{O1}R^{S1}, R^{N1}, R^{N2}, R^{N7a}, R^{N7b}, R^{C1} and R^{S2a} are as previously defined. It is further preferred that R⁷ is preferably selected from an optionally substituted C₅₋₂₀ aryl group, OR^{O1}, NR^{N1}R^{N2}, NR^{N7a}C(O)R^{C1} and NR^{N7b}SO₂R^{S2a}.

If R⁷ is OR^{O1}, then preferably R^{O1} is a C₁₋₇ alkyl group, which may be substituted.

If R⁷ is NR^{N1}R^{N2}, then preferably R^{N2} is selected from H and C₁₋₄ alkyl (e.g. methyl) and more preferably is H. If R^{N1} is C₁₋₇ alkyl, it is preferably selected from C₃₋₇ cycloalkyl. If R^{N1} is C₅₋₂₀ aryl, it is preferably selected from C₅₋₁₀ aryl and more preferably C₅₋₆ aryl (e.g. phenyl, pyrrolyl, pyridyl, furanyl, thiophenyl, pyrazinyl, pyrimidinyl, thiazolyl, imidazolyl, triazolyl, oxadiazolyl). Particularly preferred groups include phenyl, pyridyl, pyrrolyl, and thiophenyl. The aforementioned groups are optionally substituted, and in some embodiments are preferably substituted. Substituent groups may include, but are not limited to, C₁₋₇ alkyl, C₃₋₂₀ heterocyclyl, C₅₋₂₀ aryl, carboxy, ester, hydroxy, aryloxy, cyano, halo, nitro, and amino.

If R⁷ is NR^{N1}R^{N2}, then preferably R^{N2} is selected from H and C₁₋₄ alkyl (e.g. methyl) and more preferably is H. If R^{N1}is C₁₋₇ alkyl, it is preferably selected from C₃₋₇ cycloalkyl. If R^{N1} is C₅₋₂₀ aryl, it is preferably selected from C₅₋₁₀ aryl (e.g. phenyl, pyrrolyl, pyridyl, pyrazolyl, furanyl, thiophenyl, pyrazinyl, pyrimidinyl, tetrazolyl, thiazolyl, indazolyl, imidazolyl, triazolyl, oxadiazolyl) and more preferably C₅₋₆ aryl (e.g. phenyl, pyrrolyl, pyridyl, pyrazolyl, furanyl, thiophenyl, pyrazinyl, pyrimidinyl, tetrazolyl, thiazolyl, imidazolyl, triazolyl, oxadiazolyl). Particularly preferred groups include furyl, phenyl, pyridyl, pyrrolyl, pyrazolyl and thiophenyl. The aforementioned groups are optionally substituted, and in some embodiments are preferably substituted. Substituent groups may include, but are not limited to, C₁₋₇ alkyl, C₃₋₂₀ heterocyclyl, C₅₋₂₀ aryl, carboxy, ester, ether (eg C₁₋₇alkoxy), hydroxy, aryloxy, cyano, halo, nitro, amido, sulfonyl, sulfonylamino, amino sulfonyl and amino.

If R⁷ is NR^{N7a}C(═O)R^{C1}, then R^{N7a} is preferably H. R^{C1} may be an optionally substituted C₅₋₂₀ aryl group (e.g. phenyl, imidazolyl, quinoxalinyl), C₃₋₂₀ heterocyclyl, C₁₋₇ alkyl (e.g. propenyl, methyl (substituted with thiophenyl)) or NR^{N8}R^{N9}. R^{N8} is preferably hydrogen, and R^{N9} is preferably C₁₋₇ alkyl (e.g. ethyl).

If R⁷ is NR^{N7b}SO₂R^{S2a}, then R^{N7b} is preferably H. R^{S2a} is preferably C₁₋₇ alkyl (e.g. methyl).

If R⁷ is a C₅₋₂₀ aryl group, it is preferably a C₅₋₁₀ aryl and more preferably C₅₋₆ aryl group. Most preferably R⁷ is an optionally substituted phenyl group, wherein the optional substituents are preferably selected from halo, hydroxyl, C₁₋₇ alkyl and C₁₋₇ alkoxy.

If R⁷ is a C₅₋₂₀ aryl group, it is preferably an optionally substituted C₅₋₁₀ aryl and more preferably an optionally substituted C₅₋₆ aryl group. Most preferably it is an optionally substituted phenyl group, wherein the optional substituents are preferably selected from halo, hydroxyl, C₁₋₇ alkyl, C₁₋₇ alkoxy, C₅₋₆arylamino and C₁₋₇alkylamino and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino and C₁₋₇alkylamino.

In one embodiment, R⁷ is an optionally substituted C₅₋₁₀ aryl group, wherein the optional substituents are selected from cyano, halo, hydroxyl, and C₁₋₇ alkyl and C₁₋₇ alkoxy (wherein the alkyl groups may be optionally substituted by one or more groups selected from halo, hydroxyl, C₁₋₇ alkoxy, amino and C₅₋₆ aryl). In another embodiment, R⁷ is an optionally substituted C₅₋₆ aryl group, wherein the optional substituents are selected from cyano, halo, hydroxyl, and C₁₋₇ alkyl and C₁₋₇ alkoxy (wherein the alkyl groups may be optionally substituted by one or more groups selected from halo, hydroxyl, C₁₋₇ alkoxy, amino and C₅₋₆ aryl). In a further embodiment R⁷ is a thiophenyl group or a phenyl group optionally substituted by one or more groups selected from chloro, hydroxyl, methyl, methoxy, ethoxy, i-propoxy, benzyloxy and hydroxymethyl. In a further embodiment R⁷ is 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group.

If R⁷ is a 5 to 20 membered heteroaryl group, it is preferably an optionally substituted 5 to 10 membered heteroaryl and more preferably an optionally substituted 5 or 6 membered heteroaryl group.

In one embodiment, R⁷ is an optionally substituted C₅₋₂₀ aryl group or an optionally substituted 5 to 20 membered heteroaryl group, wherein the optional substituents are preferably selected from halo, hydroxyl, cyano, C₁₋₇ alkyl, C₁₋₇alkoxy, sulfonamino (for example —NHS(═O)₂C₁₋₇alkyl)amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, —NHS(═O)₂C₁₋₇alkyl, C₅₋₆arylamino, di-(C₁₋₇alkyl)amino and C₁₋₇alkylamino.

In one embodiment, R⁷ is an optionally substituted phenyl group, wherein the optional substituents are preferably selected from halo, hydroxyl, cyano, C₁₋₇ alkyl, C₁₋₇alkoxy, sulfonamino (for example —NHS(═O)₂C₁₋₇alkyl)amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, —NHS(═O)₂C₁₋₇alkyl, C₅₋₆arylamino, di-(C₁₋₇alkyl)amino and C₁₋₇alkylamino.

In one embodiment, R⁷ is an optionally substituted phenyl group, wherein the optional substituents are preferably selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino.

In one embodiment, R⁷ is an optionally substituted phenyl group, wherein the optional substituents are preferably selected from fluoro, hydroxyl, cyano, nitro, methyl, methoxy, —OCH₂CH₃, —NH₂, —NHSO₂CH₃, —CH₂NHSO₂CH₃, —OCHF₂, —CH₂OH, —CO₂H, —CONH₂, —CONHMe, —CONHEt, —CONHCH(CH₃)₂, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl.

In one embodiment, R⁷ is an optionally substituted phenyl group, wherein the optional substituents are preferably selected from fluoro, hydroxyl, cyano, nitro, methyl, methoxy, —CH₂OH, —CO₂H, —CONH₂, —CONHMe, —CONHEt, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl.

In one embodiment, R⁷ is an optionally substituted phenyl group, wherein the optional substituents are preferably selected from methoxy, —OCH₂CH₃, —NH₂, —NHSO₂CH₃, —CH₂NHSO₂CH₃, —OCHF₂, —CH₂OH, —CONH₂, —CONHMe and —CONHCH(CH₃)₂.

In one embodiment R⁷ is an optionally substituted 5 or 6 membered nitrogen containing heteroaryl group such as a pyridine group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CO₂NH₂, —CO₂NHC₁₋₇alkyl, —CO₂N(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino.

In one embodiment, R⁷ is a pyridinyl group optionally substituted halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CO₂NH₂, —CO₂NHC₁₋₇alkyl, —CO₂N(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino.

In one embodiment, R⁷ is a pyridinyl group optionally substituted with NH₂.

In one embodiment, R⁷ is an optionally substituted phenyl group selected from

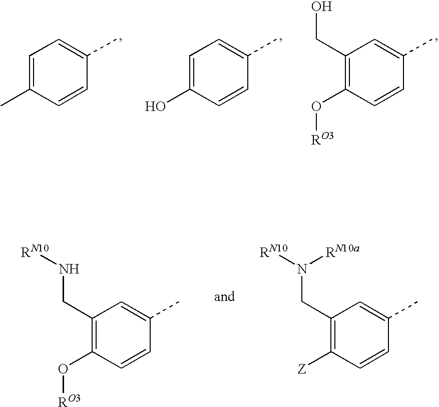

wherein

### Z is H, F or OR^{O3};

R^{O3} is selected from hydrogen or an optionally substituted C₁₋₆ alkyl group;
R^{N10} is selected from hydrogen, C(O)R^{C2}, C(S)R^{C3}, SO₂R^{S3}, an optionally substituted C₅₋₂₀ heterocyclyl group, an optionally substituted C₅₋₂₀ aryl group, or an optionally substituted C₁₋₁₀ alkyl group where R^{C2} and R^{C3} are selected from H, an optionally substituted C₅₋₂₀ aryl group, an optionally substituted C₅₋₂₀ heterocyclyl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heterocyclyl group, an optionally substituted C₅₋₂₀ aryl group or R^{N11} and R^{N12} together with the nitrogen to which they are bound form a heterocyclic ring containing between 3 and 8 ring atoms; and R^{S3} is selected from H, an optionally substituted C₅₋₂₀ aryl group, an optionally substituted C₅₋₂₀ heteroaryl group, or an optionally substituted C₁₋₇ alkyl group;
R^{N10a} is selected from hydrogen or an optionally substituted C₁₋₁₀ alkyl group; or
R^{N10} and R^{N10a} together with the nitrogen to which they are bound form an optionally substituted heterocyclic ring containing between 3 and 8 ring atoms.

In one embodiment, R⁷ is an optionally substituted phenyl group selected from

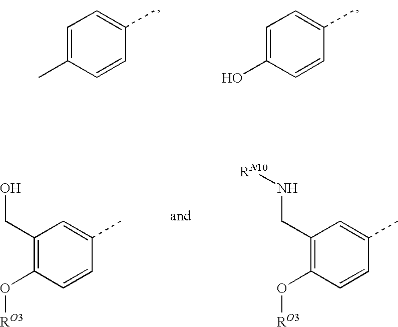

wherein
R^{O3} is selected from hydrogen or an optionally substituted C₁₋₆ alkyl group; and
R^{N10} is selected from C(O)R^{C2}, C(S)R^{C3}, SO₂R^{S3}, an optionally substituted C₅₋₂₀ heteroaryl group, an optionally substituted C₅₋₂₀ aryl group, or an optionally substituted C₁₋₁₀ alkyl group where R^{C2} and R^{C3} are selected from H, an optionally substituted C₅₋₂₀ aryl group, an optionally substituted C₅₋₂₀ heteroaryl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heteroaryl group, an optionally substituted C₅₋₂₀ aryl group or R^{N11} and R^{N12} together with the nitrogen to which they are bound form a heterocyclic ring containing between 3 and 8 ring atoms; and R^{S3} is selected from H, an optionally substituted C₅₋₂₀ aryl group, an optionally substituted C₅₋₂₀ heteroaryl group, or an optionally substituted C₁₋₇ alkyl group.

In one embodiment, R⁷ is

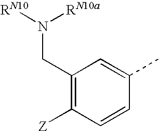

wherein

### Z is H, F or OR^{O3};

R^{N10} is selected from hydrogen, C(O)R^{C2}, an optionally substituted C₅₋₂₀ heteroaryl group, an optionally substituted C₅₋₂₀ aryl group, or an optionally substituted C₁₋₁₀ alkyl group where R^{C2} are selected from H, an optionally substituted C₅₋₂₀ aryl group, an optionally substituted C₅₋₂₀ heterocyclyl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heterocycyl group, an optionally substituted C₅₋₂₀ aryl group or R^{N11} and R^{N12} together with the nitrogen to which they are bound form a heterocyclic ring containing between 3 and 8 ring atoms;
R^{N10a} is selected from hydrogen or an optionally substituted C₁₋₁₀ alkyl group; or
R^{N10} and R^{N10a} together with the nitrogen to which they are bound form an optionally substituted heterocyclic ring containing between 3 and 8 ring atoms.

In one embodiment, R⁷ is

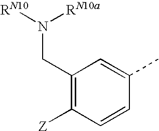

wherein

### Z is H, F or OR^{O3};

R^{N10} is selected from hydrogen, C(O)R^{C2}, an optionally substituted C₅₋₆ heteroaryl group, an optionally substituted C₆ aryl group, or an optionally substituted C₁₋₁₀ alkyl group where R^{C2} are selected from CH₃ or CH₂OH;
R^{N10a} is selected from hydrogen or an optionally substituted C₁₋₁₀ alkyl group; or
R^{N10} and R^{N10a} together with the nitrogen to which they are bound form an optionally substituted heterocyclic ring containing between 3 and 8 ring atoms;
and where the optional substituents are selected from cyano, halo, hydroxyl, C₁₋₇alkyloxy, C₁₋₇alkylamino and di-C₁₋₇alkylamino.

In one embodiment, R⁷ is

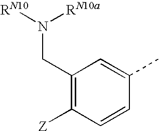

wherein

### Z is H, F or OR^{O3};

R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cyclopropyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms; and where the optional substituents are selected from halo, hydroxyl, C₁₋₇alkyloxy.

In a further embodiment of the invention R⁷ is selected from

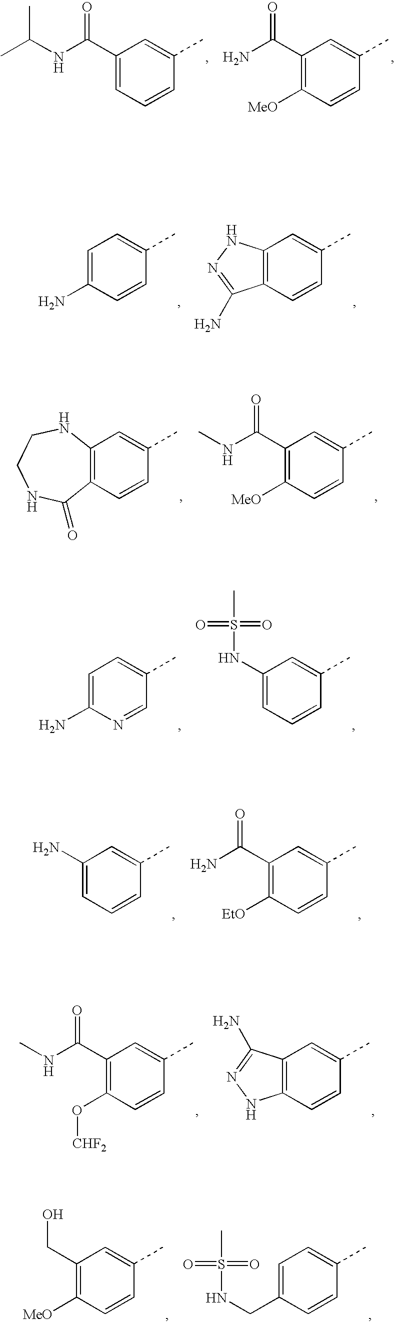

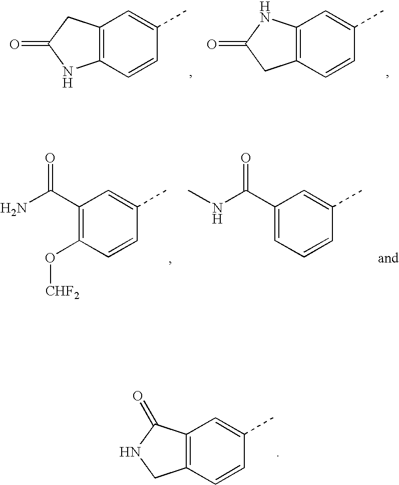

### R^{N10}

R^{N10} is preferably selected from C(═S)R^{C3}, an optionally substituted C₅₋₂₀ heteroaryl group, an optionally substituted C₅₋₂₀ aryl group, and an optionally substituted C₁₋₁₀ alkyl group where R^{C3} is as previously defined.

If R^{N10} is C(═S)R^{C3}, then preferably R^{C3} is NR^{N11}R^{N12}, where R^{N11} and R^{N12} together with the nitrogen to which they are bound form a heterocyclic ring containing between 3 and 8 ring atoms.

If R^{N10} is a C₅₋₂₀ heteroaryl group, it is preferably a C₅₋₁₀ heteroaryl group and more preferably C₅₋₆ heteroaryl group. Most preferably it is an optionally substituted pyrazole group, wherein the optional substituents are preferably selected from halo, hydroxyl, C₁₋₇ alkyl and C₁₋₇ alkoxy.

If R^{N10} is a C₅₋₂₀ aryl group, it is preferably a C₅₋₁₀ aryl and more preferably C₅₋₆ aryl group. Most preferably it is an optionally substituted phenyl group, wherein the optional substituents are preferably selected from halo, hydroxyl, C₁₋₇ alkyl and C₁₋₇ alkoxy.

If R^{N10} is a C₁₋₁₀ alkyl group, it is preferably a C₁₋₁₀ alkyl group and more preferably C₁₋₁₀ alkyl group. Most preferably it is an optionally substituted C₁₋₆ alkyl group, wherein the optional substituents are preferably selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₇alkoxycarbonylamino.

### R^{O3}

R^{O3} is preferably an optionally substituted C₁₋₆ alkyl group. More preferably R^{O3} is an unsubstituted C₁₋₃ alkyl group, preferably a methyl group.

### R^{N3} and R^{N4}

R^{N3} and R^{N4} together with the nitrogen to which they are bound preferably form a heterocyclic ring containing between 5 and 7 ring atoms, which may optionally be substituted. Preferred optionally substituted groups include, but are not limited, to morpholino, thiomorpholino, piperidinyl, piperazinyl (preferably N-substituted), homopiperazinyl (preferably N-substituted) and pyrrolidinyl.

More preferably the group formed is morpholino or thiomorpholino, which are preferably unsubstituted. The most preferred group is morpholino.

### R²

In one embodiment R² is OR^{O2} where R^{O2} is an optionally substituted C₁₋₇alkyl group.

In one embodiment R² is OR² where R^{O2} is —CH₃, —CH₂CH₃, —CH₂CH₂OH, —CH₂CH₂OCH₃, or —CH(CH₃)CH₂N(CH₃)₂.

In one embodiment R² is selected from NR^{N5}R^{N6}, an optionally substituted C₅₋₂₀ heteroaryl group, and an optionally substituted C₅₋₂₀ aryl group.

In another embodiment R² is selected from NR^{N5}R^{N6}, an optionally substituted C₅₋₆ heteroaryl group, and an optionally substituted C₆ aryl group.

In a further embodiment R² is phenyl group optionally substituted with one or more groups selected from hydroxyl, amino, nitro, carboxyl, formyl, cyano, methyl, amido, methyl, methoxymethyl and hydroxymethyl.

Preferably R² is NR^{N5}R^{N6}, where R^{N5} and R^{N6} are as previously defined, and more preferably R^{N5} and R^{N6} together with the nitrogen to which they are bound form a heterocyclic ring containing between 3 and 8 ring atoms, which may optionally be substituted. The ring preferably has from 5 to 7 ring atoms. Preferred optionally substituted groups include, but are not limited, to morpholino, thiomorpholino, piperadinyl, piperazinyl (preferably N-substituted), homopiperazinyl (preferably N-substituted) and pyrrolidinyl.

Preferably R² is NR^{N5}R^{N6}, where R^{N5} and R^{N6} are as previously defined, and more preferably R^{N5} and R^{N6} together with the nitrogen to which they are bound form a heterocyclic ring containing between 3 and 8 ring atoms, which may optionally be substituted. The ring preferably has from 5 to 7 ring atoms. Preferred optionally substituted groups include, but are not limited, to imidazolyl, morpholino, thiomorpholino, piperadinyl, homopiperadinyl, piperazinyl (preferably N-substituted), homopiperazinyl (preferably N-substituted) and pyrrolidinyl.

Preferred N-substituents for the piperazinyl and homopiperazinyl groups include esters, in particular, esters bearing a C₁₋₇ alkyl group as an ester substituent, e.g. —C(═O)OCH₃, —C(═O)OCH₂CH₃ and —C(═O)OC(CH₃)₃.

Preferred N-substituents for the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃.

Preferred C-substituents for the groups include C₁₋₄ alkyl, preferably methyl. The groups may bear one or more substituents, for example one or two substituents.

Preferred C-substituents for the groups include phenyl, ester, amide and C₁₋₄ alkyl, preferably methyl, aminomethyl, hydroxymethyl or hydroxyethyl. The groups may bear one or more substituents, for example one or two substituents.

More preferred groups are morpholino and piperidinyl. These are preferably substituted with one or two methyl substituents. If these groups bear two methyl substituents, these are preferably on separate carbon atoms. Particularly preferred groups include:

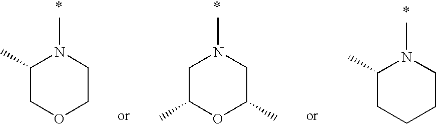

In one embodiment R² is NR^{N5}R^{N6} where R^{N5} and R^{N6} together with the nitrogen to which they are bound form a heterocyclic ring containing 5 to 7 ring atoms which may be optionally be substituted, wherein the optional substituents are selected from amino, cyano, halo, hydroxyl, ester, a C₃₋₇ cycloalkyl ring, a C₆carboaryl ring, a heterocyclic ring containing 5 to 7 ring atoms and C₁₋₇ saturated alkyl and C₁₋₇ saturated alkoxy (wherein the heterocyclic ring, the cycloalkyl ring, the carboaryl ring, the saturated alkyl and alkoxy groups may be optionally substituted by one or more groups selected from halo, hydroxyl, C₁₋₇ alkoxy, amino and C₅₋₆ aryl)

In one embodiment R² is NR^{N5}R^{N6} where R^{N5} and R^{N6} together with the nitrogen to which they are bound form a heterocyclic ring containing between 5 to 7 ring atoms which may be optionally be substituted, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl)

In one embodiment R² is NR^{N5}R^{N6}, where R^{N5} is an optionally substituted C₁₋₇alkyl group or an optionally substituted phenyl group, and R^{N6} is hydrogen.

In one embodiment R² is NR^{N5}R^{N6}, where R^{N5} is —CH(CH₃)CH₂OCH₃, cyclopentyl or a phenyl group, and R^{N6} is hydrogen.

More preferred groups are morpholino and piperadinyl. These are preferably substituted with one or more alkyl substituents, for example methyl or ethyl substituents. More preferably these are substituted with one or two methyl substituents. If these groups bear two methyl substituents, these are preferably on separate carbon atoms. Particularly preferred groups include methylmorpholino groups, dimethylmorpholino groups and methyl piperidinyl groups, for example:

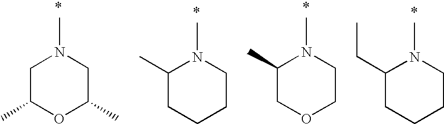

Preferred R² groups are pyrrolidinyl, morpholino, piperadinyl and homopiperadinyl groups. More preferred groups are morpholino and piperadinyl. These are preferably substituted with one or more alkyl substituents, for example methyl or ethyl substituents. More preferably these are substituted with one or two methyl substituents. If these groups bear two methyl substituents, these are preferably on separate carbon atoms. The alkyl substituents may also be optionally substituted. Examples of optional substituents of the alkyl substitutents include halo, hydroxy, ether or amino. Particularly preferred groups include methylmorpholino groups, dimethylmorpholino groups and methyl piperidinyl groups, for example:

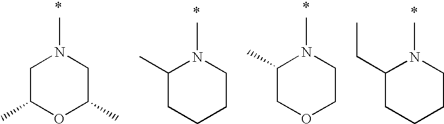

Preferred R² groups are pyrrolidinyl, morpholino, piperadinyl and homopiperadinyl groups. More preferred groups are morpholino and piperadinyl. These are preferably substituted with one or more alkyl substituents, for example methyl or ethyl substituents. More preferably these are substituted with one or two methyl substituents. If these groups bear two methyl substituents, these are preferably on separate carbon atoms. The alkyl substituents may also be optionally substituted. Examples of optional substituents of the alkyl substitutents include halo, hydroxy, ether or amino. Particularly preferred groups include methylmorpholino groups, dimethylmorpholino groups and methyl piperidinyl groups, for example:

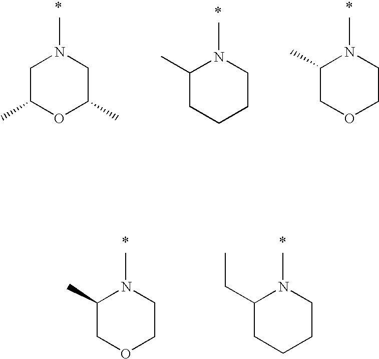

Further preferred R² groups are optionally substituted pyrrolidinyl, morpholino, piperadinyl and homopiperadinyl wherein the optional substituents are selected from hydroxyl, C₁₋₇ alkyl, C₁₋₇alkoxy, amino (for example —NH₂, C₅₋₆arylamino, C₁₋₇alkylamino, and di-(C₁₋₇alkyl)amino), amido (for example —CONH₂, —CONHC₁₋₇alkyl, —CON(C₁₋₇alkyl)₂), ester (for example —CO₂C₁₋₇alkyl), C₆aryl and 3 to 7 membered heterocyclyl group and wherein the substitutent alkyl, alkoxy, aryl or heterocyclyl groups may be further optionally substituted by one or more groups selected from halo, hydroxyl, C₁₋₇ alkyl, C₁₋₇ alkoxy, —NH₂, di-(C₁₋₇alkyl)amino and C₁₋₇alkylamino. More preferred groups are morpholino, piperadinyl and homopiperadinyl which may be optionally substituted by one or more groups selected from hydroxyl, methyl, ethyl, —CO₂Me, —CO₂Et, —CH₂OH, —CH₂Ome, —CH₂NMe₂, —CONH₂, —CONHMe, —CONMe₂, phenyl, pyrrolidinyl, morpholino and piperadinyl.

In a further embodiment of the invention R² is selected from

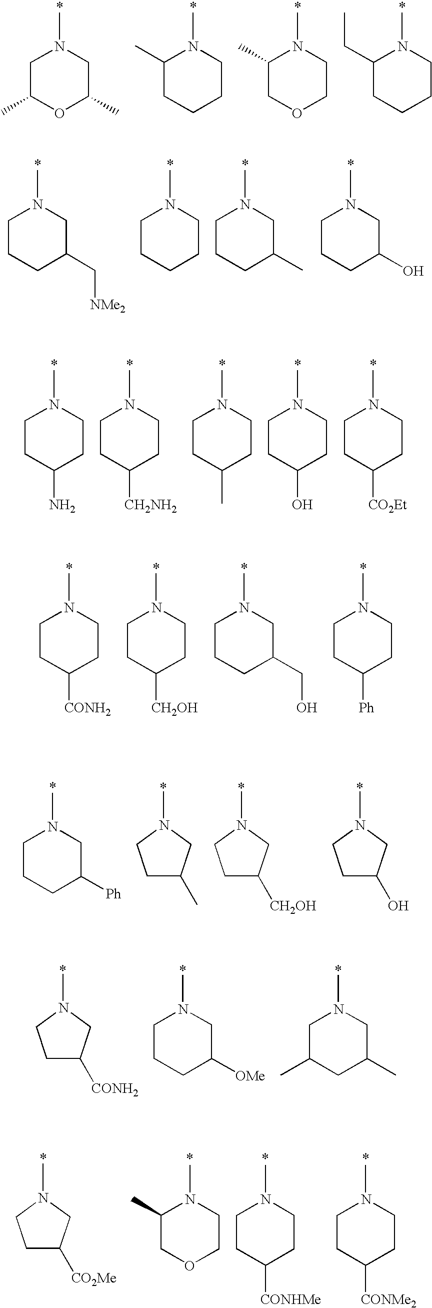

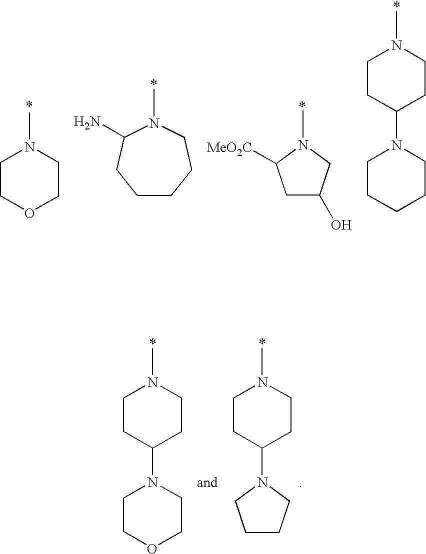

In a further embodiment of the invention R² is selected from

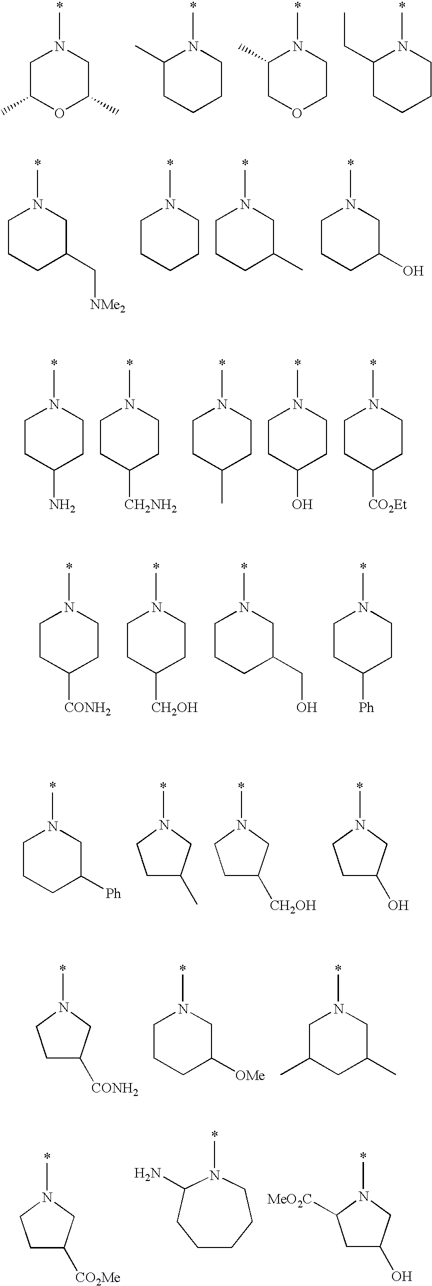

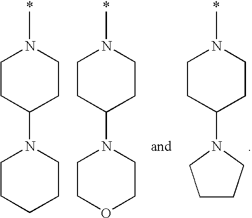

In a further embodiment of the invention R² is selected from

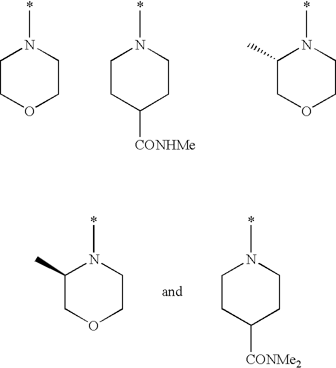

In an embodiment of the invention, there is provided a subset of compounds of formula (I), and pharmaceutically acceptable salts thereof, in which:
only one of X⁵, X⁶ and X⁸ is N;
R⁷ is selected from an optionally substituted C₅₋₂₀ aryl group, OR^{O1}, NR^{N1}R^{N2}, NR^{N7a}C(═O)R^{C1} and NR^{N7b}SO₂R^{S2a};
R^{N3} and R^{N4} together with the nitrogen to which they are bound form a heterocyclic ring containing between 5 and 7 ring atoms, which may optionally be substituted; and
R² is selected from NR^{N5}R^{N6}, an optionally substituted C₅₋₂₀ heteroaryl group, and an optionally substituted C₅₋₂₀ aryl group.

In another embodiment, there is provided a subset of compounds of formula (I), and pharmaceutically acceptable salts thereof, in which:
only one of X⁵, X⁶ and X⁸ is N;
R⁷ is an optionally substituted C₅₋₆ aryl group, wherein the optional substituents are selected from cyano, halo, hydroxyl, and C₁₋₇ alkyl and C₁₋₇ alkoxy (wherein the alkyl groups may be optionally substituted by one or more groups selected from halo, hydroxyl, C₁₋₇ alkoxy, amino and C₅₋₆ aryl);
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is selected from NR^{N5}R^{N6}, an optionally substituted C₅₋₆ heteroaryl group, and an optionally substituted C₆ aryl group.

In another embodiment, there is provided a subset of compounds of formula (I), and pharmaceutically acceptable salts thereof, in which:
only one of X⁵, X⁶ and X⁸ is N;
R⁷ is an optionally substituted C₅₋₆ aryl group, wherein the optional substituents are selected from cyano, halo, hydroxyl, and C₁₋₇ alkyl and C₁₋₇ alkoxy (wherein the alkyl groups may be optionally substituted by one or more groups selected from halo, hydroxyl, C₁₋₇ alkoxy, amino and C₅₋₆ aryl);
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form a heterocyclic ring containing between 5 to 7 ring atoms which may be optionally be substituted.

In a further embodiment, there is provided a subset of compounds of formula (I), and pharmaceutically acceptable salts thereof, in which:
only one of X⁵, X⁶ and X⁸ is N;
R⁷ is an optionally substituted C₅₋₆ aryl group, wherein the optional substituents are selected from cyano, halo, hydroxyl, and C₁₋₇ alkyl and C₁₋₇ alkoxy (wherein the alkyl groups may be optionally substituted by one or more groups selected from halo, hydroxyl, C₁₋₇ alkoxy, amino and C₅₋₆ aryl);
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form an optionally substituted morpholino, thiomorpholino, piperidinyl, piperazinyl (preferably N-substituted), homopiperazinyl (preferably N-substituted) or pyrrolidinyl group.

In a further embodiment, there is provided a subset of compounds of formula (I), and pharmaceutically acceptable salts thereof, in which:
X⁸ is N;
R⁷ is a thiophenyl group or a phenyl group optionally substituted by one or more groups selected from chloro, hydroxyl, methyl, methoxy, ethoxy, i-propoxy, benzyloxy and hydroxymethyl;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form an optionally substituted morpholino, thiomorpholino, piperidinyl, piperazinyl (preferably N-substituted), homopiperazinyl (preferably N-substituted) or pyrrolidinyl group.

In a further embodiment, there is provided a subset of compounds of formula (I), and pharmaceutically acceptable salts thereof, in which:
X⁸ is N;
R⁷ is a thiophenyl group or a phenyl group optionally substituted by one or more groups selected from chloro, hydroxyl, methyl, methoxy, ethoxy, i-propoxy, benzyloxy and hydroxymethyl;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form a morpholino, thiomorpholino, piperidinyl, piperazinyl (preferably N-substituted), homopiperazinyl (preferably N-substituted) or pyrrolidinyl group optionally substituted on carbon with one or more C₁₋₄alkyl groups.

In a further embodiment, there is provided a subset of compounds of formula (I), and pharmaceutically acceptable salts thereof, in which:
X⁸ is N;
R⁷ is a thiophenyl group or a phenyl group optionally substituted by one or more groups selected from chloro, hydroxyl, methyl, methoxy, ethoxy, i-propoxy, benzyloxy and hydroxymethyl;
R^{N3} and R^{N4} together with the nitrogen to which they are bound preferably form a morpholino or thiomorpholino; and
R² is NR^{N5}R^{N6} where R^{N5} and R^{N6} together with the nitrogen to which they are bound form a morpholino, thiomorpholino, piperidinyl, piperazinyl (preferably N-substituted), homopiperazinyl (preferably N-substituted) or pyrrolidinyl group optionally substituted on carbon with one or more C₁₋₄alkyl groups.

In a further embodiment, there is provided a subset of compounds of formula (I), and pharmaceutically acceptable salts thereof, in which:
X⁸ is N;
R⁷ is a 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group;
R^{N3} and R^{N4} together with the nitrogen to which they are bound preferably form a morpholino or thiomorpholino; and
R² is NR^{N5}R^{N6} where R^{N5} and R^{N6} together with the nitrogen to which they are bound form a

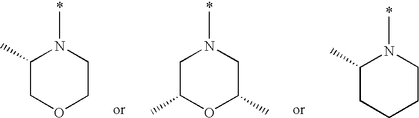

group.

In a further embodiment, there is provided a subset of compounds of formula (I), and pharmaceutically acceptable salts thereof, in which:
X⁸ is N;
R⁷ is a 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group;
R^{N3} and R^{N4} together with the nitrogen to which they are bound preferably form an unsubstituted morpholino; and
R² is NR^{N5}R^{N6} where R^{N5} and R^{N6} together with the nitrogen to which they are bound form a

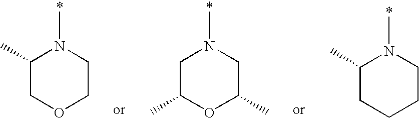

group.

In an embodiment of the invention, there is provided a subset of compounds of formula I(A), and pharmaceutically acceptable salts thereof, in which:
only one of X⁵, X⁶ and X⁸ is N;
R^{N10} is selected from C(═S)R^{C3}, an optionally substituted C₅₋₂₀ heteroaryl group, an optionally substituted C₅₋₂₀ aryl group, and an optionally substituted C₁₋₁₀ alkyl group where R^{C3} is as previously defined;
R^{O3} is an optionally substituted C₁₋₆ alkyl group;
R^{N3} and R^{N4} together with the nitrogen to which they are bound form a heterocyclic ring containing between 5 and 7 ring atoms, which may optionally be substituted; and
R² is selected from NR^{N5}R^{N6}, an optionally substituted C₅₋₂₀ heteroaryl group, and an optionally substituted C₅₋₂₀ aryl group.

In another embodiment, there is provided a subset of compounds of formula I(A), and pharmaceutically acceptable salts thereof, in which:
only one of X⁵, X⁶ and X⁸ is N;
R^{N10} is C(═S)R^{C3}, an optionally substituted C₅₋₆ heteroaryl group, an optionally substituted C₅₋₆ aryl group or an optionally substituted C₁₋₁₀ alkyl group where R^{C3} is NR^{N11}R^{N12} and where R^{N11} and R^{N12} together with the nitrogen to which they are bound form a heterocyclic ring containing between 3 and 8 ring atoms;
R^{O3} is an unsubstituted C₁₋₃ alkyl group;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is selected from NR^{N5}R^{N6}, an optionally substituted C₅₋₆ heteroaryl group, and an optionally substituted C₆ aryl group.

In another embodiment, there is provided a subset of compounds of formula I(A), and pharmaceutically acceptable salts thereof, in which:
only one of X⁵, X⁶ and X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₇alkoxycarbonylamino;
R^{O3} is a methyl group;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form a heterocyclic ring containing between 5 to 7 ring atoms which may be optionally be substituted.

In a further embodiment, there is provided a subset of compounds of formula I(A), and pharmaceutically acceptable salts thereof, in which:
only one of X⁵, X⁶ and X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₇alkoxycarbonylamino;
R^{O3} is a methyl group;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form an optionally substituted morpholino, thiomorpholino, piperidinyl, piperazinyl (preferably N-substituted), homopiperazinyl (preferably N-substituted) or pyrrolidinyl group.

In a further embodiment, there is provided a subset of compounds of formula I(A), and pharmaceutically acceptable salts thereof, in which:
X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₁₇alkoxycarbonylamino;
R^{O3} is a methyl group;
R^{N3} and R^{N4} together with the nitrogen to which they are bound preferably form an optionally substituted morpholino, thiomorpholino, piperidinyl, piperazinyl (preferably N-substituted), homopiperazinyl (preferably N-substituted) or pyrrolidinyl group; and
R² is NR^{N5}R^{N6} where R^{N5} and R^{N6} together with the nitrogen to which they are bound form an optionally substituted morpholino, thiomorpholino, piperidinyl, piperazinyl (preferably N-substituted), homopiperazinyl (preferably N-substituted) or pyrrolidinyl group.

In a further embodiment, there is provided a subset of compounds of formula I(A), and pharmaceutically acceptable salts thereof, in which:
X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₁₇alkoxycarbonylamino;
R^{O3} is a methyl group;
R^{N3} and R^{N4} together with the nitrogen to which they are bound preferably form a morpholino or thiomorpholino group; and
R² is NR^{N5}R^{N6} where R^{N5} and R^{N6} together with the nitrogen to which they are bound form morpholino, thiomorpholino, piperidinyl, piperazinyl (preferably N-substituted), homopiperazinyl (preferably N-substituted) or pyrrolidinyl group optionally substituted on carbon by one or more C₁₋₄alkyl groups.

In a further embodiment, there is provided a subset of compounds of formula I(A), and pharmaceutically acceptable salts thereof, in which:
X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₁₇alkoxycarbonylamino;
R^{O3} is a methyl group;
R^{N3} and R^{N4} together with the nitrogen to which they are bound preferably form a morpholino or thiomorpholino group; and
R² is NR^{N5}R^{N6} where R^{N5} and R^{N6} together with the nitrogen to which they are bound form a

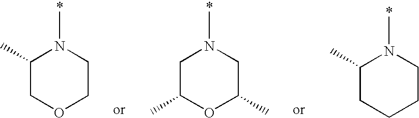

group.

In a further embodiment, there is provided a subset of compounds of formula I(A), and pharmaceutically acceptable salts thereof, in which:
X⁸ is N;
R^{N10} is a C(═S)NR^{N11}R^{N12} group where R^{N11} and R^{N12} together with the nitrogen to which they are bound form a heterocyclic ring containing between 3 and 8 ring atoms, or a pyrazole group optionally substituted with one or more groups selected from halo, hydroxyl, C₁₋₇ alkyl and C₁₋₇ alkoxy, or a phenyl group optionally substituted with one or more groups selected from halo, hydroxyl, C₁₋₇ alkyl and C₁₋₇ alkoxy, or a C₁₋₆ alkyl group optionally substituted with one or more groups selected from halo, hydroxyl, C₁₋₇ alkyl, ether, for example C₁₋₇ alkoxy, thioether, for example C₁₋₇ alkylthio, C₅₋₂₀ aryl, C₃₋₂₀ heterocyclyl, C₅₋₂₀ heteroaryl, cyano, ester, for example —C(═O)OR where R is C₁₋₇alkyl, and amino, for example C₁₋₇alkylamino, di-C₁₋₇alkylamino and C₁₋₇alkoxycarbonylamino;
R^{O3} is a methyl group;
R^{N3} and R^{N4} together with the nitrogen to which they are bound preferably form an unsubstituted morpholino group; and
R² is NR^{N5}R^{N6} where R^{N5} and R^{N6} together with the nitrogen to which they are bound form a

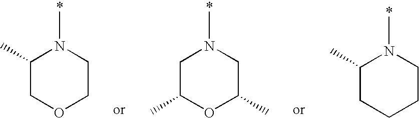

group.

In an embodiment of the invention, there is provided a subset of compounds of Formula I(B) or I(B)i, and pharmaceutically acceptable salts thereof, in which:
only one of X⁵, X⁶ and X⁸ is N;
R⁷ is selected from an optionally substituted C₅₋₂₀ aryl group, an optionally substituted 5- to 20-membered heteroaryl group, OR^{O1}, NR^{N1}R^{N2}, NR^{N7a}C(═O)R^{C1} and NR^{N7b}SO₂R^{S2a}; and
R² is selected from OR^{O2}, NR^{N5}R^{N6}, an optionally substituted C₅₋₂₀ heteroaryl group, and an optionally substituted C₅₋₂₀ aryl group.

In another embodiment, there is provided a subset of compounds of Formula I(B), or I(B)i, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is selected from OR^{O2}, NR^{N5}R^{N6}, an optionally substituted C₅₋₆ heteroaryl group, and an optionally substituted C₆ aryl group.

In another embodiment, there is provided a subset of compounds of Formula I(B), or I(B)i, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is NR^{N5}R^{N6} where R^{N5} and R^{N6} together with the nitrogen to which they are bound form a heterocyclic ring containing between 5 to 7 ring atoms which may be optionally be substituted, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).

In a further embodiment, there is provided a subset of compounds of Formula I(B), or I(B)i, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In an embodiment of the invention, there is provided a subset of compounds of Formula I(B), I(B)i or I(B)ii, and pharmaceutically acceptable salts thereof, in which:
only one of X⁵, X⁶ and X⁸ is N;
R⁷ is selected from an optionally substituted C₅₋₂₀ aryl group, an optionally substituted 5- to 20-membered heteraryl group, OR^{O1}, NR^{N1}R^{N2}, NR^{N7a}C(═O)R^{C1} and NR^{N7b}SO₂R^{S2a}; and
R² is selected from OR^{O2}, NR^{N5}R^{N6}, an optionally substituted C₅₋₂₀ heteroaryl group, and an optionally substituted C₅₋₂₀ aryl group.

In another embodiment, there is provided a subset of compounds of Formula I(B), I(B)i or I(B)ii, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is selected from OR^{O2}, NR^{N5}R^{N6}, an optionally substituted C₅₋₆ heteroaryl group, and an optionally substituted C₆ aryl group.

In another embodiment, there is provided a subset of compounds of Formula I(B), I(B)i or I(B)ii, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is NR^{N5}R^{N6} where R^{N5} and R^{N6} together with the nitrogen to which they are bound form a heterocyclic ring containing between 5 to 7 ring atoms which may be optionally be substituted, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).

In a further embodiment, there is provided a subset of compounds of Formula I(B), I(B)i or I(B)ii, and pharmaceutically acceptable salts thereof, in which:
only one of X⁵, X⁶ and X⁸ is N;
R⁷ is an optionally substituted C₅₋₆ aryl group or an optionally substituted 5 or 6 membered heteraryl group, wherein the optional substituents are selected from halo, hydroxyl, cyano, C₁₋₇ alkyl, C₁₋₇alkoxy, amino (for example —NH₂, C₅₋₆arylamino, C₁₋₇alkylamino, and di-(C₁₋₇alkyl)amino), and amido (for example —CONH₂, —CONHC₁₋₇alkyl, —CON(C₁₋₇alkyl)₂ and —CONHheterocycyl) and wherein the substitutent alkyl, alkoxy, or aryl groups may be further optionally substituted by one or more groups selected from halo, hydroxyl, C₁₋₇ alkyl, C₁₋₇ alkoxy, C₅₋₆aryl, C₅₋₆arylamino, di-(C₁₋₇alkyl)amino and C₁₋₇alkylamino; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from fluoro, hydroxyl, cyano, nitro, methyl, methoxy, —OCH₂CH₃, —NH₂, —NHSO₂CH₃, —CH₂NHSO₂CH₃, —OCHF₂, —CH₂OH, —CO₂H, —CONH₂, —CONHMe, —CONHEt, —CONHCH(CH₃)₂, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is NR^{N5}R^{N6} where R^{N5} and R^{N6} together with the nitrogen to which they are bound form a heterocyclic ring containing 5 to 7 ring atoms which may be optionally be substituted, wherein the optional substituents are selected from amino, cyano, halo, hydroxyl, ester, a C₃₋₇ cycloalkyl ring, a C₆carboaryl ring, a heterocyclic ring containing 5 to 7 ring atoms and C₁₋₇ saturated alkyl and C₁₋₇ saturated alkoxy (wherein the heterocyclic ring, the cycloalkyl ring, the carboaryl ring, the saturated alkyl and alkoxy groups may be optionally substituted by one or more groups selected from halo, hydroxyl, C₁₋₇ alkoxy, amino and C₅₋₆ aryl).

In a further embodiment, there is provided a subset of compounds of Formula I(B), I(B)i or I(B)ii, and pharmaceutically acceptable salts thereof, in which:
X⁵ and X⁶ are each CH; X⁸ is N;

R⁷ is an optionally substituted phenyl or pyridinyl group, wherein the optional substituents are preferably selected from fluoro, hydroxyl, cyano, nitro, methyl, methoxy, —OCH₂CH₃, —NH₂, —NHSO₂CH₃, —CH₂NHSO₂CH₃, —OCHF₂, —CH₂OH, —CO₂H, —CONH₂, —CONHMe, —CONHEt, —CONHCH(CH₃)₂, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In a further embodiment, there is provided a subset of compounds of Formula I(B), or I(B)i, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from —NH₂, fluoro, hydroxyl, cyano, nitro, methyl, methoxy, —CH₂OH, —CO₂H, —CONH₂, —CONHMe, —CONHEt, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from —NH₂, fluoro, hydroxyl, cyano, nitro, methyl, methoxy, —CH₂OH, —CO₂H, —CONH₂, —CONHMe, —CONHEt, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from fluoro, hydroxyl, cyano, nitro, methyl, methoxy, —OCH₂CH₃, —NH₂, —NHSO₂CH₃, —CH₂NHSO₂CH₃, —OCHF₂, —CH₂OH, —CO₂H, —CONH₂, —CONHMe, —CONHEt, —CONHCH(CH₃)₂, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and R² is a group selected from

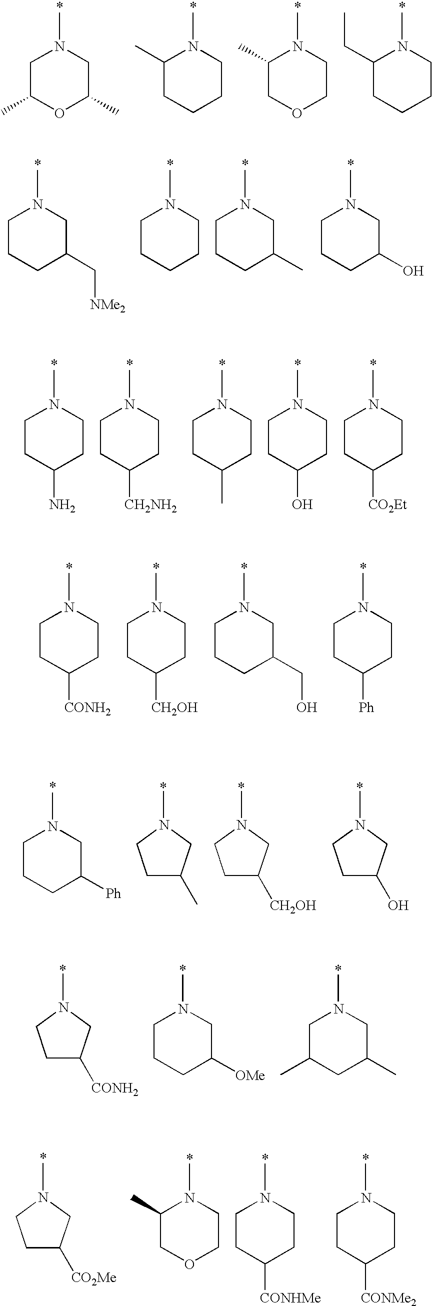

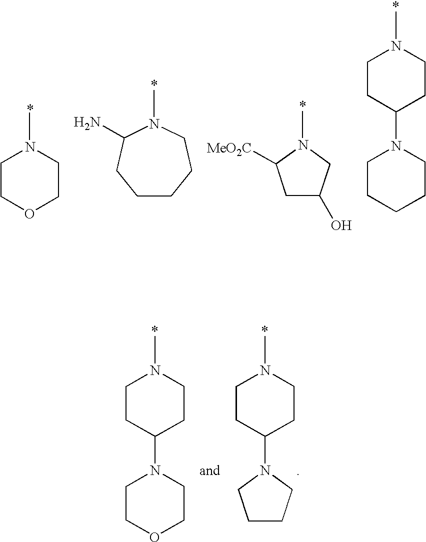

In a further embodiment, there is provided a subset of compounds of Formula I(B), or I(B)i, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from —NH₂, fluoro, hydroxyl, cyano, nitro, methyl, methoxy, —CH₂OH, —CO₂H, —CONH₂, —CONHMe, —CONHEt, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is a group selected from

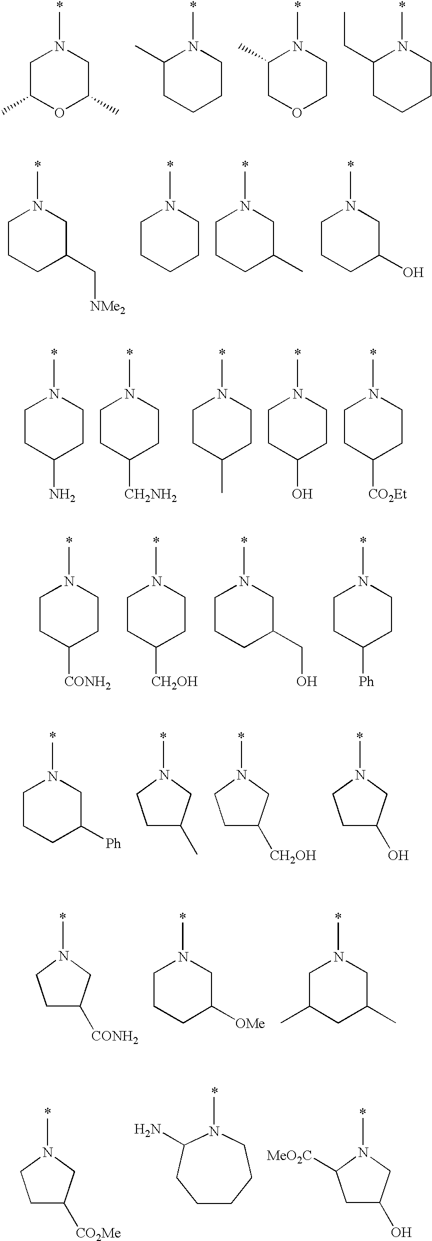

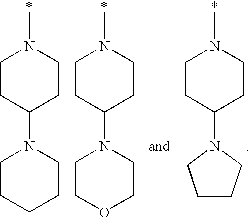

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is an optionally substituted phenyl or pyridinyl group, wherein the optional substituents are preferably selected from —NH₂, fluoro, hydroxyl, cyano, nitro, methyl, methoxy, —CH₂OH, —CO₂H, —CONH₂, —CONHMe, —CONHEt, —CONHCH₂CH₂F, —CONHCH₂CHF₂, —CONHCH₂CH₂OH, —CONMeEt, —CONMe₂, N-methylpiperazinylcarbonyl and 4-hydroxypiperidinylcarbonyl; and
R² is a group selected from

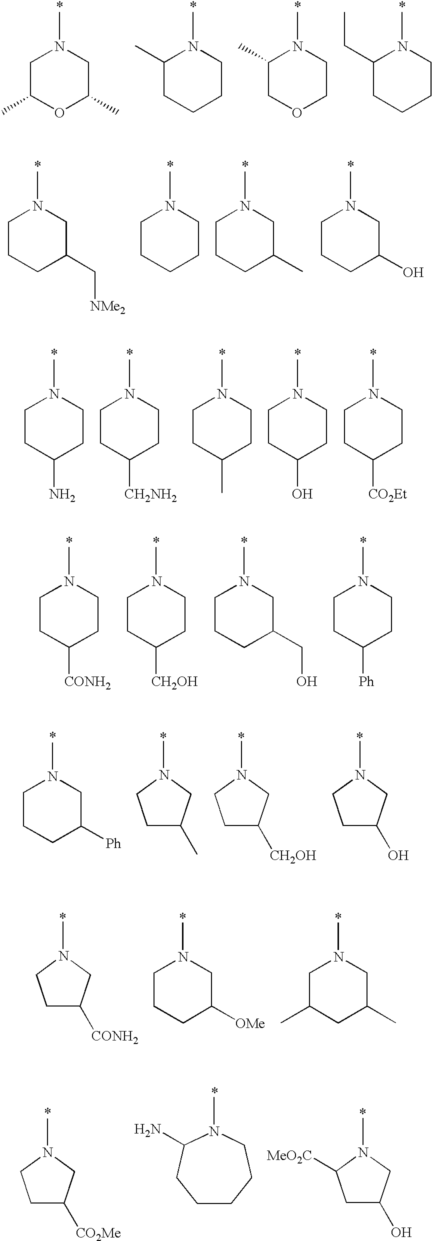

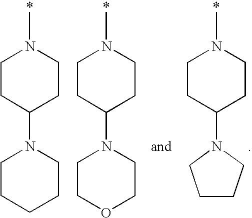

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is a 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group; and
R² is NR^{N5}R^{N6} where R^{N5} and R^{N6} together with the nitrogen to which they are bound form a

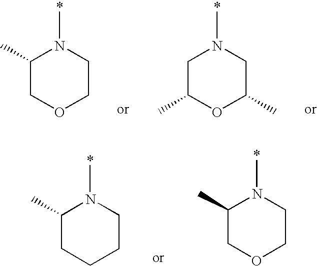

group.

In a further embodiment, there is provided a subset of compounds of Formula I(B), or I(B)i, and pharmaceutically acceptable salts thereof, in which:
X⁵ and X⁶ are each CH;
X⁸ is N;
R⁷ is a 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group; and
R² is NR^{N5}R^{N6} where R^{N5} and R^{N6} together with the nitrogen to which they are bound form a

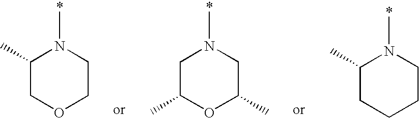

group.

In a further embodiment, there is provided a subset of compounds of Formula I(B), I(B)i or I(B)ii, and pharmaceutically acceptable salts thereof, in which:
X⁵ and X⁶ are each CH;
X⁸ is N;
R⁷ is a 4-chlorophenyl, 4-methylphenyl, 4-methoxyphenyl, 3-hydroxymethyl-4-methoxy-phenyl, 3,5-dimethoxy-4-hydroxyphenyl, 4-hydroxyphenyl, 3-hydroxyphenyl or a 3-hydroxymethylphenyl group; and
R² is NR^{N5}R^{N6} where R^{N5} and R^{N6} together with the nitrogen to which they are bound form a

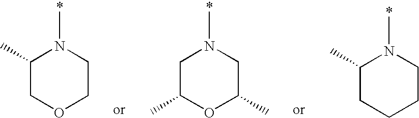

group.

In a further embodiment, there is provided a subset of compounds of Formula I(B), I(B)i or I(B)ii, and pharmaceutically acceptable salts thereof, in which:
X⁵ and X⁶ are each CH;
X⁸ is N;

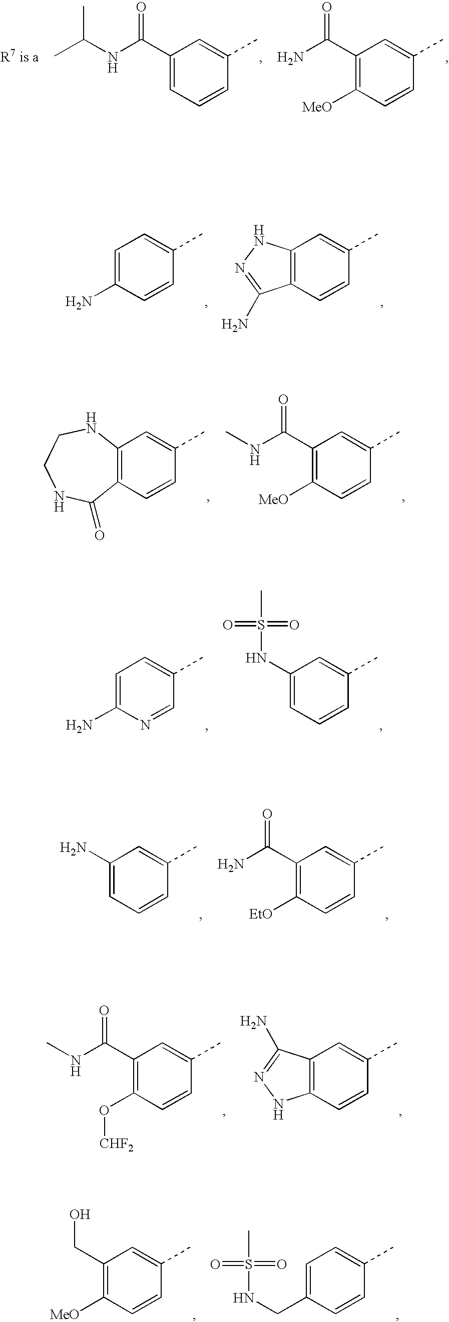

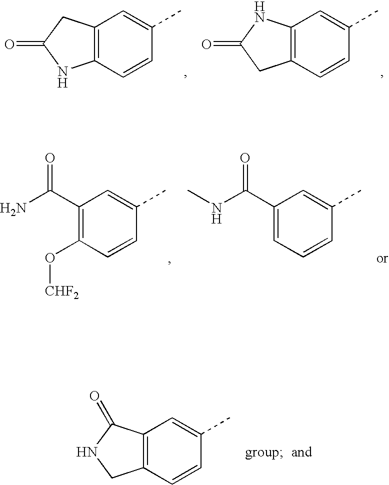

R² is NR^{N5}R^{N6} where R^{N5} and R^{N6} together with the nitrogen to which they are bound form a

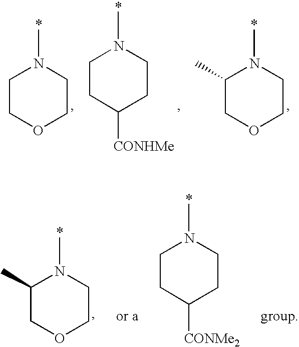

In an embodiment of the invention, there is provided a subset of compounds of Formula I(B) or I(B)i wherein the compound is a compound of formula (II) or (IIa), and pharmaceutically acceptable salts thereof,

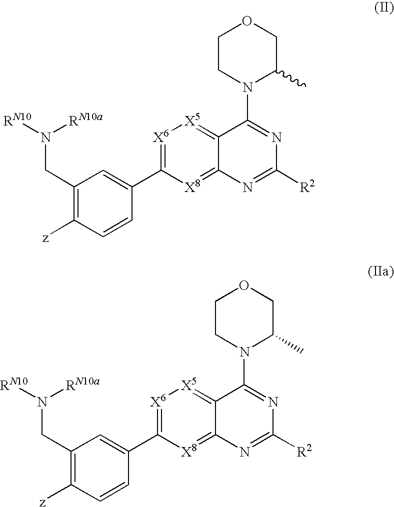

wherein:
only one of X⁵, X⁶ and X⁸ is N, and the others are CH;
Z is H, F or OR^{O3};
R^{N10} is selected from hydrogen, C(O)R^{C2}, an optionally substituted C₅₋₂₀ heteroaryl group, an optionally substituted C₅₋₂₀ aryl group, or an optionally substituted C₁₋₁₀ alkyl group where R^{C2} are selected from H, an optionally substituted C₅₋₂₀ aryl group, an optionally substituted C₅₋₂₀ heterocyclyl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heterocycyl group, an optionally substituted C₅₋₂₀ aryl group or R^{N11} and R^{N12} together with the nitrogen to which they are bound form a heterocyclic ring containing between 3 and 8 ring atoms;
R^{N10a} is selected from hydrogen or an optionally substituted C₁₋₁₀ alkyl group; or
R^{N10} and R^{N10a} together with the nitrogen to which they are bound form an optionally substituted heterocyclic ring containing between 3 and 8 ring atoms;
R^{O3} is an optionally substituted C₁₋₆ alkyl group; and
R² is selected from NR^{N5}R^{N6}, an optionally substituted C₅₋₂₀ heteroaryl group, and an optionally substituted C₅₋₂₀ aryl group.

In another embodiment, there is provided a subset of compounds of formula (II) or (IIa), and pharmaceutically acceptable salts thereof, in which:
only one of X⁵, X⁶ and X⁸ is N, and the others are CH;
Z is H, F or OR^{O3}
R^{N10} is R^{N10} is selected from hydrogen, C(O)R^{C2}, an optionally substituted C₅₋₆ heteroaryl group, an optionally substituted C₆ aryl group, or an optionally substituted C₁₋₁₀ alkyl group where R^{C2} are selected from CH₃ or CH₂OH where the optional substituents are selected from cyano, halo, hydroxyl, C₁₋₇alkyloxy, C₁₋₇alkylamino and di-C₁₋₇alkylamino;
R^{N10a} is selected from hydrogen or an optionally substituted C₁₋₁₀ alkyl group where the optional substituents are selected from cyano, halo, hydroxyl, C₁₋₇alkyloxy, C₁₋₇alkylamino and di-C₁₋₇alkylamino; or
R^{N10} and R^{N10a} together with the nitrogen to which they are bound form an optionally substituted heterocyclic ring containing between 3 and 8 ring atoms, where the optional substituents are selected from cyano, halo, hydroxyl, C₁₋₇alkyloxy, C₁₋₇alkylamino and di-C₁₋₇alkylamino;
R^{O3} is an unsubstituted C₁₋₃ alkyl group; and
R² is selected from NR^{N5}R^{N6}, an optionally substituted C₅₋₆ heteroaryl group, and an optionally substituted C₆ aryl group.

In another embodiment, there is provided a subset of compounds of formula (II) or (IIa), and pharmaceutically acceptable salts thereof, in which:
only one of X⁵, X⁶ and X⁸ is N, and the others are CH;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cyclopropyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form a heterocyclic ring containing between 5 to 7 ring atoms which may be optionally be substituted, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).

In a further embodiment, there is provided a subset of compounds of formula (II) or (IIa), and pharmaceutically acceptable salts thereof, in which:
only one of X⁵, X⁶ and X⁸ is N, and the others are CH;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.

In a further embodiment, there is provided a subset of compounds of formula (II) or (IIa), and pharmaceutically acceptable salts thereof, in which:
X⁵ and X⁶ are each CH;
X⁸ is N;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form an optionally substituted morpholino, thiomorpholino, piperidinyl, homopiperidinyl, piperazinyl (preferably N-substituted), homopiperazinyl (preferably N-substituted) or pyrrolidinyl group, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).

In a further embodiment, there is provided a subset of compounds of formula (II) or (IIa), and pharmaceutically acceptable salts thereof, in which:
X⁵ and X⁶ are each CH;
X⁸ is N;
Z is H, F or OR^{O3}
R^{N10} is a R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is a group selected from

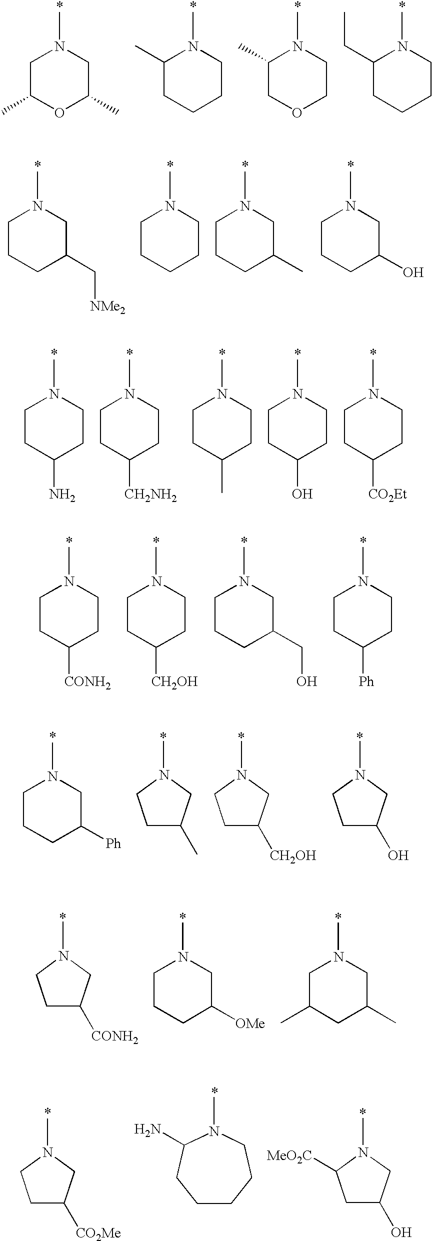

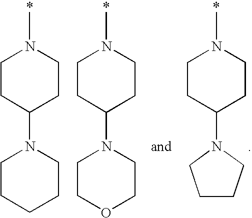

In an embodiment of the invention, there is provided a subset of compounds of Formula I(B), I(B)i or I(B)ii wherein the compound is a compound of formula (II), (IIa) or (IIb), and pharmaceutically acceptable salts thereof,

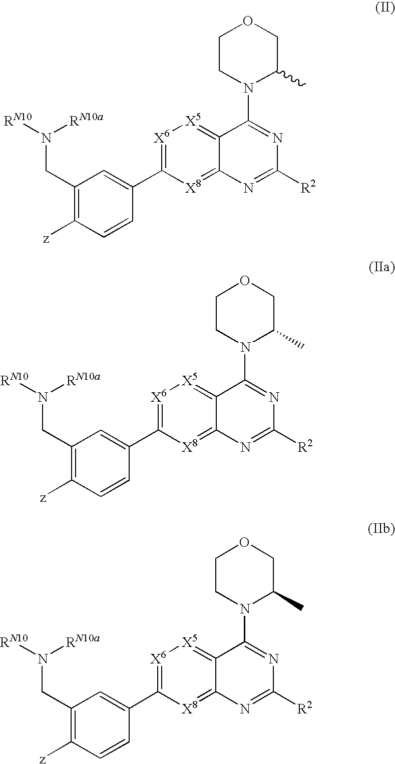

wherein:
only one of X⁵, X⁶ and X⁸ is N, and the others are CH;
Z is H, F or OR^{O3};
R^{N10} is selected from hydrogen, C(O)R^{C2}, an optionally substituted C₅₋₂₀ heteroaryl group, an optionally substituted C₅₋₂₀ aryl group, or an optionally substituted C₁₋₁₀ alkyl group where R^{C2} are selected from H, an optionally substituted C₅₋₂₀ aryl group, an optionally substituted C₅₋₂₀ heterocyclyl group, an optionally substituted C₁₋₇ alkyl group or NR^{N11}R^{N12}, where R^{N11} and R^{N12} are independently selected from H, an optionally substituted C₁₋₇ alkyl group, an optionally substituted C₅₋₂₀ heterocycyl group, an optionally substituted C₅₋₂₀ aryl group or R^{N11} and R^{N12} together with the nitrogen to which they are bound form a heterocyclic ring containing between 3 and 8 ring atoms;
R^{N10a} is selected from hydrogen or an optionally substituted C₁₋₁₀ alkyl group; or
R^{N10} and R^{N10a} together with the nitrogen to which they are bound form an optionally substituted heterocyclic ring containing between 3 and 8 ring atoms;
R^{O3} is an optionally substituted C₁₋₆ alkyl group; and
R² is selected from NR^{N5}R^{N6}, an optionally substituted C₅₋₂₀ heteroaryl group, and an optionally substituted C₅₋₂₀ aryl group.
In another embodiment, there is provided a subset of compounds of formula (II), (IIa) or (IIb), and pharmaceutically acceptable salts thereof, in which:
only one of X⁵, X⁶ and X⁸ is N, and the others are CH;
Z is H, F or OR^{O3}

R^{N10} is R^{N10} is selected from hydrogen, C(O)R^{C2}, an optionally substituted C₅₋₆ heteroaryl group, an optionally substituted C₆ aryl group, or an optionally substituted C₁₋₁₀ alkyl group where R^{C2} are selected from CH₃ or CH₂OH where the optional substituents are selected from cyano, halo, hydroxyl, C₁₋₇alkyloxy, C₁₋₇alkylamino and di-C₁₋₇alkylamino;
R^{N10a} is selected from hydrogen or an optionally substituted C₁₋₁₀ alkyl group where the optional substituents are selected from cyano, halo, hydroxyl, C₁₋₇alkyloxy, C₁₋₇alkylamino and di-C₁₋₇alkylamino; or
R^{N10} and R^{N10a} together with the nitrogen to which they are bound form an optionally substituted heterocyclic ring containing between 3 and 8 ring atoms, where the optional substituents are selected from cyano, halo, hydroxyl, C₁₋₇alkyloxy, C₁₋₇alkylamino and di-C₁₋₇alkylamino;
R^{O3} is an unsubstituted C₁₋₃ alkyl group; and

R² is selected from NR^{N5}R^{N6}, an optionally substituted C₅₋₆ heteroaryl group, and an optionally substituted C₆ aryl group.
In another embodiment, there is provided a subset of compounds of formula (II), (IIa) or (IIb), and pharmaceutically acceptable salts thereof, in which:
only one of X⁵, X⁶ and X⁸ is N, and the others are CH;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form a heterocyclic ring containing between 5 to 7 ring atoms which may be optionally be substituted, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).
In a further embodiment, there is provided a subset of compounds of formula (II), (IIa) or (IIb), and pharmaceutically acceptable salts thereof, in which:
only one of X⁵, X⁶ and X⁸ is N, and the others are CH;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form an optionally substituted imidazolyl, morpholino, thiomorpholino, piperadinyl, homopiperadinyl, piperazinyl (preferably N-substituted), homopiperazinyl (preferably N-substituted) or pyrrolidinyl, wherein optional N-substituents on the piperazinyl and homopiperazinyl groups include C₁₋₇alkyl groups or esters, in particular, esters bearing a C₁₋₇ alkyl group as an ester substituent, e.g. —C(═O)OCH₃, —C(═O)OCH₂CH₃ and —C(═O)OC(CH₃)₃, and optional C-substituents for the imidazolyl, morpholino, thiomorpholino, piperadinyl, homopiperadinyl, piperazinyl, homopiperazinyl or pyrrolidinyl groups include phenyl, ester, amide and C₁₋₄ alkyl, preferably methyl, aminomethyl, hydroxymethyl or hydroxyethyl.
In a further embodiment, there is provided a subset of compounds of formula (II), (IIa) or (IIb), and pharmaceutically acceptable salts thereof, in which:
X⁵ and X⁶ are each CH;
X⁸ is N;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form an optionally substituted morpholino, thiomorpholino, piperidinyl, homopiperidinyl, piperazinyl (preferably N-substituted), homopiperazinyl (preferably N-substituted) or pyrrolidinyl group, wherein the optional substituents are selected from cyano, halo, hydroxyl, and C₁₋₇ saturated alkyl and C₁₋₇ saturated alkoxy (wherein the saturated alkyl and alkoxy groups may be optionally substituted by one or more groups selected from halo, hydroxyl, C₁₋₇ alkoxy, amino and C₅₋₆ aryl).
In a further embodiment, there is provided a subset of compounds of formula (II), (IIa) or (IIb), and pharmaceutically acceptable salts thereof, in which:
X⁵ and X⁶ are each CH;
X⁸ is N;

Z is H, F or OR^{O3}
R^{N10} is a R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and

R² is a group selected from

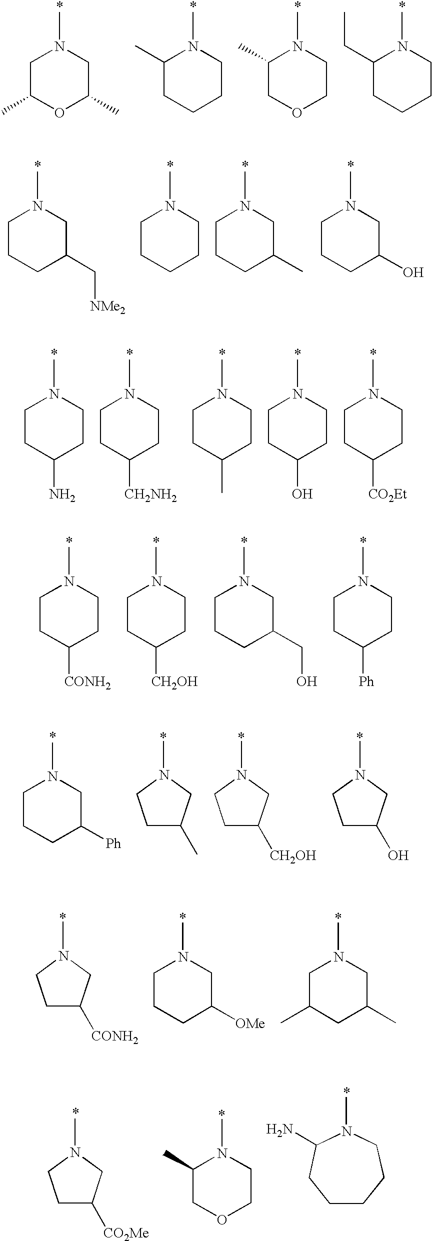

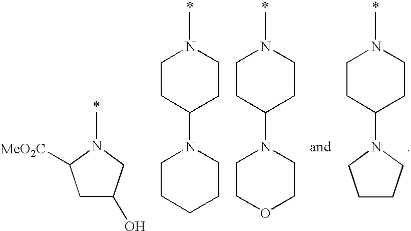

In a further embodiment, there is provided a subset of compounds of formula (II), (IIa) or (IIb), and pharmaceutically acceptable salts thereof, in which:
X⁵ and X⁶ are each CH;
X⁸ is N;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form a

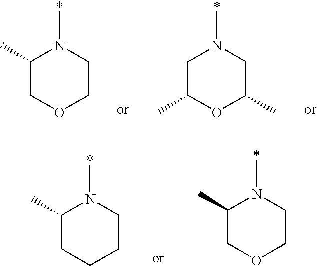

group.
In a further embodiment, there is provided a subset of compounds of formula (II), (IIa) or (IIb), and pharmaceutically acceptable salts thereof, in which:
X⁵ and X⁶ are each CH;
X⁸ is N;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and
R² is NR^{N5}R^{N6} where R^{N5} and R^{N6} together with the nitrogen to which they are bound form a

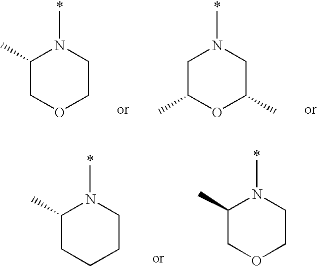

group.
In a further embodiment, there is provided a subset of compounds of formula (II), (IIa) or (IIb), and pharmaceutically acceptable salts thereof, in which:
X⁵ and X⁶ are each CH;
X⁸ is N;
Z is H, F or OR^{O3}
R^{N10} is selected from hydrogen, —C(O)CH₃, —C(O)CH₂OH, —CH₃, —CH₂CH₃, —CH₂CH₂OH, —CH(CH₃)₂, —CH₂CH₂OMe, —CH₂C(CH₃)₂, —CH₂CH₂C(CH₃)₂, —CH(CH₃)CH₂C(CH₃)₂, —CH₂CH₂CH₂N(CH₃)₂, cycloproyl, cyclopentyl, cyclohexyl, cycloheptyl, —CH₂cyclopropyl, methylcyclohexyl, cyanocyclohexyl, pyrazolyl, hydroxypyrrolidinyl, —CH₂imidazole;
R^{N10a} is hydrogen; or
R^{N10} and R^{N10a} together with the nitrogen to which they are bound form an optionally substituted heterocyclic ring containing between 5 or 6 ring atoms, where the optional substituents are selected from halo, hydroxyl, C₁₋₇alkyloxy;
R^{O3} is a methyl group; and

R² is NR^{N5}R^{N6} where R^{N5} and R^{N6} together with the nitrogen to which they are bound form a

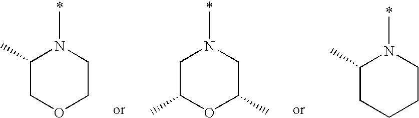

group.

### General Synthesis

Compounds of formula I can be represented by Formula 1:

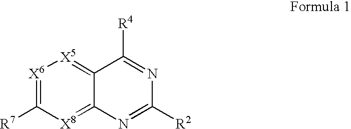

wherein R⁴ represents NR^{N3}R^{N4}.

Compounds of Formula 1 can be synthesised from compounds of Formula 2:

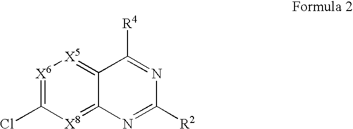

When R⁷ is NR^{N1}R^{N2}, this is by reaction with R⁷H. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent.

Therefore, according to a further aspect of the present invention there is provided a process for the preparation of a compound of formula I, from a compound of Formula 2:

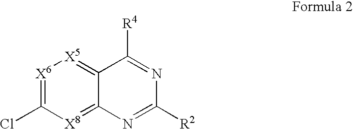

wherein:
R⁴ is NR^{N3}R^{N4} where R^{N3} and R^{N4}, together with the nitrogen to which they are bound, form a heterocyclic ring containing between 3 and 8 ring atoms;
R² is selected from H, halo, OR^{O2}, SR^{S2b}, NR^{N5}R^{N6}, an optionally substituted C₅₋₂₀ heteroaryl group, and an optionally substituted C₅₋₂₀ aryl group,
wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group, and R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms, comprising
(a) when R⁷ is NR^{N1}R^{N2}, reaction of the compound of formula 2 with R⁷H; or
(b) when R⁷ is an optionally substituted C₃₋₂₀ heterocyclyl group or C₅₋₂₀ aryl group, reaction of the compound of formula 2 with R⁷B(OAlk)₂, where each Alk is independently C₁₋₇ alkyl or together with the oxygen to which they are attached form a C₅₋₇ heterocyclyl group, or
(c) when R⁷ is an amide, urea or sulfonamide group, reaction of a compound of formula 2 with ammonia followed by reaction of the resulting primary amine with the appropriate acid chloride, isocyanate or sulfonyl chloride, or
(d) when R⁷ is OR^{O1} or SR^{S1}, by reaction of the compound of formula 1 in the presence of base in the appropriate alcohol or thiol solvent.

Compounds of formula I(A) can be synthesised by reaction a compound of Formula Ia:

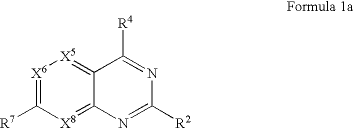

wherein R⁴ represents NR^{N3}R^{N4}, and

### R⁷ is

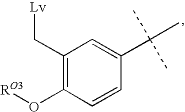

wherein Lv is a leaving group, such as a halogen, for example chlorine, or an OSO₂R group, where R is alkyl or aryl, such as methyl, by reaction with R^{N10}NH₂.

Compounds of Formula 1a can be synthesised by reaction of a compound of Formula 1b

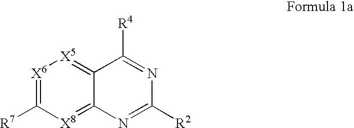

wherein R⁴ represents NR^{N3}R^{N4}, and

### R⁷ is

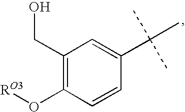

with an alkyl or aryl sulphonyl chloride in the presence of a base.

Compounds of Formula 1b can be synthesised by reacting a compound of Formula 2:

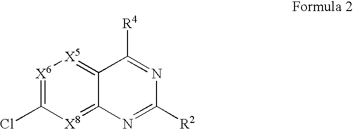

with R⁷B(OAlk)₂, where each Alk is independently C₁₋₇ alkyl or together with the oxygen to which they are attached form a C₅₋₇ heterocyclyl group.

Compounds of Formula 2 can be synthesised from compounds of Formula 3:

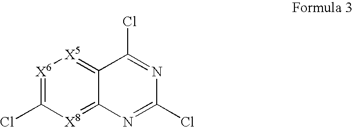

by reaction with HR⁴ (HNR^{N3}R^{N4}) followed by reaction with HR².

Compounds of Formula 3 can be synthesised from compounds of Formula 4:

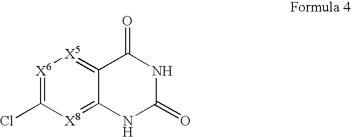

by treatment with POCl₃ and N,N-diisopropylamine, for example.

Compounds of Formula 4 can be synthesised from compounds of Formula 5:

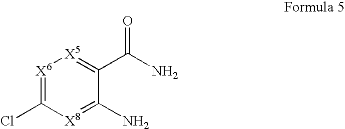

by treatment with oxalyl chloride, for example.

Compounds of Formula 5 can be synthesised from compounds of Formula 6, for example by reaction with liquid ammonia followed by reaction with thionyl chloride and ammonia gas:

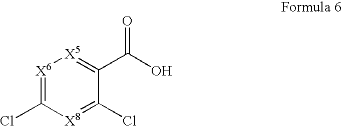

Alternatively, Compounds of Formula 1 can be synthesised from compounds of Formula 2A:

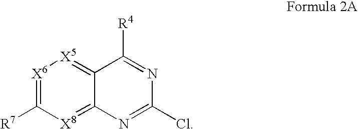

When R² is NR^{N5}R^{N6}, this is by reaction with R²H. When R² is an optionally substituted C₃₋₂₀ heterocyclyl group or C₅₋₂₀ aryl group, this is by reaction with R²B(OAlk)₂, where each Alk is independently C₁₋₇ alkyl or together with the oxygen to which they are attached form a C₅₋₇ heterocyclyl group. When R² is OR^{O2} or SR^{S2b}, this is by reaction with potassium carbonate in the appropriate alcohol or thiol solvent.

Therefore, according to a further aspect of the present invention there is provided a process for the preparation of a compound of formula 1 from a compound of formula 2A:

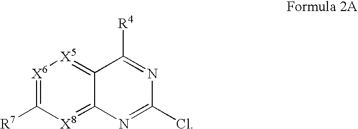

wherein
R⁴ is NR^{N3}R^{N4} where R^{N3} and R^{N4}, together with the nitrogen to which they are bound, form a heterocyclic ring containing between 3 and 8 ring atoms; and
R⁷ is selected from halo, OR^{O1}, SR^{S1}, NR^{N1}R^{N2}, NR^{N7a}C(═O)R^{C1}, NR^{N7b}SO₂R^{S2a}, an optionally substituted C₅₋₂₀ heteroaryl group, or an optionally substituted C₅₋₂₀ aryl group,
where R^{O1} and R^{S1} are selected from H, an optionally substituted C₅₋₂₀ aryl group, an optionally substituted C₅₋₂₀ heteroaryl group, or an optionally substituted C₁₋₇ alkyl group; R^{N1} and R^{N2} are independently selected from H, an optionally substituted C₁₋₇ alkyl group, an optionally substituted C₅₋₂₀ heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted C₅₋₂₀ heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted C₅₋₂₀ heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted C₅₋₂₀heteroaryl group, or an optionally substituted C₁₋₇ alkyl group; and
R^{N7a} and R^{N7b} are selected from H and a C₁₋₄ alkyl group; comprising
(a) when R² is NR^{N5}R^{N6}, reacting a compound of formula 2A with R²H, or
(b) when R² is an optionally substituted C₃₋₂₀ heterocyclyl group or C₅₋₂₀ aryl group, by reacting a compound of formula 2A with R²B(OAlk)₂, where each Alk is independently C₁₋₇ alkyl or together with the oxygen to which they are attached form a C₅₋₇ heterocyclyl group, or
(c) when R² is OR^{O2} or SR^{S2b}, by reacting a compound of formula 2A in the presence of a base in the appropriate alcohol or thiol solvent.

Compounds of Formula 2A can be synthesised from compounds of Formula 3:

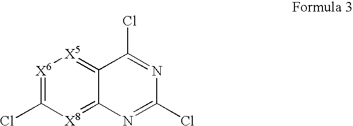

by reaction with HR⁴ (HNR^{N3}R^{N4}) followed by reaction with HR⁷ or HR⁷ equivalent. For example, when R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of formula I(B) can be represented by Formula 1.1:

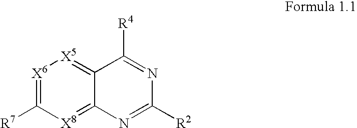

wherein R⁴ represents

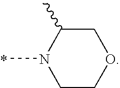

Compounds of Formula 1.1 can be synthesised from compounds of Formula 2.1:

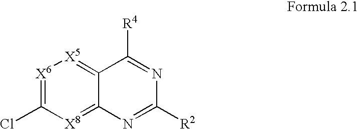

wherein R⁴ represents

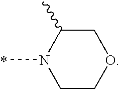

When R⁷ is NR^{N1}R^{N2}, this is by reaction with R⁷H. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of Formula 2.1 can be synthesised from compounds of Formula 3:

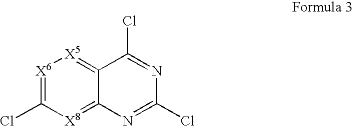

by reaction with HR⁴ (eg.

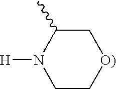

followed by reaction with HR².

Alternatively compounds of Formula 1 and Formula 1.1 can be synthesised from compounds of Formula 7:

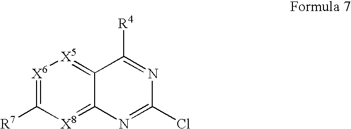

by reaction with HR².

Compounds of Formula 7 can be synthesised from compounds of Formula 8:

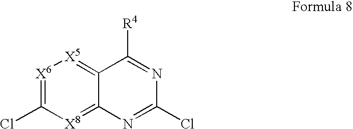

When R⁷ is NR^{N1}R^{N2}, this is by reaction with R⁷H. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of Formula 8 can be synthesised from compounds of Formula 3:

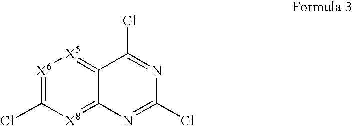

by reaction with HR⁴ (eg.

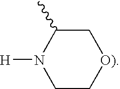

When R⁷ is

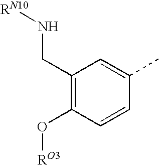

the Compound of Formula II can be prepared by reaction a compound of Formula 1.2:

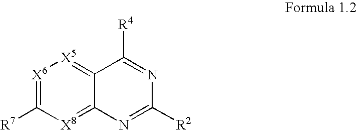

wherein R⁴ represents

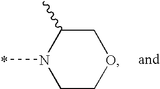

### R⁷ is

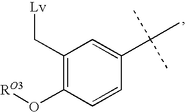

wherein Lv is a leaving group, such as a halogen, for example chlorine, or a OSO₂ group, where R is alkyl or aryl, such as methyl, by reaction with R^{N10}NH₂.
Compounds of Formula 1.2 can be synthesised by reaction of a compound of Formula 1.3

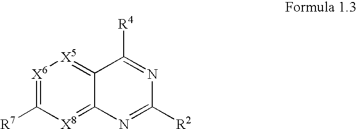

wherein R⁴ represents

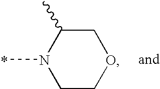

### R⁷ is

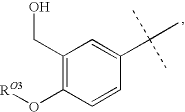

with an alkyl or aryl sulphonyl chloride in the presence of a base.

For Example:

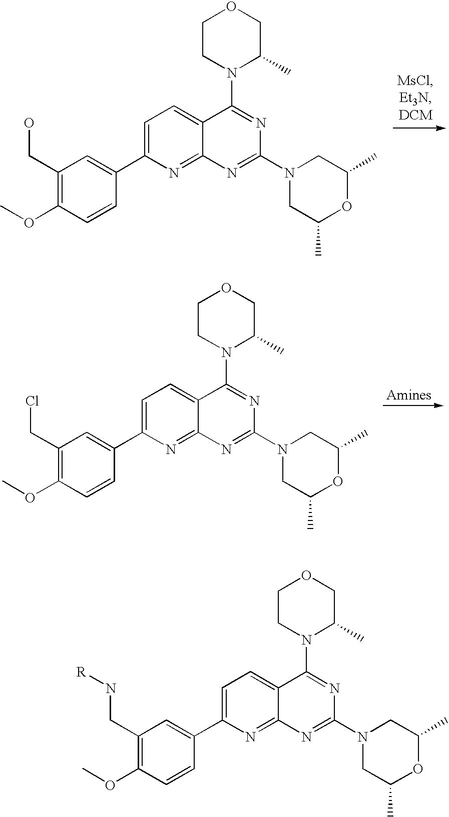

Compounds of Formula 1.3 can be prepared by reaction with R⁷B(OAlk)₂, where each Alk is independently C₁₋₇ alkyl or together with the oxygen to which they are attached form a C₅₋₇ heterocyclyl group.

In one embodiment the mTOR-selective inhibitor is selected from any one of
[5-[2,4-bis[(3S)-3-methylmorpholin-4-yl]pyrido[5,6-e]pyrimidin-7-yl]-2-methoxyphenyl]methanol;
[5-[2-[(2S,6R)-2,6-dimethylmorpholin-4-yl]-4-[(3S)-3-methylmorpholin-4-yl]pyrido[6,5-d]pyrimidin-7-yl]-2-methoxyphenyl]methanol;
[5-[2-(2-ethylpiperidin-1-yl)-4-[(3S)-3-methylmorpholin-4-yl]pyrido[6,5-d]pyrimidin-7-yl]-2-methoxyphenyl]methanol;
2,4-bis[(3S)-3-methylmorpholin-4-yl]-7-phenylpyrido[3,2-e]pyrimidine
4-[2,4-bis[(3S)-3-methylmorpholin-4-yl]pyrido[5,6-e]pyrimidin-7-yl]phenol;
2,4-bis[(3S)-3-methylmorpholin-4-yl]-7-(4-methylphenyl)pyrido[2,3-d]pyrimidine
[4-[2,4-bis[(3S)-3-methylmorpholin-4-yl]pyrido[5,6-e]pyrimidin-7-yl]phenyl]methanol;
3-[2,4-bis[(3S)-3-methylmorpholin-4-yl]pyrido[5,6-e]pyrimidin-7-yl]phenol
[3-[2,4-bis[(3S)-3-methylmorpholin-4-yl]pyrido[5,6-e]pyrimidin-7-yl]phenyl]methanol;
2-[(2S,6R)-2,6-dimethylmorpholin-4-yl]-4-[(3S)-3-methylmorpholin-4-yl]-7-phenylpyrido[2,3-d]pyrimidine;
2-[(2S,6R)-2,6-dimethylmorpholin-4-yl]-4-[(3S)-3-methylmorpholin-4-yl]-7-thiophen-3-ylpyrido[2,3-d]pyrimidine;
[2-methoxy-5-[4-[(3S)-3-methylmorpholin-4-yl]-2-morpholin-4-ylpyrido[5,6-e]pyrimidin-7-yl]phenyl]methanol;
7-(5-methoxypyridin-3-yl)-2,4-bis[(3S)-3-methylmorpholin-4-yl]pyrido[3,2-e]pyrimidine;
7-(6-methoxypyridin-3-yl)-2,4-bis[(3S)-3-methylmorpholin-4-yl]pyrido[3,2-e]pyrimidine;
7-(2-methoxypyridin-3-yl)-2,4-bis[(3S)-3-methylmorpholin-4-yl]pyrido[3,2-e]pyrimidine;
7-(2-fluoropyridin-3-yl)-2,4-bis[(3S)-3-methylmorpholin-4-yl]pyrido[3,2-e]pyrimidine;
2,4-bis[(3S)-3-methylmorpholin-4-yl]-7-(6-morpholin-4-ylpyridin-3-yl)pyrido[3,2-e]pyrimidine;
7-(2-methoxypyrimidin-5-yl)-2,4-bis[(3S)-3-methylmorpholin-4-yl]pyrido[3,2-e]pyrimidine;
2,4-bis[(3S)-3-methylmorpholin-4-yl]-7-(1-methylpyrazol-4-yl)pyrido[3,2-e]pyrimidine;
7-(2,4-dimethoxypyrimidin-5-yl)-2,4-bis[(3S)-3-methylmorpholin-4-yl]pyrido[3,2-e]pyrimidine;
5-[2,4-bis[(3S)-3-methylmorpholin-4-yl]pyrido[5,6-e]pyrimidin-7-yl]pyridin-2-amine;
7-(6-chloropyridin-2-yl)-2,4-bis[(3S)-3-methylmorpholin-4-yl]pyrido[3,2-e]pyrimidine;
7-(2-chloropyridin-4-yl)-2,4-bis[(3S)-3-methylmorpholin-4-yl]pyrido[3,2-e]pyrimidine;
7-(6-fluoro-2-methylpyridin-3-yl)-2,4-bis[(3S)-3-methylmorpholin-4-yl]pyrido[2,3-d]pyrimidine;
7-(5-chloropyridin-3-yl)-2,4-bis[(3S)-3-methylmorpholin-4-yl]pyrido[3,2-e]pyrimidine;
7-(3,5-dimethyl-1,2-oxazol-4-yl)-2,4-bis[(3S)-3-methylmorpholin-4-yl]pyrido[2,3-d]pyrimidine;
7-(4-fluorophenyl)-2,4-bis[(3S)-3-methylmorpholin-4-yl]pyrido[3,2-e]pyrimidine
5-[2,4-bis[(3S)-3-methylmorpholin-4-yl]pyrido[5,6-e]pyrimidin-7-yl]-2-fluorobenzamide;
5-[2,4-bis[(3S)-3-methylmorpholin-4-yl]pyrido[6,5-d]pyrimidin-7-yl]-2-fluoro-N-propylbenzamide;
5-[2,4-bis[(3S)-3-methylmorpholin-4-yl]pyrido[5,6-e]pyrimidin-7-yl]-2-fluorobenzoic acid;
5-[2,4-bis[(3S)-3-methylmorpholin-4-yl]pyrido[5,6-e]pyrimidin-7-yl]-2-fluoro-N-methylbenzamide;
5-[2,4-bis[(3S)-3-methylmorpholin-4-yl]pyrido[6,5-d]pyrimidin-7-yl]-N-ethyl-2-fluorobenzamide;
5-[2,4-bis[(3S)-3-methylmorpholin-4-yl]pyrido[5,6-e]pyrimidin-7-yl]-2-fluoro-N-(2-hydroxyethyl)benzamide;
7-(6-fluoropyridin-3-yl)-2,4-bis[(3S)-3-methylmorpholin-4-yl]pyrido[3,2-e]pyrimidine;
methyl 5-[2,4-bis[(3S)-3-methylmorpholin-4-yl]pyrido[5,6-e]pyrimidin-7-yl]pyridine-3-carboxylate;
7-(2-chloro-3-fluoropyridin-4-yl)-2,4-bis[(3S)-3-methylmorpholin-4-yl]pyrido[3,2-e]pyrimidine;
4-[2,4-bis[(3S)-3-methylmorpholin-4-yl]pyrido[5,6-e]pyrimidin-7-yl]-N-methylbenzamide;
3-[2,4-bis[(3S)-3-methylmorpholin-4-yl]pyrido[5,6-e]pyrimidin-7-yl]-N-propan-2-ylbenzamide;
2,4-bis[(3S)-3-methylmorpholin-4-yl]-7-(4-methylsulfonylphenyl)pyrido[3,2-e]pyrimidine;
2,4-bis[(3S)-3-methylmorpholin-4-yl]-7-(3-methylsulfonylphenyl)pyrido[3,2-e]pyrimidine;
4-[2,4-bis[(3S)-3-methylmorpholin-4-yl]pyrido[5,6-e]pyrimidin-7-yl]benzamide
3-[2,4-bis[(3S)-3-methylmorpholin-4-yl]pyrido[5,6-e]pyrimidin-7-yl]-N-methylbenzamide;
3-[2,4-bis[(3S)-3-methylmorpholin-4-yl]pyrido[5,6-e]pyrimidin-7-yl]-N-(2-hydroxyethyl)benzenesulfonamide;
2,4-bis[(3S)-3-methylmorpholin-4-yl]-7-pyridin-4-ylpyrido[3,2-e]pyrimidine;
3-[2,4-bis[(3S)-3-methylmorpholin-4-yl]pyrido[5,6-e]pyrimidin-7-yl]benzamide;
N-[3-[2,4-bis[(3S)-3-methylmorpholin-4-yl]pyrido[5,6-e]pyrimidin-7-yl]phenyl]methanesulfonamide;
7-(2-fluoropyridin-4-yl)-2,4-bis[(3S)-3-methylmorpholin-4-yl]pyrido[3,2-e]pyrimidine;
5-[2,4-bis[(3S)-3-methylmorpholin-4-yl]pyrido[5,6-e]pyrimidin-7-yl]-2-fluorobenzonitrile;
3-[2,4-bis[(3S)-3-methylmorpholin-4-yl]pyrido[5,6-e]pyrimidin-7-yl]benzonitrile;
3-[2,4-bis[(3S)-3-methylmorpholin-4-yl]pyrido[5,6-e]pyrimidin-7-yl]benzoic acid;
7-(2,6-difluoropyridin-4-yl)-2,4-bis[(3S)-3-methylmorpholin-4-yl]pyrido[3,2-e]pyrimidine;
4-[2,4-bis[(3S)-3-methylmorpholin-4-yl]pyrido[5,6-e]pyrimidin-7-yl]aniline methyl 3-[2,4-bis[(3S)-3-methylmorpholin-4-yl]pyrido[5,6-e]pyrimidin-7-yl]benzoate;
7-[3-(methoxymethyl)phenyl]-2,4-bis[(3S)-3-methylmorpholin-4-yl]pyrido[3,2-e]pyrimidine;
[5-[2,4-bis[(3S)-3-methylmorpholin-4-yl]pyrido[5,6-e]pyrimidin-7-yl]-2-fluorophenyl]methanol;
3-[2,4-bis[(3S)-3-methylmorpholin-4-yl]pyrido[5,6-e]pyrimidin-7-yl]benzenesulfonamide;
3-[2,4-bis[(3S)-3-methylmorpholin-4-yl]pyrido[5,6-e]pyrimidin-7-yl]-N-(2-methoxyethyl)benzamide;
[3-[2,4-bis[(3S)-3-methylmorpholin-4-yl]pyrido[5,6-e]pyrimidin-7-yl]phenyl]-(4-methylpiperazin-1-yl)methanone;
methyl 5-[2,4-bis[(3S)-3-methylmorpholin-4-yl]pyrido[5,6-e]pyrimidin-7-yl]-2-methoxybenzoate;
2,4-bis[(3S)-3-methylmorpholin-4-yl]-7-[3-(1H-tetrazol-5-yl)phenyl]pyrido[3,2-e]pyrimidine;
2,4-bis[(3S)-3-methylmorpholin-4-yl]-7-[4-(1H-tetrazol-5-yl)phenyl]pyrido[3,2-e]pyrimidine;
7-[4-fluoro-3-(1H-tetrazol-5-yl)phenyl]-2,4-bis[(3S)-3-methylmorpholin-4-yl]pyrido[3,2-e]pyrimidine;
N-[5-[2,4-bis[(3S)-3-methylmorpholin-4-yl]pyrido[5,6-e]pyrimidin-7-yl]-2-fluorophenyl]methanesulfonamide;
4-[2,4-bis[(3S)-3-methylmorpholin-4-yl]pyrido[5,6-e]pyrimidin-7-yl]benzonitrile;
7-(1H-indol-5-yl)-2,4-bis[(3S)-3-methylmorpholin-4-yl]pyrido[3,2-e]pyrimidine;
2,4-bis[(3S)-3-methylmorpholin-4-yl]-7-(4-nitrophenyl)pyrido[3,2-e]pyrimidine;
7-(4-methoxyphenyl)-2,4-bis[(3S)-3-methylmorpholin-4-yl]pyrido[3,2-e]pyrimidine;
7-(4-chlorophenyl)-2,4-bis[(3S)-3-methylmorpholin-4-yl]pyrido[3,2-e]pyrimidine;
2,4-bis[(3S)-3-methylmorpholin-4-yl]-7-(2-methylphenyl)pyrido[2,3-d]pyrimidine;
2,4-bis[(3S)-3-methylmorpholin-4-yl]-7-(3-methylphenyl)pyrido[2,3-d]pyrimidine;
7-(3-fluoro-4-methoxyphenyl)-2,4-bis[(3S)-3-methylmorpholin-4-yl]pyrido[3,2-e]pyrimidine;
7-(3-methoxyphenyl)-2,4-bis[(3S)-3-methylmorpholin-4-yl]pyrido[3,2-e]pyrimidine;
2,4-bis[(3S)-3-methylmorpholin-4-yl]-7-pyridin-3-ylpyrido[3,2-e]pyrimidine;
7-furan-3-yl-2,4-bis[(3S)-3-methylmorpholin-4-yl]pyrido[3,2-e]pyrimidine;
4-[2-[(2S,6R)-2,6-dimethylmorpholin-4-yl]-4-[(3S)-3-methylmorpholin-4-yl]pyrido[6,5-d]pyrimidin-7-yl]benzonitrile;
[3-[2-[(2S,6R)-2,6-dimethylmorpholin-4-yl]-4-[(3S)-3-methylmorpholin-4-yl]pyrido[6,5-d]pyrimidin-7-yl]phenyl]methanol;
[4-[2-[(2S,6R)-2,6-dimethylmorpholin-4-yl]-4-[(3S)-3-methylmorpholin-4-yl]pyrido[6,5-d]pyrimidin-7-yl]phenyl]methanol;
3-[2-[(2S,6R)-2,6-dimethylmorpholin-4-yl]-4-[(3S)-3-methylmorpholin-4-yl]pyrido[6,5-d]pyrimidin-7-yl]phenol;
2-[(2R,6S)-2,6-dimethylmorpholin-4-yl]-4-[(3S)-3-methylmorpholin-4-yl]-7-(2-methylphenyl)pyrido[2,3-d]pyrimidine;
2-[(2R,6S)-2,6-dimethylmorpholin-4-yl]-4-[(3S)-3-methylmorpholin-4-yl]-7-(3-methylphenyl)pyrido[2,3-d]pyrimidine;
2-[(2S,6R)-2,6-dimethylmorpholin-4-yl]-7-(3-fluoro-4-methoxyphenyl)-4-[(3S)-3-methylmorpholin-4-yl]pyrido[2,3-d]pyrimidine;
2-[(2S,6R)-2,6-dimethylmorpholin-4-yl]-7-(3-methoxyphenyl)-4-[(3S)-3-methylmorpholin-4-yl]pyrido[2,3-d]pyrimidine;
2-[(2S,6R)-2,6-dimethylmorpholin-4-yl]-4-[(3S)-3-methylmorpholin-4-yl]-7-pyridin-4-ylpyrido[2,3-d]pyrimidine;
2-[(2S,6R)-2,6-dimethylmorpholin-4-yl]-7-furan-3-yl-4-[(3S)-3-methylmorpholin-4-yl]pyrido[2,3-d]pyrimidine;
4-[2-[(2S,6R)-2,6-dimethylmorpholin-4-yl]-4-[(3S)-3-methylmorpholin-4-yl]pyrido[6,5-d]pyrimidin-7-yl]phenol;
2-[(2S,6R)-2,6-dimethylmorpholin-4-yl]-7-(1H-indol-5-yl)-4-[(3S)-3-methylmorpholin-4-yl]pyrido[2,3-d]pyrimidine;
2-[(2S,6R)-2,6-dimethylmorpholin-4-yl]-4-[(3S)-3-methylmorpholin-4-yl]-7-(4-nitrophenyl)pyrido[2,3-d]pyrimidine;
2-[(2S,6R)-2,6-dimethylmorpholin-4-yl]-7-(4-methoxyphenyl)-4-[(3S)-3-methylmorpholin-4-yl]pyrido[2,3-d]pyrimidine;
5-[2,4-bis[(3S)-3-methylmorpholin-4-yl]pyrido[5,6-e]pyrimidin-7-yl]-2-hydroxybenzoic acid;
6-[2,4-bis[(3S)-3-methylmorpholin-4-yl]pyrido[5,6-e]pyrimidin-7-yl]-3H-quinazolin-4-one;
6-[2,4-bis[(3S)-3-methylmorpholin-4-yl]pyrido[5,6-e]pyrimidin-7-yl]-4H-1,4-benzoxazin-3-one;
5-[2,4-bis[(3S)-3-methylmorpholin-4-yl]pyrido[5,6-e]pyrimidin-7-yl]-2-methoxybenzonitrile;
5-[2,4-bis[(3S)-3-methylmorpholin-4-yl]pyrido[6,5-d]pyrimidin-7-yl]-2-ethoxybenzamide;
5-[2,4-bis[(3S)-3-methylmorpholin-4-yl]pyrido[6,5-d]pyrimidin-7-yl]-2-ethoxybenzonitrile;
7-(1H-indazol-5-yl)-2,4-bis[(3S)-3-methylmorpholin-4-yl]pyrido[3,2-e]pyrimidine;
7-[2,4-bis[(3S)-3-methylmorpholin-4-yl]pyrido[5,6-e]pyrimidin-7-yl]-2H-phthalazin-1-one;
5-[2,4-bis[(3S)-3-methylmorpholin-4-yl]pyrido[5,6-e]pyrimidin-7-yl]-2-propan-2-yloxybenzonitrile;
4-[2,4-bis[(3S)-3-methylmorpholin-4-yl]pyrido[5,6-e]pyrimidin-7-yl]-2-(hydroxymethyl)phenol;
5-[2,4-bis[(3S)-3-methylmorpholin-4-yl]pyrido[5,6-e]pyrimidin-7-yl]-1,3-dihydroindol-2-one;
4-[2,4-bis[(3S)-3-methylmorpholin-4-yl]pyrido[5,6-e]pyrimidin-7-yl]-2-fluoro-N-methylbenzamide;
5-[2,4-bis[(3S)-3-methylmorpholin-4-yl]pyrido[5,6-e]pyrimidin-7-yl]-2-propan-2-yloxybenzamide;
5-[2,4-bis[(3S)-3-methylmorpholin-4-yl]pyrido[5,6-e]pyrimidin-7-yl]-2-hydroxybenzamide;
5-[2,4-bis[(3S)-3-methylmorpholin-4-yl]pyrido[5,6-e]pyrimidin-7-yl]pyridine-2-carboxamide;
4-[2,4-bis[(3S)-3-methylmorpholin-4-yl]pyrido[5,6-e]pyrimidin-7-yl]pyridin-2-amine;
4-[2,4-bis[(3S)-3-methylmorpholin-4-yl]pyrido[5,6-e]pyrimidin-7-yl]-2-fluorobenzamide;
5-[2,4-bis[(3S)-3-methylmorpholin-4-yl]pyrido[5,6-e]pyrimidin-7-yl]-1H-pyridin-2-one;
5-[2,4-bis[(3S)-3-methylmorpholin-4-yl]pyrido[5,6-e]pyrimidin-7-yl]-N-methylpyridin-2-amine;
5-[2,4-bis[(3S)-3-methylmorpholin-4-yl]pyrido[5,6-e]pyrimidin-7-yl]-N,N-dimethylpyridin-2-amine;
8-[2,4-bis[(3S)-3-methylmorpholin-4-yl]pyrido[5,6-e]pyrimidin-7-yl]-1,2,3,4-tetrahydro-1,4-benzodiazepin-5-one;
7-[2,4-bis[(3S)-3-methylmorpholin-4-yl]pyrido[5,6-e]pyrimidin-7-yl]-1,2,3,4-tetrahydro-1,4-benzodiazepin-5-one;
5-[2,4-bis[(3S)-3-methylmorpholin-4-yl]pyrido[5,6-e]pyrimidin-7-yl]-2-(difluoromethoxy)benzamide;
5-[2,4-bis[(3S)-3-methylmorpholin-4-yl]pyrido[5,6-e]pyrimidin-7-yl]-2-(difluoromethoxy)-N-methylbenzamide;
4-[2,4-bis[(3S)-3-methylmorpholin-4-yl]pyrido[5,6-e]pyrimidin-7-yl]-2-methoxybenzamide;
4-[2,4-bis[(3S)-3-methylmorpholin-4-yl]pyrido[5,6-e]pyrimidin-7-yl]-2-methoxy-N-methylbenzamide;
2-methoxy-N-methyl-5-[4-[(3S)-3-methylmorpholin-4-yl]-2-morpholin-4-ylpyrido[5,6-e]pyrimidin-7-yl]benzamide;
6-[2,4-bis[(3S)-3-methylmorpholin-4-yl]pyrido[5,6-e]pyrimidin-7-yl]-1H-indazol-3-amine;
N-[4-[2,4-bis[(3S)-3-methylmorpholin-4-yl]pyrido[5,6-e]pyrimidin-7-yl]pyridin-2-yl]acetamide;
2-methoxy-5-[4-[(3S)-3-methylmorpholin-4-yl]-2-thiomorpholin-4-ylpyrido[5,6-e]pyrimidin-7-yl]benzamide;
2-methoxy-5-[4-[(3S)-3-methylmorpholin-4-yl]-2-(4-methylpiperazin-1-yl)pyrido[5,6-e]pyrimidin-7-yl]benzamide;
2-methoxy-5-[4-[(3S)-3-methylmorpholin-4-yl]-2-morpholin-4-ylpyrido[5,6-e]pyrimidin-7-yl]benzamide;
N-[3-[2,4-bis[(3S)-3-methylmorpholin-4-yl]-8-oxidopyrido[5,6-e]pyrimidin-8-ium-7-yl]phenyl]methanesulfonamide;
3-[2,4-bis[(3S)-3-methylmorpholin-4-yl]pyrido[5,6-e]pyrimidin-7-yl]aniline;
5-[2,4-bis[(3S)-3-methylmorpholin-4-yl]pyrido[5,6-e]pyrimidin-7-yl]-1H-indazol-3-amine;
6-[2,4-bis[(3S)-3-methylmorpholin-4-yl]pyrido[5,6-e]pyrimidin-7-yl]-2,3-dihydroisoindol-1-one;
N-[[4-[2,4-bis[(3S)-3-methylmorpholin-4-yl]pyrido[5,6-e]pyrimidin-7-yl]phenyl]methyl]methanesulfonamide;
4-[2,4-bis[(3S)-3-methylmorpholin-4-yl]pyrido[6,5-d]pyrimidin-7-yl]-N-ethyl-2-fluorobenzamide;
7-[2,4-bis[(3S)-3-methylmorpholin-4-yl]pyrido[5,6-e]pyrimidin-7-yl]-3,4-dihydro-1H-1,4-benzodiazepine-2,5-dione;
7-[2,4-bis[(3S)-3-methylmorpholin-4-yl]pyrido[5,6-e]pyrimidin-7-yl]-3H-quinazolin-4-one;
6-[2,4-bis[(3S)-3-methylmorpholin-4-yl]pyrido[5,6-e]pyrimidin-7-yl]-1,3-dihydroindol-2-one;
N-[4-[2,4-bis[(3S)-3-methylmorpholin-4-yl]pyrido[5,6-e]pyrimidin-7-yl]phenyl]methanesulfonamide;
5-[2,4-bis[(3S)-3-methylmorpholin-4-yl]pyrido[5,6-e]pyrimidin-7-yl]-2-methoxyaniline;
2,4-bis[(3S)-3-methylmorpholin-4-yl]-7-(1H-pyrrolo[3,2-e]pyridin-5-yl)pyrido[3,2-e]pyrimidine;
[5-[2,4-bis[(3R)-3-methylmorpholin-4-yl]pyrido[5,6-e]pyrimidin-7-yl]-2-methoxyphenyl]methanol;
5-[2,4-bis[(3S)-3-methylmorpholin-4-yl]pyrido[5,6-e]pyrimidin-7-yl]-2,3-dihydroisoindol-1-one;
N-[3-[4-[(3S)-3-methylmorpholin-4-yl]-2-morpholin-4-ylpyrido[5,6-e]pyrimidin-7-yl]phenyl]methanesulfonamide;
3-[4-[(3S)-3-methylmorpholin-4-yl]-2-(4-methylpiperazin-1-yl)pyrido[5,6-e]pyrimidin-7-yl]benzamide;
7-(4-chlorophenyl)-4-[(3S)-3-methylmorpholin-4-yl]-2-(4-methylpiperazin-1-yl)pyrido[3,2-e]pyrimidine;
3-[2-[4-(aminomethyl)piperidin-1-yl]-4-[(3S)-3-methylmorpholin-4-yl]pyrido[5,6-e]pyrimidin-7-yl]benzamide;
3-[2-[3-(hydroxymethyl)piperidin-1-yl]-4-[(3S)-3-methylmorpholin-4-yl]pyrido[5,6-e]pyrimidin-7-yl]benzamide;
5-[4-[(3S)-3-methylmorpholin-4-yl]-2-(4-pyridin-4-ylpiperidin-1-yl)pyrido[5,6-e]pyrimidin-7-yl]pyridin-2-amine;
3-[2-[2-(hydroxymethyl)piperidin-1-yl]-4-[(3S)-3-methylmorpholin-4-yl]pyrido[5,6-e]pyrimidin-7-yl]benzamide;
[1-[7-(6-aminopyridin-3-yl)-4-[(3S)-3-methylmorpholin-4-yl]pyrido[3,2-e]pyrimidin-2-yl]piperidin-2-yl]methanol;
3-[2-[4-(hydroxymethyl)piperidin-1-yl]-4-[(3S)-3-methylmorpholin-4-yl]pyrido[5,6-e]pyrimidin-7-yl]benzamide;
3-[2-(4-aminopiperidin-1-yl)-4-[(3S)-3-methylmorpholin-4-yl]pyrido[5,6-e]pyrimidin-7-yl]benzamide;
2-[1-[7-(6-aminopyridin-3-yl)-4-[(3S)-3-methylmorpholin-4-yl]pyrido[3,2-e]pyrimidin-2-yl]piperidin-4-yl]ethanol;
3-[2,4-bis[(3S)-3-methylmorpholin-4-yl]pyrido[5,6-e]pyrimidin-7-yl]-N-(1H-pyrazol-3-yl)benzamide;
[3-[2,4-bis[(3S)-3-methylmorpholin-4-yl]pyrido[5,6-e]pyrimidin-7-yl]phenyl]-piperazin-1-ylmethanone;
3-[2,4-bis[(3S)-3-methylmorpholin-4-yl]pyrido[5,6-e]pyrimidin-7-yl]-N,N-dimethylbenzamide;
[3-[2,4-bis[(3S)-3-methylmorpholin-4-yl]pyrido[5,6-e]pyrimidin-7-yl]phenyl]-[(3R)-3-hydroxypiperidin-1-yl]methanone;
3-[2,4-bis[(3S)-3-methylmorpholin-4-yl]pyrido[5,6-e]pyrimidin-7-yl]-N-(2-fluoroethyl)benzamide;
3-[2,4-bis[(3S)-3-methylmorpholin-4-yl]pyrido[5,6-e]pyrimidin-7-yl]-N-(2,2,2-trifluoroethyl)benzamide;
[3-[2,4-bis[(3S)-3-methylmorpholin-4-yl]pyrido[5,6-e]pyrimidin-7-yl]phenyl]-[(3R)-3-hydroxypyrrolidin-1-yl]methanone;
[3-[2,4-bis[(3S)-3-methylmorpholin-4-yl]pyrido[5,6-e]pyrimidin-7-yl]phenyl]-[(3S)-3-hydroxypyrrolidin-1-yl]methanone;
3-[2,4-bis[(3S)-3-methylmorpholin-4-yl]pyrido[5,6-e]pyrimidin-7-yl]-N-(oxan-4-yl)benzamide;
3-[2,4-bis[(3S)-3-methylmorpholin-4-yl]pyrido[5,6-e]pyrimidin-7-yl]-N-methyl-N-propan-2-ylbenzamide;
3-[2,4-bis[(3S)-3-methylmorpholin-4-yl]pyrido[5,6-e]pyrimidin-7-yl]-N-(2-methoxyethyl)-N-methylbenzamide;
3-[2,4-bis[(3S)-3-methylmorpholin-4-yl]pyrido[5,6-e]pyrimidin-7-yl]-N-(2,2-difluoroethyl)benzamide;
3-[2,4-bis[(3S)-3-methylmorpholin-4-yl]pyrido[6,5-d]pyrimidin-7-yl]-N-ethyl-N-methylbenzamide;
3-[2,4-bis[(3S)-3-methylmorpholin-4-yl]pyrido[6,5-d]pyrimidin-7-yl]-N,N-diethylbenzamide;
3-[2,4-bis[(3S)-3-methylmorpholin-4-yl]pyrido[5,6-e]pyrimidin-7-yl]-N-methoxy-N-methylbenzamide;
3-[2,4-bis[(3S)-3-methylmorpholin-4-yl]pyrido[5,6-e]pyrimidin-7-yl]-N-methyl-N-prop-2-enylbenzamide;
3-[2,4-bis[(3S)-3-methylmorpholin-4-yl]pyrido[5,6-e]pyrimidin-7-yl]-N-methyl-N-prop-2-ynylbenzamide;
3-[2,4-bis[(3S)-3-methylmorpholin-4-yl]pyrido[5,6-e]pyrimidin-7-yl]-N-tert-butyl-N-methylbenzamide;
[3-[2,4-bis[(3S)-3-methylmorpholin-4-yl]pyrido[6,5-d]pyrimidin-7-yl]phenyl]-(2-methylpyrrolidin-1-yl)methanone;
5-[2,4-bis[(3S)-3-methylmorpholin-4-yl]pyrido[5,6-e]pyrimidin-7-yl]-2-methoxy-N,N-dimethylbenzamide;
5-[2,4-bis[(3S)-3-methylmorpholin-4-yl]pyrido[6,5-d]pyrimidin-7-yl]-N-ethyl-2-methoxy-N-methylbenzamide;
5-[2,4-bis[(3S)-3-methylmorpholin-4-yl]pyrido[5,6-e]pyrimidin-7-yl]-2-fluoro-N,N-dimethylbenzamide;
5-[2,4-bis[(3S)-3-methylmorpholin-4-yl]pyrido[6,5-d]pyrimidin-7-yl]-N,N-diethyl-2-fluorobenzamide;
5-[2,4-bis[(3S)-3-methylmorpholin-4-yl]pyrido[5,6-e]pyrimidin-7-yl]-2-fluoro-N-methyl-N-propan-2-ylbenzamide;
5-[2,4-bis[(3S)-3-methylmorpholin-4-yl]pyrido[5,6-e]pyrimidin-7-yl]-2-fluoro-N-methoxy-N-methylbenzamide;
5-[2,4-bis[(3S)-3-methylmorpholin-4-yl]pyrido[5,6-e]pyrimidin-7-yl]-2-fluoro-N-methyl-N-prop-2-enylbenzamide;
5-[2,4-bis[(3S)-3-methylmorpholin-4-yl]pyrido[6,5-d]pyrimidin-7-yl]-N-ethyl-2-fluoro-N-methylbenzamide;
5-[2,4-bis[(3S)-3-methylmorpholin-4-yl]pyrido[5,6-e]pyrimidin-7-yl]-2-fluoro-N-methyl-N-prop-2-ynylbenzamide;
4-[2,4-bis[(3S)-3-methylmorpholin-4-yl]pyrido[5,6-e]pyrimidin-7-yl]-N,N-dimethylbenzamide;
5-[2,4-bis[(3S)-3-methylmorpholin-4-yl]pyrido[5,6-e]pyrimidin-7-yl]-2-methoxy-N-methylbenzamide;
1-[4-[2,4-bis[(3S)-3-methylmorpholin-4-yl]pyrido[5,6-e]pyrimidin-7-yl]phenyl]-N-methylmethanamine;
[4-[2,4-bis[(3S)-3-methylmorpholin-4-yl]pyrido[5,6-e]pyrimidin-7-yl]phenyl]methanamine;
(3R)-1-[[4-[2,4-bis[(3S)-3-methylmorpholin-4-yl]pyrido[5,6-e]pyrimidin-7-yl]phenyl]methyl]pyrrolidin-3-ol;
(3S)-1-[[4-[2,4-bis[(3S)-3-methylmorpholin-4-yl]pyrido[5,6-e]pyrimidin-7-yl]phenyl]methyl]pyrrolidin-3-ol;
N-[[4-[2,4-bis[(3S)-3-methylmorpholin-4-yl]pyrido[5,6-e]pyrimidin-7-yl]phenyl]methyl]oxan-4-amine;
2-[[4-[2,4-bis[(3S)-3-methylmorpholin-4-yl]pyrido[5,6-e]pyrimidin-7-yl]phenyl]methylamino]ethanol;
7-[3-(ethoxymethyl)phenyl]-2,4-bis[(3S)-3-methylmorpholin-4-yl]pyrido[2,3-d]pyrimidine;
2-[[3-[2,4-bis[(3S)-3-methylmorpholin-4-yl]pyrido[5,6-e]pyrimidin-7-yl]phenyl]methylamino]ethanol;
[3-[2,4-bis[(3S)-3-methylmorpholin-4-yl]pyrido[5,6-e]pyrimidin-7-yl]phenyl]methanamine;
N-[[3-[2,4-bis[(3S)-3-methylmorpholin-4-yl]pyrido[5,6-e]pyrimidin-7-yl]phenyl]methyl]cyclopropanamine;
N-[[3-[2,4-bis[(3S)-3-methylmorpholin-4-yl]pyrido[5,6-e]pyrimidin-7-yl]phenyl]methyl]propan-2-amine;
1-[3-[2,4-bis[(3S)-3-methylmorpholin-4-yl]pyrido[5,6-e]pyrimidin-7-yl]phenyl]-N-methylmethanamine;
N-[[3-[2,4-bis[(3S)-3-methylmorpholin-4-yl]pyrido[5,6-e]pyrimidin-7-yl]phenyl]methyl]oxan-4-amine;
1-[3-[2,4-bis[(3S)-3-methylmorpholin-4-yl]pyrido[5,6-e]pyrimidin-7-yl]phenyl]-N-(cyclopropylmethyl)methanamine;
N-[[3-[2,4-bis[(3S)-3-methylmorpholin-4-yl]pyrido[5,6-e]pyrimidin-7-yl]phenyl]methyl]-2-methoxyethanamine;
N-[[3-[2,4-bis[(3S)-3-methylmorpholin-4-yl]pyrido[5,6-e]pyrimidin-7-yl]phenyl]methyl]-1H-pyrazol-3-amine;
7-[3-(imidazol-1-ylmethyl)phenyl]-2,4-bis[(3S)-3-methylmorpholin-4-yl]pyrido[3,2-e]pyrimidine;
(3R)-1-[[3-[2,4-bis[(3S)-3-methylmorpholin-4-yl]pyrido[5,6-e]pyrimidin-7-yl]phenyl]methyl]pyrrolidin-3-ol;
(3S)-1-[[3-[2,4-bis[(3S)-3-methylmorpholin-4-yl]pyrido[5,6-e]pyrimidin-7-yl]phenyl]methyl]pyrrolidin-3-ol;
2,4-bis[(3S)-3-methylmorpholin-4-yl]-7-[3-(methylsulfonylmethyl)phenyl]pyrido[3,2-e]pyrimidine;
[5-[2,4-bis[(3S)-3-methylmorpholin-4-yl]pyrido[5,6-e]pyrimidin-7-yl]-2-fluorophenyl]methanamine;
N-[[5-[2,4-bis[(3S)-3-methylmorpholin-4-yl]pyrido[5,6-e]pyrimidin-7-yl]-2-fluorophenyl]methyl]cyclopropanamine;
N-[[5-[2,4-bis[(3S)-3-methylmorpholin-4-yl]pyrido[5,6-e]pyrimidin-7-yl]-2-fluorophenyl]methyl]propan-2-amine;
1-[5-[2,4-bis[(3S)-3-methylmorpholin-4-yl]pyrido[5,6-e]pyrimidin-7-yl]-2-fluorophenyl]-N-methylmethanamine;
N-[[5-[2,4-bis[(3S)-3-methylmorpholin-4-yl]pyrido[5,6-e]pyrimidin-7-yl]-2-fluorophenyl]methyl]oxan-4-amine;
1-[5-[2,4-bis[(3S)-3-methylmorpholin-4-yl]pyrido[5,6-e]pyrimidin-7-yl]-2-fluorophenyl]-N-(cyclopropylmethyl)methanamine;
N-[[5-[2,4-bis[(3S)-3-methylmorpholin-4-yl]pyrido[5,6-e]pyrimidin-7-yl]-2-fluorophenyl]methyl]-2-methoxyethanamine;
N-[[5-[2,4-bis[(3S)-3-methylmorpholin-4-yl]pyrido[5,6-e]pyrimidin-7-yl]-2-fluorophenyl]methyl]-2-fluoroethanamine;
2-[[5-[2,4-bis[(3S)-3-methylmorpholin-4-yl]pyrido[5,6-e]pyrimidin-7-yl]-2-fluorophenyl]methylamino]ethanol;
(3R)-1-[[5-[2,4-bis[(3S)-3-methylmorpholin-4-yl]pyrido[5,6-e]pyrimidin-7-yl]-2-fluorophenyl]methyl]pyrrolidin-3-ol;
7-[4-fluoro-3-(piperazin-1-ylmethyl)phenyl]-2,4-bis[(3S)-3-methylmorpholin-4-yl]pyrido[3,2-e]pyrimidine;
1-[5-[2,4-bis[(3S)-3-methylmorpholin-4-yl]pyrido[5,6-e]pyrimidin-7-yl]-2-fluorophenyl]-N,N-dimethylmethanamine;
7-[4-fluoro-3-[(4-methylpiperazin-1-yl)methyl]phenyl]-2,4-bis[(3S)-3-methylmorpholin-4-yl]pyrido[3,2-e]pyrimidine;
(3R)-1-[[5-[2,4-bis[(3S)-3-methylmorpholin-4-yl]pyrido[5,6-e]pyrimidin-7-yl]-2-fluorophenyl]methyl]piperidin-3-ol;
N-[[5-[2,4-bis[(3S)-3-methylmorpholin-4-yl]pyrido[5,6-e]pyrimidin-7-yl]-2-fluorophenyl]methyl]-N-methylpropan-2-amine;
N-[[5-[2,4-bis[(3S)-3-methylmorpholin-4-yl]pyrido[5,6-e]pyrimidin-7-yl]-2-fluorophenyl]methyl]-2-methoxy-N-methylethanamine;
(3S)-1-[[5-[2,4-bis[(3S)-3-methylmorpholin-4-yl]pyrido[5,6-e]pyrimidin-7-yl]-2-fluorophenyl]methyl]pyrrolidin-3-ol;
N-[[5-[2,4-bis[(3S)-3-methylmorpholin-4-yl]pyrido[5,6-e]pyrimidin-7-yl]-2-methoxyphenyl]methyl]cyclopropanamine;
N-[[5-[2,4-bis[(3S)-3-methylmorpholin-4-yl]pyrido[5,6-e]pyrimidin-7-yl]-2-methoxyphenyl]methyl]propan-2-amine;
N-[[5-[2,4-bis[(3S)-3-methylmorpholin-4-yl]pyrido[5,6-e]pyrimidin-7-yl]-2-methoxyphenyl]methyl]cyclopentanamine;
N-[[5-[2,4-bis[(3S)-3-methylmorpholin-4-yl]pyrido[5,6-e]pyrimidin-7-yl]-2-methoxyphenyl]methyl]cyclohexanamine;
N-[[5-[2,4-bis[(3S)-3-methylmorpholin-4-yl]pyrido[5,6-e]pyrimidin-7-yl]-2-methoxyphenyl]methyl]-1-thiophen-2-ylmethanamine;
N-[[5-[2,4-bis[(3S)-3-methylmorpholin-4-yl]pyrido[5,6-e]pyrimidin-7-yl]-2-methoxyphenyl]methyl]oxan-4-amine;
N-[[5-[2,4-bis[(3S)-3-methylmorpholin-4-yl]pyrido[5,6-e]pyrimidin-7-yl]-2-methoxyphenyl]methyl]-1-ethynylcyclohexan-1-amine;
N-[[5-[2,4-bis[(3S)-3-methylmorpholin-4-yl]pyrido[5,6-e]pyrimidin-7-yl]-2-methoxyphenyl]methyl]-3,3-dimethylbutan-1-amine;
N-[[5-[2,4-bis[(3S)-3-methylmorpholin-4-yl]pyrido[5,6-e]pyrimidin-7-yl]-2-methoxyphenyl]methyl]-3-methylbutan-1-amine;
N-[[5-[2,4-bis[(3S)-3-methylmorpholin-4-yl]pyrido[5,6-e]pyrimidin-7-yl]-2-methoxyphenyl]methyl]-4-methylpentan-2-amine;
N-[[5-[2,4-bis[(3S)-3-methylmorpholin-4-yl]pyrido[6,5-d]pyrimidin-7-yl]-2-methoxyphenyl]methyl]-N′,N′-diethylpropane-1,3-diamine;
N-[[5-[2,4-bis[(3S)-3-methylmorpholin-4-yl]pyrido[6,5-d]pyrimidin-7-yl]-2-methoxyphenyl]methyl]-2-methylcyclohexan-1-amine;
1-[5-[2,4-bis[(3S)-3-methylmorpholin-4-yl]pyrido[5,6-e]pyrimidin-7-yl]-2-methoxyphenyl]-N-(cyclopropylmethyl)methanamine;
N-[[5-[2,4-bis[(3S)-3-methylmorpholin-4-yl]pyrido[6,5-d]pyrimidin-7-yl]-2-methoxyphenyl]methyl]butan-1-amine;
N-[[5-[2,4-bis[(3S)-3-methylmorpholin-4-yl]pyrido[5,6-e]pyrimidin-7-yl]-2-methoxyphenyl]methyl]-2-methylpropan-1-amine;
2-[[5-[2,4-bis[(3S)-3-methylmorpholin-4-yl]pyrido[5,6-e]pyrimidin-7-yl]-2-methoxyphenyl]methylamino]ethanol;
N-[[5-[2,4-bis[(3S)-3-methylmorpholin-4-yl]pyrido[5,6-e]pyrimidin-7-yl]-2-methoxyphenyl]methyl]-2-methoxyethanamine;
N-[[5-[2,4-bis[(3S)-3-methylmorpholin-4-yl]pyrido[6,5-d]pyrimidin-7-yl]-2-methoxyphenyl]methyl]-4-methylcyclohexan-1-amine;
N-[[5-[2,4-bis[(3S)-3-methylmorpholin-4-yl]pyrido[5,6-e]pyrimidin-7-yl]-2-methoxyphenyl]methyl]-2-(4-methoxyphenyl)ethanamine;
N-[[5-[2,4-bis[(3S)-3-methylmorpholin-4-yl]pyrido[5,6-e]pyrimidin-7-yl]-2-methoxyphenyl]methyl]-2-(3H-imidazol-4-yl)ethanamine;
N-[[5-[2,4-bis[(3S)-3-methylmorpholin-4-yl]pyrido[5,6-e]pyrimidin-7-yl]-2-methoxyphenyl]methyl]cyclooctanamine;
N-[[3-[2,4-bis[(3S)-3-methylmorpholin-4-yl]pyrido[5,6-e]pyrimidin-7-yl]phenyl]methyl]acetamide;
N-[[3-[2,4-bis[(3S)-3-methylmorpholin-4-yl]pyrido[5,6-e]pyrimidin-7-yl]phenyl]methyl]-2-methoxyacetamide;
N-[[3-[2,4-bis[(3S)-3-methylmorpholin-4-yl]pyrido[5,6-e]pyrimidin-7-yl]phenyl]methyl]cyclopropanecarboxamide;
N-[[3-[2,4-bis[(3S)-3-methylmorpholin-4-yl]pyrido[5,6-e]pyrimidin-7-yl]phenyl]methyl]-2-methylpropanamide;
N-[[3-[2,4-bis[(3S)-3-methylmorpholin-4-yl]pyrido[5,6-e]pyrimidin-7-yl]phenyl]methyl]cyclobutanecarboxamide;
N-[[3-[2,4-bis[(3S)-3-methylmorpholin-4-yl]pyrido[5,6-e]pyrimidin-7-yl]phenyl]methyl]-3-methylbutanamide;
N-[[3-[2,4-bis[(3S)-3-methylmorpholin-4-yl]pyrido[5,6-e]pyrimidin-7-yl]phenyl]methyl]-2,2,2-trifluoroacetamide;
N-[[3-[2,4-bis[(3S)-3-methylmorpholin-4-yl]pyrido[5,6-e]pyrimidin-7-yl]phenyl]methyl]-N-methylacetamide;
N-[[3-[2,4-bis[(3S)-3-methylmorpholin-4-yl]pyrido[5,6-e]pyrimidin-7-yl]phenyl]methyl]-2-methoxy-N-methylacetamide;
N-[[3-[2,4-bis[(3S)-3-methylmorpholin-4-yl]pyrido[5,6-e]pyrimidin-7-yl]phenyl]methyl]-N-methylcyclopropanecarboxamide;
N-[[3-[2,4-bis[(3S)-3-methylmorpholin-4-yl]pyrido[5,6-e]pyrimidin-7-yl]phenyl]methyl]-N,2-dimethylpropanamide;
N-[[3-[2,4-bis[(3S)-3-methylmorpholin-4-yl]pyrido[5,6-e]pyrimidin-7-yl]phenyl]methyl]-N-methylcyclobutanecarboxamide;
N-[[3-[2,4-bis[(3S)-3-methylmorpholin-4-yl]pyrido[5,6-e]pyrimidin-7-yl]phenyl]methyl]-N,3-dimethylbutanamide;
N-[[3-[2,4-bis[(3S)-3-methylmorpholin-4-yl]pyrido[5,6-e]pyrimidin-7-yl]phenyl]methyl]-2,2,2-trifluoro-N-methylacetamide;
N-[[3-[2,4-bis[(3S)-3-methylmorpholin-4-yl]pyrido[5,6-e]pyrimidin-7-yl]phenyl]methyl]-N-methylmethanesulfonamide;
N-[[3-[2,4-bis[(3S)-3-methylmorpholin-4-yl]pyrido[6,5-d]pyrimidin-7-yl]phenyl]methyl]-N-methylethanesulfonamide;
N-[[4-[2,4-bis[(3S)-3-methylmorpholin-4-yl]pyrido[5,6-e]pyrimidin-7-yl]phenyl]methyl]acetamide;
N-[[4-[2,4-bis[(3S)-3-methylmorpholin-4-yl]pyrido[5,6-e]pyrimidin-7-yl]phenyl]methyl]-N-methylacetamide;
N-[[4-[2,4-bis[(3S)-3-methylmorpholin-4-yl]pyrido[5,6-e]pyrimidin-7-yl]phenyl]methyl]-2-methoxy-N-methylacetamide;
N-[[5-[2,4-bis[(3S)-3-methylmorpholin-4-yl]pyrido[5,6-e]pyrimidin-7-yl]-2-fluorophenyl]methyl]acetamide;
N-[[5-[2,4-bis[(3S)-3-methylmorpholin-4-yl]pyrido[5,6-e]pyrimidin-7-yl]-2-fluorophenyl]methyl]-2-methoxyacetamide;
N-[[5-[2,4-bis[(3S)-3-methylmorpholin-4-yl]pyrido[5,6-e]pyrimidin-7-yl]-2-fluorophenyl]methyl]cyclopropanecarboxamide;
N-[[5-[2,4-bis[(3S)-3-methylmorpholin-4-yl]pyrido[5,6-e]pyrimidin-7-yl]-2-fluorophenyl]methyl]-2-methylpropanamide;
N-[[5-[2,4-bis[(3S)-3-methylmorpholin-4-yl]pyrido[5,6-e]pyrimidin-7-yl]-2-fluorophenyl]methyl]cyclobutanecarboxamide;
N-[[5-[2,4-bis[(3S)-3-methylmorpholin-4-yl]pyrido[5,6-e]pyrimidin-7-yl]-2-fluorophenyl]methyl]-3-methylbutanamide;
N-[[5-[2,4-bis[(3S)-3-methylmorpholin-4-yl]pyrido[5,6-e]pyrimidin-7-yl]-2-fluorophenyl]methyl]-2,2,2-trifluoroacetamide;
[3-[2,4-bis[(3S)-3-methylmorpholin-4-yl]pyrido[5,6-e]pyrimidin-7-yl]furan-2-yl]methanol;
2-[[5-[2,4-bis[(3S)-3-methylmorpholin-4-yl]pyrido[5,6-e]pyrimidin-7-yl]furan-2-yl]methylamino]ethanol;
N-[[5-[2,4-bis[(3S)-3-methylmorpholin-4-yl]pyrido[5,6-e]pyrimidin-7-yl]furan-2-yl]methyl]-N′,N′-dimethylethane-1,2-diamine;
1-[5-[2,4-bis[(3S)-3-methylmorpholin-4-yl]pyrido[5,6-e]pyrimidin-7-yl]furan-2-yl]-N-methylmethanamine;
N-[[5-[2,4-bis[(3S)-3-methylmorpholin-4-yl]pyrido[6,5-d]pyrimidin-7-yl]furan-2-yl]methyl]ethanamine;
N-[[5-[2,4-bis[(3S)-3-methylmorpholin-4-yl]pyrido[5,6-e]pyrimidin-7-yl]furan-2-yl]methyl]cyclopropanamine;
N-[[5-[2,4-bis[(3S)-3-methylmorpholin-4-yl]pyrido[5,6-e]pyrimidin-7-yl]furan-2-yl]methyl]oxan-4-amine;
1-[5-[2,4-bis[(3S)-3-methylmorpholin-4-yl]pyrido[5,6-e]pyrimidin-7-yl]furan-2-yl]-N-(cyclopropylmethyl)methanamine;
N-[[5-[2,4-bis[(3S)-3-methylmorpholin-4-yl]pyrido[5,6-e]pyrimidin-7-yl]furan-2-yl]methyl]-2-methoxyethanamine;
N-[[5-[2,4-bis[(3S)-3-methylmorpholin-4-yl]pyrido[5,6-e]pyrimidin-7-yl]furan-2-yl]methyl]-1H-pyrazol-3-amine;
2-[[5-[2,4-bis[(3S)-3-methylmorpholin-4-yl]pyrido[5,6-e]pyrimidin-7-yl]furan-2-yl]methylamino]acetamide;
N-[[5-[2,4-bis[(3S)-3-methylmorpholin-4-yl]pyrido[5,6-e]pyrimidin-7-yl]furan-2-yl]methyl]propan-2-amine;
3-[2,4-bis[(3S)-3-methylmorpholin-4-yl]pyrido[5,6-e]pyrimidin-7-yl]-N-(2-hydroxyethyl)benzamide;
5-[2,4-bis[(3S)-3-methylmorpholin-4-yl]pyrido[5,6-e]pyrimidin-7-yl]-N-(2-hydroxyethyl)-2-methoxybenzamide;
5-[2,4-bis[(3S)-3-methylmorpholin-4-yl]pyrido[5,6-e]pyrimidin-7-yl]-N-methylpyridine-2-carboxamide;
5-[2,4-bis[(3S)-3-methylmorpholin-4-yl]pyrido[5,6-e]pyrimidin-7-yl]-N,N-dimethylpyridine-2-carboxamide;
7-[4-fluoro-3-(methoxymethyl)phenyl]-2,4-bis[(3S)-3-methylmorpholin-4-yl]pyrido[3,2-e]pyrimidine;
4-[2,4-bis[(3S)-3-methylmorpholin-4-yl]pyrido[5,6-e]pyrimidin-7-yl]-1-methylpyridin-2-one;
N-[3-[2,4-bis[(3S)-3-methylmorpholin-4-yl]pyrido[5,6-e]pyrimidin-7-yl]phenyl]-N-methylmethanesulfonamide;
2-[(2S,6R)-2,6-dimethylmorpholin-4-yl]-4-[(3S)-3-methylmorpholin-4-yl]-7-phenylpteridine;
2-[(2R,6S)-2,6-dimethylmorpholin-4-yl]-4-[(3S)-3-methylmorpholin-4-yl]-7-(4-methylphenyl)pteridine;
2,4-bis[(3S)-3-methylmorpholin-4-yl]-7-(4-methylphenyl)pteridine;
2,4-bis[(3S)-3-methylmorpholin-4-yl]-7-phenylpteridine;
2-chloro-4-[(3S)-3-methylmorpholin-4-yl]-7-(4-methylphenyl)pteridine;
7-(2-methoxypyridin-4-yl)-2,4-bis[(3S)-3-methylmorpholin-4-yl]pyrido[3,2-e]pyrimidine;
2-[5-[2,4-bis[(3S)-3-methylmorpholin-4-yl]pyrido[5,6-e]pyrimidin-7-yl]pyridin-2-yl]oxy-N,N-dimethylethanamine;
4-[2,4-bis[(3S)-3-methylmorpholin-4-yl]pyrido[5,6-e]pyrimidin-7-yl]-1H-pyridin-2-one;
4-[2,4-bis[(3S)-3-methylmorpholin-4-yl]pyrido[5,6-e]pyrimidin-7-yl]pyridine-2-carbonitrile;
4-[2,4-bis[(3S)-3-methylmorpholin-4-yl]pyrido[5,6-e]pyrimidin-7-yl]pyridine-2-carboxamide;
5-[2,4-bis[(3S)-3-methylmorpholin-4-yl]pyrido[5,6-e]pyrimidin-7-yl]pyridine-2-carbonitrile;
4-[2,4-bis[(3S)-3-methylmorpholin-4-yl]pyrido[5,6-e]pyrimidin-7-yl]-N-methylpyridin-2-amine;
5-[2,4-bis[(3S)-3-methylmorpholin-4-yl]pyrido[5,6-e]pyrimidin-7-yl]-2-methoxybenzoic acid;
5-[2,4-bis[(3S)-3-methylmorpholin-4-yl]pyrido[5,6-e]pyrimidin-7-yl]-2-methoxybenzamide;
N-[3-[2,4-bis[(3S)-3-methylmorpholin-4-yl]pyrido[5,6-e]pyrimidin-7-yl]phenyl]-2-hydroxyethanesulfonamide;
N-[3-[2,4-bis[(3S)-3-methylmorpholin-4-yl]pyrido[5,6-e]pyrimidin-7-yl]phenyl]propane-2-sulfonamide;
N-[3-[2,4-bis[(3S)-3-methylmorpholin-4-yl]pyrido[5,6-e]pyrimidin-7-yl]phenyl]oxolane-2-carboxamide;
N-[5-[2,4-bis[(3S)-3-methylmorpholin-4-yl]pyrido[5,6-e]pyrimidin-7-yl]pyridin-2-yl]acetamide;
2-[(2R,6S)-2,6-dimethylmorpholin-4-yl]-4-[(3S)-3-methylmorpholin-4-yl]-7-phenoxypyrido[2,3-d]pyrimidine;
[2-methoxy-5-[4-[(3S)-3-methylmorpholin-4-yl]-2-[(2R)-2-methylpiperidin-1-yl]pyrido[6,5-d]pyrimidin-7-yl]phenyl]methanol;
[5-[2-amino-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-piperidin-1-ylpyrido[5,6-e]pyrimidin-7-yl]phenyl]methanol;
[5-[2-(azepan-1-yl)-4-[(3S)-3-methylmorpholin-4-yl]pyrido[5,6-e]pyrimidin-7-yl]-2-methoxyphenyl]methanol;
[5-[2-(3-aminoazepan-1-yl)-4-[(3S)-3-methylmorpholin-4-yl]pyrido[5,6-e]pyrimidin-7-yl]-2-methoxyphenyl]methanol;
3-[1-[7-[3-(hydroxymethyl)-4-methoxyphenyl]-4-[(3S)-3-methylmorpholin-4-yl]pyrido[3,2-e]pyrimidin-2-yl]piperidin-4-yl]phenol;
2-[1-[7-[3-(hydroxymethyl)-4-methoxyphenyl]-4-[(3S)-3-methylmorpholin-4-yl]pyrido[3,2-e]pyrimidin-2-yl]piperidin-4-yl]ethanol;
[2-methoxy-5-[4-[(3S)-3-methylmorpholin-4-yl]-2-[3-(trifluoromethyl)piperidin-1-yl]pyrido[5,6-e]pyrimidin-7-yl]phenyl]methanol;
[2-methoxy-5-[4-[(3S)-3-methylmorpholin-4-yl]-2-(3-methylpiperidin-1-yl)pyrido[6,5-d]pyrimidin-7-yl]phenyl]methanol;
[2-methoxy-5-[4-[(3S)-3-methylmorpholin-4-yl]-2-(3-phenylpiperidin-1-yl)pyrido[5,6-e]pyrimidin-7-yl]phenyl]methanol;
1-[7-[3-(hydroxymethyl)-4-methoxyphenyl]-4-[(3S)-3-methylmorpholin-4-yl]pyrido[3,2-e]pyrimidin-2-yl]piperidin-3-ol;
[5-[2-(2-iodoimidazol-1-yl)-4-[(3S)-3-methylmorpholin-4-yl]pyrido[5,6-e]pyrimidin-7-yl]-2-methoxyphenyl]methanol;
[5-[2-anilino-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-(pyrimidin-4-ylamino)pyrido[5,6-e]pyrimidin-7-yl]phenyl]methanol;
[2-methoxy-5-[4-[(3S)-3-methylmorpholin-4-yl]-2-(pyridin-3-ylamino)pyrido[5,6-e]pyrimidin-7-yl]phenyl]methanol;
[2-methoxy-5-[4-[(3S)-3-methylmorpholin-4-yl]-2-(pyridin-2-ylamino)pyrido[5,6-e]pyrimidin-7-yl]phenyl]methanol;
[2-methoxy-5-[4-[(3S)-3-methylmorpholin-4-yl]-2-pyrrolidin-1-ylpyrido[5,6-e]pyrimidin-7-yl]phenyl]methanol;
ethyl 1-[7-[3-(hydroxymethyl)-4-methoxyphenyl]-4-[(3S)-3-methylmorpholin-4-yl]pyrido[2,3-d]pyrimidin-2-yl]piperidine-3-carboxylate;
[5-[2-[4-(aminomethyl)piperidin-1-yl]-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-(4-methylpiperidin-1-yl)pyrido[6,5-d]pyrimidin-7-yl]phenyl]methanol;
ethyl 1-[7-[3-(hydroxymethyl)-4-methoxyphenyl]-4-[(3S)-3-methylmorpholin-4-yl]pyrido[2,3-d]pyrimidin-2-yl]piperidine-4-carboxylate;
1-[7-[3-(hydroxymethyl)-4-methoxyphenyl]-4-[(3S)-3-methylmorpholin-4-yl]pyrido[3,2-e]pyrimidin-2-yl]piperidine-4-carboxamide;
[1-[7-[3-(hydroxymethyl)-4-methoxyphenyl]-4-[(3S)-3-methylmorpholin-4-yl]pyrido[3,2-e]pyrimidin-2-yl]piperidin-4-yl]-(4-methoxyphenyl)methanone;
[5-[2-[4-(hydroxymethyl)piperidin-1-yl]-4-[(3S)-3-methylmorpholin-4-yl]pyrido[5,6-e]pyrimidin-7-yl]-2-methoxyphenyl]methanol;
[5-[2-[3-(hydroxymethyl)piperidin-1-yl]-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-(4-piperidin-1-ylpiperidin-1-yl)pyrido[5,6-e]pyrimidin-7-yl]phenyl]methanol;
methyl (2S,4R)-4-hydroxy-1-[7-[3-(hydroxymethyl)-4-methoxyphenyl]-4-[(3S)-3-methylmorpholin-4-yl]pyrido[3,2-e]pyrimidin-2-yl]pyrrolidine-2-carboxylate;
(3S)-1-[7-[3-(hydroxymethyl)-4-methoxyphenyl]-4-[(3S)-3-methylmorpholin-4-yl]pyrido[3,2-e]pyrimidin-2-yl]pyrrolidin-3-ol;
[2-methoxy-5-[4-[(3S)-3-methylmorpholin-4-yl]-2-(4-morpholin-4-ylpiperidin-1-yl)pyrido[5,6-e]pyrimidin-7-yl]phenyl]methanol;
[5-[2-(4-aminopiperidin-1-yl)-4-[(3S)-3-methylmorpholin-4-yl]pyrido[5,6-e]pyrimidin-7-yl]-2-methoxyphenyl]methanol;
[5-[2-(3,5-dimethylpiperidin-1-yl)-4-[(3S)-3-methylmorpholin-4-yl]pyrido[6,5-d]pyrimidin-7-yl]-2-methoxyphenyl]methanol;
[2-methoxy-5-[4-[(3S)-3-methylmorpholin-4-yl]-2-[4-(trifluoromethyl)piperidin-1-yl]pyrido[5,6-e]pyrimidin-7-yl]phenyl]methanol;
[5-[2-(3,4,4a,5,6,7,8,8a-octahydro-1H-isoquinolin-2-yl)-4-[(3S)-3-methylmorpholin-4-yl]pyrido[5,6-e]pyrimidin-7-yl]-2-methoxyphenyl]methanol;
methyl (2S)-1-[7-[3-(hydroxymethyl)-4-methoxyphenyl]-4-[(3S)-3-methylmorpholin-4-yl]pyrido[3,2-e]pyrimidin-2-yl]pyrrolidine-2-carboxylate;
[2-methoxy-5-[4-[(3S)-3-methylmorpholin-4-yl]-2-(4-pyrrolidin-1-ylpiperidin-1-yl)pyrido[5,6-e]pyrimidin-7-yl]phenyl]methanol;
[2-methoxy-5-[2-(3-methoxypiperidin-1-yl)-4-[(3S)-3-methylmorpholin-4-yl]pyrido[5,6-e]pyrimidin-7-yl]phenyl]methanol;
(4-chlorophenyl)-[1-[7-[3-(hydroxymethyl)-4-methoxyphenyl]-4-[(3S)-3-methylmorpholin-4-yl]pyrido[3,2-e]pyrimidin-2-yl]piperidin-4-yl]methanone;
[5-[2-(4-benzylpiperidin-1-yl)-4-[(3S)-3-methylmorpholin-4-yl]pyrido[5,6-e]pyrimidin-7-yl]-2-methoxyphenyl]methanol;
1-[7-[3-(hydroxymethyl)-4-methoxyphenyl]-4-[(3S)-3-methylmorpholin-4-yl]pyrido[3,2-e]pyrimidin-2-yl]-4-phenylpiperidin-4-ol;
1-[7-[3-(hydroxymethyl)-4-methoxyphenyl]-4-[(3S)-3-methylmorpholin-4-yl]pyrido[3,2-e]pyrimidin-2-yl]-4-phenylpiperidine-4-carbonitrile;
(2S)-1-[7-[3-(hydroxymethyl)-4-methoxyphenyl]-4-[(3S)-3-methylmorpholin-4-yl]pyrido[3,2-e]pyrimidin-2-yl]pyrrolidine-2-carboxamide;
[2-methoxy-5-[4-[(3S)-3-methylmorpholin-4-yl]-2-(2-methylpyrrolidin-1-yl)pyrido[6,5-d]pyrimidin-7-yl]phenyl]methanol;
[5-[2-[4-(4-chlorophenyl)piperidin-1-yl]-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-[3-(3-methyl-1,2,4-oxadiazol-5-yl)piperidin-1-yl]pyrido[6,5-d]pyrimidin-7-yl]phenyl]methanol;
[5-[2-[3-(dimethylaminomethyl)piperidin-1-yl]-4-[(3S)-3-methylmorpholin-4-yl]pyrido[5,6-e]pyrimidin-7-yl]-2-methoxyphenyl]methanol;
4-chloro-N-cyclopropyl-N-[1-[7-[3-(hydroxymethyl)-4-methoxyphenyl]-4-[(3S)-3-methylmorpholin-4-yl]pyrido[3,2-e]pyrimidin-2-yl]piperidin-4-yl]benzenesulfonamide;
[5-[2-(3-aminopiperidin-1-yl)-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-(4-methylpentan-2-ylamino)pyrido[5,6-e]pyrimidin-7-yl]phenyl]methanol;
[5-[2-(cyclopentylamino)-4-[(3S)-3-methylmorpholin-4-yl]pyrido[5,6-e]pyrimidin-7-yl]-2-methoxyphenyl]methanol;
[2-methoxy-5-[2-(1-methoxypropan-2-ylamino)-4-[(3S)-3-methylmorpholin-4-yl]pyrido[5,6-e]pyrimidin-7-yl]phenyl]methanol;
[5-[2-imidazol-1-yl-4-[(3S)-3-methylmorpholin-4-yl]pyrido[5,6-e]pyrimidin-7-yl]-2-methoxyphenyl]methanol;
methyl 3-[7-[3-(hydroxymethyl)-4-methoxyphenyl]-4-[(3S)-3-methylmorpholin-4-yl]pyrido[3,2-e]pyrimidin-2-yl]imidazole-4-carboxylate;
3-[7-[3-(hydroxymethyl)-4-methoxyphenyl]-4-[(3S)-3-methylmorpholin-4-yl]pyrido[2,3-d]pyrimidin-2-yl]-5-methylimidazole-4-carbaldehyde;
[5-[2-(4-bromoimidazol-1-yl)-4-[(3S)-3-methylmorpholin-4-yl]pyrido[5,6-e]pyrimidin-7-yl]-2-methoxyphenyl]methanol;
[5-[2-(4-bromo-2-methylimidazol-1-yl)-4-[(3S)-3-methylmorpholin-4-yl]pyrido[6,5-d]pyrimidin-7-yl]-2-methoxyphenyl]methanol;
[5-[2-(5-ethyl-2-methylpiperidin-1-yl)-4-[(3S)-3-methylmorpholin-4-yl]pyrido[6,5-d]pyrimidin-7-yl]-2-methoxyphenyl]methanol;
[2-methoxy-5-[4-[(3S)-3-methylmorpholin-4-yl]-2-(4-phenylpiperidin-1-yl)pyrido[5,6-e]pyrimidin-7-yl]phenyl]methanol;
[2-methoxy-5-[4-[(3S)-3-methylmorpholin-4-yl]-2-(2-phenylmorpholin-4-yl)pyrido[5,6-e]pyrimidin-7-yl]phenyl]methanol;
[5-[2-(2,5-dimethylpyrrolidin-1-yl)-4-[(3S)-3-methylmorpholin-4-yl]pyrido[6,5-d]pyrimidin-7-yl]-2-methoxyphenyl]methanol;
[2-methoxy-5-[4-[(3S)-3-methylmorpholin-4-yl]-2-[(2R)-2-methylpiperidin-1-yl]pyrido[6,5-d]pyrimidin-7-yl]phenyl]methanol;
[2-methoxy-5-[4-[(3S)-3-methylmorpholin-4-yl]-2-(4-pyridin-4-ylpiperidin-1-yl)pyrido[5,6-e]pyrimidin-7-yl]phenyl]methanol;
(3S)-1-[7-[3-(hydroxymethyl)-4-methoxyphenyl]-4-[(3S)-3-methylmorpholin-4-yl]pyrido[3,2-e]pyrimidin-2-yl]piperidin-3-ol;
(3R)-1-[7-[3-(hydroxymethyl)-4-methoxyphenyl]-4-[(3S)-3-methylmorpholin-4-yl]pyrido[3,2-e]pyrimidin-2-yl]piperidin-3-ol;
[5-[2-(2,3-dihydro-1,4-benzoxazin-4-yl)-4-[(3S)-3-methylmorpholin-4-yl]pyrido[5,6-e]pyrimidin-7-yl]-2-methoxyphenyl]methanol;
[5-[2-[2-(hydroxymethyl)piperidin-1-yl]-4-[(3S)-3-methylmorpholin-4-yl]pyrido[5,6-e]pyrimidin-7-yl]-2-methoxyphenyl]methanol;
(3R)-1-[7-[3-(hydroxymethyl)-4-methoxyphenyl]-4-[(3S)-3-methylmorpholin-4-yl]pyrido[3,2-e]pyrimidin-2-yl]pyrrolidin-3-ol;
[2-methoxy-5-[2-[[(2R)-1-methoxypropan-2-yl]amino]-4-[(3S)-3-methylmorpholin-4-yl]pyrido[5,6-e]pyrimidin-7-yl]phenyl]methanol;
[2-methoxy-5-[2-methylamino-4-[(3S)-3-methylmorpholin-4-yl]pyrido[5,6-e]pyrimidin-7-yl]phenyl]methanol;
[5-[2-ethylamino-4-[(3S)-3-methylmorpholin-4-yl]pyrido[6,5-d]pyrimidin-7-yl]-2-methoxyphenyl]methanol;
[2-methoxy-5-[4-[(3S)-3-methylmorpholin-4-yl]-2-(propan-2-ylamino)pyrido[5,6-e]pyrimidin-7-yl]phenyl]methanol;
2-[[7-[3-(hydroxymethyl)-4-methoxyphenyl]-4-[(3S)-3-methylmorpholin-4-yl]pyrido[3,2-e]pyrimidin-2-yl]amino]ethanol;
[5-[2-(2-fluoroethylamino)-4-[(3S)-3-methylmorpholin-4-yl]pyrido[5,6-e]pyrimidin-7-yl]-2-methoxyphenyl]methanol;
2-[[7-[3-(hydroxymethyl)-4-methoxyphenyl]-4-[(3S)-3-methylmorpholin-4-yl]pyrido[3,2-e]pyrimidin-2-yl]amino]acetamide;
[2-methoxy-5-[2-(2-methylaminoethylamino)-4-[(3S)-3-methylmorpholin-4-yl]pyrido[5,6-e]pyrimidin-7-yl]phenyl]methanol;
(2R)-2-[[7-[3-(hydroxymethyl)-4-methoxyphenyl]-4-[(3S)-3-methylmorpholin-4-yl]pyrido[3,2-e]pyrimidin-2-yl]amino]propan-1-ol;
(2S)-2-[[7-[3-(hydroxymethyl)-4-methoxyphenyl]-4-[(3S)-3-methylmorpholin-4-yl]pyrido[3,2-e]pyrimidin-2-yl]amino]propan-1-ol;
[2-methoxy-5-[2-(2-methoxyethylamino)-4-[(3S)-3-methylmorpholin-4-yl]pyrido[5,6-e]pyrimidin-7-yl]phenyl]methanol;
[5-[2-(2,2-difluoroethylamino)-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-piperazin-1-ylpyrido[5,6-e]pyrimidin-7-yl]phenyl]methanol;
[5-[2-(2-dimethylaminoethylamino)-4-[(3S)-3-methylmorpholin-4-yl]pyrido[5,6-e]pyrimidin-7-yl]-2-methoxyphenyl]methanol;
2-[[7-[3-(hydroxymethyl)-4-methoxyphenyl]-4-[(3S)-3-methylmorpholin-4-yl]pyrido[3,2-e]pyrimidin-2-yl]amino]-2-methylpropan-1-ol;
2-[[7-[3-(hydroxymethyl)-4-methoxyphenyl]-4-[(3S)-3-methylmorpholin-4-yl]pyrido[3,2-e]pyrimidin-2-yl]amino]propane-1,3-diol;
[5-[2-[2-(hydroxymethyl)imidazol-1-yl]-4-[(3S)-3-methylmorpholin-4-yl]pyrido[5,6-e]pyrimidin-7-yl]-2-methoxyphenyl]methanol;
4-[7-[3-(hydroxymethyl)-4-methoxyphenyl]-4-[(3S)-3-methylmorpholin-4-yl]pyrido[3,2-e]pyrimidin-2-yl]piperazin-2-one;
[2-methoxy-5-[4-[(3S)-3-methylmorpholin-4-yl]-2-(4-methylpiperazin-1-yl)pyrido[5,6-e]pyrimidin-7-yl]phenyl]methanol;
[2-methoxy-5-[4-[(3S)-3-methylmorpholin-4-yl]-2-thiomorpholin-4-ylpyrido[5,6-e]pyrimidin-7-yl]phenyl]methanol;
2-[4-[7-[3-(hydroxymethyl)-4-methoxyphenyl]-4-[(3S)-3-methylmorpholin-4-yl]pyrido[3,2-e]pyrimidin-2-yl]piperazin-1-yl]ethanol;
[5-[2-(bis(2-methoxyethyl)amino)-4-[(3S)-3-methylmorpholin-4-yl]pyrido[5,6-e]pyrimidin-7-yl]-2-methoxyphenyl]methanol;
2-[2-hydroxyethyl-[7-[3-(hydroxymethyl)-4-methoxyphenyl]-4-[(3S)-3-methylmorpholin-4-yl]pyrido[3,2-e]pyrimidin-2-yl]amino]ethanol;
[5-[2-[(2S)-2-(hydroxymethyl)pyrrolidin-1-yl]-4-[(3S)-3-methylmorpholin-4-yl]pyrido[5,6-e]pyrimidin-7-yl]-2-methoxyphenyl]methanol;
[5-[2-[(2R)-2-(hydroxymethyl)pyrrolidin-1-yl]-4-[(3S)-3-methylmorpholin-4-yl]pyrido[5,6-e]pyrimidin-7-yl]-2-methoxyphenyl]methanol;
[2-methoxy-5-[2-(2-methylimidazol-1-yl)-4-[(3S)-3-methylmorpholin-4-yl]pyrido[6,5-d]pyrimidin-7-yl]phenyl]methanol;
4-[7-[3-(hydroxymethyl)-4-methoxyphenyl]-4-[(3S)-3-methylmorpholin-4-yl]pyrido[3,2-e]pyrimidin-2-yl]-3-methylpiperazin-2-one;
(2R)-2-[[7-[3-(hydroxymethyl)-4-methoxyphenyl]-4-[(3S)-3-methylmorpholin-4-yl]pyrido[3,2-e]pyrimidin-2-yl]amino]propanamide;
(2S)-2-[[7-[3-(hydroxymethyl)-4-methoxyphenyl]-4-[(3S)-3-methylmorpholin-4-yl]pyrido[3,2-e]pyrimidin-2-yl]amino]propanamide;
2-[[7-[3-(hydroxymethyl)-4-methoxyphenyl]-4-[(3S)-3-methylmorpholin-4-yl]pyrido[3,2-e]pyrimidin-2-yl]-methylamino]ethanol;
2-[[7-[3-(hydroxymethyl)-4-methoxyphenyl]-4-[(3S)-3-methylmorpholin-4-yl]pyrido[3,2-e]pyrimidin-2-yl]-propan-2-ylamino]ethanol;
[5-[2-(1-dimethylaminopropan-2-ylamino)-4-[(3S)-3-methylmorpholin-4-yl]pyrido[5,6-e]pyrimidin-7-yl]-2-methoxyphenyl]methanol;
[2-methoxy-5-[2-methoxy-4-[(3S)-3-methylmorpholin-4-yl]pyrido[5,6-e]pyrimidin-7-yl]phenyl]methanol;
[5-[2-ethoxy-4-[(3S)-3-methylmorpholin-4-yl]pyrido[6,5-d]pyrimidin-7-yl]-2-methoxyphenyl]methanol;
2-[7-[3-(hydroxymethyl)-4-methoxyphenyl]-4-[(3S)-3-methylmorpholin-4-yl]pyrido[3,2-e]pyrimidin-2-yl]oxyethanol;
[2-methoxy-5-[2-(2-methoxyethoxy)-4-[(3S)-3-methylmorpholin-4-yl]pyrido[5,6-e]pyrimidin-7-yl]phenyl]methanol;
[5-[2-(2-dimethylaminoethoxy)-4-[(3S)-3-methylmorpholin-4-yl]pyrido[5,6-e]pyrimidin-7-yl]-2-methoxyphenyl]methanol;
[5-[2-(1-dimethylaminopropan-2-yloxy)-4-[(3S)-3-methylmorpholin-4-yl]pyrido[5,6-e]pyrimidin-7-yl]-2-methoxyphenyl]methanol;
[2-methoxy-5-[4-[(3S)-3-methylmorpholin-4-yl]-2-(2-methylsulfonylethoxy)pyrido[5,6-e]pyrimidin-7-yl]phenyl]methanol;
[2-methoxy-5-[2-(2-methylaminoethoxy)-4-[(3S)-3-methylmorpholin-4-yl]pyrido[5,6-e]pyrimidin-7-yl]phenyl]methanol;
3-[7-[3-(hydroxymethyl)-4-methoxyphenyl]-4-[(3S)-3-methylmorpholin-4-yl]pyrido[3,2-e]pyrimidin-2-yl]oxypropanenitrile;
[2-methoxy-5-[4-[(3S)-3-methylmorpholin-4-yl]-2-(oxazinan-2-yl)pyrido[5,6-e]pyrimidin-7-yl]phenyl]methanol;
7-(4-chlorophenyl)-4-[(3S)-3-methylmorpholin-4-yl]-2-[(2S)-2-methylpiperidin-1-yl]pyrido[2,3-d]pyrimidine;
7-(4-chlorophenyl)-2-(2-ethylpiperidin-1-yl)-4-[(3S)-3-methylmorpholin-4-yl]pyrido[2,3-d]pyrimidine;
7-(4-chlorophenyl)-4-[(3S)-3-methylmorpholin-4-yl]-2-[(2R)-2-methylpiperidin-1-yl]pyrido[2,3-d]pyrimidine;
7-(4-chlorophenyl)-2-[(2S,6R)-2,6-dimethylmorpholin-4-yl]-4-[(3S)-3-methylmorpholin-4-yl]pyrido[2,3-d]pyrimidine;
7-(4-chlorophenyl)-4-[(3S)-3-methylmorpholin-4-yl]-2-morpholin-4-ylpyrido[3,2-e]pyrimidine;
1-[7-(4-chlorophenyl)-4-[(3S)-3-methylmorpholin-4-yl]pyrido[3,2-e]pyrimidin-2-yl]piperidine-4-carboxamide;
1-[7-(6-aminopyridin-3-yl)-4-[(3S)-3-methylmorpholin-4-yl]pyrido[3,2-e]pyrimidin-2-yl]-N-methylpiperidine-4-carboxamide;
5-[4-[(3S)-3-methylmorpholin-4-yl]-2-morpholin-4-ylpyrido[5,6-e]pyrimidin-7-yl]pyridin-2-amine;
1-[7-(6-aminopyridin-3-yl)-4-[(3S)-3-methylmorpholin-4-yl]pyrido[3,2-e]pyrimidin-2-yl]-N,N-dimethylpiperidine-4-carboxamide;
[5-[2,4-bis[(3R)-3-methylmorpholin-4-yl]pyrido[5,6-e]pyrimidin-7-yl]-2-methoxyphenyl]methanol;
[5-[2,4-bis[(3S)-3-methylmorpholin-4-yl]pyrido[5,6-e]pyrimidin-7-yl]-2-methoxyphenyl]methanol;
[5-[2-(furan-2-yl)-4-[(3S)-3-methylmorpholin-4-yl]pyrido[5,6-e]pyrimidin-7-yl]-2-methoxyphenyl]methanol;
3-[7-[3-(hydroxymethyl)-4-methoxyphenyl]-4-[(3S)-3-methylmorpholin-4-yl]pyrido[3,2-e]pyrimidin-2-yl]benzaldehyde;
[3-[7-[3-(hydroxymethyl)-4-methoxyphenyl]-4-[(3S)-3-methylmorpholin-4-yl]pyrido[3,2-e]pyrimidin-2-yl]phenyl]methanol;
[2-methoxy-5-[4-[(3S)-3-methylmorpholin-4-yl]-2-(3-phenylphenyl)pyrido[5,6-e]pyrimidin-7-yl]phenyl]methanol;
[5-[2-[3-(aminomethyl)phenyl]-4-[(3S)-3-methylmorpholin-4-yl]pyrido[5,6-e]pyrimidin-7-yl]-2-methoxyphenyl]methanol;
[5-[2-(3-aminophenyl)-4-[(3S)-3-methylmorpholin-4-yl]pyrido[5,6-e]pyrimidin-7-yl]-2-methoxyphenyl]methanol;
3-[7-[3-(hydroxymethyl)-4-methoxyphenyl]-4-[(3S)-3-methylmorpholin-4-yl]pyrido[3,2-e]pyrimidin-2-yl]-5-nitrobenzoic acid;
methyl 3-[7-[3-(hydroxymethyl)-4-methoxyphenyl]-4-[(3S)-3-methylmorpholin-4-yl]pyrido[3,2-e]pyrimidin-2-yl]benzoate;
[2-methoxy-5-[4-[(3S)-3-methylmorpholin-4-yl]-2-(3-nitrophenyl)pyrido[5,6-e]pyrimidin-7-yl]phenyl]methanol;
[2-methoxy-5-[4-[(3S)-3-methylmorpholin-4-yl]-2-pyridin-3-ylpyrido[5,6-e]pyrimidin-7-yl]phenyl]methanol;
3-[7-[3-(hydroxymethyl)-4-methoxyphenyl]-4-[(3S)-3-methylmorpholin-4-yl]pyrido[3,2-e]pyrimidin-2-yl]benzamide;
[2-methoxy-5-[2-[3-(methoxymethyl)phenyl]-4-[(3S)-3-methylmorpholin-4-yl]pyrido[5,6-e]pyrimidin-7-yl]phenyl]methanol;
[2-methoxy-5-[4-[(3S)-3-methylmorpholin-4-yl]-2-[3-(trifluoromethyl)phenyl]pyrido[5,6-e]pyrimidin-7-yl]phenyl]methanol;
[2-methoxy-5-[4-[(3S)-3-methylmorpholin-4-yl]-2-(3-methylphenyl)pyrido[6,5-d]pyrimidin-7-yl]phenyl]methanol;
[2-methoxy-5-[2-(3-methoxyphenyl)-4-[(3S)-3-methylmorpholin-4-yl]pyrido[5,6-e]pyrimidin-7-yl]phenyl]methanol;
[3-[2-(6-methoxypyridin-3-yl)-4-[(3S)-3-methylmorpholin-4-yl]pyrido[5,6-e]pyrimidin-7-yl]phenyl]methanol;
[3-[4-[(3S)-3-methylmorpholin-4-yl]-2-pyridin-4-ylpyrido[5,6-e]pyrimidin-7-yl]phenyl]methanol;
[3-[4-[(3S)-3-methylmorpholin-4-yl]-2-pyridin-3-ylpyrido[5,6-e]pyrimidin-7-yl]phenyl]methanol;
[3-[2-(5-methoxypyridin-3-yl)-4-[(3S)-3-methylmorpholin-4-yl]pyrido[5,6-e]pyrimidin-7-yl]phenyl]methanol;
[3-[2-(2-methoxypyridin-3-yl)-4-[(3S)-3-methylmorpholin-4-yl]pyrido[5,6-e]pyrimidin-7-yl]phenyl]methanol;
[3-[2-(2-fluoropyridin-3-yl)-4-[(3S)-3-methylmorpholin-4-yl]pyrido[5,6-e]pyrimidin-7-yl]phenyl]methanol;
[3-[4-[(3S)-3-methylmorpholin-4-yl]-2-(1-methylpyrazol-4-yl)pyrido[5,6-e]pyrimidin-7-yl]phenyl]methanol;
[3-[4-[(3S)-3-methylmorpholin-4-yl]-2-(1,3,5-trimethylpyrazol-4-yl)pyrido[6,5-d]pyrimidin-7-yl]phenyl]methanol;
7-(4-chlorophenyl)-4-[(3S)-3-methylmorpholin-4-yl]-2-pyridin-3-ylpyrido[3,2-e]pyrimidine;
N-[7-[3-(hydroxymethyl)-4-methoxyphenyl]-4-[(3S)-3-methylmorpholin-4-yl]pyrido[2,3-d]pyrimidin-2-yl]-3-methylbenzenesulfonamide;
N-[7-[3-(hydroxymethyl)-4-methoxyphenyl]-4-[(3S)-3-methylmorpholin-4-yl]pyrido[3,2-e]pyrimidin-2-yl]benzamide;
[1-[[4-[(3S)-3-methylmorpholin-4-yl]-7-phenylpyrido[3,2-e]pyrimidin-2-yl]amino]-1-oxopropan-2-yl]acetate;
[2-methoxy-5-[4-[(3S)-3-methylmorpholin-4-yl]pyrido[5,6-e]pyrimidin-7-yl]phenyl]methanol;
[2-methoxy-5-[2-[(3S)-3-methylmorpholin-4-yl]-4-morpholin-4-ylpyrido[6,5-d]pyrimidin-7-yl]phenyl]methanol;
4-[2-[(2S,6R)-2,6-dimethylmorpholin-4-yl]-4-morpholin-4-ylpyrido[6,5-d]pyrimidin-7-yl]-2-(hydroxymethyl)phenol;
[5-[2-(2-ethylpiperidin-1-yl)-4-morpholin-4-ylpyrido[6,5-d]pyrimidin-7-yl]-2-methoxyphenyl]methanol;
2-[(2S,6R)-2,6-dimethylmorpholin-4-yl]-4-morpholin-4-yl-7-phenylpyrido[2,3-d]pyrimidine;
2-[(2S,6R)-2,6-dimethylmorpholin-4-yl]-4-morpholin-4-yl-7-thiophen-3-ylpyrido[2,3-d]pyrimidine;
N-[3-[2-[(3S)-3-methylmorpholin-4-yl]-4-morpholin-4-ylpyrido[6,5-d]pyrimidin-7-yl]phenyl]methanesulfonamide;
5-[2-[(3S)-3-methylmorpholin-4-yl]-4-morpholin-4-ylpyrido[6,5-d]pyrimidin-7-yl]pyridin-2-amine; and
2-methoxy-N-methyl-5-[2-[(3S)-3-methylmorpholin-4-yl]-4-morpholin-4-ylpyrido[6,5-d]pyrimidin-7-yl]benzamide, or a pharmaceutically acceptable salt thereof.

In one embodiment the mTOR-selective inhibitor is selected from any one of
[5-[2-(2,6-dimethylmorpholin-4-yl)-4-morpholin-4-ylpyrido[6,5-d]pyrimidin-7-yl]-2-methoxyphenyl]methanol;
7-(4-chlorophenyl)-2-(2,6-dimethylmorpholin-4-yl)-4-morpholin-4-ylpyrido[2,3-d]pyrimidine;
2-(2,6-dimethylmorpholin-4-yl)-4-morpholin-4-yl-7-phenylpyrido[2,3-d]pyrimidine;
2-(2,6-dimethylmorpholin-4-yl)-7-(4-methylphenyl)-4-morpholin-4-ylpyrido[3,2-e]pyrimidine;
2-(2,6-dimethylmorpholin-4-yl)-7-(4-methoxyphenyl)-4-morpholin-4-ylpyrido[2,3-d]pyrimidine;
[2-methoxy-5-[2-(3-methylmorpholin-4-yl)-4-morpholin-4-ylpyrido[6,5-d]pyrimidin-7-yl]phenyl]methanol;
7-(4-chlorophenyl)-2-(2-methylpiperidin-1-yl)-4-morpholin-4-ylpyrido[3,2-e]pyrimidine;
2-(2-methylpiperidin-1-yl)-4-morpholin-4-yl-7-phenylpyrido[3,2-e]pyrimidine;
2-(2,6-dimethylmorpholin-4-yl)-4-morpholin-4-yl-7-thiophen-3-ylpyrido[2,3-d]pyrimidine;
[2-methoxy-5-[2-(2-methylpiperidin-1-yl)-4-morpholin-4-ylpyrido[6,5-d]pyrimidin-7-yl]phenyl]methanol;
4-[2-(2,6-dimethylmorpholin-4-yl)-4-morpholin-4-ylpyrido[6,5-d]pyrimidin-7-yl]-2,6-dimethoxyphenol;
4-[2-(2,6-dimethylmorpholin-4-yl)-4-morpholin-4-ylpyrido[5,6-e]pyrimidin-7-yl]phenol;
3-[2-(2,6-dimethylmorpholin-4-yl)-4-morpholin-4-ylpyrido[5,6-e]pyrimidin-7-yl]phenol;
4-[2-(2,6-dimethylmorpholin-4-yl)-4-morpholin-4-ylpyrido[5,6-e]pyrimidin-7-yl]benzonitrile;
2-[2-hydroxyethyl-[7-[3-(hydroxymethyl)-4-methoxyphenyl]-4-morpholin-4-ylpyrido[2,3-d]pyrimidin-2-yl]amino]propan-1-ol;
[5-[4-(2,6-dimethylmorpholin-4-yl)-2-(3-methylmorpholin-4-yl)pyrido[5,6-e]pyrimidin-7-yl]-2-methoxyphenyl]methanol;
[3-[2-(2,6-dimethylmorpholin-4-yl)-4-morpholin-4-ylpyrido[5,6-e]pyrimidin-7-yl]phenyl]methanol;
2-(2,6-dimethylmorpholin-4-yl)-7-(furan-2-yl)-4-morpholin-4-ylpyrido[2,3-d]pyrimidine;
7-(4-methylphenyl)-2-(2-methylpiperidin-1-yl)-4-morpholin-4-ylpyrido[3,2-e]pyrimidine;
2-(2,6-dimethylmorpholin-4-yl)-7-(5-methylthiophen-2-yl)-4-morpholin-4-ylpyrido[3,2-e]pyrimidine
7-(4-methoxyphenyl)-2-(2-methylpiperidin-1-yl)-4-morpholin-4-ylpyrido[3,2-e]pyrimidine;
2-(2,6-dimethylmorpholin-4-yl)-7-(4-methylthiophen-3-yl)-4-morpholin-4-ylpyrido[3,2-e]pyrimidine;
2-(2,6-dimethylmorpholin-4-yl)-7-(3-fluoro-4-methoxyphenyl)-4-morpholin-4-ylpyrido[2,3-d]pyrimidine;
2-(2,6-dimethylmorpholin-4-yl)-7-(1H-indol-5-yl)-4-morpholin-4-ylpyrido[3,2-e]pyrimidine;
2-(2,6-dimethylmorpholin-4-yl)-7-(5-methylthiophen-3-yl)-4-morpholin-4-ylpyrido[2,3-d]pyrimidine;
2-(2,6-dimethylmorpholin-4-yl)-4-morpholin-4-yl-7-(4-nitrophenyl)pyrido[3,2-e]pyrimidine;
2-(2,6-dimethylmorpholin-4-yl)-7-furan-3-yl-4-morpholin-4-ylpyrido[2,3-d]pyrimidine
N-[3-[2-(2,6-dimethylmorpholin-4-yl)-4-morpholin-4-ylpyrido[5,6-e]pyrimidin-7-yl]phenyl]acetamide;
2-(2,6-dimethylmorpholin-4-yl)-4-morpholin-4-yl-7-pyridin-4-ylpyrido[2,3-d]pyrimidine;
2-(2,6-dimethylmorpholin-4-yl)-7-(1-methylpyrazol-4-yl)-4-morpholin-4-ylpyrido[3,2-e]pyrimidine;
[5-[4-(2,6-dimethylmorpholin-4-yl)-2-morpholin-4-ylpyrido[5,6-e]pyrimidin-7-yl]-2-methoxyphenyl]methanol;
[5-[2-(bis(2-methoxyethyl)amino)-4-morpholin-4-ylpyrido[5,6-e]pyrimidin-7-yl]-2-methoxyphenyl]methanol;
1-[5-[2-(2,6-dimethylmorpholin-4-yl)-4-morpholin-4-ylpyrido[5,6-e]pyrimidin-7-yl]thiophen-2-yl]ethanone;
7-(3,4-dimethoxyphenyl)-2-(2,6-dimethylmorpholin-4-yl)-4-morpholin-4-ylpyrido[3,2-e]pyrimidine;
3-[2-(2,6-dimethylmorpholin-4-yl)-4-morpholin-4-ylpyrido[5,6-e]pyrimidin-7-yl]benzamide;
2-(2,6-dimethylmorpholin-4-yl)-7-(3-methylphenyl)-4-morpholin-4-ylpyrido[2,3-d]pyrimidine;
7-(1,3-benzodioxol-5-yl)-2-(2,6-dimethylmorpholin-4-yl)-4-morpholin-4-ylpyrido[2,3-d]pyrimidine;
7-(5-chlorothiophen-2-yl)-2-(2,6-dimethylmorpholin-4-yl)-4-morpholin-4-ylpyrido[2,3-d]pyrimidine;
methyl 3-[2-(2,6-dimethylmorpholin-4-yl)-4-morpholin-4-ylpyrido[5,6-e]pyrimidin-7-yl]benzoate;
2-(2,6-dimethylmorpholin-4-yl)-4-morpholin-4-yl-7-naphthalen-1-ylpyrido[3,2-e]pyrimidine;
2-(2,6-dimethylmorpholin-4-yl)-4-morpholin-4-yl-7-[4-(trifluoromethoxy)phenyl]pyrido[2,3-d]pyrimidine;
5-[2-(2,6-dimethylmorpholin-4-yl)-4-morpholin-4-ylpyrido[5,6-e]pyrimidin-7-yl]thiophene-2-carbaldehyde;
1-[4-[2-(2,6-dimethylmorpholin-4-yl)-4-morpholin-4-ylpyrido[5,6-e]pyrimidin-7-yl]phenyl]ethanone;
5-[2-(2,6-dimethylmorpholin-4-yl)-4-morpholin-4-ylpyrido[5,6-e]pyrimidin-7-yl]thiophene-2-carboxylic acid;
2-(2,6-dimethylmorpholin-4-yl)-4-morpholin-4-yl-7-(3-morpholin-4-ylphenyl)pyrido[2,3-d]pyrimidine;
7-(2,3-dihydro-1,4-benzodioxin-6-yl)-2-(2,6-dimethylmorpholin-4-yl)-4-morpholin-4-ylpyrido[2,3-d]pyrimidine;
3-[2-(2,6-dimethylmorpholin-4-yl)-4-morpholin-4-ylpyrido[5,6-e]pyrimidin-7-yl]benzonitrile;
2-(2,6-dimethylmorpholin-4-yl)-7-[3-(methoxymethyl)phenyl]-4-morpholin-4-ylpyrido[2,3-d]pyrimidine;
7-(1-benzothiophen-2-yl)-2-(2,6-dimethylmopholin-4-yl)-4-morpholin-4-ylpyrido[3,2-e]pyrimidine;
2-(2,6-dimethylmorpholin-4-yl)-4-morpholin-4-yl-7-pyridin-3-ylpyrido[2,3-d]pyrimidine;
2-(2,6-dimethylmorpholin-4-yl)-4-morpholin-4-yl-7-quinolin-8-ylpyrido[2,3-d]pyrimidine;
2-(2,6-dimethylmorpholin-4-yl)-4-morpholin-4-yl-7-pyrimidin-5-ylpyrido[3,2-e]pyrimidine;
7-(3,4-dihydro-2H-1,5-benzodioxepin-7-yl)-2-(2,6-dimethylmorpholin-4-yl)-4-morpholin-4-ylpyrido[2,3-d]pyrimidine;
4-[2-(2,6-dimethylmorpholin-4-yl)-4-morpholin-4-ylpyrido[5,6-e]pyrimidin-7-yl]-2-(hydroxymethyl)phenol;
[5-[2-(2-ethylpiperidin-1-yl)-4-morpholin-4-ylpyrido[5,6-e]pyrimidin-7-yl]-2-methoxyphenyl]methanol;
2-(2,6-dimethylmorpholin-4-yl)-4-morpholin-4-yl-7-(4-propan-2-yloxyphenyl)pyrido[2,3-d]pyrimidine;
7-(4-chlorophenyl)-2-(3-methylmorpholin-4-yl)-4-morpholin-4-ylpyrido[2,3-d]pyrimidine;
2-(2,6-dimethylmorpholin-4-yl)-4-morpholin-4-yl-7-(4-phenylmethoxyphenyl)pyrido[3,2-e]pyrimidine;
N-[[3-[2-(2,6-dimethylmorpholin-4-yl)-4-morpholin-4-ylpyrido[6,5-d]pyrimidin-7-yl]phenyl]methylidene]hydroxylamine;
N-[1-[3-[2-(2,6-dimethylmorpholin-4-yl)-4-morpholin-4-ylpyrido[6,5-d]pyrimidin-7-yl]phenyl]ethylidene]hydroxylamine;
[3-[2-(2,6-dimethylmorpholin-4-yl)-4-morpholin-4-ylpyrido[5,6-e]pyrimidin-7-yl]furan-2-yl]methanol;
[2-methoxy-5-(4-morpholin-4-ylpyrido[6,5-d]pyrimidin-7-yl)phenyl]methanol;
7-[4-(cyclohexylmethoxy)phenyl]-2-(2,6-dimethylmorpholin-4-yl)-4-morpholin-4-ylpyrido[3,2-e]pyrimidine;
7-[4-(1-cyclohex-3-enyloxy)phenyl]-2-(2,6-dimethylmorpholin-4-yl)-4-morpholin-4-ylpyrido[2,3-d]pyrimidine;
3-[4-[2-(2,6-dimethylmorpholin-4-yl)-4-morpholin-4-ylpyrido[5,6-e]pyrimidin-7-yl]phenoxy]propan-1-ol;
2-(2,6-dimethylmorpholin-4-yl)-4-morpholin-4-yl-7-(4-propoxyphenyl)pyrido[3,2-e]pyrimidine;
2-(2,6-dimethylmorpholin-4-yl)-7-[4-(2,2-dimethylpropoxy)phenyl]-4-morpholin-4-ylpyrido[2,3-d]pyrimidine;
3-[4-[2-(2,6-dimethylmorpholin-4-yl)-4-morpholin-4-ylpyrido[5,6-e]pyrimidin-7-yl]phenoxy]-2,2-dimethylpropan-1-ol;
7-(4-but-2-enoxyphenyl)-2-(2,6-dimethylmorpholin-4-yl)-4-morpholin-4-ylpyrido[2,3-d]pyrimidine;
2-(2,6-dimethylmorpholin-4-yl)-7-(4-ethoxyphenyl)-4-morpholin-4-ylpyrido[2,3-d]pyrimidine;
7-(4-chlorophenyl)-2-(2-ethylpiperidin-1-yl)-4-morpholin-4-ylpyrido[3,2-e]pyrimidine;
2-(2,6-dimethylmorpholin-4-yl)-4-morpholin-4-yl-7-[4-(oxolan-2-ylmethoxy)phenyl]pyrido[2,3-d]pyrimidine;
7-(4-chlorophenyl)-2-(2-methylpiperidin-1-yl)-4-morpholin-4-ylpyrido[3,2-e]pyrimidine;
2-(2,6-dimethylmorpholin-4-yl)-4-morpholin-4-yl-7-[4-(1-phenylethoxy)phenyl]pyrido[3,2-e]pyrimidine;
7-[4-[(3,4-difluorophenyl)methoxy]phenyl]-2-(2,6-dimethylmorpholin-4-yl)-4-morpholin-4-ylpyrido[3,2-e]pyrimidine;
2-(2,6-dimethylmorpholin-4-yl)-7-[4-(4-methylpentoxy)phenyl]-4-morpholin-4-ylpyrido[3,2-e]pyrimidine;
2-(2,6-dimethylmorpholin-4-yl)-4-morpholin-4-yl-N-phenylpyrido[6,5-d]pyrimidin-7-amine;
2-(2,6-dimethylmorpholin-4-yl)-4-morpholin-4-yl-N-(3-phenoxyphenyl)pyrido[5,6-e]pyrimidin-7-amine;
4-[[2-(2,6-dimethylmorpholin-4-yl)-4-morpholin-4-ylpyrido[6,5-d]pyrimidin-7-yl]amino]-3-hydroxybenzoic acid;
2-(2,6-dimethylmorpholin-4-yl)-N,N-dimethyl-4-morpholin-4-ylpyrido[5,6-e]pyrimidin-7-amine;
2-(2,6-dimethylmorpholin-4-yl)-N-methyl-4-morpholin-4-ylpyrido[5,6-e]pyrimidin-7-amine;
2-(2,6-dimethylmorpholin-4-yl)-N-isoquinolin-5-yl-4-morpholin-4-ylpyrido[5,6-e]pyrimidin-7-amine;
methyl 4-[[2-(2,6-dimethylmorpholin-4-yl)-4-morpholin-4-ylpyrido[6,5-d]pyrimidin-7-yl]amino]benzoate;
4-[[2-(2,6-dimethylmorpholin-4-yl)-4-morpholin-4-ylpyrido[6,5-d]pyrimidin-7-yl]amino]benzonitrile;
2-(2-methylpiperidin-1-yl)-4-morpholin-4-yl-N-phenylpyrido[5,6-e]pyrimidin-7-amine;
2-(2,6-dimethylmorpholin-4-yl)-N-(4-methylphenyl)-4-morpholin-4-ylpyrido[5,6-e]pyrimidin-7-amine;
N-(4-chlorophenyl)-2-(2,6-dimethylmorpholin-4-yl)-4-morpholin-4-ylpyrido[5,6-e]pyrimidin-7-amine;
N-(2-chloro-6-methyl-4-nitrophenyl)-2-(2,6-dimethylmorpholin-4-yl)-4-morpholin-4-ylpyrido[6,5-d]pyrimidin-7-amine;
[3-[[2-(2,6-dimethylmorpholin-4-yl)-4-morpholin-4-ylpyrido[6,5-d]pyrimidin-7-yl]amino]phenyl]-phenylmethanone;
2-(2,6-dimethylmorpholin-4-yl)-4-morpholin-4-yl-N-(3-propan-2-ylphenyl)pyrido[5,6-e]pyrimidin-7-amine;
2-(2,6-dimethylmorpholin-4-yl)-4-morpholin-4-yl-N-quinolin-8-ylpyrido[5,6-e]pyrimidin-7-amine;
2-(2,6-dimethylmorpholin-4-yl)-N-[4-[2-(4-methoxyphenyl)ethenyl]phenyl]-4-morpholin-4-ylpyrido[5,6-e]pyrimidin-7-amine;
2-(2,6-dimethylmorpholin-4-yl)-4-morpholin-4-yl-N-(4-pyrrol-1-ylphenyl)pyrido[5,6-e]pyrimidin-7-amine;
2-(2,6-dimethylmorpholin-4-yl)-4-morpholin-4-yl-N-(1,2,4-triazol-4-yl)pyrido[5,6-e]pyrimidin-7-amine;
N-[4-(2-chlorophenyl)-1,3-thiazol-2-yl]-2-(2,6-dimethylmorpholin-4-yl)-4-morpholin-4-ylpyrido[5,6-e]pyrimidin-7-amine;
2-(2,6-dimethylmorpholin-4-yl)-N-(3-methylpyridin-4-yl)-4-morpholin-4-ylpyrido[6,5-d]pyrimidin-7-amine;
N-(4,6-difluoropyrimidin-2-yl)-2-(2,6-dimethylmorpholin-4-yl)-4-morpholin-4-ylpyrido[5,6-e]pyrimidin-7-amine;
2-(2,6-dimethylmorpholin-4-yl)-N-(2-methylprop-2-enyl)-4-morpholin-4-ylpyrido[5,6-e]pyrimidin-7-amine;
2-(2,6-dimethylmorpholin-4-yl)-N-(1-methylpiperidin-4-yl)-4-morpholin-4-ylpyrido[6,5-d]pyrimidin-7-amine;
2-(2,6-dimethylmorpholin-4-yl)-N-(5-methyl-1,2-oxazol-3-yl)-4-morpholin-4-ylpyrido[5,6-e]pyrimidin-7-amine;
2-(2,6-dimethylmorpholin-4-yl)-N-[5-methyl-2-(4-methylphenyl)pyrazol-3-yl]-4-morpholin-4-ylpyrido[5,6-e]pyrimidin-7-amine;
N-(3,4-difluorophenyl)-2-(2,6-dimethylmorpholin-4-yl)-4-morpholin-4-ylpyrido[6,5-d]pyrimidin-7-amine;
5-chloro-2-[[2-(2,6-dimethylmorpholin-4-yl)-4-morpholin-4-ylpyrido[5,6-e]pyrimidin-7-yl]amino]phenol;
N-[2-(2,6-dimethylmorpholin-4-yl)-4-morpholin-4-ylpyrido[5,6-e]pyrimidin-7-yl]-N′,N′-diethylbenzene-1,4-diamine;
N-(4,5-difluoro-2-nitrophenyl)-2-(2,6-dimethylmorpholin-4-yl)-4-morpholin-4-ylpyrido[5,6-e]pyrimidin-7-amine;
2-(2,6-dimethylmorpholin-4-yl)-N-(2-methylsulfanyl-1,3-benzothiazol-6-yl)-4-morpholin-4-ylpyrido[6,5-d]pyrimidin-7-amine;
2-(2,6-dimethylmorpholin-4-yl)-N-[4-(1,3-dithiolan-2-yl)phenyl]-4-morpholin-4-ylpyrido[5,6-e]pyrimidin-7-amine;
N-cyclohexyl-2-(2,6-dimethylmorpholin-4-yl)-4-morpholin-4-ylpyrido[6,5-d]pyrimidin-7-amine;
N-cyclopentyl-2-(2,6-dimethylmorpholin-4-yl)-4-morpholin-4-ylpyrido[6,5-d]pyrimidin-7-amine;
N-cyclopropyl-2-(2,6-dimethylmorpholin-4-yl)-4-morpholin-4-ylpyrido[5,6-e]pyrimidin-7-amine;
N-cyclobutyl-2-(2,6-dimethylmorpholin-4-yl)-4-morpholin-4-ylpyrido[5,6-e]pyrimidin-7-amine;
N-(cyclopropylmethyl)-2-(2,6-dimethylmorpholin-4-yl)-4-morpholin-4-ylpyrido[6,5-d]pyrimidin-7-amine;
2-(2,6-dimethylmorpholin-4-yl)-4-morpholin-4-yl-N-propan-2-ylpyrido[5,6-e]pyrimidin-7-amine;
2-(2,6-dimethylmorpholin-4-yl)-N-(4-methylcyclohexyl)-4-morpholin-4-ylpyrido[5,6-e]pyrimidin-7-amine;
2-(2,6-dimethylmorpholin-4-yl)-4-morpholin-4-yl-N-prop-2-enylpyrido[5,6-e]pyrimidin-7-amine;
methyl 5-[[2-(2,6-dimethylmorpholin-4-yl)-4-morpholin-4-ylpyrido[5,6-e]pyrimidin-7-yl]amino]furan-2-carboxylate;
2-(2,6-dimethylmorpholin-4-yl)-N-(5-methyl-1,3,4-oxadiazol-2-yl)-4-morpholin-4-ylpyrido[5,6-e]pyrimidin-7-amine;
2-[[2-(2,6-dimethylmorpholin-4-yl)-4-morpholin-4-ylpyrido[6,5-d]pyrimidin-7-yl]amino]-5-phenylfuran-3-carbonitrile;
2-(2,6-dimethylmorpholin-4-yl)-4-morpholin-4-yl-N-thiophen-3-ylpyrido[5,6-e]pyrimidin-7-amine;
5-[[2-(2,6-dimethylmorpholin-4-yl)-4-morpholin-4-ylpyrido[5,6-e]pyrimidin-7-yl]amino]-3H-imidazole-4-carbonitrile;
methyl 4-[[2-(2,6-dimethylmorpholin-4-yl)-4-morpholin-4-ylpyrido[6,5-d]pyrimidin-7-yl]amino]-1-methylpyrrole-2-carboxylate;
N-(2,6-dimethoxypyridin-3-yl)-2-(2,6-dimethylmorpholin-4-yl)-4-morpholin-4-ylpyrido[5,6-e]pyrimidin-7-amine;
2-(2,6-dimethylmorpholin-4-yl)-4-morpholin-4-yl-N-(9H-purin-8-yl)pyrido[6,5-d]pyrimidin-7-amine;
2-(2,6-dimethylmorpholin-4-yl)-4-morpholin-4-yl-N-(1,7-naphthyridin-8-yl)pyrido[5,6-e]pyrimidin-7-amine;
2-(2,6-dimethylmorpholin-4-yl)-4-morpholin-4-yl-N-(2-pyridin-3-ylpyrimidin-4-yl)pyrido[5,6-e]pyrimidin-7-amine;
2-(2,6-dimethylmorpholin-4-yl)-N-[4-(furan-2-yl)pyrimidin-2-yl]-4-morpholin-4-ylpyrido[6,5-d]pyrimidin-7-amine;
2-[[2-(2,6-dimethylmorpholin-4-yl)-4-morpholin-4-ylpyrido[6,5-d]pyrimidin-7-yl]amino]quinolin-8-ol;
2-(2,6-dimethylmorpholin-4-yl)-4-morpholin-4-yl-N-pyridin-3-ylpyrido[6,5-d]pyrimidin-7-amine;
2-(2,6-dimethylmorpholin-4-yl)-4-morpholin-4-yl-N-pyridin-4-ylpyrido[6,5-d]pyrimidin-7-amine;
2-(2,6-dimethylmorpholin-4-yl)-4-morpholin-4-yl-N-pyrimidin-4-ylpyrido[6,5-d]pyrimidin-7-amine;
2-(2,6-dimethylmorpholin-4-yl)-4-morpholin-4-yl-N-pyrazin-2-ylpyrido[6,5-d]pyrimidin-7-amine;
2-(2,6-dimethylmorpholin-4-yl)-N-(5-methylpyridin-2-yl)-4-morpholin-4-ylpyrido[5,6-e]pyrimidin-7-amine;
2-(2,6-dimethylmorpholin-4-yl)-N-(6-methylpyridin-2-yl)-4-morpholin-4-ylpyrido[5,6-e]pyrimidin-7-amine;
N-[2-(2,6-dimethylmorpholin-4-yl)-4-morpholin-4-ylpyrido[6,5-d]pyrimidin-7-yl]-6,6-dimethyl-1,5,7,8-tetrahydropyrazolo[5,4-b]quinolin-3-amine;
2-(2,6-dimethylmorpholin-4-yl)-N-(3-methylpyridin-2-yl)-4-morpholin-4-ylpyrido[5,6-e]pyrimidin-7-amine;
7-(2,6-dimethylmorpholin-4-yl)-4-morpholin-4-ylpyrido[3,2-e]pyrimidine;
2-(2,6-dimethylmorpholin-4-yl)-4-morpholin-4-ylpyrido[6,5-d]pyrimidin-7-amine;
2-(2-methylpiperidin-1-yl)-4-morpholin-4-ylpyrido[5,6-e]pyrimidin-7-amine;
3-[2-(2,6-dimethylmorpholin-4-yl)-4-morpholin-4-ylpyrido[5,6-e]pyrimidin-7-yl]-1-ethylurea;
N-[2-(2,6-dimethylmorpholin-4-yl)-4-morpholin-4-ylpyrido[6,5-d]pyrimidin-7-yl]benzamide;
N-[2-(2,6-dimethylmorpholin-4-yl)-4-morpholin-4-ylpyrido[5,6-e]pyrimidin-7-yl]cyclopentanecarboxamide;
N-[2-(2,6-dimethylmorpholin-4-yl)-4-morpholin-4-ylpyrido[6,5-d]pyrimidin-7-yl]but-2-enamide;
N-[2-(2,6-dimethylmorpholin-4-yl)-4-morpholin-4-ylpyrido[5,6-e]pyrimidin-7-yl]-2-phenylsulfanylacetamide;
N-[2-(2,6-dimethylmorpholin-4-yl)-4-morpholin-4-ylpyrido[5,6-e]pyrimidin-7-yl]quinoxaline-2-carboxamide;
N-[2-(2,6-dimethylmorpholin-4-yl)-4-morpholin-4-ylpyrido[5,6-e]pyrimidin-7-yl]acetamide;
5-amino-N-[2-(2,6-dimethylmorpholin-4-yl)-4-morpholin-4-ylpyrido[5,6-e]pyrimidin-7-yl]-1H-imidazole-4-carboxamide;
N-[2-(2,6-dimethylmorpholin-4-yl)-4-morpholin-4-ylpyrido[5,6-e]pyrimidin-7-yl]methanesulfonamide;
7-butoxy-2-(2,6-dimethylmorpholin-4-yl)-4-morpholin-4-ylpyrido[3,2-e]pyrimidine;
N-[[5-[2-(2,6-dimethylmorpholin-4-yl)-4-morpholin-4-ylpyrido[6,5-d]pyrimidin-7-yl]-2-methoxyphenyl]methyl]cyclobutanamine;
N-[[5-[2-(2,6-dimethylmorpholin-4-yl)-4-morpholin-4-ylpyrido[5,6-e]pyrimidin-7-yl]-2-methoxyphenyl]methyl]cyclopropanamine;
N-[[5-[2-(2,6-dimethylmorpholin-4-yl)-4-morpholin-4-ylpyrido[5,6-e]pyrimidin-7-yl]-2-methoxyphenyl]methyl]-2-(3H-imidazol-4-yl)ethanamine;
2-[[5-[2-(2,6-dimethylmorpholin-4-yl)-4-morpholin-4-ylpyrido[5,6-e]pyrimidin-7-yl]-2-methoxyphenyl]methylamino]ethanol;
N-[[5-[2-(2,6-dimethylmorpholin-4-yl)-4-morpholin-4-ylpyrido[6,5-d]pyrimidin-7-yl]-2-methoxyphenyl]methyl]-2-(4-methoxyphenyl)ethanamine;
1-[5-[2-(2,6-dimethylmorpholin-4-yl)-4-morpholin-4-ylpyrido[6,5-d]pyrimidin-7-yl]-2-methoxyphenyl]-N-methylmethanamine;
N-(cyclopropylmethyl)-1-[5-[2-(2,6-dimethylmorpholin-4-yl)-4-morpholin-4-ylpyrido[5,6-e]pyrimidin-7-yl]-2-methoxyphenyl]methanamine;
N-[[5-[2-(2,6-dimethylmorpholin-4-yl)-4-morpholin-4-ylpyrido[5,6-e]pyrimidin-7-yl]-2-methoxyphenyl]methyl]-N′,N′-diethylpropane-1,3-diamine;
N-[[5-[2-(2,6-dimethylmorpholin-4-yl)-4-morpholin-4-ylpyrido[5,6-e]pyrimidin-7-yl]-2-methoxyphenyl]methyl]oxan-4-amine;
N-[[5-[2-(2,6-dimethylmorpholin-4-yl)-4-morpholin-4-ylpyrido[6,5-d]pyrimidin-7-yl]-2-methoxyphenyl]methyl]-1-thiophen-2-ylmethanamine;
N-[[5-[2-(2,6-dimethylmorpholin-4-yl)-4-morpholin-4-ylpyrido[6,5-d]pyrimidin-7-yl]-2-methoxyphenyl]methyl]cyclopentanamine;
N-[[5-[2-(2,6-dimethylmorpholin-4-yl)-4-morpholin-4-ylpyrido[5,6-e]pyrimidin-7-yl]-2-methoxyphenyl]methyl]butan-1-amine;
N-[[5-[2-(2,6-dimethylmorpholin-4-yl)-4-morpholin-4-ylpyrido[6,5-d]pyrimidin-7-yl]-2-methoxyphenyl]methyl]-3-methylbutan-1-amine;
N-[[5-[2-(2,6-dimethylmorpholin-4-yl)-4-morpholin-4-ylpyrido[5,6-e]pyrimidin-7-yl]-2-methoxyphenyl]methyl]cyclooctanamine;
N-[[5-[2-(2,6-dimethylmorpholin-4-yl)-4-morpholin-4-ylpyrido[5,6-e]pyrimidin-7-yl]-2-methoxyphenyl]methyl]cyclohexanamine;
N-[[5-[2-(2,6-dimethylmorpholin-4-yl)-4-morpholin-4-ylpyrido[5,6-e]pyrimidin-7-yl]-2-methoxyphenyl]methyl]-4-methylcyclohexan-1-amine;
N-[[5-[2-(2,6-dimethylmorpholin-4-yl)-4-morpholin-4-ylpyrido[5,6-e]pyrimidin-7-yl]-2-methoxyphenyl]methyl]-3,3-dimethylbutan-1-amine;
N-[[5-[2-(2,6-dimethylmorpholin-4-yl)-4-morpholin-4-ylpyrido[5,6-e]pyrimidin-7-yl]-2-methoxyphenyl]methyl]propan-2-amine;
N-[[5-[2-(2,6-dimethylmorpholin-4-yl)-4-morpholin-4-ylpyrido[5,6-e]pyrimidin-7-yl]-2-methoxyphenyl]methyl]-3-ethoxypropan-1-amine;
N-[[5-[2-(2,6-dimethylmorpholin-4-yl)-4-morpholin-4-ylpyrido[5,6-e]pyrimidin-7-yl]-2-methoxyphenyl]methyl]-4-methylpentan-2-amine;
N-[[5-[2-(2,6-dimethylmorpholin-4-yl)-4-morpholin-4-ylpyrido[5,6-e]pyrimidin-7-yl]-2-methoxyphenyl]methyl]-2-methylpropan-1-amine;
N-[[5-[2-(2,6-dimethylmorpholin-4-yl)-4-morpholin-4-ylpyrido[5,6-e]pyrimidin-7-yl]-2-methoxyphenyl]methyl]-2-methoxyethanamine;
N-[[5-[2-(2,6-dimethylmorpholin-4-yl)-4-morpholin-4-ylpyrido[6,5-d]pyrimidin-7-yl]-2-methoxyphenyl]methyl]-2-methylcyclohexan-1-amine;
N-[[5-[2-(2,6-dimethylmorpholin-4-yl)-4-morpholin-4-ylpyrido[6,5-d]pyrimidin-7-yl]-2-methoxyphenyl]methyl]-1-ethynylcyclohexan-1-amine;
2-[[5-[2-(2,6-dimethylmorpholin-4-yl)-4-morpholin-4-ylpyrido[5,6-e]pyrimidin-7-yl]-2-methoxyphenyl]methylamino]-3-(4-nitrophenyl)propanoic acid;
1-(2,3-dimethoxyphenyl)-N-[[5-[2-(2,6-dimethylmorpholin-4-yl)-4-morpholin-4-ylpyrido[5,6-e]pyrimidin-7-yl]-2-methoxyphenyl]methyl]methanamine;
2-[[[5-[2-(2,6-dimethylmorpholin-4-yl)-4-morpholin-4-ylpyrido[5,6-e]pyrimidin-7-yl]-2-methoxyphenyl]methylamino]methyl]cyclohexan-1-ol;
2-[[[5-[2-(2,6-dimethylmorpholin-4-yl)-4-morpholin-4-ylpyrido[6,5-d]pyrimidin-7-yl]-2-methoxyphenyl]methylamino]methyl]cycloheptan-1-ol;
ethyl 2-[[5-[2-(2,6-dimethylmorpholin-4-yl)-4-morpholin-4-ylpyrido[5,6-e]pyrimidin-7-yl]-2-methoxyphenyl]methylamino]cyclohexane-1-carboxylate;
2-[2-[[5-[2-(2,6-dimethylmorpholin-4-yl)-4-morpholin-4-ylpyrido[5,6-e]pyrimidin-7-yl]-2-methoxyphenyl]methylamino]ethylsulfanyl]ethanol;
2-[[5-(dimethylaminomethyl)furan-2-yl]methylsulfanyl]-N-[[5-[2-(2,6-dimethylmorpholin-4-yl)-4-morpholin-4-ylpyrido[6,5-d]pyrimidin-7-yl]-2-methoxyphenyl]methyl]ethanamine;
prop-2-enyl 2-[[5-[2-(2,6-dimethylmorpholin-4-yl)-4-morpholin-4-ylpyrido[5,6-e]pyrimidin-7-yl]-2-methoxyphenyl]methylamino]-4-methylpentanoate;
N-[[5-[2-(2,6-dimethylmorpholin-4-yl)-4-morpholin-4-ylpyrido[5,6-e]pyrimidin-7-yl]-2-methoxyphenyl]methyl]-2,3-dimethylcyclohexan-1-amine;
N-[[5-[2-(2,6-dimethylmorpholin-4-yl)-4-morpholin-4-ylpyrido[6,5-d]pyrimidin-7-yl]-2-methoxyphenyl]methyl]-1-(4-fluorophenyl)methanamine;
N-[[5-[2-(2,6-dimethylmorpholin-4-yl)-4-morpholin-4-ylpyrido[5,6-e]pyrimidin-7-yl]-2-methoxyphenyl]methyl]-2-(1-methylpyrrolidin-2-yl)ethanamine;
1-[5-[2-(2,6-dimethylmorpholin-4-yl)-4-morpholin-4-ylpyrido[6,5-d]pyrimidin-7-yl]-2-methoxyphenyl]-N-(oxolan-2-ylmethyl)methanamine;
N-[[5-[2-(2,6-dimethylmorpholin-4-yl)-4-morpholin-4-ylpyrido[5,6-e]pyrimidin-7-yl]-2-methoxyphenyl]methyl]-2-ethylsulfanylethanamine;
N-[[5-[2-(2,6-dimethylmorpholin-4-yl)-4-morpholin-4-ylpyrido[5,6-e]pyrimidin-7-yl]-2-methoxyphenyl]methyl]-1H-pyrazol-3-amine;
N-[[5-[2-(2,6-dimethylmorpholin-4-yl)-4-morpholin-4-ylpyrido[5,6-e]pyrimidin-7-yl]-2-methoxyphenyl]methyl]piperidine-1-carbothioamide;
N-[[5-[2-(2,6-dimethylmorpholin-4-yl)-4-morpholin-4-ylpyrido[5,6-e]pyrimidin-7-yl]-2-methoxyphenyl]methyl]-2-thiophen-2-ylethanamine;
methyl 2-[[5-[2-(2,6-dimethylmorpholin-4-yl)-4-morpholin-4-ylpyrido[5,6-e]pyrimidin-7-yl]-2-methoxyphenyl]methylamino]-2-phenylacetate;
[3-[[[5-[2-(2,6-dimethylmorpholin-4-yl)-4-morpholin-4-ylpyrido[5,6-e]pyrimidin-7-yl]-2-methoxyphenyl]methylamino]methyl]phenyl]methanamine;
2-[[5-[2-(2,6-dimethylmorpholin-4-yl)-4-morpholin-4-ylpyrido[6,5-d]pyrimidin-7-yl]-2-methoxyphenyl]methylamino]propan-1-ol;
2-[[5-[2-(2,6-dimethylmorpholin-4-yl)-4-morpholin-4-ylpyrido[6,5-d]pyrimidin-7-yl]-2-methoxyphenyl]methylamino]-3-(4-hydroxyphenyl)propanoic acid;
tert-butyl N-[5-[[5-[2-(2,6-dimethylmorpholin-4-yl)-4-morpholin-4-ylpyrido[6,5-d]pyrimidin-7-yl]-2-methoxyphenyl]methylamino]pentyl]carbamate;
N-[[5-[2-(2,6-dimethylmorpholin-4-yl)-4-morpholin-4-ylpyrido[5,6-e]pyrimidin-7-yl]-2-methoxyphenyl]methyl]-1-(3-nitrophenyl)methanamine;
N-[[5-[2-(2,6-dimethylmorpholin-4-yl)-4-morpholin-4-ylpyrido[6,5-d]pyrimidin-7-yl]-2-methoxyphenyl]methyl]-1-(4-nitrophenyl)ethanamine;
N-[[5-[2-(2,6-dimethylmorpholin-4-yl)-4-morpholin-4-ylpyrido[5,6-e]pyrimidin-7-yl]-2-methoxyphenyl]methyl]-2,3-dihydro-1H-inden-1-amine;
1-(2,4-dichlorophenyl)-N-[[5-[2-(2,6-dimethylmorpholin-4-yl)-4-morpholin-4-ylpyrido[5,6-e]pyrimidin-7-yl]-2-methoxyphenyl]methyl]methanamine;
1-(2-chlorophenyl)-N-[[5-[2-(2,6-dimethylmorpholin-4-yl)-4-morpholin-4-ylpyrido[5,6-e]pyrimidin-7-yl]-2-methoxyphenyl]methyl]methanamine;
N-[[5-[2-(2,6-dimethylmorpholin-4-yl)-4-morpholin-4-ylpyrido[5,6-e]pyrimidin-7-yl]-2-methoxyphenyl]methyl]-4-morpholin-4-ylaniline;
N-[[5-[2-(2,6-dimethylmorpholin-4-yl)-4-morpholin-4-ylpyrido[5,6-e]pyrimidin-7-yl]-2-methoxyphenyl]methyl]-1,1-diphenylmethanamine;
N-[[5-[2-(2,6-dimethylmorpholin-4-yl)-4-morpholin-4-ylpyrido[5,6-e]pyrimidin-7-yl]-2-methoxyphenyl]methyl]-4-phenylmethoxyaniline;
3-[2-[[5-[2-(2,6-dimethylmorpholin-4-yl)-4-morpholin-4-ylpyrido[5,6-e]pyrimidin-7-yl]-2-methoxyphenyl]methylamino]-1-hydroxyethyl]phenol;
N-[[5-[2-(2,6-dimethylmorpholin-4-yl)-4-morpholin-4-ylpyrido[5,6-e]pyrimidin-7-yl]-2-methoxyphenyl]methyl]-3-phenylpropan-1-amine;
2-[[5-[2-(2,6-dimethylmorpholin-4-yl)-4-morpholin-4-ylpyrido[6,5-d]pyrimidin-7-yl]-2-methoxyphenyl]methylamino]-4-methylsulfanylbutan-1-ol;
N-[[5-[2-(2,6-dimethylmorpholin-4-yl)-4-morpholin-4-ylpyrido[5,6-e]pyrimidin-7-yl]-2-methoxyphenyl]methyl]-1-methoxy-3-phenylpropan-2-amine;
N-[[5-[2-(2,6-dimethylmorpholin-4-yl)-4-morpholin-4-ylpyrido[6,5-d]pyrimidin-7-yl]-2-methoxyphenyl]methyl]-4-pentoxyaniline;
1-(1,3-benzodioxol-5-yl)-N-[[5-[2-(2,6-dimethylmorpholin-4-yl)-4-morpholin-4-ylpyrido[5,6-e]pyrimidin-7-yl]-2-methoxyphenyl]methyl]methanamine;
3-amino-5-[7-[3-(hydroxymethyl)-4-methoxyphenyl]-4-morpholin-4-ylpyrido[2,3-d]pyrimidin-2-yl]benzoic acid;
3-[7-[3-(hydroxymethyl)-4-methoxyphenyl]-4-morpholin-4-ylpyrido[2,3-d]pyrimidin-2-yl]-5-nitrobenzoic acid;
[2-methoxy-5-[4-morpholin-4-yl-2-(3-nitrophenyl)pyrido[5,6-e]pyrimidin-7-yl]phenyl]methanol;
[2-methoxy-5-[2-(3-methylphenyl)-4-morpholin-4-ylpyrido[5,6-e]pyrimidin-7-yl]phenyl]methanol;
[2-methoxy-5-[2-(3-methoxyphenyl)-4-morpholin-4-ylpyrido[6,5-d]pyrimidin-7-yl]phenyl]methanol;
[5-[2-(furan-2-yl)-4-morpholin-4-ylpyrido[5,6-e]pyrimidin-7-yl]-2-methoxyphenyl]methanol;
3-[7-[3-(hydroxymethyl)-4-methoxyphenyl]-4-morpholin-4-ylpyrido[3,2-e]pyrimidin-2-yl]benzaldehyde;
[2-methoxy-5-[2-[3-(methoxymethyl)phenyl]-4-morpholin-4-ylpyrido[5,6-e]pyrimidin-7-yl]phenyl]methanol;
3-[7-[3-(hydroxymethyl)-4-methoxyphenyl]-4-morpholin-4-ylpyrido[2,3-d]pyrimidin-2-yl]phenol;
3-[7-[3-(hydroxymethyl)-4-methoxyphenyl]-4-morpholin-4-ylpyrido[2,3-d]pyrimidin-2-yl]benzonitrile;
3-[7-[3-(hydroxymethyl)-4-methoxyphenyl]-4-morpholin-4-ylpyrido[2,3-d]pyrimidin-2-yl]benzamide;
[5-[2-(3-aminophenyl)-4-morpholin-4-ylpyrido[5,6-e]pyrimidin-7-yl]-2-methoxyphenyl]methanol;
[3-[7-(4-chlorophenyl)-4-morpholin-4-ylpyrido[3,2-e]pyrimidin-2-yl]phenyl]methanol;
[5-[2-[3-(aminomethyl)phenyl]-4-morpholin-4-ylpyrido[5,6-e]pyrimidin-7-yl]-2-methoxyphenyl]methanol;
[2-methoxy-5-[4-morpholin-4-yl-2-(3-phenylphenyl)pyrido[5,6-e]pyrimidin-7-yl]phenyl]methanol;
7-(4-chlorophenyl)-4-morpholin-4-yl-2-[3-(trifluoromethyl)phenyl]pyrido[3,2-e]pyrimidine;
[5-[2-[3-(hydroxymethyl)-4-methoxyphenyl]-4-morpholin-4-ylpyrido[5,6-e]pyrimidin-7-yl]-2-methoxyphenyl]methanol;
[2-methoxy-5-[4-morpholin-4-yl-2-[3-(trifluoromethyl)phenyl]pyrido[5,6-e]pyrimidin-7-yl]phenyl]methanol;
2-(4-chlorophenyl)-7-(2,6-dimethylmorpholin-4-yl)-4-morpholin-4-ylpyrido[2,3-d]pyrimidine;
7-(4-chlorophenyl)-4-morpholin-4-yl-2-pyridin-3-ylpyrido[3,2-e]pyrimidine;
7-(4-chlorophenyl)-2-(3-methoxyphenyl)-4-morpholin-4-ylpyrido[3,2-e]pyrimidine;
7-(4-chlorophenyl)-4-morpholin-4-yl-2-(3-nitrophenyl)pyrido[2,3-d]pyrimidine;
3-[7-(4-chlorophenyl)-4-morpholin-4-ylpyrido[3,2-e]pyrimidin-2-yl]benzaldehyde;
7-(4-chlorophenyl)-2-(furan-2-yl)-4-morpholin-4-ylpyrido[3,2-e]pyrimidine;
7-(4-chlorophenyl)-2-(3-methylphenyl)-4-morpholin-4-ylpyrido[2,3-d]pyrimidine;
7-(4-chlorophenyl)-2-[3-(methoxymethyl)phenyl]-4-morpholin-4-ylpyrido[2,3-d]pyrimidine;
7-(4-chlorophenyl)-4-morpholin-4-yl-2-(3-phenylphenyl)pyrido[3,2-e]pyrimidine;
3-[7-(4-chlorophenyl)-4-morpholin-4-ylpyrido[2,3-d]pyrimidin-2-yl]benzonitrile;
3-[7-(4-chlorophenyl)-4-morpholin-4-ylpyrido[2,3-d]pyrimidin-2-yl]benzamide;
methyl 3-[7-(4-chlorophenyl)-4-morpholin-4-ylpyrido[3,2-e]pyrimidin-2-yl]benzoate; and
[3-[7-[3-(hydroxymethyl)-4-methoxyphenyl]-4-morpholin-4-ylpyrido[2,3-d]pyrimidin-2-yl]phenyl]methanol, or a pharmaceutically acceptable salt thereof.

In one embodiment the mTOR-selective inhibitor is selected from any one of
5-[2,4-bis[(3S)-3-methylmorpholin-4-yl]pyrido[5,6-e]pyrimidin-7-yl]-2-methoxy-N-methylbenzamide;
4-[2,4-bis[(3S)-3-methylmorpholin-4-yl]pyrido[5,6-e]pyrimidin-7-yl]aniline;
6-[2,4-bis[(3S)-3-methylmorpholin-4-yl]pyrido[5,6-e]pyrimidin-7-yl]-1H-indazol-3-amine;
8-[2,4-bis[(3S)-3-methylmorpholin-4-yl]pyrido[5,6-e]pyrimidin-7-yl]-1,2,3,4-tetrahydro-1,4-benzodiazepin-5-one;
5-[2,4-bis[(3S)-3-methylmorpholin-4-yl]pyrido[5,6-e]pyrimidin-7-yl]-2-methoxy-N-methylbenzamide;
5-[2,4-bis[(3S)-3-methylmorpholin-4-yl]pyrido[5,6-e]pyrimidin-7-yl]pyridin-2-amine;
N-[3-[2,4-bis[(3S)-3-methylmorpholin-4-yl]pyrido[5,6-e]pyrimidin-7-yl]phenyl]methanesulfonamide;
3-[2,4-bis[(3S)-3-methylmorpholin-4-yl]pyrido[5,6-e]pyrimidin-7-yl]aniline;
5-[2,4-bis[(3S)-3-methylmorpholin-4-yl]pyrido[6,5-d]pyrimidin-7-yl]-2-ethoxybenzamide;
5-[2,4-bis[(3S)-3-methylmorpholin-4-yl]pyrido[5,6-e]pyrimidin-7-yl]-2-(difluoromethoxy)-N-methylbenzamide;
5-[2,4-bis[(3S)-3-methylmorpholin-4-yl]pyrido[5,6-e]pyrimidin-7-yl]-1H-indazol-3-amine;
[5-[2,4-bis[(3S)-3-methylmorpholin-4-yl]pyrido[5,6-e]pyrimidin-7-yl]-2-methoxyphenyl]methanol;
N-[[4-[2,4-bis[(3S)-3-methylmorpholin-4-yl]pyrido[5,6-e]pyrimidin-7-yl]phenyl]methyl]methanesulfonamide;
5-[2,4-bis[(3S)-3-methylmorpholin-4-yl]pyrido[5,6-e]pyrimidin-7-yl]-1,3-dihydroindol-2-one;
6-[2,4-bis[(3S)-3-methylmorpholin-4-yl]pyrido[5,6-e]pyrimidin-7-yl]-1,3-dihydroindol-2-one;
3-[2,4-bis[(3S)-3-methylmorpholin-4-yl]pyrido[5,6-e]pyrimidin-7-yl]-N-methylbenzamide;
5-[2,4-bis[(3S)-3-methylmorpholin-4-yl]pyrido[5,6-e]pyrimidin-7-yl]-2-(difluoromethoxy)benzamide;
6-[2,4-bis[(3S)-3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol, or a pharmaceutically acceptable salt thereof.

In one embodiment the mTOR-selective inhibitor is [5-[2,4-bis[(3S)-3-methylmorpholin-4-yl]pyrido[5,6-e]pyrimidin-7-yl]-2-methoxyphenyl]methanol, or a pharmaceutically acceptable salt thereof.

Compounds have been named with the aid of computer software (Lexichem 1.6 from Openeye).

In another embodiment the mTOR-selective inhibitor may inhibit gene expression, for example by interfering with mRNA stability or translation. In one embodiment the mTOR-selective inhibitor is selected from for example siRNA or shRNA.

It may be convenient or desirable to prepare, purify, and/or handle a corresponding salt of the inhibitor, for example, a pharmaceutically-acceptable salt. A suitable pharmaceutically-acceptable salt of a MEK inhibitor or a mTOR-selective inhibitor may be, for example, an acid-addition salt which is sufficiently basic, for example an acid-addition salt with an inorganic or organic acid. Such acid-addition salts include but are not limited to, fumarate, methanesulfonate, hydrochloride, hydrobromide, citrate and maleate salts and salts formed with phosphoric and sulfuric acid. A suitable pharmaceutically-acceptable salt of a MEK inhibitor or a mTOR-selective inhibitor may be, for example, a salt which is sufficiently acidic, for example an alkali or alkaline earth metal salt. Such alkali or alkaline earth metal salts include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

In one embodiment the MEK inhibitor is AZD6244 hydrogen sulphate salt. AZD6244 hydrogen sulphate salt may be synthesised according to the process described in International Patent Publication Number WO07/076,245.

In another aspect of the present invention there provided a combination product, as defined herein, comprising
a MEK inhibitor, or a pharmaceutically acceptable salt thereof,
linked to a mTOR-selective inhibitor, or a pharmaceutically acceptable salt thereof,
   in association with a pharmaceutically acceptable adjuvant, diluent or carrier.

The combination product of the present invention is expected to produce a synergistic or beneficial effect through the production of an anti-cancer effect in a patient, which is accordingly useful in the treatment of cancer in a patient. A beneficial effect is achieved if the effect is therapeutically superior, as measured by, for example, the extent of the response, the response rate, the time to disease progression or the survival period, to that achievable on dosing one or other of the components of the combination treatment at its conventional dose. The beneficial effect may be synergistic, if the combined effect is therapeutically superior to the sum of the individual effect achievable with a MEK inhibitor or a mTOR-selective inhibitor. Further, a beneficial effect is obtained if an effect is achieved in a group of patients that does not respond (or responds poorly) to a MEK inhibitor or a mTOR-selective inhibitor alone. In addition, the effect is defined as affording a beneficial effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, a beneficial effect is deemed to be achieved if a conventional dose of a MEK inhibitor or a mTOR-selective inhibitor may be reduced without detriment to one or more of: the extent of the response, the response rate, the time to disease progression and survival data, in particular without detriment to the duration of the response, but with fewer and/or less troublesome side-effects than those that occur when conventional doses of each component are used.

In another aspect of the present invention there is provided a method of treating cancer, which comprises administration of a therapeutically effective amount of a combination product, as defined herein, to a patient having or suspected of having cancer. In one embodiment the MEK inhibitor, or a pharmaceutically acceptable salt thereof, is administered sequentially, separately and/or simultaneously with the mTOR-selective inhibitor, or a pharmaceutically acceptable salt thereof. In one embodiment the method additionally comprises selecting a patient in need of treatment of cancer, and administration to the patient of a therapeutically effective dose of a combination product, as defined herein.

In another aspect of the present invention there is provided a method of inhibiting MEK and mTOR, which comprises administration of a therapeutically effective amount of a combination product, as defined herein, to a patient. In one embodiment the MEK inhibitor, or a pharmaceutically acceptable salt thereof, is administered sequentially, separately and/or simultaneously with the mTOR-selective inhibitor, or a pharmaceutically acceptable salt thereof. In one embodiment the method additionally comprises selecting a patient in need of MEK and/or mTOR inhibition, and administration to the patient of a therapeutically effective dose of a combination product, as defined herein.

In another aspect of the present invention there is provided a method of treating immuno-suppression, immune-tolerance, autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye, which comprises administration of a therapeutically effective amount of a combination product, as defined herein, to a patient having or suspected of having any one or more of the above conditions. In one embodiment the MEK inhibitor, or a pharmaceutically acceptable salt thereof, is administered sequentially, separately and/or simultaneously with the mTOR-selective inhibitor, or a pharmaceutically acceptable salt thereof. In one embodiment the method additionally comprises selecting a patient in need of treatment for one or more of the above conditions, and administration to the patient of a therapeutically effective dose of a combination product, as defined herein.

In another aspect of the present invention there is provided a combination product, as defined herein, for use in the production of an anti-cancer effect in a patient, which is accordingly useful in the treatment of cancer. In one embodiment there is provided use of a combination product, as defined herein, in the treatment of cancer.

In another aspect of the present invention there is provided a combination product, as defined herein, for use in the inhibition of MEK and/or mTOR in a patient, which is accordingly useful in the treatment of cancer.

In another aspect of the present invention there is provided a combination product, as defined herein, for use in the inhibition of MEK and/or mTOR.

In another aspect of the present invention there is provided a combination product, as defined herein, for use in the treatment for one or more of immuno-suppression, immune-tolerance, autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye.

In another aspect of the present invention there is provided use of a combination product, as defined herein, in the production of an anti-cancer effect in a patient, which is accordingly useful in the treatment of cancer. In one embodiment there is provided use of a combination product, as defined herein, in the treatment of cancer.

In another aspect of the present invention there is provided use of a combination product, as defined herein, in the inhibition of MEK and/or mTOR in a patient, which is accordingly useful in the treatment of cancer.

In another aspect of the present invention there is provided use of a combination product, as defined herein, for the inhibition of MEK and/or mTOR.

In another aspect of the present invention there is provided use of a combination product, as defined herein, in the treatment for one or more of immuno-suppression, immune-tolerance, autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye.

In another aspect of the present invention there is provided use of a combination product, as defined herein, in the manufacture of a medicament for use in the production of an anti-cancer effect in a patient, which is accordingly useful in the treatment of cancer.

In another aspect of the present invention there is provided use of a combination product, as defined herein, in the manufacture of a medicament for use in the inhibition of MEK and/or mTOR in a patient, which is accordingly useful in the treatment of cancer.

In another aspect of the present invention there is provided use of a combination product, as defined herein, in the manufacture of a medicament for use for the inhibition of MEK and/or mTOR.

In another aspect of the present invention there is provided use of a combination product, as defined herein, in the manufacture of a medicament for use in the treatment for one or more of immuno-suppression, immune-tolerance, autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye.

In one embodiment there is provided a method or use as described hereinabove wherein the patient is not resistant to MEK inhibition.

In one embodiment there is provided a method or use as described hereinabove wherein the patient is not resistant to mTOR inhibition.

The combination product of the present invention is expected to be particularly useful for the treatment patients with cancers, including, but not limited to, non-solid tumours such as leukaemia, for example acute myeloid leukaemia, multiple myeloma, haematologic malignancies or lymphoma, and also solid tumours and their metastases such as melanoma (in particular metastatic melanoma), non-small cell lung cancer, glioma, hepatocellular (liver) carcinoma, glioblastoma, carcinoma of the thyroid, cholangiocarcinoma, bile duct, bone, gastric, brain/CNS, head and neck, hepatic, stomach, prostate, breast, renal, testicular, ovarian, cervix, skin, cervical, lung, muscle, neuronal, oesophageal, bladder, lung, uterine, vulval, endometrial, kidney, colon, colorectal, pancreatic, pleural/peritoneal membranes, salivary gland, epidermoid tumours haematological malignancies.

The combination product of the present invention is expected to be particularly useful for the treatment patients with hematopoietic tumours of lymphoid lineage, including acute lymphocytic leukaemia, B-cell lymphoma and Burketts lymphoma; hematopoietic tumours of myeloid lineage, including acute and chronic myelogenous leukaemias and promyelocytic leukaemia; tumours of mesenchymal origin, including fibro sarcoma and rhabdomyo sarcoma; and other tumours, including melanoma, seminoma, tetratocarcinoma, neuroblastoma and glioma.

The combination product of the present invention is expected to be especially useful for the treatment patients with lung cancer, melanoma, breast cancer, gastric cancer, colorectal cancer, hepatocellular (liver) carcinoma, ovarian cancer, thyroid cancer, pancreatic cancer, liver cancer, and their metastases, and also for the treatment of patients with acute myeloid leukaemia or multiple myeloma.

The combination product of the present invention is also expected to be particularly useful for the treatment of patients with a tumour which is ameliorated by the inhibition of mTOR.

The combination product of the present invention is also expected to be particularly useful for the treatment of patients with a tumour which is associated with the Ras-Raf-MEK-ERK pathway or which is dependent alone, or in part, on the biological activity of the Ras-Raf-MEK-ERK pathway.

The combination product of the present invention is also expected to be particularly useful for the treatment of patients with a tumour which is associated with MEK or which is dependent alone, or in part, on the biological activity of MEK.

The combination product of the present invention is also expected to be particularly useful for the treatment of patients with a tumour which is associated with the PI3K/AKT pathway or which is dependent alone, or in part, on the biological activity of the PI3K/AKT pathway.

The combination product of the present invention is also expected to be particularly useful for the treatment of patients with a tumour which is associated with mTOR or which is dependent alone, or in part, on the biological activity of mTOR.

The dosage of the MEK inhibitor and/or the mTOR-selective inhibitor for a given patient will be determined by the attending physician, taking into consideration various factors known to modify the action of drugs including severity and type of disease, body weight, sex, diet, time and route of administration, other medications and other relevant clinical factors. Therapeutically effective dosages may be determined by either in vitro or in vivo methods.

The therapeutically effective amount of a MEK inhibitor or a mTOR-selective inhibitor, as described herein, to be used will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.0001 mg/kg to up to 250 mg/kg or more, depending on the factors mentioned above. Typically, the clinician will administer the combination product, as defined herein, until a dosage is reached that achieves the desired effect. Where separate formulations are administered, the sequence in which the MEK inhibitor, or pharmaceutically acceptable salt thereof, and the mTOR-selective inhibitor, or pharmaceutically acceptable salt thereof, may be administered (i.e. whether and at what point sequential, separate and/or simultaneous administration takes place) may be determined by the physician or skilled person.

In another aspect of the present invention there is provided use of a combination product of the invention in the preparation of a medicament for administration to a patient with cancer, wherein the administration of the medicament comprises from about 0.01 mg/kg to up to 250 mg/kg or more, daily, every 2 days, every 3 days, every 4 days, every 5 days, every 6 days or weekly. Administration of the medicament may take place as hereinbefore described, for example separate formulations of a MEK inhibitor and a mTOR-selective inhibitor may be administered sequentially, separately and/or simultaneously.

In another aspect of the present invention there is provided use of a combination product of the invention in the preparation of a medicament for administration to a patient for the inhibition of MEK and/or mTOR in the patient, wherein the administrative pattern of the medicament comprises from about 0.01 mg/kg to up to 250 mg/kg or more, daily, every 2 days, every 3 days, every 4 days, every 5 days, every 6 days or weekly. Administration of the medicament may take place as hereinbefore described, for example separate formulations of a MEK inhibitor and a mTOR-selective inhibitor may be administered sequentially, separately and/or simultaneously.

In another aspect of the present invention there is provided use of a combination product of the invention in the preparation of a medicament for administration to a patient with one or more of immuno-suppression, immune-tolerance, autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye, wherein the administrative pattern of the medicament comprises from about 0.01 mg/kg to up to 250 mg/kg or more, daily, every 2 days, every 3 days, every 4 days, every 5 days, every 6 days or weekly. Administration of the medicament may take place as hereinbefore described, for example separate formulations of a MEK inhibitor and a mTOR-selective inhibitor may be administered sequentially, separately and/or simultaneously.

In the above methods and uses, the combination product may be any combination product according to any of the definitions herein.

The combination product of the present invention may be used as a sole therapy or may involve additional surgery or radiotherapy or an additional chemotherapeutic agent or a therapeutic antibody.

Such chemotherapeutic agents may include one or more of the following categories of anti tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 50α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZDO530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB I antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras signalling inhibitors such as farnesyl transferase inhibitors, for example sorafenib (BAY 43-9006)), inhibitors of cell signalling through AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment.

Anti-cancer effects which are accordingly useful in the treatment of cancer in a patient include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a combination product of the present invention is administered to a patient in need of treatment for cancer, said combination product, as defined herein, will produce an effect, as measured by, for example, one or more of: the extent of the anti-tumour effect, the response rate, the time to disease progression and the survival rate. Anti-cancer effects include prophylactic treatment as well as treatment of existing disease.

A formulation of a MEK inhibitor or a mTOR-selective inhibitor (each of which is an “active compound”), comprises a MEK inhibitor or a mTOR-selective inhibitor, as defined herein, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. A combined preparation of a MEK inhibitor and a mTOR-selective inhibitor comprises a MEK inhibitor and a mTOR-selective inhibitor, as defined herein, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides formulations, as defined above, and methods of making a pharmaceutical composition comprising admixing a MEK inhibitor or a mTOR-selective inhibitor together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th edition, pub. Lippincott, Williams & Wilkins, 2000 or Handbook of Pharmaceutical Excipients, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The following terms, unless otherwise indicated, shall be understood to have the following meanings:

An inhibitor may be a polypeptide, nucleic acid, carbohydrate, lipid, small molecular weight compound, an oligonucleotide, an oligopeptide, siRNA, antisense, a recombinant protein, an antibody, a peptibody, or conjugates or fusion proteins thereof. For a review of siRNA see Milhavet O, Gary D S, Mattson M P. (Pharmacol Rev. 2003 December; 55(4):629-48. For a review of antisense see Opalinska J B, Gewirtz A M. Sci STKE. 2003 Oct. 28; 2003 (206): pe47.

A small molecular weight compound refers to a compound with a molecular weight of less than 2000 Daltons, 1000 Daltons, 700 Daltons or 500 Daltons.

An mTOR-selective inhibitor is selective for mTOR over other kinases. An mTOR-selective inhibitor is selective for mTOR over PI3K. An mTOR-selective inhibitor is any inhibitor of the biological activity of wild-type or any mutant form of mTOR.

A patient is any warm-blooded animal, such as a human.

The term treatment includes therapeutic and/or prophylactic treatment.

The MEK inhibitor AZD6244 can be prepared according to the process described in International Patent Publication Number WO03/077914, in particular according to the process described in Example 10. The AZD6244 hydrogen sulphate salt can be prepared according to the process described in International Patent Publication Number WO07/076,245.

The MEK inhibitor 4-(4-Bromo-2-fluorophenylamino)-N-(2-hydroxyethoxy)-1,5-dimethyl-6-oxo-1,6-dihydropyridazine-3-carboxamide can be prepared according to the following method

Step A: Preparation of diethyl 2-(2-methylhydrazono)malonate: To a solution of diethyl ketomalonate (95 g, 546 mmol) in EtOH (600 mL) (2 L 3-neck flask equipped with thermocouple, ° C. (internal temperature, heated by a heating mantle) and stirred for 6 hours. The reaction mixture was cooled to room temperature and stirred overnight. The reaction mixture was concentrated under reduced pressure to give the crude material along with solid precipitates that was purified by a silica gel plug (3:2 hexanes:EtOAc) to afford 81 g (74%) of the desired product. N₂ line, condenser and mechanical stirrer) was added MeNHNH₂ (32 mL, 600 mmol) in one portion at room temperature. The reaction mixture was warmed to 60

Step B: Preparation of diethyl 2-(2-methyl-2-propionylhydrazono)malonate: To a solution of 2-(2-methylhydrazono)malonate (100 g, 494 mmol) in THF (1 L) at 0° C. was added LiHMDS (643 mL, 643 mmol) by an addition funnel over 45 minutes. The reaction mixture was stirred for 45 minutes at 0° C. Propionyl chloride (51.6 mL, 593 mmol) was added in one portion). The resulting mixture was warmed to room temperature and stirred for 20 hours. The reaction mixture was quenched with saturated aqueous NH₄Cl (85 mL) and water (85 mL). The reaction mixture was concentrated under reduced pressure and additional water (300 mL) was added. The resulting mixture was extracted with EtOAc (3×250 mL). The combined organic layers were washed with saturated aqueous NaHCO₃ (2×250 mL) followed by brine (250 mL), dried over MgSO₄, filtered, and concentrated under reduced pressure to give 112 g (88%) of the crude product that was used directly in the next step without further purification.

Step C: Preparation of 4-hydroxy-1,5-dimethyl-6-oxo-1,6-dihydropyridazine-3-carboxylic acid: To a solution of LiHMDS (331 mL, 331 mmol, 1 M solution in THF) in THF (430 mL) at −78° C. was added a solution of 2-(2-methyl-2-propionylhydrazono)malonate (21.40 g, 82.86 mmol) in THF (10 mL). The resulting mixture was slowly warmed to −40° C. over 1 hour and stirred for 1.5 hours at −40° C. To the reaction mixture was added water (500 mL) at −40° C. The reaction mixture was warmed to room temperature and stirred for 3 hours. The reaction mixture was concentrated under reduced pressure, quenched with 6 N aqueous HCl at 0° C., and acidified to pH 1 to 2. The resulting mixture was stirred for 16 hours at room temperature. The precipitates were filtered off and triturated with CH₂Cl₂ to afford 7.21 g (47%) of the desired product. The filtrate was extracted with EtOAc (3×). The combined organic layers were washed with water, dried over MgSO₄, filtered, and concentrated under reduced pressure to give the crude material that was triturated with CH₂Cl₂ to afford additional 3.56 g (23%) of the desired product. The aqueous layer was extracted again with EtOAc (3×). The combined organic layers were washed with water, dried over MgSO₄, filtered, and concentrated under reduced pressure to give the crude material that was triturated with CH₂Cl₂ to afford additional 1.32 g (9%) of the desired product. A total of 12.09 g (79%) of the desired product was obtained.

Step D: Preparation of 4-chloro-1,5-dimethyl-6-oxo-1,6-dihydropyridazine-3-carboxylic acid: A mixture of 4-hydroxy-1,5-dimethyl-6-oxo-1,6-dihydropyridazine-3-carboxylic acid (35.4 g, 192 mmol), catalytic amount of DMF (3 drop), and POCl₃ (178 mL, 1.92 mol) was heated for 2 days at 90° C., and then the POCl₃ was removed under reduced pressure. The crude material was quenched with ice, and the reaction mixture was stirred for 2 hours at room temperature. The precipitates formed out of the solution was filtered off and washed with ether. The precipitates collected were triturated with ether to afford 11.7 g (30%) of the desired product. The filtrate was extracted with EtOAc (2×). The combined organic layers were dried over MgSO₄, filtered, and concentrated under reduced pressure to give the crude product that was triturated with ether and dried under reduced pressure to afford additional 9.56 g (24%) of the desired product. A total of 21.29 g (55%) of the desired product was obtained.

Step E: Preparation of 4-(4-bromo-2-fluorophenylamino)-1,5-dimethyl-6-oxo-1,6-dihydropyridazine-3-carboxylic acid: To a solution of 4-bromo-2-fluoroaniline (22.6 g, 116 mmol) in THF (165 mL) at −78° C. was slowly added a solution of LiHMDS (174 mL, 174 mmol, 1 M solution in THF). The resulting mixture was stirred for 1 hour at −78° C. To this mixture was added 4-chloro-1,5-dimethyl-6-oxo-1,6-dihydropyridazine-3-carboxylic acid (11.0 g, 54.4 mmol) as a solid at −78° C. The reaction mixture was slowly warmed to room temperature and stirred for 21 hour. The reaction was quenched and acidified with 10% aqueous HCl (250 mL) at 0° C. To this mixture was added water (100 mL), EtOAc (350 mL), and brine (50 mL). The reaction mixture was warmed to room temperature and stirred for 30 minutes. The organic layer was separated and the acidic aqueous layer was extracted with EtOAc (2×300 mL). The combined organic layers were dried over MgSO₄, filtered, and concentrated under reduced pressure to give the crude material that was triturated with ether (5×), filtered, washed with ether, and dried under reduced pressure to afford 14.51 g (75%) of the desired product.

Step F: Preparation of 4-(4-bromo-2-fluorophenylamino)-1,5-dimethyl-6-oxo-N-(2-(vinyloxy)ethoxy)-1,6-dihydropyridazine-3-carboxamide: To a suspension of 4-(4-bromo-2-fluorophenylamino)-1,5-dimethyl-6-oxo-1,6-dihydropyridazine-3-carboxylic acid (14.51 g, 40.74 mmol) and HOBt (11.01 g, 81.48 mmol) in DMF (165 mL) was added EDCI (15.62 g, 81.48 mmol) at room temperature. The resulting mixture was stirred for 1.5 hours. O-(2-(Vinyloxy)ethyl)hydroxylamine (8.36 mL, 81.48 mmol) and TEA (11.36 mL, 81.48 mmol) was added to the activated ester at room temperature. After stirring for 1.5 hours, the reaction mixture was diluted with EtOAc and washed with saturated aqueous NH₄Cl, brine, saturated aqueous NaHCO₃ (2×), and brine. The organic layer was separated, dried over MgSO₄, filtered, and concentrated under reduced pressure to give the crude product that was used directly without further purification.

Step G: Preparation of 4-(4-bromo-2-fluorophenylamino)-N-(2-hydroxyethoxy)-1,5-dimethyl-6-oxo-1,6-dihydropyridazine-3-carboxamide: A mixture of 4-(4-bromo-2-fluorophenylamino)-1,5-dimethyl-6-oxo-N-(2-(vinyloxy)ethoxy)-1,6-dihydropyridazine-3-carboxamide (17.98 g, 40.75 mmol) and 6 N aqueous HCl (13.58 mL, 81.50 mmol) in EtOH/THF (50 mL/50 mL) was stirred for 3 hours at room temperature. The reaction mixture was concentrated under reduced pressure and diluted with water (50 mL). The resulting mixture was extracted with EtOAc (2×). The combined organic layers were dried over MgSO₄, filtered, and concentrated under reduced pressure to give the crude material that was purified by silica gel flash column chromatography (100% CH₂Cl₂ to 2.5% MeOH in CH₂Cl₂) to afford 9.41 g (56% for two steps) of the desired product. MS APCI (−) m/z 413, 415 (M−1, Br pattern) detected; ¹H NMR (400 MHz, CD₃OD) δ 7.38 (dd, 1H), 7.27 (d, 1H), 6.79 (t, 1H), 3.99 (t, 2H), 3.80 (s, 3H), 3.74 (t, 2H), 1.77 (s, 3H).

The MEK inhibitor 2-(2-fluoro-4-iodophenylamino)-N-(2-hydroxyethoxy)-1,5-dimethyl-6-oxo-1,6-dihydropyridine-3-carboxamide can be prepared according to the following method

Step A. Preparation of 2-chloro-6-oxo-1,6-dihydro-pyridine-3-carboxylic acid: 2-Chloro-6-oxo-1,6-dihydro-pyridine-3-carboxylic acid was prepared from dichloronicotinic acid (3.00 g, 15.6 mmol, Aldrich) according to the procedure described in U.S. Pat. No. 3,682,932 to yield 1.31 g (48%) of the desired product.

Step B. Preparation of 2-chloro-1-methyl-6-oxo-1,6-dihydro-pyridine-3-carboxylic acid methyl ester: To a solution of 2-chloro-6-oxo-1,6-dihydro-pyridine-3-carboxylic acid (0.644 g, 3.71 mmol) in DMF (20 mL) was added lithium hydride (95%, 0.078 g, 9.28 mmol) and the reaction mixture was stirred for 40 minutes under N₂. Methyl iodide (0.508 mL, 1.16 g, 8.16 mmol) was then added and the reaction mixture was stirred for an additional 45 minutes. The reaction mixture was quenched with 2 M HCl until the pH was 6-7. The reaction mixture was diluted with EtOAc and saturated NaCl and the layers separated. The aqueous layer was back extracted with EtOAc (1×). The combined organic layers were dried (Na₂SO₄) and concentrated under reduced pressure to yield a crude yellow solid. HPLC analysis showed two products in a 4:1 ratio that were separated by flash column chromatography (methylene chloride/EtOAc, 15:1 to 10:1) to give 0.466 g (62%) pure desired product as a white crystalline solid. Step C. Preparation of methyl 5-bromo-2-chloro-1-methyl-6-oxo-1,6-dihydropyridine-3-carboxylate: To a solution of methyl 2-chloro-1-methyl-6-oxo-1,6-dihydropyridine-3-carboxylate (0.100 g, 0.496 mmol) in DMF (5 mL) was added N-bromosuccinimide (0.177 g, 0.992 mmol) and the reaction mixture was stirred for 4 hours at room temperature under N₂. The reaction mixture was quenched with saturated sodium bisulfite and then diluted with EtOAc and H₂O and the layers separated. The aqueous layer was back extracted with EtOAc (2×). The combined organic layers were dried (Na₂SO₄) and concentrated under reduced pressure to yield a yellow solid in quantitative yield.

Step D. Preparation of methyl 2-chloro-1,5-dimethyl-6-oxo-1,6-dihydropyridine-3-carboxylate: To a suspension of methyl 5-bromo-2-chloro-1-methyl-6-oxo-1,6-dihydropyridine-3-carboxylate (0.400 g, 1.43 mmol) and 1,1′-bis(diphenylphosphino)ferrocenedichloropalladium(II) (0.0587 g, 0.0713 mmol) in dioxane (8 mL) at 0° C. under N₂ was added dimethylzinc (0.713 mL, 1.43 mmol, 2 M solution in toluene). The reaction mixture was immediately heated to 100° C. for 30 minutes. The reaction mixture was cooled to 0° C. and quenched with MeOH (0.800 mL). The reaction mixture was diluted with EtOAc and washed with 1 M HCl. The aqueous layer was back extracted with EtOAc (1×). The combined organic layers were washed with saturated NaCl, dried (Na₂SO₄) and concentrated under reduced pressure to a dark yellow gum. Purification by flash column chromatography (methylene chloride/EtOAc, 15:1) gave 0.164 g (53%) pure desired product as a yellow crystalline solid.

Step E: Preparation of methyl-(2-fluoro-4-iodophenylamino)-1,5-dimethyl-6-oxo-1,6-dihydropyridine-3-carboxylate: To a solution of 2-fluoro-4-iodobenzenamine (0.058 g, 0.31 mmol) in THF (2 mL) at −78° C. under N₂ was added lithium bis(trimethylsilyl)amide (0.56 mL, 0.56 mmol, 1 M solution in hexanes) dropwise. The reaction mixture was stirred for one hour at −78° C. Methyl 2-chloro-1,5-dimethyl-6-oxo-1,6-dihydropyridine-3-carboxylate 0.060 g, 0.28 mmol) was then added dropwise as a solution in THF (1 mL) and the reaction mixture was stirred for 25 minutes at −78° C. The reaction mixture was quenched by the addition of H₂O and the pH was adjusted with 0.1M HCl and then diluted with EtOAc and saturated NaCl and the layers separated. The aqueous layer was back extracted with EtOAc (1×). The combined EtOAc layers were dried (Na₂SO₄) and concentrated under reduced pressure. Purification by flash column chromatography (methylene chloride/EtOAc, 20:1) gave 0.086 g (84%) pure desired product as a white crystalline solid. MS ESI (+) m/z 417 (M+1) detected; ¹H NMR (400 MHz, CDCl₃) δ 9.56 (s, 1H), 7.79 (s, 1H), 7.49 (d, 1H), 7.36 (d, 1H), 6.43 (t, 1H), 3.85 (s, 3H), 3.30 (s, 3H), 2.15 (s, 3H).

Step F: Preparation of 2-(2-fluoro-4-iodophenylamino)-1,5-dimethyl-6-oxo-N-(2-(vinyloxy)ethoxy)-1,6-dihydropyridine-3-carboxamide: To a solution of methyl 2-(2-fluoro-4-iodophenylamino)-1,5-dimethyl-6-oxo-1,6-dihydropyridine-3-carboxylate (0.500 g, 1.20 mmol) in THF (60 mL) was added O-(2-vinyloxy-ethyl)-hydroxylamine (0.149 g, 1.44 mmol). The solution was cooled to 0° C. and lithium bis(trimethylsilyl)amide (4.81 ml, 4.81 mmol) (1 M solution in hexanes) was added dropwise. The reaction mixture was warmed to room temperature. After stirring for 10 minutes the reaction mixture was quenched by the addition of 1 M HCl and partitioned between EtOAc and saturated NaCl. The layers were separated and the organic layer was dried (Na₂SO₄) and concentrated under reduced pressure to yield a crude yellow solid that was used without purification in the next step.

Step G: Preparation of 2-(2-fluoro-4-iodophenylamino)-N-(2-hydroxyethoxy)-1,5-dimethyl-6-oxo-1,6-dihydropyridine-3-carboxamide: To a solution of crude 2-(2-fluoro-4-iodophenylamino)-1,5-dimethyl-6-oxo-N-(2-(vinyloxy)ethoxy)-1,6-dihydropyridine-3-carboxamide (0.585 g, 1.20 mmol) in ethanol (10 mL) was added aqueous 2 M HCl (3 mL). The reaction mixture was stirred for 45 minutes at room temperature. The pH of the reaction mixture was adjusted to pH 7 with 1 M NaOH. The reaction mixture was diluted with EtOAc and H₂O. The organic layer was separated and washed with saturated NaCl. The combined aqueous layers were back extracted with EtOAc (1×). The combined organic layers were dried (Na₂SO₄) and concentrated under reduced pressure. Purification by silica gel flash column chromatography (methylene chloride/MeOH, 15:1) gave 2-(2-fluoro-4-iodophenylamino)-N-(2-hydroxyethoxy)-1,5-dimethyl-6-oxo-1,6-dihydropyridine-3-carboxamide (0.421 g; 76% over two steps) as a pale yellow solid. MS ESI (+) m/z 462 (M+1) pattern detected; ¹H NMR (400 MHz, CDCl₃) δ 9.77 (s, 1H), 8.50 (s, 1H), 7.47 (d, 1H), 7.36 (d, 1H), 6.43 (t, 1H), 4.04 (br s, 2H), 3.85 (br s, 1H), 3.74 (br s, 2H), 3.29 (s, 3H), 2.14 (s, 3H). MS ESI (+) m/z 462 (M+1) pattern detected.

The invention will now be illustrated by the following non-limiting examples, which are provided for illustrative purposes only and are not to be construed as limiting upon the teachings herein, in which:

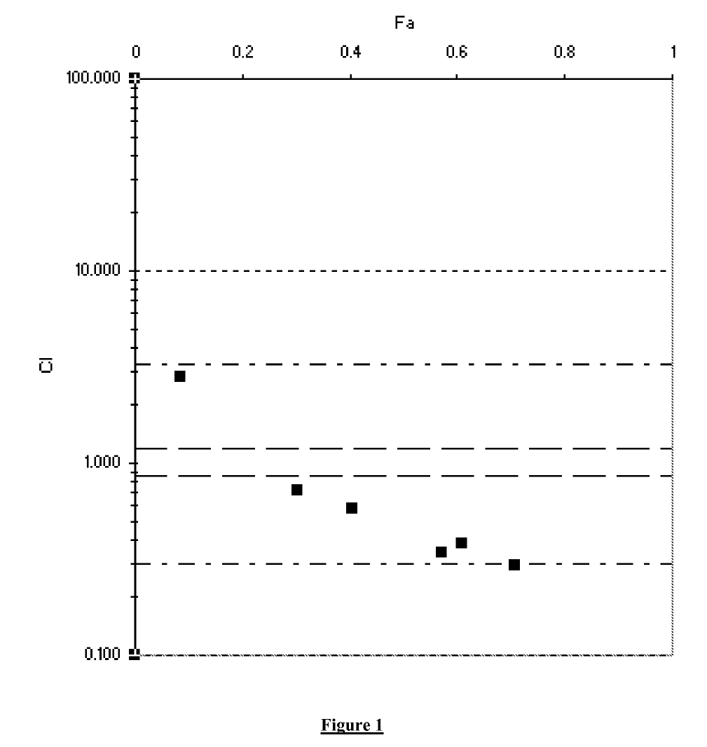

FIG. 1. Combination Index showing concurrent combination of AZD6244 with Compound A in the A2058 cell line using a 96-hour MTS viable cell number endpoint.

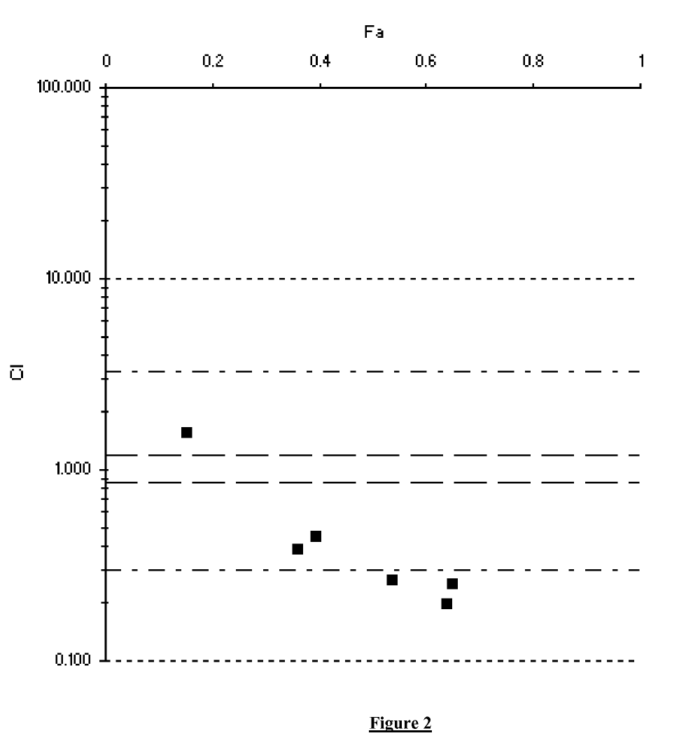

FIG. 2. Combination Index showing concurrent combination of 2-(2-fluoro-4-iodophenylamino)-N-(2-hydroxyethoxy)-1,5-dimethyl-6-oxo-1,6-dihydropyridine-3-carboxamide with Compound A in the A2058 cell line using a 96-hour MTS viable cell number endpoint.

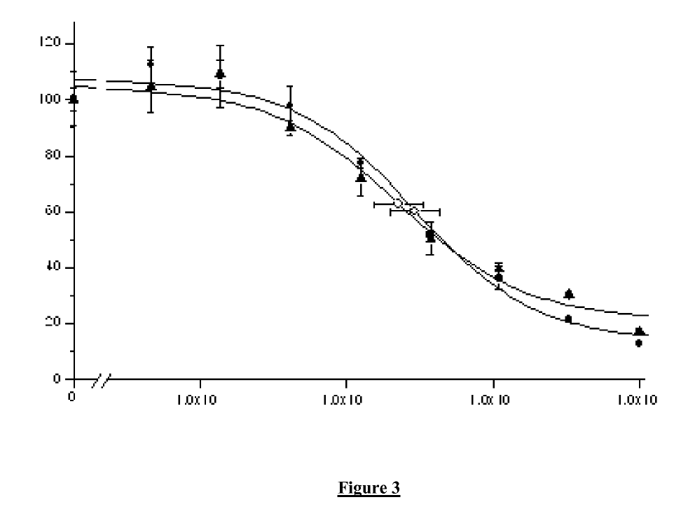

FIG. 3. Curve shift analysis plot showing concurrent combination of AZD6244 with Rapamycin 300 nM in the A2058 cell line using a 96-hour MTS viable cell number endpoint; % cell viability against concentration. Diamonds represent AZD6244 monotherapy; triangles represent the combination.

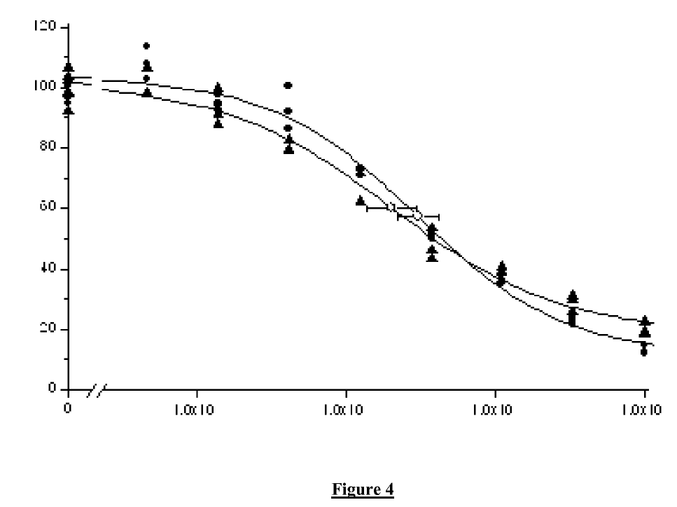

FIG. 4. Curve shift analysis plot showing concurrent combination of AZD6244 with Rapamycin 3 nM in the A2058 cell line using a 96-hour MTS viable cell number endpoint; % cell viability against concentration. Diamonds represent AZD6244 monotherapy; triangles represent the combination.

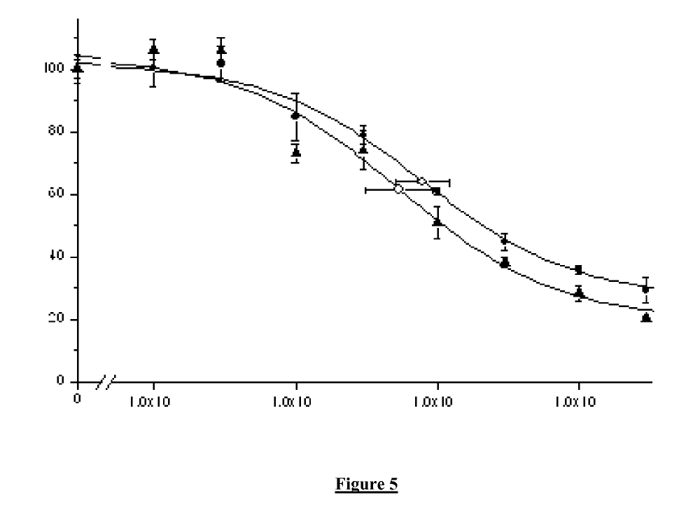

FIG. 5. Curve shift analysis plot showing concurrent combination of 2-(2-fluoro-4-iodophenylamino)-N-(2-hydroxyethoxy)-1,5-dimethyl-6-oxo-1,6-dihydropyridine-3-carboxamide with Rapamycin 300 nM in the A2058 cell line using a 96-hour MTS viable cell number endpoint; % cell viability as % of control against concentration. Diamonds represent 2-(2-fluoro-4-iodophenylamino)-N-(2-hydroxyethoxy)-1,5-dimethyl-6-oxo-1,6-dihydropyridine-3-carboxamide monotherapy; triangles represent the combination.

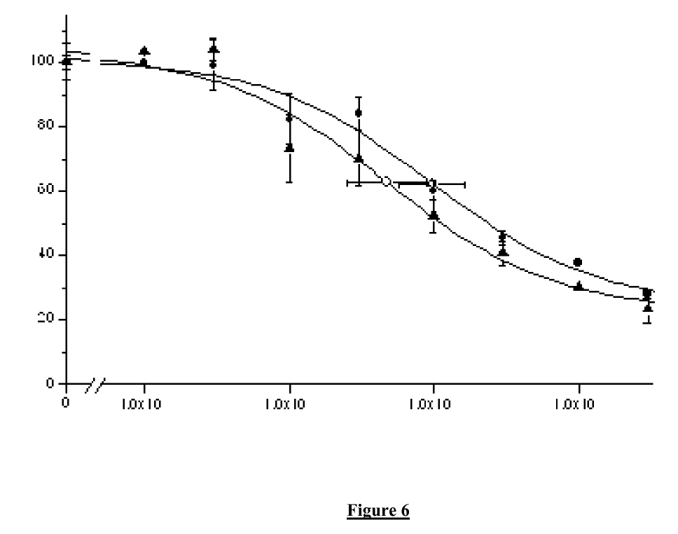

FIG. 6. Curve shift analysis plot showing concurrent combination of 2-(2-fluoro-4-10 iodophenylamino)-N-(2-hydroxyethoxy)-1,5-dimethyl-6-oxo-1,6-dihydropyridine-3-carboxamide with Rapamycin 3 nM in the A2058 cell line using a 96-hour MTS viable cell number endpoint; % cell viability as % of control against concentration. Diamonds represent 2-(2-fluoro-4-iodophenylamino)-N-(2-hydroxyethoxy)-1,5-dimethyl-6-oxo-1,6-dihydropyridine-3-carboxamide monotherapy; triangles represent the combination.

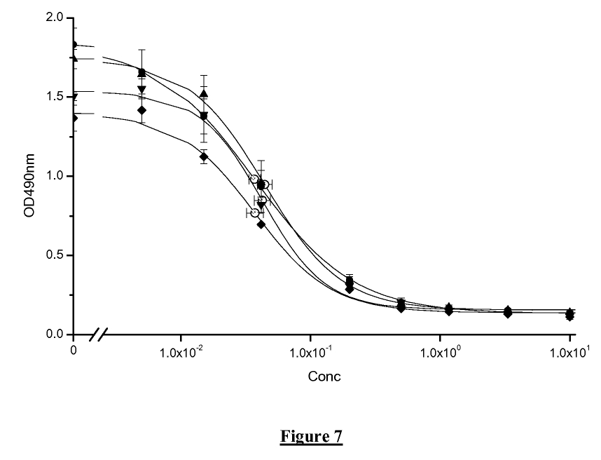

FIG. 7. Curve shift analysis plot showing combination of AZD6244 with Compound A in the A549 cell line using a 96-hour MTS viable cell number endpoint; % cell viability as % of control against concentration. Circles represent Compound A monotherapy; triangles represent Compound A+20 nM AZD6244; inverted triangles represent Compound A+333 nM AZD6244; diamonds represent Compound A+1000 nM AZD6244.

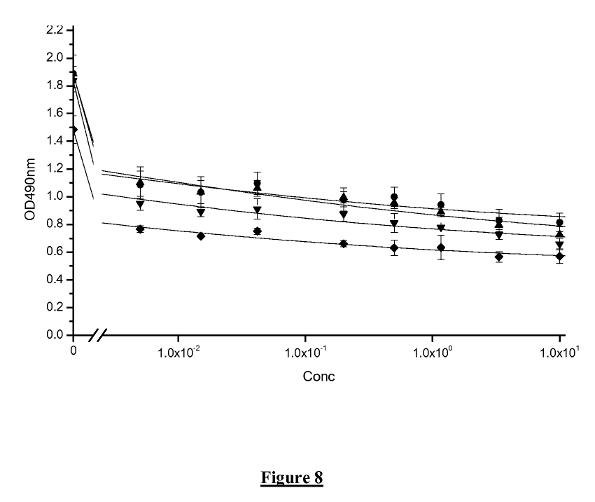

FIG. 8. Curve shift analysis plot showing the combination of AZD6244 with Rapamycin in the A549 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

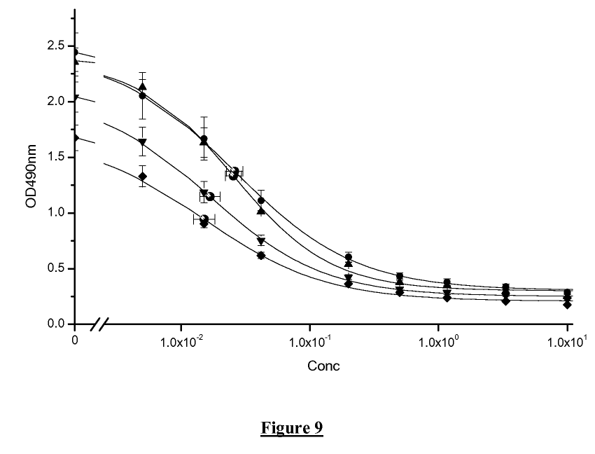

FIG. 9. Curve shift analysis plot showing combination of AZD6244 with Compound A in the NCI-H460 cell line using a 96-hour MTS viable cell number endpoint; % cell viability as % of control against concentration. Circles represent Compound A monotherapy; triangles represent Compound A+20 nM AZD6244; inverted triangles represent Compound A+333 nM AZD6244; diamonds represent Compound A+1000 nM AZD6244.

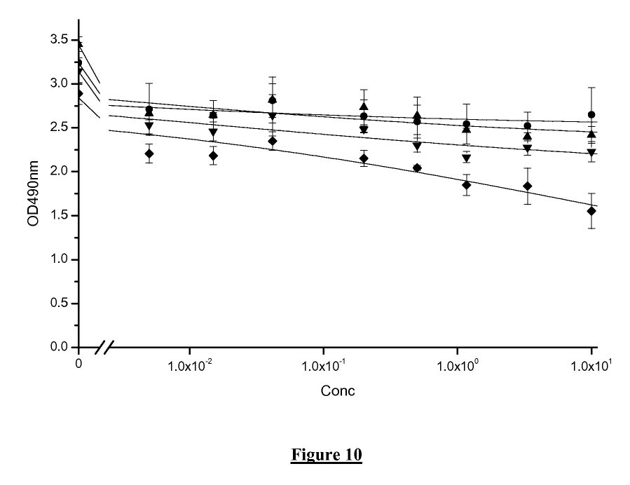

FIG. 10. Curve shift analysis plot showing the combination of AZD6244 with Rapamycin in the NCI-H460 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

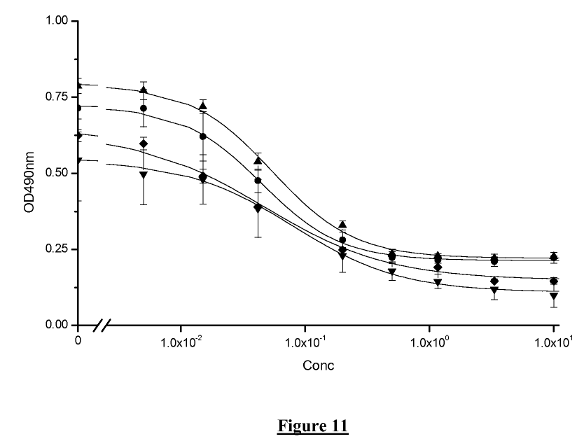

FIG. 11. Curve shift analysis plot showing combination of AZD6244 with Compound A in the NCI-H23 cell line using a 96-hour MTS viable cell number endpoint; % cell viability as % of control against concentration. Circles represent Compound A monotherapy; triangles represent Compound A+20 nM AZD6244; inverted triangles represent Compound A+333 nM AZD6244; diamonds represent Compound A+1000 nM AZD6244.

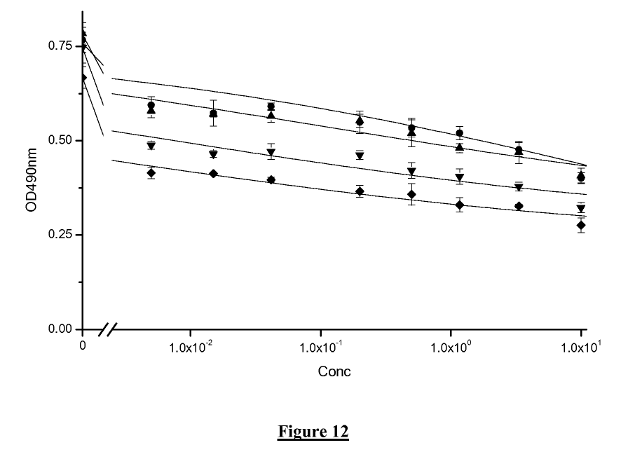

FIG. 12. Curve shift analysis plot showing the combination of AZD6244 with Rapamycin in the NCI-H23 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

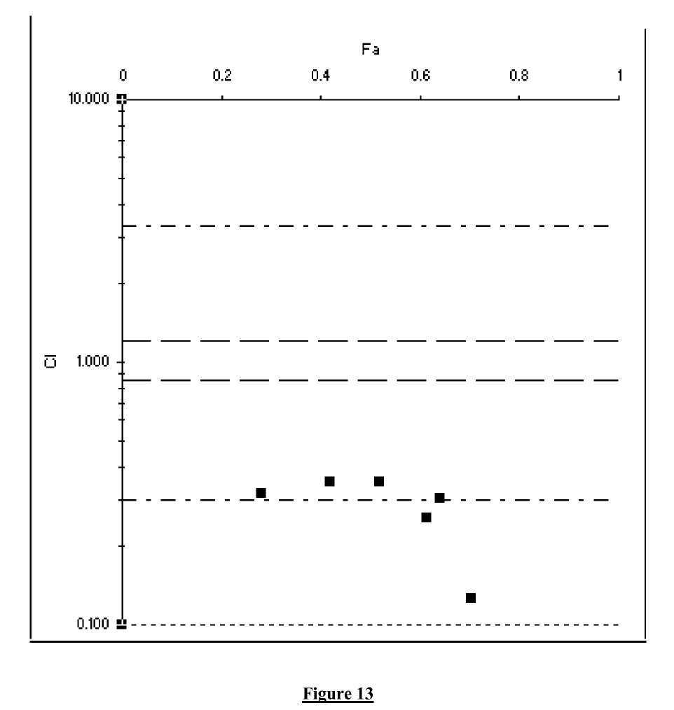

FIG. 13. Combination Index showing combination of AZD6244 with Compound A in the NCI-H2291 cell line using a 96-hour MTS viable cell number endpoint.

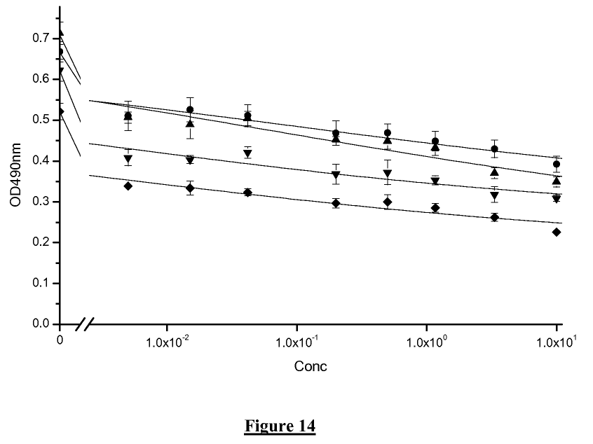

FIG. 14. Curve shift analysis plot showing the combination of AZD6244 with Rapamycin in the NCI-H2291 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

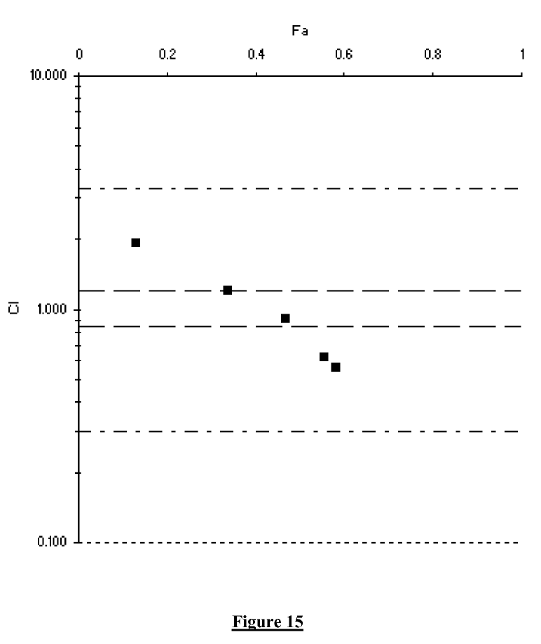

FIG. 15. Combination Index showing combination of AZD6244 with Compound A in the NCI-H727 cell line using a 96-hour MTS viable cell number endpoint.

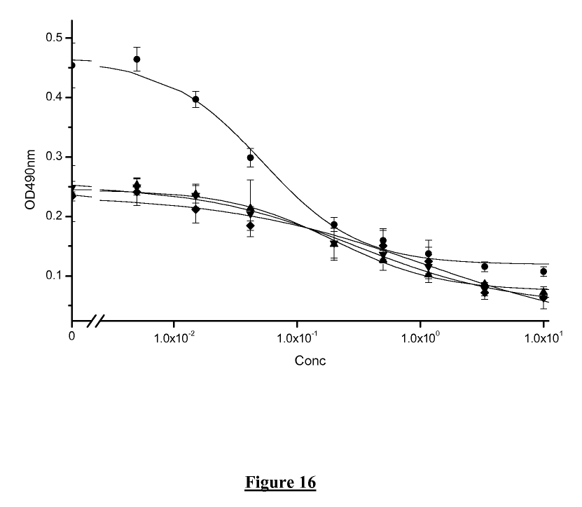

FIG. 16. Curve shift analysis plot showing the combination of AZD6244 with Rapamycin in the NCI-H727 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

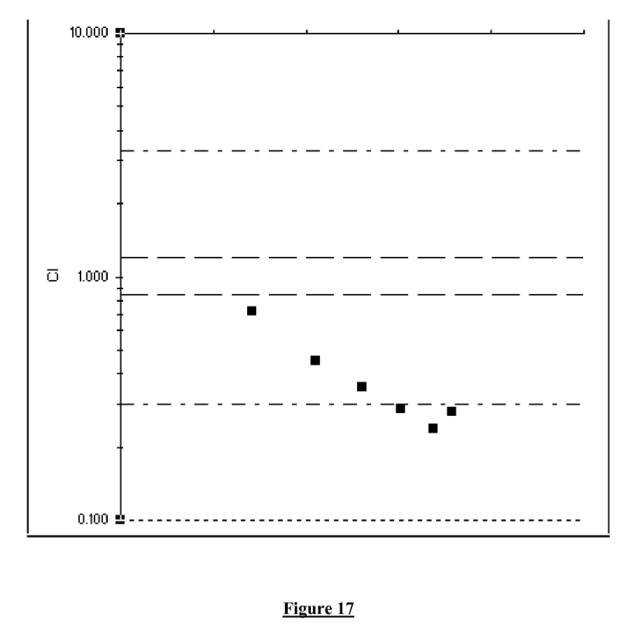

FIG. 17. Combination Index showing combination of AZD6244 with Compound A in the Calu-6 cell line using a 96-hour MTS viable cell number endpoint.

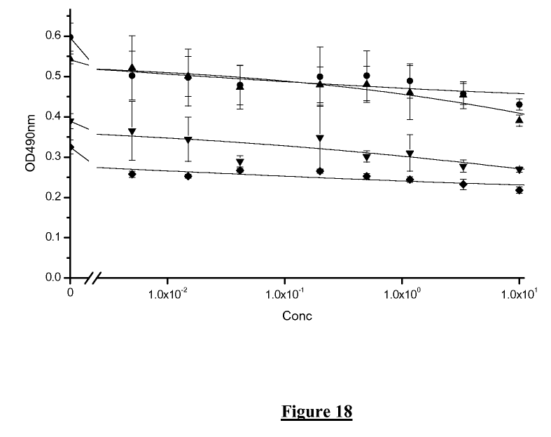

FIG. 18. Curve shift analysis plot showing the combination of AZD6244 with Rapamycin in the Calu-6 cell line using a 96-hour MTS viable cell number endpoint; % cell viability as % of control against concentration. Circles represent Rapamycin monotherapy; triangles represent Rapamycin+20 nM AZD6244; inverted triangles represent Rapamycin+333 nM AZD6244; diamonds represent Rapamycin+1000 nM AZD6244.

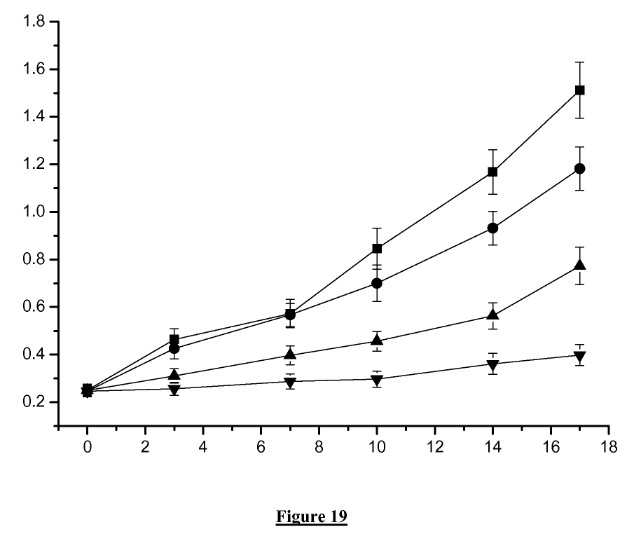

FIG. 19. Combination of AZD6244 and Compound A in HCT-116 xenografts; tumour volume in cm³ against days of dosing. Squares represent vehicle; circles represent Compound A monotherapy; triangles represent AZD6244 monotherapy; inverted triangles represent Compound A and AZD6244 in combination.

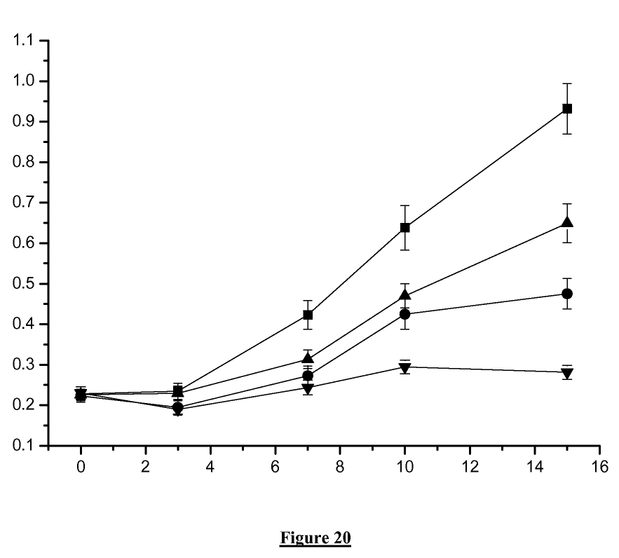

FIG. 20. Combination of AZD6244 and Compound A in LoVo xenografts; tumour volume in cm³ against days of dosing. Squares represent vehicle; circles represent Compound A monotherapy; triangles represent AZD6244 monotherapy; inverted triangles represent Compound A and AZD6244 in combination.

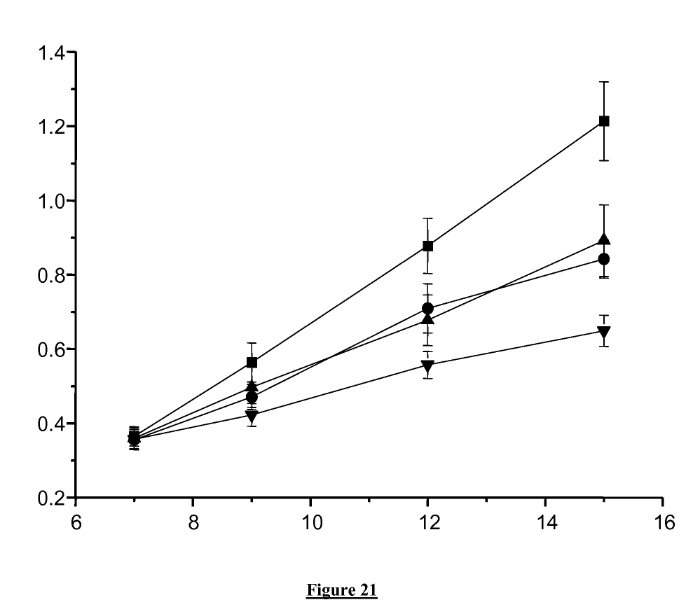

FIG. 21. Combination of AZD6244 and Rapamycin in LoVo xenografts; tumour volume in cm³ against days of dosing. Squares represent vehicle; circles represent AZD6244 monotherapy; triangles represent Rapamycin monotherapy; inverted triangles represent AZD6244 and Rapamycin in combination.

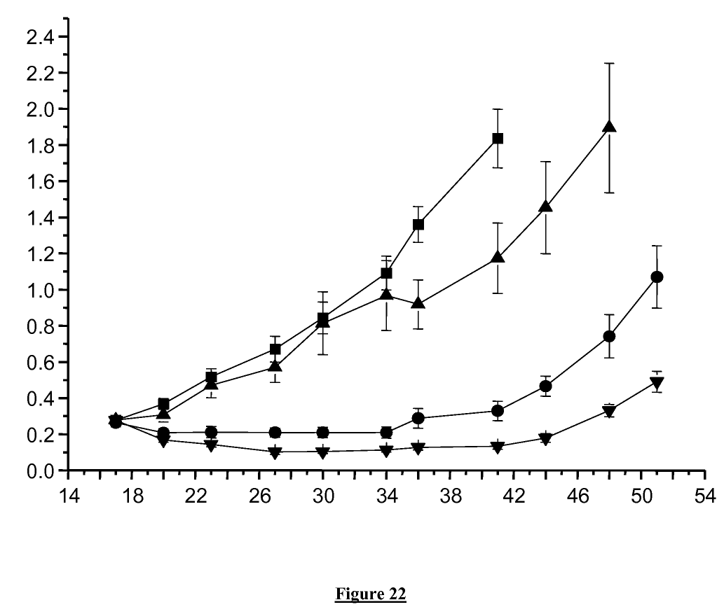

FIG. 22. Combination of AZD6244 and Compound A in Calu-6 xenografts; tumour volume in cm³ against days of dosing. Squares represent vehicle; circles represent AZD6244 monotherapy; triangles represent Compound A monotherapy; inverted triangles represent Compound A and AZD6244 in combination.

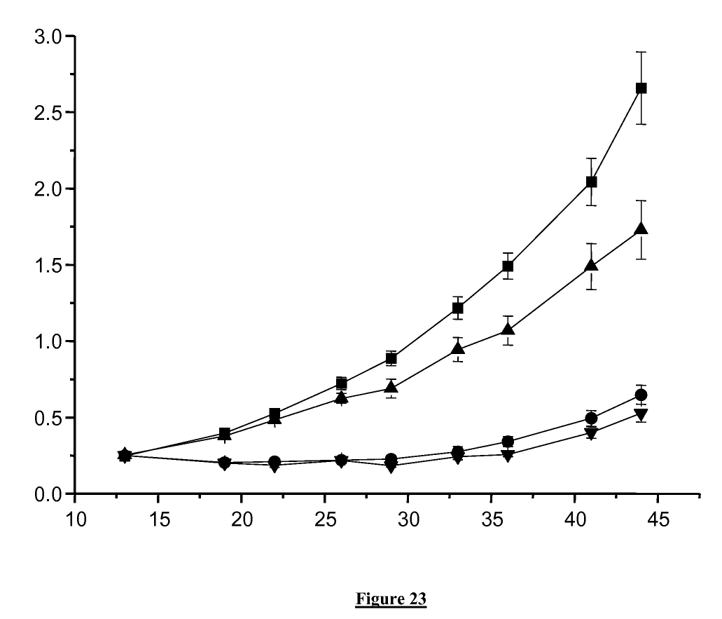

FIG. 23. Combination of AZD6244 and Rapamycin in Calu-6 xenografts; tumour volume in cm³ against days of dosing. Squares represent vehicle; circles represent AZD6244 monotherapy; triangles represent Rapamycin monotherapy; inverted triangles represent AZD6244 and Rapamycin in combination.

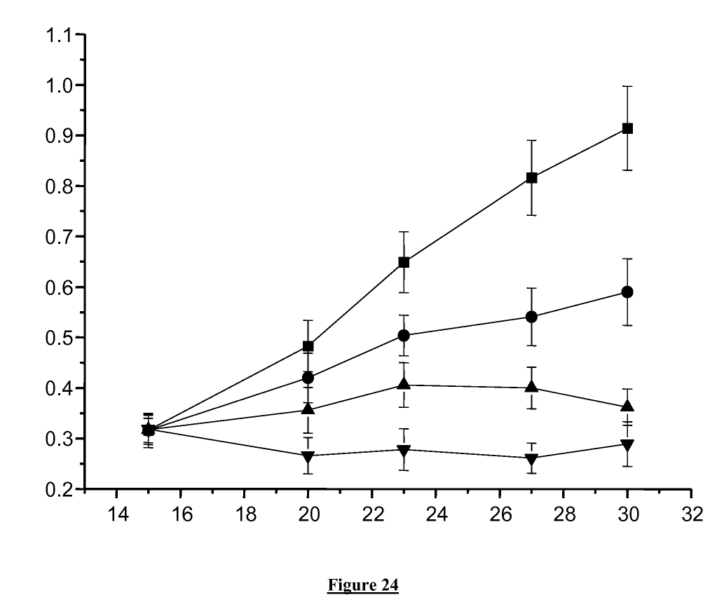

FIG. 24. Combination of AZD6244 and Compound A in A549a xenografts; tumour volume in cm³ against days of dosing. Squares represent vehicle; circles represent AZD6244 monotherapy; triangles represent Compound A monotherapy; inverted triangles represent Compound A and AZD6244 in combination.

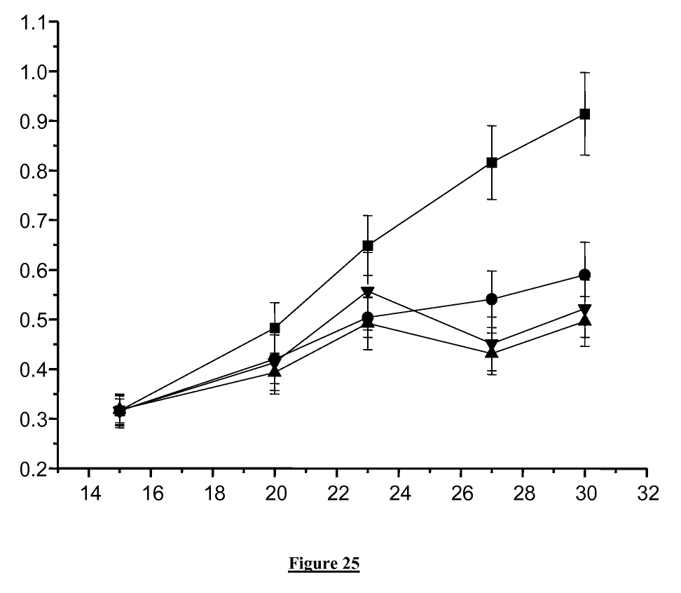

FIG. 25. Combination of AZD6244 and Rapamycin in A549a xenografts; tumour volume in cm³ against days of dosing. Squares represent vehicle; circles represent AZD6244 monotherapy; triangles represent Rapamycin monotherapy; inverted triangles represent AZD6244 and Rapamycin in combination.

### EXAMPLES

### General Experimental Methods

Thin Layer chromatography was carried out using Merck Kieselgel 60 F₂₅₄ glass backed plates. The plates were visualized by the use of a UV lamp (254 nm). Silica gel 60 (particle sizes 40-63 μm) supplied by E.M. Merck was employed for flash chromatography. ¹H NMR spectra were recorded at 300 MHz on a Bruker DPX-300 instrument. Chemical shifts were referenced relative to tetramethylsilane.

### Purification of Samples

The samples were purified on Gilson LC units. Mobile phase A—0.1% aqueous TFA, mobile phase B—Acetonitrile; flow rate 6 ml/min; Gradient—typically starting at 90% A/10% B for 1 minute, rising to 97% after 15 minutes, holding for 2 minutes, then back to the starting conditions. Column: Jones Chromatography Genesis 4 μm, C18 column, 10 mm×250 mm. Peak acquisition based on UV detection at 254 nm.

### Identification of Samples

Mass spectra were recorded on a Finnegan LCQ instrument in positive ion mode. Mobile phase A—0.1% aqueous formic acid. Mobile phase B—Acetonitrile; Flowrate 2 ml/min; Gradient—starting at 95% A/5% B for 1 minute, rising to 98% B after 5 minutes and holding for 3 minutes before returning to the starting conditions. Column: Varies, but always C18 50 mm×4.6 mm (currently Genesis C18 4 μm. Jones Chromatography). PDA detection Waters 996, scan range 210-400 nm.

### QC Method QC2-Long

Mass spectra were recorded on a Waters ZQ instrument in Electrospray ionisation mode. Mobile phase A—0.1% aqueous formic acid. Mobile phase B—0.1% Formic acid in acetonitrile; Flowrate 2 ml/min; Gradient—starting at 95% A/5% B, rising to 95% B after 20 minutes and holding for 3 minutes before returning to the starting conditions. Column: Varies, but always C18 50 mm×4.6 mm (currently Genesis C18 4u 50 mm×4.6 mm, Hichrom Ltd). PDA detection Waters 996, scan range 210-400 nm.

### Microwave Synthesis

Reactions were carried out using a Personal Chemistry™ Emrys Optimiser microwave synthesis unit with robotic arm. Power range between. 0-300 W at 2.45 GHz. Pressure range between 0-20 bar; temperature increase between 2-5° C./sec; temp range 60-250° C.

### General Procedure for the Synthesis of 2,4,7-Substituted Pyridopyrimidine Derivatives:

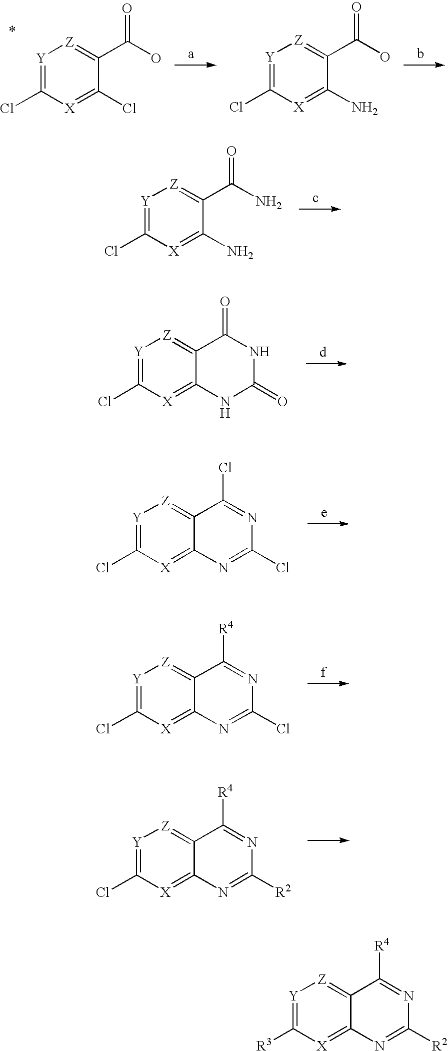

*2-amino-6-chloronicotinic acid—X═N, Y═C, Z=C
*3-amino-chloroisonicotinic acid —X═C, Y═N, Z=C
*3-Amino-chloropyridine-2-carboxylic acid —X═C, Y═C, Z=N
a) NH₃, 14 bar; b) (i) SOCl₂, THF, r.t., (ii) NH₃ c) Oxalyl chloride, Toluene, A; d) DIPEA, POCl₃, Toluene or Anisole, Δ;e) Appropriate amine, diisopropylethylamine, CH₂Cl₂ or Anisole; f) Appropriate amine, diiosopropylethyl amine, DMA, 70° C.;

### Synthesis of 2,4,7-Substituted Pyridopyrimidine Derivatives

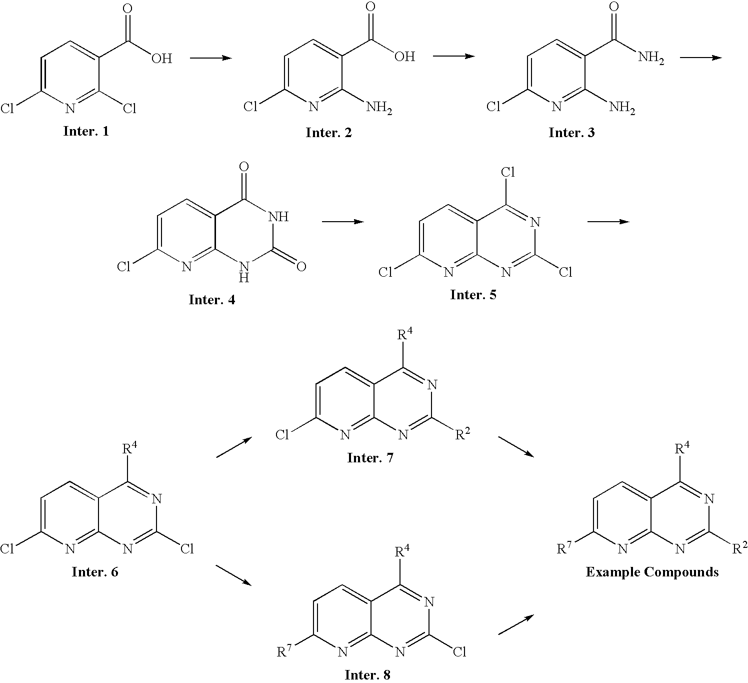

### Intermediates:

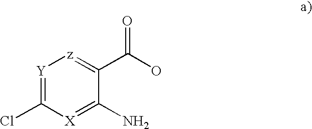

To the appropriate amino acid (1 equiv) was added liquid ammonia (sufficient to make a 0.6M solution of substrate in ammonia). The suspension was sealed in a pressure vessel which was then heated slowly to 130° C. It was noted that at this temperature a pressure of 18 bar was observed. This temperature and pressure was maintained for a further 16 hours whereupon the mixture was cooled to room temperature. The pressure vessel was opened and the reaction poured into ice cold water (1 reaction volume). The resulting solution was acidified to pH 1-2 using concentrated HCl which caused a precipitate to form. The acidic mixture was allowed to warm to room temperature and was stirred like this for a further 30 min The suspension was then extracted with diethyl ether (3×400 ml). The combined organic extracts were then filtered and the filtrate concentrated in vacuo to give a white solid which was dried further over P₂O₅ to give the title compound (typically 80-90% yield and 90%+pure) in suitably pure form to be used without any further purification.

### 2-Amino-6-chloronicotinic Acid (Inter. 2)

To 2,6-dichloronicotinic acid (Inter. 1) (1 equiv) was added liquid ammonia (sufficient to make a 0.6M solution of substrate in ammonia). The suspension was sealed in a pressure vessel which was then heated slowly to 130° C. It was noted that at this temperature a pressure of 18 bar was observed. This temperature and pressure was maintained for a further 16 hours whereupon the mixture was cooled to room temperature. The pressure vessel was opened and the reaction poured into ice cold water (1 reaction volume). The resulting solution was acidified to pH 1-2 using concentrated HCl which caused a precipitate to form. The acidic mixture was allowed to warm to room temperature and was stirred like this for a further 30 minutes. The suspension was then extracted with diethyl ether (3×400 ml). The combined organic extracts were then filtered and the filtrate concentrated in vacuo to give a white solid which was dried further over P₂O₅ to give the title compound (90% yield and 96% pure) in suitably pure form to be used without any further purification. m/z (LC-MS, ESP): 173 [M+H]⁺R/T=3.63 mins

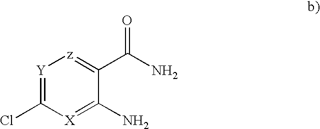

To a 0.3 M solution of amino acid (1 equiv) in anhydrous THF, under an inert atmosphere, was added thionyl chloride (3.3 equiv) in a dropwise fashion. The reaction mixture was stirred at room temperature for 2 hours. After this time the reaction was concentrated in vacuo to give a crude yellow solid residue. The crude solid was dissolved in THF (equal to initial reaction volume) and concentrated in vacuo again to give a yellow solid residue. The residue was dissolved once more in THF and concentrated as before to give a solid residue which was then dissolved in THF (to give a solution of 0.3M) and ammonia gas bubbled through the solution for 1 hour. The resultant precipitate was removed by filtration and the filtrate concentrated in vacuo to give a yellow precipitate which was triturated with water at 50° C. then dried to give the title compound (typically 90-95%) yield and suitably clean enough to be used without any further purification.

### 2-Amino-6-chloronicotinamide (Inter. 3)

To a 0.3 M solution of 2-amino-6-chloronicotinic acid (Inter. 2) (1 equiv) in anhydrous THF, under an inert atmosphere, was added thionyl chloride (3.3 equiv) in a dropwise fashion. The reaction mixture was stirred at room temperature for 2 hours. After this time the reaction was concentrated in vacuo to give a crude yellow solid residue. The crude solid was dissolved in THF (equal to initial reaction volume) and concentrated in vacuo again to give a yellow solid residue. The residue was dissolved once more in THF and concentrated as before to give a solid residue which was then dissolved in THF (to give a solution of 0.3M) and ammonia gas bubbled through the solution for 1 hour. The resultant precipitate was removed by filtration and the filtrate concentrated in vacuo to give a yellow precipitate which was triturated with water at 50° C. then dried to give the title compound (92% yield, 93% purity), suitably clean to be used without any further purification. m/z (LC-MS, ESP): 172 [M+H]⁺R/T=3.19 mins

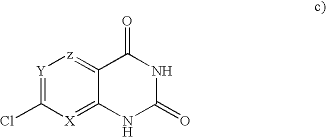

To a stirred solution (0.06 M) of substrate (1 equiv) in anhydrous toluene under an inert atmosphere was added oxalyl chloride (1.2 equiv) in a dropwise manner. The resulting mixture was then heated to reflux (115° C.) for 4 hours whereupon it was cooled and stirred for a further 16 hours. The crude reaction mixture was then concentrated to half its volume in vacuo and filtered to give the desired product in suitably pure form to be used without any further purification.

### 7-Chloro-1H-pyrido[2,3-d]pyrimidine-2,4-dione (Inter. 4)

To a stirred solution (0.06 M) of 2-amino-6-chloronicotinamide (Inter. 3) (1 equiv) in anhydrous toluene under an inert atmosphere was added oxalyl chloride (1.2 equiv) in a dropwise manner. The resulting mixture was then heated to reflux (115° C.) for 4 hours whereupon it was cooled and stirred for a further 16 hours. The crude reaction mixture was then concentrated to half its volume in vacuo and filtered to give the desired product in suitably pure form (95% yield, 96% purity) to be used without any further purification. m/z (LC-MS, ESP): 196 [M−H]⁻R/T=3.22 mins

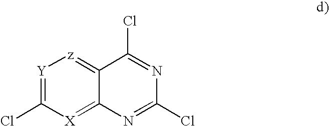

To a stirred 0.5 M suspension of the appropriate dione (1 equiv) in anhydrous toluene under an inert atmosphere was slowly added diisopropylethylamine (3 equiv). The reaction mixture was then heated to 70° C. for 30 minutes and then cooled to room temperature prior to the addition of POCl₃ (3 equiv). The reaction was then heated to 100° C. for 2.5 hours before being cooled and concentrated in vacuo to give a crude slurry which was then suspended in EtOAc and filtered through a thin pad of Celite™. The filtrate was concentrated in vacuo to give a brown, oil which was dissolved in CH₂Cl₂ and stirred over silica gel for 30 minutes. After this time the silica was removed by filtration, the filtrate concentrated and the crude residue purified by flash chromatography (SiO₂) to give the title compound in analytically pure form.

### 2,4,7-Trichloro-pyrido[2,3-d]pyrimidine (Inter. 5)

To a stirred 0.5 M suspension of the dione (Inter. 4) (1 equiv.) in anhydrous toluene under an inert atmosphere was slowly added diisopropylethylamine (3 equiv.). The reaction mixture was then heated to 70° C. for 30 minutes and then cooled to room temperature prior to the addition of POCl₃ (3 equivalents). The reaction was then heated to 100° C. for 2.5 hours before being cooled and concentrated in vacuo to give a crude slurry which was then suspended in EtOAc and filtered through a thin pad of Celite™. The filtrate was concentrated in vacuo to give a brown, oil which was dissolved in CH₂Cl₂ and stirred over silica gel for 30 minutes. After this time the silica was removed by filtration, the filtrate concentrated and the crude residue purified by flash chromatography (SiO₂) to give the title compound in analytically pure form (48% yield, 96% purity). m/z (LC-MS, ESP): 234 [M+H]⁺ R/T=4.21 mins

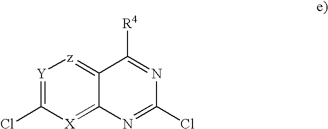

To a cooled (0-5° C.) stirred solution (0.1 M) of the appropriate trichloro-substrate (1 equiv) in CH₂Cl₂ was added diisopropylethylamine (1 equiv) in a dropwise fashion. The appropriate amine (1 equiv) was then added to the reaction mixture portionwise over the period of 1 hour. The solution was maintained at room temperature with stirring for a further 1 hour before the mixture was washed with water (2×1 reaction volume). The aqueous extracts were combined and extracted with CH₂Cl₂ (2×1 reaction volume). The organic extracts were then combined, dried (sodium sulphate), filtered and concentrated in vacuo to give an oily residue which solidified upon prolonged drying. The solid was triturated with diethylether and then filtered and the cake washed with cold diethyl ether to leave the title compound in suitable clean form to be used without any further purification.

### 4-Amino-2,7-dichloropyridopyrimidines (Inter. 6)

To a cooled (0-5° C.) stirred solution (0.1 M) of the trichloro substrate (Inter. 5)(1 equiv.) in CH₂Cl₂ was added diisopropylethylamine (1 equiv.) in a dropwise fashion. The appropriate amine (1 equiv.) was then added to the reaction mixture portionwise over the period of 1 hour. The solution was maintained at room temperature with stirring for a further 1 hour before the mixture was washed with water (2×1 reaction volume). The aqueous extracts were combined and extracted with CH₂Cl₂ (2×1 reaction volume). The organic extracts were then combined, dried (sodium sulphate), filtered and concentrated in vacuo to give an oily residue which solidified upon prolonged drying. The solid was triturated with diethylether and then filtered and the cake washed with cold diethyl ether to leave the title compound in suitable clean form to be used without any further purification.

Inter. 6a: 2,7-Dichloro-4-morpholin-4-yl-pyrido[2,3-d]pyrimidine; R⁴=morpholino; (92% yield, 90% purity) m/z (LC-MS, ESP): 285 [M+H]⁺R/T=3.90 mins
Inter. 6b: 2,7-Dichloro-4-((2S,6R)-2,6-dimethyl-morpholin-4-yl)-pyrido[2,3-d]pyrimidine; R⁴=(2R,6S)-2,6-Dimethyl-morpholino; (99% yield, 90% purity) m/z (LC-MS, ESP): 313 [M+H]⁺R/T=4.39 mins
Inter. 6c: 2,7-Dichloro-4-((S)-3-methyl-morpholin-4-yl)-pyrido[2,3-d]pyrimidine—R⁴=(S)-3-Methyl-morpholine, X═N, Y═C, Z=C: (87% yield, 92% purity) m/z (LC-MS, ESP): 301 [M+H]⁺ R/T=4.13 min
Inter. 6c: 2,7-Dichloro-4-((R)-3-methyl-morpholin-4-yl)-pyrido[2,3-d]pyrimidine—R⁴=(R)-3-Methyl-morpholine: (99% yield, 94% purity) m/z (LC-MS, ESP): 301 [M+H]⁺R/T=3.49 min

Alternatively, to a stirred 0.47 M suspension of the appropriate dione (1 equiv) in anhydrous anisole under an inert atmosphere was added POCl₃ (2.6 equiv). The mixture was heated to 55° C. and then diisopropylethylamine (2.6 equiv) was slowly added. The reaction mixture was then heated to 85-90° C. for 30 minutes. Water was added in portions (0.15 equiv), and the reaction mixture was held at 85-90° C. for a further 30 minutes. The reaction was cooled to 50° C., and then 15% of the anisole solvent was removed by vacuum distillation. The mixture was then cooled to −5° C. and diisopropylethylamine (1.1 equiv) was added. A 4.9M solution of the appropriate amine (1.05 equiv) in anisole was then added to the reaction mixture continuously over a period of 1 hour. The solution was then warmed to 30° C. and the reaction monitored by HPLC until reaction completion.

One third of the resulting mixture from the above reaction was then added over 10 min to a stirred mixture of 1.95M aqueous potassium hydroxide (3.9 equiv) and i-butanol (6.9 equiv) at 60° C. The stirring was stopped, the phases were allowed to separate, and the aqueous phase was removed. Stirring was resumed, and 1.95M aqueous potassium hydroxide (3.9 equiv) was added to the retained organic phase. The second third of the resulting reaction mixture from the reaction above was then added over 10 min at 60° C. Again, stirring was stopped, the phases were allowed to separate, and the aqueous phase was removed. Stirring was resumed, and 1.95M aqueous potassium hydroxide (3.9 equiv) was added to the retained organic phase. The remaining third of the resulting reaction mixture from the reaction above was then added over 10 min at 60° C. Again, stirring was stopped, the phases were allowed to separate, and the aqueous phase was removed. Water was then added to the organic phase with stirring, and the stirred mixture heated to 75° C. Stirring was stopped, the phases were allowed to separate, and the aqueous phase was removed. The resulting organic phase was stirred and allowed to cool to 30° C., and then as the mixture was heated to 60° C. heptane (11.5 equiv) was added over 20 min when the mixture was around 40° C. After being heated to 60° C., the mixture was cooled over 2.5 h to 10° C. After 30 min, the resulting slurry was filtered off, washed with a 10:1 heptane:anisole mixture (2×1.4 equiv) and then washed with heptane (2×1.4 equiv). The solid was then dried in a vacuum oven at 50° C. to leave the title compound in suitable clean form to be used without any further purification.

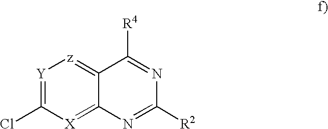

To a solution (0.2 M) of the appropriate dichloro-substrate (1 equiv) in anhydrous dimethyl acetamide under an inert atmosphere was added diisopropylethylamine (1 equiv) followed by the appropriate amine (1 equiv). The resulting mixture was heated for 48 hours at 70° C. before being cooled to ambient temperature. The reaction was diluted with CH₂Cl₂ (1 reaction volume) and then washed with water (3×1 reaction volumes). The organic extract was concentrated in vacuo to give a syrup which was dissolved in EtOAc (1 reaction volume) and washed with saturated brine solution before being dried (sodium sulphate) and concentrated in vacuo to give an oil. The crude residue was purified by flash chromatography (SiO₂, eluted with EtOAc:Hex (7:3) going to (1:1)) to give the title compound as a yellow solid that was suitably clean to be used without any further purification.

### 2,4-Diamino-7-chloropyridopyrimidines (Inter. 7)

To a solution (0.2 M) of the appropriate dichloro-substrate (Inter. 6a or 6b) (1 equiv) in anhydrous dimethyl acetamide under an inert atmosphere was added diisopropylethylamine (1 equiv) followed by the appropriate amine (1 equiv.). The resulting mixture was heated for 48 hours at 70° C. before being cooled to ambient temperature. The reaction was diluted with CH₂Cl₂ (1 reaction volume) and then washed with water (3×1 reaction volumes). The organic extract was concentrated in vacuo to give a syrup which was dissolved in EtOAC (1 reaction volume) and washed with saturated brine solution before being dried (sodium sulphate) and concentrated in vacuo to give an oil. The crude residue was purified by flash chromatography (SiO₂, eluted with EtOAc:Hex (7:3) going to (1:1)) to give the title compound as a yellow solid that was suitably clean to be used without any further purification.

Inter. 7a: 7-Chloro-2-((2S,6R)-2,6-dimethyl-morpholin-4-yl)-4-morpholin-4-yl-pyrido[2,3-d]pyrimidine; R⁴=morpholine, R²=cis-dimethylmorpholine; (45% yield, 85% purity) m/z (LC-MS, ESP): 348 [M+H]⁺R/T=4.16 mins
Inter. 7b: 7-Chloro-4-(2-methyl-piperidin-1-yl)-2-morpholin-4-yl-1-pyrido[2,3-d]pyrimidine; R⁴=morpholine, R²=2-methylpiperidine; (57% yield, 95% purity) m/z (LC-MS, ESP): 348.1 [M+H]⁺R/T=3.42 mins
Inter. 7c: 7-Chloro-4-((2S,6R)-2,6-dimethyl-morpholin-4-yl)-2-((S)-3-methyl-morpholin-4-yl)pyrido[2,3-d]pyrimidine (intermediate for compound 11k:) R⁴=cis-dimethylmorpholine, R²=(S)-3-Methyl-morpholine; (48% yield, 90% purity) m/z (LC-MS, ESP): 378 [M+H]⁺R/T=3.74 mins
Inter. 7d: 7-Chloro-2-((S)-3-methyl-morpholin-4-yl)-4-morpholin-4-yl-pyrido[2,3-d]pyrimidine (Intermediate for compound 11a): R⁴=morpholine, R²=(S)-3-Methyl-morpholine; (70% yield, 97% purity) m/z (LC-MS, ESP): 350 [M+H]⁺R/T=3.44 mins
Inter. 7e: 7-Chloro-2-(2-ethyl-piperidin-1-yl)-4-morpholin-4-yl-pyrido[2,3-d]pyrimidine (intermediate for compound 11ay): R⁴=morpholine, R²=2-Ethyl-piperidine; (56% yield, 95% purity) m/z (LC-MS, ESP): 362 [M+H]⁺R/T=3.78 mins
Inter. 7f: 7-Chloro-4-((S)-3-methyl-morpholin-4-yl)-2-((S)-3-methyl-morpholin-4-yl)-pyrido[2,3-d]pyrimidine—R⁴=(S)-3-Methyl-morpholine, R²=(S)-3-Methyl-morpholine, X═N, Y═C, Z=C: (71% yield, 90% purity) m/z (LC-MS, ESP): 364 [M+H]⁺R/T=3.52 min
Inter. 7g: 7-Chloro-2-(2-ethyl-piperidin-1-yl)-4-((S)-3-methyl-morpholin-4-yl)-pyrido[2,3-d]pyrimidine—R⁴═(S)-3-Methyl-morpholine, R²=2-Ethyl-piperidine, X═N, Y═C, Z=C: (51% yield, 98% purity) m/z (LC-MS, ESP): 376 [M+H]⁺R/T=3.88 min
Inter. 7h: 7-Chloro-4-((S)-3-methyl-morpholin-4-yl)-2-morpholin-4-yl-pyrido[2,3-d]pyrimidine, —R⁴=(S)-3-Methyl-morpholine, R²=morpholine, X═N, Y═C, Z=C: (72% yield, 96% purity) m/z (LC-MS, ESP): 350 [M+H]⁺R/T=3.45 min
Inter. 7i: 7-Chloro-2-((2S,6R)-2,6-dimethyl-morpholin-4-yl)-4-((S)-3-methyl-morpholin-4-yl-pyrido[2,3-d]pyrimidine—R⁴=(S)-3-Methyl-morpholine, R²=cis-dimethylmorpholine: (33% yield) m/z (LC-MS, ESP): 378 [M+H]⁺R/T=3.68 min
Inter. 7j: 7-Chloro-4-((R)-3-methyl-morpholin-4-yl)-2-((R)-3-methyl-morpholin-4-yl)-pyrido[2,3-d]pyrimidine, —R⁴=R²=(R)-3-Methyl-morpholine: (48% yield, 100% purity) m/z (LC-MS, ESP): 364 [M+H]⁺R/T=2.80 min
To a 0.33 M solution of 2,7-dichloro-4-((S)-3-methyl-morpholin-4-yl)-pyrido[2,3-d]pyrimidine (1 equiv) in N,N-dimethylacetamide was added Hunig's base (1 equiv) followed by the appropriate amine (1.1 equiv). The reaction mixture was heated 40° C. for 1 hour. After this time the reaction was allowed to cool, diluted with EtOAc (1 reaction volume) and then washed with water (1 reaction volume). The aqueous fraction was removed and extracted further with EtOAc (2×1 reaction volume). The combined organic extracts were dried (MgSO₄), filtered and concentrated in vacuo to give a crude oily residue which was purified by flash chromatography (SiO₂) using EtOAc/Hexanes as eluent which furnished the desired products in a suitably clean form.
Inter. 7k: 7-Chloro-4-((S)-3-methyl-morpholin-4-yl)-2-thiomorpholin-4-yl-pyrido[2,3-d]pyrimidine: (30% yield, 100% purity) m/z (LC-MS, ESP): 366.4[M+H]⁺R/T=3.00 min
Inter. 7l: 7-Chloro-4-((S)-3-methyl-morpholin-4-yl)-2-(4-methyl-piperazin-1-yl)-pyrido[2,3-d]pyrimidine: (32% yield, 95% purity) m/z (LC-MS, ESP): 363.4[M+H]⁺R/T=2.37 min

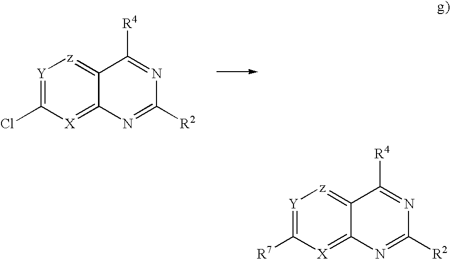

The appropriate chloro-substrate (1 equiv) was dissolved in a toluene/ethanol (1:1) solution (0.02 M). Sodium carbonate (2 equiv) and the appropriate pinacolate boron ester or boronic acid (1 equiv) were then added followed by tetrakis(triphenylphosphine) palladium⁰ (0.1 equiv). The reaction vessel was sealed and the mixture exposed to microwave radiation (140° C., medium absorption setting) for 30 minutes. Upon completion the samples were filtered through a silica cartridge, washed with EtOAc and then concentrated in vacuo. The crude residue was then purified by preparative HPLC to give the desired products.

### 4-amino-7-aryl-2-chloropyridopyrimidines (Inter. 8)

To a solution (0.09 M) of the appropriate boronic acid or ester (1 equiv) in water (1 volume) was added the appropriate 2,7-dichloro-4-amino pyridopyrimidine (1 equiv) (Inter. 6a or 6b) potassium carbonate (2.5 equiv) and acetonitrile (1 volume). The mixture was degassed by bubbling nitrogen through the solution while sonicating for 15 minutes before the addition of by tetrakis(triphenylphosphine) palladium (0.03 equiv). The mixture was degassed for a further 5 minutes before heating under an inter atmosphere at 95° C. for 2 hours. Upon completion, the reaction was cooled to room temperature and filtered under vacuum. The filtrate was concentrated in vacuo to give a solid residue which was dissolved in CH₂Cl₂ (1 volume) and washed with water (1 volume). The organic extract was then dried (MgSO₄), filtered and concentrated in vacuo to give an amorphous solid which was triturated with Et₂O to leave the desired product as a fine powder.

Inter. 8a (R⁴=Morpholine, R⁷=4-chlorophenyl)

2-Chloro-7-(4-chloro-phenyl)-4-morpholin-4-yl-pyrido[2,3-d]pyrimidine; ¹H NMR (300 MHz, Solvent CDCl₃?? δppm 8.29-7.96 (m, 2H), 7.75 (d, J=8.70 Hz, 1H), 7.54-7.21 (m, 2H), 5.29 (s, 1H), 3.91 (m, 8H).

### Example 1

### Preparation of 2,4,7-Substituted Pyridopyrimidine Intermediates

### Procedures for the Synthesis of 2-Chloro-4-((S)-3-methyl-morpholin-4-yl)-7-aryll-pyrido[2,3-d]pyrimidine Derivatives

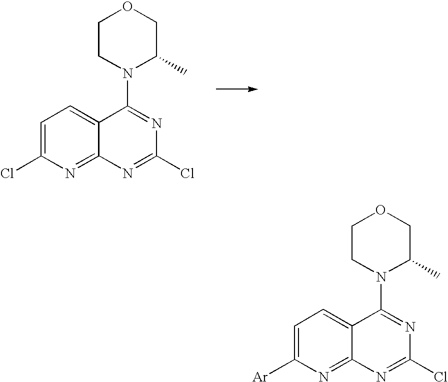

To a (0.1 M) solution of 2,7-dichloro-4-((S)-3-methyl-morpholin-4-yl)-pyrido[2,3-d]pyrimidine (1 equiv) in MeCN/H₂O (1:1 mixture) was added the appropriate pinacolate boron ester or boronic acid (1.1 equiv) and potassium carbonate (3 equiv). The mixture was degassed with nitrogen for 20 minutes before the addition of tetrakis(triphenylphosphine)palladium⁰ (0.05 equiv). The reaction was degassed for a further 5 minutes before being heated to reflux under an inert atmosphere for 3 hours. Whereupon, it was concentrated in vacuo and the crude residue partitioned between CH₂Cl₂/H₂O. The organic fraction was dried (MgSO₄), filtered and concentrated in vacuo to give an oil which was further purified by flash chromatography (SiO₂) using 5% MeOH in CH₂Cl₂ as eluent.

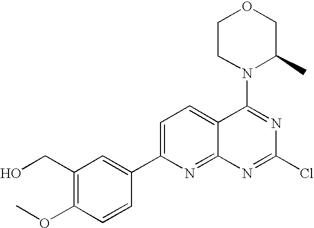

{5-[2-Chloro-4-((R)-3-methyl-morpholin-4-yl)-pyrido[2,3-d]pyrimidin-7-yl]-2-methoxy-phenyl}-methanol: (97% yield, 93% purity) m/z (LC-MS, ESP):401 [M+H]⁺, R/T=3.42 min)

### General Procedures for the Synthesis of Boronic Ester:

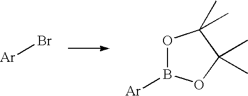

The bromo-aryl compound (1 equiv) was dissolved in dioxane (0.1 M). Bis(pinacolato)diboron (1.1 equiv), potassium acetate (3.5 equiv) and dppf (0.05 equiv) were added and the mixture was degassed with nitrogen for 20 minutes. (1,1′-Bis(diphenylphosphino)ferrocene-dichloropalladium (0.05 equiv) was added and the mixture was degassed for a further 5 minutes. The reaction mixture was heated to 120° C. for 2 hours under nitrogen. After cooling to room temperature, the reaction mixture was diluted with CH₂Cl₂ and filtered through Celite™. The filtrate was concentrated in vacuo to give a dark oil. The residue was partitioned between EtOAc and saturated aqueous sodium bicarbonate and the aqueous layer further extracted with EtOAc. The combined organic phases were dried (MgSO₄), filtered and the filtrate was concentrated in vacuo to give a residue. The residue may be purified by recrystallisation or may be purified by flash column chromatography for example on silica gel eluting with 0 to 30% ethyl acetate in hexane.

### Procedures for the Preparation of Examples 1a

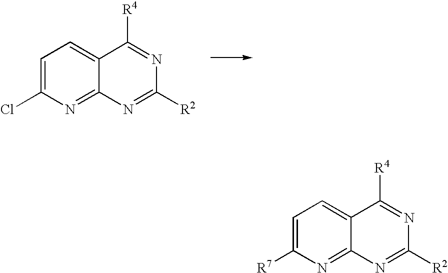 [1366] R⁴=(S)-3-methyl-morpholine
R²=(S)-3-methyl-morpholine or cis-dimethylmorpholine or 2-Ethyl-piperidine or morpholine or thiomorpholine or 4-methylpiperazine
R⁷=aryl or heteroaryl

### Procedures for the Suzuki Coupling:

The synthesis of the appropriate chloro-substrate has been described in the present document as intermediates. The appropriate pinacolate boron ester or boronic acids were prepared according to synthesis described in the present document (as intermediates) or commercially available, typically from the following suppliers:

### Sigma-Aldrich, Lancaster, Frontier Scientific, Boron Molecular, Interchim, Asymchem, Combi-blocks, Apollo Scientific, Fluorochem, ABCR, Digital Speciality Chemicals.

### Conditions A:

The appropriate chloro-substrate (1 equiv) was dissolved in a toluene/ethanol (1:1) solution (0.02 M). Sodium carbonate (2 equiv) and the appropriate pinacolate boron ester or boronic acid (1 equiv) were then added followed by tetrakis(triphenylphosphine) palladium⁰ (0.1 equiv). The reaction vessel was sealed and the mixture exposed to microwave radiation (140° C., medium absorption setting) for 30 minutes. Upon completion the samples were filtered through a silica cartridge, washed with EtOAc and then concentrated in vacuo. The crude residue was then purified by preparative HPLC to give the desired products.

### Conditions B:

A mixture of the appropriate chloro-substrate (1 equiv), potassium carbonate (2.4 equiv), the appropriate pinacolate boron ester or boronic acid (1.1 equiv) and tetrakis(triphenylphosphine) palladium⁰ (0.05 equiv) in n-butanol (0.03 M of chloro-substrate) was stirred at 120° C. for 2 hours. Upon completion the samples were filtered through a silica cartridge, washed through with CH₂Cl₂ and then concentrated in vacuo. The crude residue was then purified by preparative HPLC to give the desired products.

### Conditions C:

To a mixture of the appropriate chloro-substrate (1 equiv), potassium carbonate (2.4 equiv), and the appropriate pinacolate boron ester or boronic acid (1.1 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (150° C., medium absorption setting) for 30 minutes under nitrogen atmosphere. Upon completion the samples were filtered through a silica cartridge, washed with CH₂Cl₂ and methanol and then concentrated in vacuo. The crude residue was then purified by preparative HPLC to give the desired products.

### Conditions D:

To a mixture of the appropriate chloro-substrate (1 equiv), potassium carbonate (1.2 equiv), and the appropriate pinacolate boron ester or boronic acid (1.2 equiv) in acetonitrile/water (1:1) (0.083 M of chloro-substrate) was added tetrakis(triphenylphosphine) palladium⁰ (0.05 equiv). The reaction vessel was sealed and exposed to microwave radiation (130° C., medium absorption setting) for 25 minutes under nitrogen atmosphere. Upon completion the sample was purified by column chromatography on silica gel using a gradient MeOH/CH₂Cl₂ to afford the desired product which was recrystallised from diethyl ether.

### Conditions E:

To a mixture of the appropriate chloro-substrate (1 equiv), potassium carbonate (2.4 equiv), and the appropriate pinacolate boron ester or boronic acid (1.3 equiv) in acetonitrile/water (1:1) (0.041 M of chloro-substrate) was added tetrakis(triphenylphosphine) palladium⁰ (0.05 equiv). The reaction vessel was sealed and heated at 95° C. for 16 hours. Upon completion the reaction mixture was partitioned between aqueous HCl and CH₂Cl₂ and washed with aqueous HCl. Combined aqueous phase were extracted with CH₂Cl₂ (2×), neutralised with aqueous NaOH (2N) to give a cloudy solution that was extracted with CH₂Cl₂. Combined organic phases were washed with brine, dried (MgSO₄), filtered and concentrated in vacuo. The crude residue was purified by column chromatography on silica gel eluting with 0 to 4% MeOH in CH₂Cl₂ to give the desired product.

### Conditions F:

To a mixture of the appropriate chloro-substrate (1 equiv), potassium carbonate (2.0 equiv), and the appropriate pinacolate boron ester or boronic acid (1.5 equiv) in acetonitrile/water (1:1) (0.028 M of chloro-substrate) was added tetrakis(triphenylphosphine) palladium⁰ (0.05 equiv). The reaction vessel was sealed and heated at 120° C. for 2 hours under nitrogen atmosphere. Upon completion the reaction mixture was partitioned between water and CH₂Cl₂ and extracted with CH₂Cl₂. Combined organic phases were dried (MgSO₄), filtered and concentrated in vacuo. The crude residue was purified by column chromatography on silica gel eluting with 0 to 4% MeOH in CH₂Cl₂ to give the desired product which was recrystallised from hexane/diethyl ether.

### Conditions G:

To a mixture of the appropriate chloro-substrate (1 equiv), potassium carbonate (3.0 equiv), and the appropriate pinacolate boron ester or boronic acid (1.05 equiv) in acetonitrile/water (1:1) (0.068 M of chloro-substrate) was added tetrakis(triphenylphosphine) palladium⁰ (0.05 equiv). The reaction vessel was sealed and heated at 100° C. for 5 hours under nitrogen atmosphere. Upon completion the reaction mixture was partitioned between brine and CH₂Cl₂ and extracted with CH₂Cl₂. Combined organic phases were dried (MgSO₄), filtered and concentrated in vacuo. The crude residue was purified by column chromatography on silica gel eluting with 0 to 4% MeOH in CH₂Cl₂ to give the desired products which were recrystallised from hexane/CH₂Cl₂.

### Conditions H:

A mixture of the appropriate chloro-substrate (1 equiv), potassium carbonate (3.0 equiv), the appropriate pinacolate boron ester or boronic acid (1.1 equiv) and tetrakis(triphenylphosphine) palladium⁰ (0.05 equiv) in acetonitrile/water (0.1 M of chloro-substrate) was stirred at 100° C. for 8 hours. Upon completion the sample was concentrated in vacuo. The crude residue was then purified by preparative HPLC to give the desired product.

### Conditions I:

Conditions I were similar to conditions H apart form the heating method: 100° C. for 2 hours.

### Conditions J:

A mixture of the appropriate chloro-substrate (1 equiv), potassium carbonate (1.2 equiv), the appropriate pinacolate boron ester or boronic acid (1.2 equiv) and tetrakis(triphenylphosphine) palladium⁰ (0.05 equiv) in acetonitrile/water (0.03 M of chloro-substrate) was stirred at 100° C. for 2 hours. Upon completion the sample was concentrated in vacuo. The crude residue was then purified by preparative HPLC to give the desired product.

### Conditions K:

Conditions K were similar to conditions G apart form the heating method: 100° C. for 16 hours.

### Conditions L:

To a mixture of the appropriate chloro-substrate (1 equiv), potassium carbonate (2.5 equiv), and the appropriate pinacolate boron ester or boronic acid (1.10 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (100° C., medium absorption setting) for 90 minutes. Upon completion the reaction mixture was partly concentrated. The residue was partitioned between water and ethyl acetate and extracted with ethyl acetate and n-butanol. Combined organic phases were dried (MgSO₄), filtered and concentrated in vacuo. The crude residue was purified by column chromatography on silica gel eluting with 30 to 10% hexane in ethyl acetate to give the desired product which was recrystallised from hexane/CH₂Cl₂

### Conditions M:

A mixture of the appropriate chloro-substrate (1 equiv), cesium fluoride (3.0 equiv), the appropriate pinacolate boron ester or boronic acid (1.1 equiv) and tetrakis(triphenylphosphine) palladium⁰ (0.05 equiv) in acetonitrile/water (0.09 M of chloro-substrate) was stirred at 115° C. for 48 hours. Upon completion the sample was concentrated in vacuo to half original volume. The residue was partitioned between water and CH₂Cl₂. Organic phase was dried (MgSO₄), filtered and concentrated in vacuo. The crude residue was purified by column chromatography on silica gel eluting with 0 to 100% ethyl acetate in hexane to give the desired product.

### Conditions N:

A mixture of the appropriate chloro-substrate (1 equiv), tripotassium phosphate (1.5 equiv), the appropriate pinacolate boron ester or boronic acid (1.05 equiv) and bis(tri-t-butylphosphine) palladium (0.05 equiv) was suspended in dioxane (0.16 M of chloro-substrate). The reaction vessel was sealed and exposed to microwave radiation (170° C., medium absorption setting) for 45 minutes. Upon completion the sample was concentrated in vacuo. The residue was partitioned between water and CH₂Cl₂. The organic phase was dried (MgSO₄), filtered and concentrated in vacuo. The crude residue was purified by column chromatography on silica gel eluting with 40 to 100% ethyl acetate in hexane to give the desired product.

### Conditions O:

A mixture of the appropriate chloro-substrate (1 equiv), potassium carbonate (2.5 equiv), the appropriate pinacolate boron ester or boronic acid (1.1 equiv) and tetrakis(triphenylphosphine) palladium⁰ (0.05 equiv) in n-butanol (0.068 M of chloro-substrate) was stirred at 95° C. for 15 minutes. Upon completion, the residue was partitioned between ethyl acetate and brine. Organic phase was dried (MgSO₄), filtered and concentrated in vacuo. The crude residue was purified by column chromatography on silica gel eluting with 30 to 100% ethyl acetate in hexane to give the desired product which was recrystallised from ethyl acetate/hexane.

### Conditions P:

To a mixture of the appropriate chloro-substrate (1 equiv), potassium carbonate (2.0 equiv), and the appropriate pinacolate boron ester or boronic acid (2.0 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (120° C., medium absorption setting) for 10 minutes under nitrogen atmosphere. Upon completion the samples were filtered through a silica cartridge, washed through with CH₂Cl₂ and the concentrated in vacuo. The crude residue was then purified by preparative HPLC to give the desired product.

### Conditions Q:

A mixture of the appropriate chloro-substrate (1 equiv), potassium carbonate (2.5 equiv), the appropriate pinacolate boron ester or boronic acid (1.1 equiv) and tetrakis(triphenylphosphine) palladium⁰ (0.05 equiv) were dissolved in n-butanol (0.056 M of chloro-substrate). The reaction vessel was sealed and exposed to microwave radiation (150° C., medium absorption setting) for 30 minutes. Upon completion the samples were filtered through a silica cartridge, washed with CH₂Cl₂ and methanol and then concentrated in vacuo. The crude residue was purified by column chromatography on silica gel eluting with ethyl acetate and then 5% MeOH in CH₂Cl₂ to give the desired product.

### Conditions R:

A mixture of the appropriate chloro-substrate (1 equiv), potassium carbonate (2.5 equiv), the appropriate pinacolate boron ester or boronic acid (1.2 equiv) and tetrakis(triphenylphosphine) palladium⁰ (0.05 equiv) in acetonitrile/water (0.05 M of chloro-substrate) was stirred at 115° C. for 1.5 hours. Upon completion the crude reaction was filtered and the filtrate was concentrated in vacuo. The crude residue was purified by column chromatography on silica gel eluting with 5 to 20% MeOH in CH₂Cl₂ to give the desired product.

### Conditions S:

A mixture of the appropriate chloro-substrate (1 equiv), potassium carbonate (10.0 equiv), the appropriate pinacolate boron ester or boronic acid (1.2 equiv) and tetrakis(triphenylphosphine) palladium⁰ (0.05 equiv) in acetonitrile/water (0.1 M of chloro-substrate) was stirred at 100° C. for 2 hours. Upon completion the reaction mixture was partitioned between water and CH₂Cl₂ and extracted with CH₂Cl₂. Combined organic phases were dried (MgSO₄), filtered and concentrated in vacuo. The crude residue was purified by column chromatography on silica gel eluting with 0 to 5% MeOH in CH₂Cl₂ to give the desired product which was recrystallised from hexane/CH₂Cl₂.

### Conditions T:

A mixture of the appropriate chloro-substrate (1 equiv), potassium carbonate (2.0 equiv), the appropriate pinacolate boron ester or boronic acid (2.0 equiv) and tetrakis(triphenylphosphine) palladium⁰ (0.05 equiv) was dissolved in acetonitrile/water (0.02 M of chloro-substrate). The reaction vessel was sealed and exposed to microwave radiation (130° C., medium absorption setting) for 30 minutes. Upon completion the sample was concentrated in vacuo. The crude residue was purified by column chromatography on silica gel eluting with 0 to 5% MeOH in CH₂Cl₂ to give the desired product.

### Conditions U:

A mixture of the appropriate chloro-substrate (1 equiv), potassium carbonate (3.0 equiv), the appropriate pinacolate boron ester or boronic acid (1.0 equiv) and tetrakis(triphenylphosphine) palladium⁰ (0.05 equiv) in acetonitrile/water (0.1 M of chloro-substrate) was stirred at 110° C. for 8 hours. Upon completion the reaction mixture was partitioned between water and CH₂Cl₂ and extracted with CH₂Cl₂. Combined organic phases were washed with brine, dried (MgSO₄), filtered and concentrated in vacuo. The crude residue was purified by column chromatography on silica gel eluting with 0 to 2% MeOH in CH₂Cl₂ to give the desired product which was recrystallised from hexane/CH₂Cl₂.

### Conditions V:

A mixture of the appropriate chloro-substrate (1 equiv), cesium fluoride (3.0 equiv), the appropriate pinacolate boron ester or boronic acid (1 equiv) and tetrakis(triphenylphosphine) palladium⁰ (0.05 equiv) in acetonitrile/water (0.1 M of chloro-substrate) was stirred at 100° C. for 16 hours. The reaction mixture was partitioned between water and CH₂Cl₂ and extracted with CH₂Cl₂. The organic phase was dried (MgSO₄), filtered and concentrated in vacuo. The crude residue was purified by column chromatography on silica gel eluting with 0 to 5% MeOH in CH₂Cl₂ to give the desired product which was recrystallised from hexane/CH₂Cl₂.

### Conditions W:

A mixture of the appropriate chloro-substrate (1 equiv), potassium carbonate (2.5 equiv), the appropriate pinacolate boron ester or boronic acid (1 equiv) and tetrakis(triphenylphosphine) palladium⁰ (0.05 equiv) was dissolved in acetonitrile/water (0.04 M of chloro-substrate). The reaction vessel was sealed and exposed to microwave radiation (110° C., medium absorption setting) for 10 minutes. The crude residue was purified by column chromatography on silica gel eluting with 0 to 2% MeOH in TBME to give the desired product.

TABLE 1

| | | Retention | | | |
|---|---|---|---|---|---|
| | Purity | time | m/z | | |
| | (%) | (min) | [M + H]⁺ | Conditions | Example Structure |
| 1a | 96 | 7.66 | 466.6 | A | 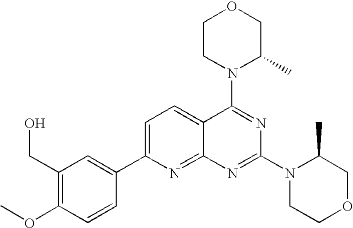 |

### Example 2

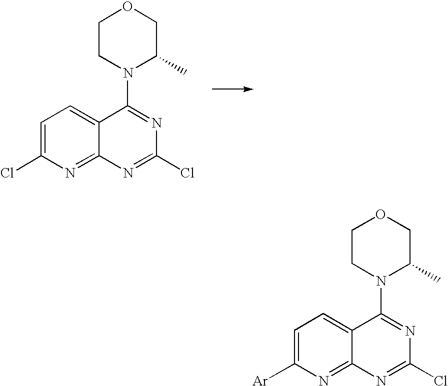

The chloro-substrate was reported in Example 1.

To a mixture of the appropriate chloro-substrate (1 equiv), potassium carbonate (2.5 equiv), and the appropriate boronic acid (1.1 equiv) in acetonitrile/water (1:1) (0.033 M of chloro-substrate) was added tetrakis(triphenylphosphine) palladium⁰ (0.05 equiv). The suspension was sonicated while degassed with nitrogen for 5 minutes then heated to 95° C. for 2 hours. Upon completion the reaction mixture was allowed to cool down to room temperature. The reaction mixture was concentrated in vacuo to half original volume. The crude residue was extracted with CH₂Cl₂ and the combined organic phases were washed with brine, dried (MgSO₄), filtered and concentrated in vacuo to give a yellow solid. The residue was sonicated in diethyl ether, collected by vacuum filtration to give the desired product as a yellow powder.

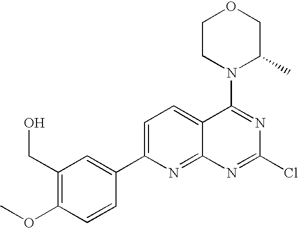

{5-[2-Chloro-4-((S)-3-methyl-morpholin-4-yl)-pyrido[2,3-d]pyrimidin-7-yl]-2-methoxy-phenyl}-methanol: (78% yield, 100% purity) m/z (LC-MS, ESP): 401 [M+H]⁺R/T=3.47 min

Alternatively, to a stirred mixture of bis(pinacolato)diboron (1.05 equiv) and potassium acetate (3 equiv) in N-methylpyrrolidine (13.5 equiv), purged with nitrogen, was added the corresponding bromobenzylalcohol (1 equiv) followed by PdCl₂(dppf) (0.02 equiv). The mixture was then heated to 60° C. and held for 10 min, then heated to 70° C. and held for 15 min and finally heated to 80° C. and held for 1 h. The appropriate chloro-substrate (1 equiv) was then added followed by PdCl₂(dppf) (0.02 equiv) and N-methylpyrrolidine (4.5 equiv). The temperature was then held at 75° C., then 4.3M aqueous potassium carbonate (3.5 equiv) was added over 13 min, then water (12 equiv) was added and the reaction was stirred at 75° C. for 90 min. Water (144 equiv) was then added slowly over 70 min with stirring while the temperature was reduced to 66° C. The temperature of the stirred mixture was then kept at 64° C. for 30 min, then cooled to 20° C. over 2.5 h, and held at 20° C. overnight. The resulting slurry was filtered, and the solid washed first with a 3:1 water:N-methylpyrrolidone mixture (18 equiv of water), then washed with water (24 equiv) and then washed with ethyl acetate (4×4.4 equiv). The solid was then dried in a vacuum oven at 50° C. to leave the title compound in suitable clean form to be used without any further purification. For example, {5-[2-Chloro-4-((S)-3-methyl-morpholin-4-yl)-pyrido[2,3-d]pyrimidin-7-yl]-2-methoxy-phenyl}-methanol: (73% yield)

### (Compounds 2a to 2b)

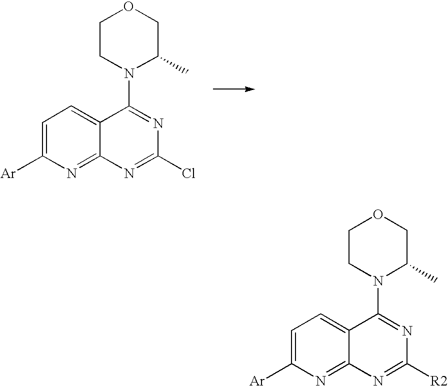

### Conditions A:

The appropriate chloro-substrate (1 equiv) was dissolved in DMA (0.04 M). Tripotassium phosphate (1.5 equiv) and the appropriate nucleophile (secondary amine) (1.5 equiv) were then added. The reaction vessel was sealed and the mixture exposed to microwave radiation (200° C., medium absorption setting) for 30 minutes. Upon completion the samples were filtered through a silica cartridge, washed with EtOAc and then concentrated in vacuo. The crude residue was then purified by preparative HPLC to give the desired products.

### Conditions B:

The appropriate chloro-substrate (1 equiv) was suspended in a propan-2-ol and aqueous ammonia (1:3) solution (0.02 M). The reaction vessel was sealed and the mixture exposed to microwave radiation (140° C., medium absorption setting) for 20 minutes. The crude residue was then purified by preparative HPLC to give the desired products.

### Conditions C:

The appropriate chloro-substrate (1 equiv) was dissolved in dioxane (0.04 M). Diisopropylethylamine (5.0 equiv) and the appropriate nucleophile (secondary amine) (1.5 equiv) were then added. The reaction vessel was sealed and the mixture exposed to microwave radiation (130° C., medium absorption setting) for 20 minutes. Upon completion the samples were concentrated in vacuo. The crude residue was then purified by preparative HPLC to give the desired products.

### Conditions D:

The appropriate chloro-substrate (1 equiv) was dissolved in dioxane (0.04 M). Tripotassium phosphate (3.0 equiv), xantphos (0.05 equiv), palladium acetate (0.05 equiv) and the appropriate nucleophile (amine) (1.5 equiv) were then added. The reaction vessel was sealed and the mixture exposed to microwave radiation (150° C., medium absorption setting) for 20 minutes. Upon completion the samples were filtered through a silica cartridge, washed with EtOAc and then concentrated in vacuo. The crude residue was then purified by preparative HPLC to give the desired products.

### Conditions E:

The appropriate chloro-substrate (1.0 equiv) was dissolved in dioxane (0.04 M). Diisopropylethylamine (5.0 equiv) and the appropriate nucleophile (secondary amine, with BOC-protected amino side chain) (1.5 equiv) were then added. The reaction vessel was sealed and the mixture exposed to microwave radiation (130° C., medium absorption setting) for 20 minutes. Upon completion the samples were concentrated in vacuo. To the crude residue was then added a 4 M solution of HCl in dioxane (0.15 M). The reaction mixtures were stirred at room temperature for 3 hours. Upon completion the samples were basified with a 2 N sodium hydroxide solution. The crude residue was then purified by preparative HPLC to give the desired products.

### Conditions F:

The appropriate nucleophile (substituted imidazole) (10.0 equiv) was dissolved in DMF (0.4 M). Sodium hydride (5.0 equiv) was then added. The reaction mixture was stirred at room temperature for 10 minutes under nitrogen and a solution of the appropriate chloro-substrate (1.0 equiv) in DMF (0.075 M) was added. The reaction vessel was sealed and the mixture exposed to microwave radiation (150° C., medium absorption setting) for 30 minutes. Upon completion the samples were filtered through a silica cartridge, eluted with CH₂Cl₂ and then concentrated in vacuo. The crude residue were then purified by preparative HPLC to give the desired products.

### Conditions G:

The appropriate chloro-substrate (1 equiv) was dissolved in dioxane (0.04 M). Diisopropylethylamine (5.0 equiv) and the appropriate nucleophile (secondary amine) (4.5 equiv) were then added. The reaction vessel was sealed and the mixture exposed to microwave radiation (130° C., medium absorption setting) for 40 minutes. Upon completion the samples were concentrated in vacuo. The crude residue was then purified by preparative HPLC to give the desired products.

### Conditions H:

The appropriate chloro-substrate (1 equiv) was dissolved in dioxane (0.04 M). Diisopropylethylamine (5.0 equiv) and the appropriate nucleophile (secondary amine) (10.0 equiv) were then added. The reaction vessel was sealed and the mixture exposed to microwave radiation (130° C., medium absorption setting) for 60 minutes. Upon completion the samples were concentrated in vacuo. The crude residue was then purified by preparative HPLC to give the desired products.

### Conditions I:

The appropriate chloro-substrate (1 equiv) was dissolved in a solution of 1% DMA in dioxane (0.04 M). Diisopropylethylamine (5.0 equiv) and the appropriate nucleophile (secondary amine) (10.0 equiv) were then added. The reaction vessel was sealed and the mixture exposed to microwave radiation (180° C., medium absorption setting) for 60 minutes. Upon completion the samples were concentrated in vacuo. The crude residue was then purified by preparative HPLC to give the desired products.

### Conditions J:

The appropriate chloro-substrate (1 equiv) was dissolved in a solution of 1% DMA in dioxane (0.04 M). Diisopropylethylamine (7.0 equiv) and the appropriate nucleophile (secondary amine) (3.0 equiv) were then added. The reaction vessel was sealed and the mixture exposed to microwave radiation (150° C., medium absorption setting) for 60 minutes. Upon completion the samples were concentrated in vacuo. The crude residue was then purified by preparative HPLC to give the desired products.

### Conditions K:

The appropriate chloro-substrate (1 equiv) was dissolved in DMF (0.075 M). Potassium carbonate (5.0 equiv) and the appropriate nucleophile (alcohol) (10.0 equiv) were then added. The reaction vessel was sealed and the mixture exposed to microwave radiation (120° C., medium absorption setting) for 20 minutes. Upon completion the samples were concentrated in vacuo. The crude residue was then purified by preparative HPLC to give the desired products.

### Conditions L:

The appropriate chloro-substrate (1 equiv) was dissolved in DMF (0.075 M). Potassium carbonate (5.0 equiv) and the appropriate nucleophile (alcohol) (20.0 equiv) were then added. The reaction vessel was sealed and the mixture exposed to microwave radiation (150° C., medium absorption setting) for 40 minutes. Upon completion the samples were concentrated in vacuo. The crude residue was then purified by preparative HPLC to give the desired products.

### Conditions M:

The appropriate chloro-substrate (1 equiv) was dissolved in DMA (0.13 M). Diisopropylethylamine (2.0 equiv) and the appropriate nucleophile (amine) (2.0 equiv) were then added. The reaction vessel was heated to 100° C. for 3 hours. Upon completion, the reaction mixture was partitioned between dichloromethane and water and the aqueous layer further extracted with dichloromethane. The combined organic phases were dried (MgSO₄), filtered and the filtrate was concentrated in vacuo to give a yellow residue which was purified by recrystallisation from diethyl ether.

### Conditions N:

5-[2-Chloro-4-((S)-3-methyl-morpholin-4-yl)-pyrido[2,3-d]pyrimidin-7-yl]-pyridin-2-ylamine (1 equiv) was dissolved in DMA (0.21 M). Diisopropylethylamine (1.0 equiv) and the appropriate nucleophile (amine) (1.1 equiv) were then added. The reaction vessel was sealed and the mixture exposed to microwave radiation (130° C., medium absorption setting) for 10 minutes. Upon completion, the reaction mixture was partitioned between dichloromethane and water and the aqueous layer further extracted with CH₂Cl₂. The combined organic phases were dried (MgSO₄), filtered and the filtrate was concentrated in vacuo to give a yellow residue which was purified by column chromatography on silica gel eluting with 0% to 10% MeOH in CH₂Cl₂ to give the desired product.

### Conditions O:

The appropriate chloro-substrate (1 equiv) was dissolved in DMA (0.16 M). Diisopropylethylamine (1.0 equiv) and the appropriate nucleophile (amine) (1.2 equiv) were then added. The reaction vessel was heated to 80° C. for 48 hours. Upon completion, the reaction mixture was partitioned between ethyl acetate and water and the organic layer washed with brine. The combined organic phases were dried (MgSO₄), filtered and the filtrate was concentrated in vacuo to give a residue which was purified by preparative HPLC to give the desired product.

### Conditions P:

The appropriate chloro-substrate (1 equiv) was dissolved in anisole (0.25 M) (10 vol). Diisopropylethylamine (1.3 equiv) and the appropriate nucleophile (amine) (1.3 equiv) were then added. The reaction vessel was heated to 125° C. and stirred for 11 h. Upon completion, the reaction mixture was allowed to cool to 50° C. Aqueous 20% citric acid solution (7 vol) was added, stirred for 5 min and then allowed to separate partitioned. The aqueous layer was removed and retained. The organic layer was then extracted with a further aliquot of aqueous 20% citric acid solution (3 vol). The organic layer discarded, and the aqueous layers combined. The combined aqueous layers were washed first with anisole (5 vol), then 50% aqueous sodium hydroxide solution (1.23 vol) was added slowly. The resulting aqueous phase was extracted with ethyl acetate (10 vol). The aqueous layer was discarded and the organic layer was washed first with 10% aqueous sodium hydroxide solution (5 vol) and then water (5 vol). The organic layer was then slurried with silicycle Si-thiourea scavenger at 50° C. for 2 h, then the scavenger was filtered off and washed with ethyl acetate (2×1 vol). The organic phase was cooled to 20° C., seeded to start crystallization and stirred until a slurry obtained. The slurry was heated to 50° C. under vacuum and ethyl acetate (3 vol) was removed by vacuum distillation. 2-Methylpentane (3.4 vol) was added and the mixture heated to 60° C. and then slowly cooled to 20° C. over 2 h. The resulting slurry was filtered, and the solid washed with 1:1 ethyl actetate:pentane (2×0.5 vol). The solid was then dried in a vacuum oven at 50° C. to leave the desired product. For example, compound 1a was obtained (50.4% yield). The crude product (1 equiv) was dissolved in DMSO (5 vol based on product weight) at 50° C. Water (2 vol) was added and the mixture stirred at 50° C. until product crystallizes. The slurry was heated to 60° C. and then water (3-vol) was added slowly over 30 min so that the temperature was maintained at 60° C. The mixture was slowly cooled to 20° C. over 2 h, and then held at 20° C. for 30 min. The resulting slurry was filtered, and the solid washed with 2:1 water:DMSO (0.5:1 vol), and then water (3×2 vol). The solid was then dried in a vacuum oven at 50° C. to leave the desired product.

TABLE 2

| | Purity | Retention | m/z | | |
|---|---|---|---|---|---|
| | (%) | time (min) | [M + H]⁺ | Conditions | Example Structure |
| 2ba | 97 | 4.03 | 466.2 | O | 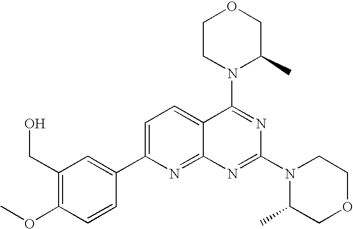 |
| 2b | 99 | 3.99 | 466.2 | O | 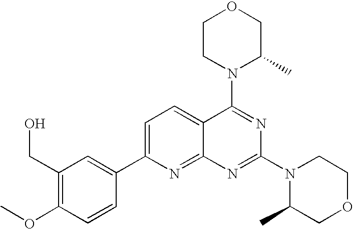 |

### NMR Data for Example 2ba

¹H NMR (300 MHz, CDCl₃?? δ ppm 8.10 (ArH, d, J=7.89 Hz, 2H), 7.97 (ArH, d, J=8.49 Hz, 1H), 7.42 (ArH, d, J=8.46 Hz, 1H), 6.98 (ArH, d, J=8.55 Hz, 1H), 4.88 (CH₂, d, J=5.25 Hz, 1H), 4.77 (CH₂OH, s, 2H), 4.56 (CH₂, d, J=13.38 Hz, 1H), 4.38-4.36 (CH₂, m, 1H), 4.02-3.51 (OCH₃+CH₂, m, 11H), 3.43-3.33 (CH₂, m, 1H), 1.47 (CH₃, d, J=6.77 Hz, 3H), 1.35 (CH₃, d, J=6.78 Hz, 3H).

¹³C NMR (75 MHz, CD₃COCD₃) δ? ppm 165.11, 162.27, 161.87, 159.54, 159.23, 134.74, 130.76, 129.41, 128.86, 128.39, 113.09, 110.32, 104.45, 71.20, 70.95, 67.17, 66.91, 61.80, 55.57, 52.82, 47.05, 44.44, 39.45, 14.74 and 14.44.

### NMR Data for Example 2b

¹H NMR (300 MHz, CDCl₃?? δ ppm 8.10 (ArH, d, J=8.76 Hz, 2H), 7.98 (ArH, d, J=8.49 Hz, 1H), 7.42 (ArH, d, J=8.46 Hz, 1H), 6.97 (ArH, d, J=8.37 Hz, 1H), 4.88 (CH₂, d, J=5.46 Hz, 1H), 4.77 (CH₂OH, s, 2H), 4.58-4.49 (CH₂, m, 1H), 4.39-4.36 (CH₂, d J=7.41 Hz, 1H), 4.02-3.51 (OCH₃+CH₂, m, 11H), 3.43-3.33 (CH₂, m, 1H), 1.48 (CH₃, d, J=6.78 Hz, 3H), 1.35 (CH₃, d, J=6.78 Hz, 3H).

¹³C NMR (75 MHz, CD₃COCD₃) δ ?ppm 165.05, 161.87, 159.45, 159.24, 134.78, 130.70, 129.44, 128.86, 128.38, 113.14, 110.33, 104.43, 71.19, 70.95, 67.16, 66.90, 61.77, 55.57, 52.82, 47.08, 44.44, 39.47, 14.76 and 14.44.

### Example 3

### In Vitro Combination Study of AZD6244 or 2-(2-fluoro-4-iodophenylamino)-N-(2-hydroxyethoxy)-1,5-dimethyl-6-oxo-1,6-dihydropyridine-3-carboxamide with the mTOR-Selective Inhibitor [5-[2,4-bis[(3S)-3-methylmorpholin-4-yl]pyrido[5,6-e]pyrimidin-7-yl]-2-methoxyphenyl]methanol (“Compound A”) Using a MTS Viable Cell Number Endpoint

The objective of this assay was to determine the in-vitro combination interaction when combining AZD6244 or 2-(2-fluoro-4-iodophenylamino)-N-(2-hydroxyethoxy)-1,5-dimethyl-6-oxo-1,6-dihydropyridine-3-carboxamide concurrently with Compound A or Rapamycin in the A2058, NCI-H727, Calu-6, NCI-H2291, NCI-H23, A549 and NCI-H460 cell lines using a 96-hour viable cell number endpoint (MTS).

The A2058 cell line was routinely cultured and assayed in DMEM (phenol red free)+10% Foetal Calf Serum (FCS)+1% glutamine. The A2058 cell line is wild type for HRAS, KRAS and NRAS and mutant for the BRAF V600E mutation.

The NCI-H727, Calu-6, NCI-H2291, NCI-H23, A549 and NCI-H460 cell lines were routinely cultured and assayed in RPMI (phenol red free)+10% Foetal Calf Serum (FCS)+1% glutamine.

MTS viable cell number assays were carried out to determine seeding densities required for 96 hours of exponential growth. Cells were seeded into 96-well plates at their pre-determined seeding density allowing for log phase growth. After 4-hours cells were dosed with monotherapy AZD6244, 2-(2-fluoro-4-iodophenylamino)-N-(2-hydroxyethoxy)-1,5-dimethyl-6-oxo-1,6-dihydropyridine-3-carboxamide, Compound A, or Rapamycin using the Hydra/ECHO 550 platform. Following incubation with compound for 96-hours viable cell number was determined using an MTS viable cell number (Promega™) endpoint.

TABLE 3
Summary of the IC₅₀ monotherapy values for AZD6244, 2-(2-fluoro-4-
iodophenylamino)-N-(2-hydroxyethoxy)-1,5-dimethyl-6-oxo-1,6-
dihydropyridine-3-carboxamide, Compound A and Rapamycin in
the cell lines tested as determined in a 96-hour viable cell number
endpoint by MTS. (Data expressed as Geomean IC₅₀ uM,
95% CIR, n = number of individual experiments).

| | IC₅₀ (95% CIR, n) | | | |
|---|---|---|---|---|
| | | 2-(2-fluoro-4- | | |
| | | iodophenylamino)- | | |
| | | N-(2- | | |
| | | hydroxyethoxy)- | | |
| | | 1,5-dimethyl- | | |
| | | 6-oxo-1,6- | Com- | |
| | | dihydropyridine- | pound | |
| Cell Line | AZD6244 | 3-carboxamide | A | Rapamycin |
| A2058 | 0.526 μM | 0.025 μM | 0.046 μM | Partial >10 μM |
| | (1.77, | (1.254, n = 5) | (2.923, | (n = 5) |
| | n = 5) | | n = 5) | |
| NCI-H727 | 0.036 μM | — | 0.068 μM | >10 μM |
| | | | | (partial effect) |
| Calu-6 | 0.257 μM | — | 0.050 μM | >10 μM |
| | | | | (partial effect) |
| NCI- | 0.704 μM | — | 0.023 μM | >10 μM |
| H2291 | | | | (partial effect) |
| NCI-H23 | 1.845 μM | — | 0.030 μM | >10 μM |
| | | | | (partial effect) |
| A549 | 12.37 μM | — | 0.037 μM | >10 μM |
| | | | | (partial effect) |
| NCI-H460 | 32.40 μM | — | 0.026 μM | >10 μM |
| | | | | (partial effect) |

Concurrent combination: Cells were seeded into 96-well plates at the above-determined seeding density allowing for log phase growth. After 4 hours cells were dosed concurrently with

AZD6244+Compound A,

2-(2-fluoro-4-iodophenylamino)-N-(2-hydroxyethoxy)-1,5-dimethyl-6-oxo-1,6-dihydropyridine-3-carboxamide+Compound A,

AZD6244+Rapamycin, or

2-(2-fluoro-4-iodophenylamino)-N-(2-hydroxyethoxy)-1,5-dimethyl-6-oxo-1,6-dihydropyridine-3-carboxamide+Rapamycin using the Hydra/ECHO 550 platform. Following incubation with compound for 96 hours, viable cell number was determined using an MTS viable cell number (Promega™) endpoint.

Non-linear MEA (Median Effect Analysis): The combination interaction between 2 active agents, each with well defined IC50 monotherapy dose responses was assessed using equal inhibitory effect ratios of the two agents (IC60, IC50, IC40, IC30, IC20, IC10). A combination index (CI)<0.85 indicates a synergistic interaction, CI 0.85-1.2 additivity and a CI>1.2 antagonism.

Curve-shift analysis for the A2058 cell line: Where only one agent was active and the other partially responsive, the combination interaction was assessed through curve shift analysis. That is a fixed dose of the partially active agent (in MTS assays) was applied across the full dose response of the active agent. Relative potency is defined as the ratio of the combination IC50 to the monotherapy IC50. A relative potency of less than 1 indicated the combination was more potent than monotherapy (p value<0.05, two sample t-test).

In cell lines where only one agent was active and the other inactive (or partially active) phenotypically (MTS) the combination interaction was assessed through Curve Shift analysis using a 3×8 matrix for cell lines. That is a fixed dose of the inactive/partially active agent was applied across the full dose response of the active agent. The dose of the inactive agent was chosen by its pharmacodynamic effect. The dose of AZD6244 was chosen by its pharmacodynamic effect against pERK 1/2(Thr202/Tyr204) (ED50 (20 nm), ED90 (333 nm) and ED95 (1000 nm). In the case of combinations between AZD6244 and Compound A, the 3×8 matrix curve shift analysis was used in NCI-H460, NCI-H23 and A549 cells in which AZD6244 is inactive (or partially active). Although Rapamycin demonstrated only partial activity and did not reach a true GI₅₀ at 10 μM, Rapamycin was treated as the active agent and an eight-point dose response plus three fixed doses of AZD6244 were used in NCI-H23, NCI-H460, NCI-H2291, A549 and Calu-6 cell lines.

FIG. 1 shows a representative Combination Index curve for treatment of the A2058 cell line with the MEK inhibitor AZD6244 and Compound A. Treatment of cells with the combination was beneficial. Synergy was observed across the dosing regime when AZD6244 and Compound A were dosed in concurrent combination at their IC₆₀, IC₅₀, IC₄₀, IC₃₀ and IC₂₀ inhibitory effect ratios.

FIG. 2 shows a representative Combination Index curve for treatment of the A2058 cell line with the MEK inhibitor 2-(2-fluoro-4-iodophenylamino)-N-(2-hydroxyethoxy)-1,5-dimethyl-6-oxo-1,6-dihydropyridine-3-carboxamide and Compound A. Treatment of cells with the combination was beneficial. Synergy was observed across the dosing regime when 2-(2-fluoro-4-iodophenylamino)-N-(2-hydroxyethoxy)-1,5-dimethyl-6-oxo-1,6-dihydropyridine-3-carboxamide and Compound A were dosed in concurrent combination at their IC₆₀, IC₅₀, IC₄₀ IC₃₀ and IC₂₀ inhibitory effect ratios.

FIG. 3 shows a representative Curve Shift Analysis Plot for treatment of the A2058 cell line with the MEK inhibitor AZD6244 and Rapamycin 300 nM. Treatment of cells with the combination yielded an additive effect.

FIG. 4 shows a representative Curve Shift Analysis Plot for treatment of the A2058 cell line with the MEK inhibitor AZD6244 and Rapamycin 3 nM. Treatment of cells with the combination yielded an additive effect.

FIG. 5 shows a representative Curve Shift Analysis Plot for treatment of the A2058 cell line with the MEK inhibitor 2-(2-fluoro-4-iodophenylamino)-N-(2-hydroxyethoxy)-1,5-dimethyl-6-oxo-1,6-dihydropyridine-3-carboxamide and Rapamycin 300 nM. Treatment of cells with the combination yielded an additive effect.

FIG. 6 shows a representative Curve Shift Analysis Plot for treatment of the A2058 cell line with the MEK inhibitor 2-(2-fluoro-4-iodophenylamino)-N-(2-hydroxyethoxy)-1,5-dimethyl-6-oxo-1,6-dihydropyridine-3-carboxamide and Rapamycin 3 nM. Treatment of cells with the combination yielded an additive effect.

FIG. 7 shows a representative Curve Shift Analysis Plot for the treatment of the A549 cell line with AZD6244 and Compound A. Treatment of the cells with the combination yielded an additive effect with evidence of synergy at higher doses of AZD6244.

FIG. 8 shows a representative Curve Shift Analysis Plot for the treatment of the A549 cell line with AZD6244 and Rapamycin. Treatment of the cells with the combination yielded a synergistic effect though the maximal inhibition of cell growth was less than observed with the combination of AZD6244 and Compound A. This is probably due to the incomplete effect of Rapamycin alone compared to Compound A. Therefore A549 cells exhibit resistance to the combination the of AZD6244 and Rapamycin even though there is a beneficial effect of the combination over either drug alone.

FIG. 9 shows a representative Curve Shift Analysis Plot for the treatment of the NCI-H460 cell line with AZD6244 and Compound A. Treatment of the cells with the combination yielded a synergistic effect.

FIG. 10 shows a representative Curve Shift Analysis Plot for the treatment of the NCI-H460 cell line with AZD6244 and Rapamycin. Treatment of the cells with the combination yielded a synergistic effect though the maximal inhibition of cell growth was less than observed with the combination of AZD6244 and Compound A. This is probably due to the incomplete effect of Rapamycin alone compared to Compound A. Therefore NCI-H460 cells exhibit resistance to the combination the of AZD6244 and Rapamycin even though there is a beneficial effect of the combination over either drug alone.

FIG. 11 shows a representative Curve Shift Analysis Plot for the treatment of the NCI-H23 cell line with AZD6244 and Compound A. Treatment of the cells with the combination yielded an additive effect at the first dose of AZD6244 with synergy at higher doses of AZD6244.

FIG. 12 shows a representative Curve Shift Analysis Plot for the treatment of the NCI-H23 cell line with AZD6244 and Rapamycin. Treatment of the cells with the combination yielded an additive effect at the first dose of AZD6244 with synergistic effect at higher doses. The maximal inhibition of cell growth was less than observed with the combination of AZD6244 and Compound A. This is probably due to the incomplete effect of Rapamycin alone compared to Compound A. Therefore NCI-H23 cells exhibit resistance to the combination the of AZD6244 and Rapamycin even though there is a beneficial effect of the combination over either drug alone.

FIG. 13 shows a representative Combination Index curve for treatment of the NCI-H2291 cell line with the MEK inhibitor AZD6244 and Compound A. Treatment of cells with the combination was beneficial. Synergy was observed across the dosing regime when AZD6244 and Compound A were dosed in concurrent combination at their IC₆₀, IC₅₀, IC₄₀, IC₃₀, IC₂₀ and IC₁₀ inhibitory effect ratios.

FIG. 14 shows a representative Curve Shift Analysis Plot for the treatment of the NCI-H2291 cell line with AZD6244 and Rapamycin. Treatment of the cells with the combination yielded a synergistic effect though the maximal inhibition of cell growth was less than observed with the combination of AZD6244 and Compound A. This is probably due to the incomplete effect of Rapamycin alone compared to Compound A. Therefore NCI-H2291 cells exhibit resistance to the combination of AZD6244 and Rapamycin even though there is a beneficial effect of the combination over either drug alone.

FIG. 15 shows a representative Combination Index curve for treatment of the NCI-H727 cell line with the MEK inhibitor AZD6244 and Compound A. Treatment of cells with the combination was beneficial. Additivity was observed across the dosing regime when AZD6244 and Compound A were dosed in concurrent combination at their IC₆₀, IC₅₀, IC₄₀, IC₃₀, IC₂₀ and IC₁₀ inhibitory effect ratios.

FIG. 16 shows a representative Curve Shift Analysis Plot for the treatment of the NCI-H727 cell line with AZD6244 and Rapamycin. Treatment of the cells with the combination yielded an additive effect.

FIG. 17 shows a representative Combination Index curve for treatment of the Calu-6 cell line with the MEK inhibitor AZD6244 and Compound A. Treatment of cells with the combination was beneficial. Additivity or synergy was observed when AZD6244 and Compound A were dosed in concurrent combination at their IC₆₀, IC₅₀, IC₄₀, IC₃₀, IC₂₀ and IC₁₀ inhibitory effect ratios with synergy observed at the higher inhibitory effect ratios.

FIG. 18 shows a representative Curve Shift Analysis Plot for the treatment of the Calu-6 cell line with AZD6244 and Rapamycin. Treatment of the cells with the combination yielded both synergistic and additive effects (depending upon the doses of the compounds) though the maximal inhibition of cell growth is largely determined by the activity of AZD6244 since Rapamycin exhibits only a partial inhibition of cell growth alone.

Table 4 summarises the Combination Indices calculated for each of the NCI-H727, Calu-6, NCI-H2291, NCI-H23, A549 and NCI-H460 cell lines in which the combination interaction was assessed through Curve Shift analysis.

TABLE 4
Summary of Combination Indices and Interaction in
the cell lines in which the combination interaction
was assessed through Curve Shift analysis.

| | | Combination | | Combination |
|---|---|---|---|---|
| Cell Line | Combination | index | P value | Interaction |
| A549 | Compound A | 0.928 | 0.195 | Additive |
| | Overall | | | |
| | +20 nM | 1.065 | 0.396 | Additive |
| | AZD6244 | | | |
| | +333 nM | 0.900 | 0.181 | Additive |
| | AZD6244 | | | |
| | +1000 nM | 0.671 | 1.02 × 10⁻⁵ | Synergistic |
| | AZD6244 | | | |
| | Rapamycin | 0.080 | 2.4 × 10⁻¹⁰ | Synergistic |
| | Overall | | | |
| | +20 nM | 0.769 | 0.552 | Synergistic |
| | AZD6244 | | | |
| | +333 nM | 0.009 | 4.695 × 10⁻¹⁵ | Synergistic |
| | AZD6244 | | | |
| | +1000 nM | 0.003 | 6.845 × 10⁻⁹ | Synergistic |
| | AZD6244 | | | |
| NCI-H460 | Compound A | 0.582 | 4.30 × 10⁻¹³ | Synergistic |
| | Overall | | | |
| | +20 nM | 0.806 | 0.004 | Synergistic |
| | AZD6244 | | | |
| | +333 nM | 0.398 | 1.23 × 10⁻²⁰ | Synergstic |
| | AZD6244 | | | |
| | +1000 nM | 0.296 | 8.06 × 10⁻²² | Synergistic |
| | AZD6244 | | | |
| | Rapamycin | 0.015 | 2.88 × 10−6 | Synergistic |
| | Overall | | | |
| | +20 nM | 0.051 | 0.004 | Synergistic |
| | AZD6244 | | | |
| | +333 nM | 0.001 | 1.52 × 10⁻⁷ | Synergstic |
| | AZD6244 | | | |
| | +1000 nM | 3.015 × 10⁻⁵ | 3.20 × 10⁻⁹ | Synergistic |
| | AZD6244 | | | |
| NCI-H23 | Compound A | 0.866 | 0.182 | Additive |
| | Overall | | | |
| | +20 nM | 1.189 | 0.213 | Additive |
| | AZD6244 | | | |
| | +333 nM | 0.667 | 0.007 | Synergistic |
| | AZD6244 | | | |
| | +1000 nM | 0.588 | 5.97 × 10⁻³ | Synergistic |
| | AZD6244 | | | |
| | Rapamycin | 0.008 | 6.85 × 10⁻¹³ | Synergistic |
| | Overall | | | |
| | +20 nM | 0.473 | 0.074 | Additive |
| | AZD6244 | | | |
| | +333 nM | 0.004 | 1.81 × 10⁻²² | Synergistic |
| | AZD6244 | | | |
| | +1000 nM | 0.0004 | 1.38 × 10⁻¹³ | Synergistic |
| | AZD6244 | | | |
| NCI- | Rapamycin | 0.102 | 4.72 × 10⁻²¹ | Synergistic |
| H2291 | Overall | | | |
| | +20 nM | 0.475 | 0.016 | Synergistic |
| | AZD6244 | | | |
| | +333 nM | 0.011 | 1.56 × 10⁻¹⁹ | Synergistic |
| | AZD6244 | | | |
| | +1000 nM | 0.005 | 6.97 × 10⁻¹³ | Synergistic |
| | AZD6244 | | | |
| NCI-H727 | Rapamycin | 0.965 | 0.647 | Additive |
| | Overall | | | |
| | +20 nM | 1.007 | 0.950 | Additive |
| | AZD6244 | | | |
| | +333 nM | 0.912 | 0.395 | Additive |
| | AZD6244 | | | |
| | +1000 nM | 0.979 | 0.849 | Additive |
| | AZD6244 | | | |
| Calu-6 | Rapamycin | 0.392 | 1.14 × 10⁻⁵ | Synergistic |
| | Overall | | | |
| | +20 nM | 0.379 | 0.135 | Additive |
| | AZD6244 | | | |
| | +333 nM | 0.391 | 7.27 × 10⁻⁶ | Synergistic |
| | AZD6244 | | | |
| | +1000 nM | 0.477 | 1.22 × 10⁻² | Additive |
| | AZD6244 | | | |

In summary, the combination of a MEK inhibitor and Compound A was found to be synergistic in the A2058, NCI-H460, NCI-H2291 and Calu-6 cell lines with both synergy and/or additive interactions detected in A549, NCI-H23 and NCI-H727 cell lines.

In addition both additive and synergistic effects were observed when AZD6244 was combined with Compound A in the A549, NCI-H23, NCI-H727, NCI-H460, NCI-H2291 and Calu-6 cell lines.

No benefit was observed when dosing AZD6244 or 2-(2-fluoro-4-iodophenylamino)-N-(2-hydroxyethoxy)-1,5-dimethyl-6-oxo-1,6-dihydropyridine-3-carboxamide concurrently with Rapamycin in the A2058 cell line. Rapamycin did not enhance the effects of AZD6244 or 2-(2-fluoro-4-iodophenylamino)-N-(2-hydroxyethoxy)-1,5-dimethyl-6-oxo-1,6-dihydropyridine-3-carboxamide.

Whilst analysis of the combination interaction between AZD6244 and Rapamycin also indicated beneficial effects (both synergistic and additive effects) the extent of the inhibition of tumour cell growth (see maximal inhibition of cell growth achieved by the combination in FIGS. 7-18) was generally less than that observed with the combination of AZD6244 and Compound A. Therefore, despite the synergistic interaction between AZD6244 and Rapamycin, some cell lines remain relatively resistant to the combination of AZD6244 and Rapamycin for example A549, NCI-H460, NCI-H23 and NCI-H2291 cells. In these cell lines regardless of the presence of AZD6244, Rapamycin failed to reach a 50% growth inhibitory effect.

### Example 4

### In Vivo Combination Study of AZD6244 or 2-(2-fluoro-4-iodophenylamino)-N-(2-hydroxyethoxy)-1,5-dimethyl-6-oxo-1,6-dihydropyridine-3-carboxamide with the mTOR-Selective Inhibitor [5-[2,4-bis[(3S)-3-methylmorpholin-4-yl]pyrido[5,6-e]pyrimidin-7-yl]-2-methoxyphenyl]methanol (“Compound A”), in the HCT-116, LoVo, A549a and CaLu6 Tumour Models

LoVo cells were grown in DMEM (Gibco) 10% FCS 1% Glutamine medium and HCT116 cells were grown in McCoys 5A 10% FCS 1% Glutamine, A549a and CaLu-6 cells were grown in RPMI1640 10% FCS 1% Glutamine. 1×10A7 cells were implanted into the flank of nude mice (LoVo no matrigel, HCT-116, Calu-6 and A549a plus 50% matrigel).

When mean tumour size reached approximately 0.2 cm³, the mice were randomized into control and treatment groups. The treatment groups received either 25 or 50 mg/kg AZD6244 (vehicle: HPMC/Tween milled overnight), or 20 mg/kg Compound A (vehicle 10% DMSO, 90% propylene glycol) by oral gavage, Rapamycin was given 4 mg/kg once weekly (10% DMSO-10% Cremaphor-10% Ethanol-70% water for injection. When administered in combination, Compound A was given 2 hours after the oral dose of the other compound. The control group received vehicle (10% DMSO 90% propylene glycol) alone, once daily by oral gavage.

Tumour volumes (measured by caliper), animal body weight and tumour condition were recorded twice weekly for the duration of the study. Mice were sacrificed by CO₂ euthanasia. The tumour volume was calculated (taking length to be the longest diameter across the tumour and width to be the corresponding perpendicular diameter using the formula: (length×width)×√(length×width)×(π/6). Growth inhibition from the start of treatment was assessed by comparison of the differences in tumour volume between control and treated groups. Because the variance in mean tumour volume data increases proportionally with volume (and is therefore disproportionate between groups), data were log-transformed to remove any size dependency before statistical evaluation.

Statistical significance was evaluated using a one-tailed, two-sample t test. To analyze the data from the combination study, the statistical tool SigmaStat has been used. A two-way ANOVA test was performed using the factors concentration of drug A and concentration of drug B. The data analyzed was Log (final tumour volume)-Log (initial tumour volume) calculated for each individual group at the end of the study. This tool is used to assess whether there is a main effect of drug A, a main effect of drug B plus a significant interaction between the two compounds A and B (eg. one compound influences the effect of the other compound) which may be interpreted as antagonism, additivity or synergism.

FIG. 19. shows the combination of AZD6244 and Compound A in HCT-116 xenografts. In the HCT-116 model, 20 mg/kg of Compound A gave a 24% reduction in geometric mean delta tumour volume (p=0.04 compared with the vehicle control), 25 mg/kg AZD6244 gave a 59% reduction in geometric mean delta tumour volume (p<0.0001) and the combination of the same doses of these two agents resulted in an 89% reduction in geometric mean delta tumour volume (p<0.0001). The combination treatment was significantly more effective than the monotherapy at reducing tumour volume (combination versus Compound A monotherapy 67% effect, p<0.001, combination versus AZD6244 monotherapy 48% effect, p=0.001). A statistically significant interaction occurs between Compound A and AZD6244 at these doses in the HCT-116 model (SigmaStat analysis P=0.007), indicating a synergistic effect.

FIG. 20. shows the combination of AZD6244 and Compound A in LoVo xenografts. In the LoVo model, 20 mg/kg of Compound A gave a 66% reduction in geometric mean delta tumour volume (p<0.001 compared with the vehicle control), 50 mg/kg AZD6244 gave a 43% reduction in geometric mean delta tumour volume (p=0.0001) and the combination of the same doses of these two agents resulted in a 93% reduction in geometric mean delta tumour volume (p<0.0001). The combination group was significantly more effective than the monotherapy groups at reducing tumour volume (combination versus Compound A monotherapy 40% effect, p<0.0001, combination versus AZD6244 monotherapy 56% effect, p=0.001). No significant interaction occurs between Compound A and AZD6244 at these doses in the LoVo model (SigmaStat P=0.187), indicating an additive effect.

FIG. 21. shows the combination of AZD6244 and Rapamycin in LoVo xenografts. In the LoVo model, 4 mg/kg Rapamycin gave a 38.2% reduction in geometric mean delta tumour volume compared with the vehicle treated control (SigmaStat analysis P=0.0059), 25 mg/kg twice daily AZD6244 gave a 38.7% reduction in geometric mean delta tumour volume compared with the vehicle treated control (SigmaStat analysis P=0.0007). The combination treatment was significantly more effective than the monotherapy; reducing tumour volume by 63.2% compared to vehicle treated control (P<0.0001), by 23% compared to AZD6244 (P 0.0007) and by 24% compared to Rapamycin (P<0.0088). SigmaStat analysis of the interaction between AZD6244 and Rapamycin at these doses in the LoVo model, indicates an additive effect.

FIG. 22. shows the combination of AZD6244 and Compound A in Calu-6 xenografts. FIG. 23. shows the combination of AZD6244 and Rapamycin in Calu-6 xenografts. In the Calu-6 model, 50 mg/kg once daily AZD6244 gave a 93.2% reduction in geometric mean delta tumour volume compared with the vehicle treated control (SigmaStat analysis P<0.0001); 20 mg/kg once daily Compound A gave a 44.9% reduction in geometric mean delta tumour volume compared with the vehicle treated control (SigmaStat analysis P=0.00273) and 4 mg/kg once weekly Rapamycin gave a 37.2% reduction in geometric mean delta tumour volume compared with the vehicle treated control (SigmaStat analysis P=0.012). The combination of AZD6244 and Compound A gave a 109.6% reduction in geometric mean delta tumour volume compared with the vehicle treated control (SigmaStat analysis P<0.0001). SigmaStat analysis of the interaction between AZD6244 and Compound A at these doses in the Calu-6 model indicates an additive effect. The combination of AZD6244 with Rapamycin gave a 99.4% reduction in geometric mean delta tumour volume compared with the vehicle treated control (SigmaStat analysis P<0.0001). SigmaStat analysis of the interaction between AZD6244 and Rapamycin at these doses in the Calu-6 model indicates an additive effect.

FIG. 24. shows the combination of AZD6244 and Compound A in A549a xenografts. FIG. 25. shows the combination of AZD6244 and Rapamycin in A549a xenografts. In the A549a model, 25 mg/kg once daily AZD6244 gave a 53.4% reduction in geometric mean delta tumour volume compared with the vehicle treated control (SigmaStat analysis P<0.0001); 20 mg/kg once daily Compound A gave a 94.1% reduction in geometric mean delta tumour volume compared with the vehicle treated control (SigmaStat analysis P<0.0001) and 4 mg/kg once weekly Rapamycin gave a 69.5% reduction in geometric mean delta tumour volume compared with the vehicle treated control (SigmaStat analysis P<0.0001). The combination of AZD6244 and Compound A gave a 106.9% reduction in geometric mean delta tumour volume compared with the vehicle treated control (SigmaStat analysis P<0.0001). SigmaStat analysis of the interaction between AZD6244 and Compound A at these doses in the A549a model indicates an additive effect. The combination of AZD6244 with Rapamycin gave a 65.7% reduction in geometric mean delta tumour volume compared with the vehicle treated control (SigmaStat analysis P<0.0001). SigmaStat analysis of the interaction between AZD6244 and Rapamycin indicates antagonism since the combination of AZD6244 and Rapamycin did not have any effect over Rapamycin used alone.

The in vivo experiments demonstrate that a combination of AZD6244 with either Compound A or with Rapamycin can achieve a greater degree of tumour growth inhibition than either monotherapy alone; however in these experiments the combination of AZD6244 and Compound A was able to achieve greater inhibition of tumour growth than the combination of AZD6244 and Rapamycin.

1. A combination product comprising
a MEK inhibitor, or a pharmaceutically acceptable salt thereof, and
a mTOR-selective inhibitor, or a pharmaceutically acceptable salt thereof, in association with a pharmaceutically acceptable adjuvant, diluent or carrier.

2. A combination product which comprises a kit of parts comprising the following components:
a MEK inhibitor, or a pharmaceutically acceptable salt thereof, in association with a pharmaceutically acceptable adjuvant, diluent or carrier; and
a mTOR-selective inhibitor, or a pharmaceutically acceptable salt thereof, in association with a pharmaceutically acceptable adjuvant, diluent or carrier,
wherein the components are provided in a form which is suitable for sequential, separate and/or simultaneous administration, and further comprising instructions to administer the components sequentially, separately and/or simultaneously.

3. A combination product according to claim 1, wherein the MEK inhibitor is AZD6244, or a pharmaceutically acceptable salt thereof.

4. A combination product according to claim 1, wherein the MEK inhibitor is AZD6244 hydrogen sulphate salt.

5. A combination product according to claim 1, wherein the MEK inhibitor is 2-(2-fluoro-4-iodophenylamino)-N-(2-hydroxyethoxy)-1,5-dimethyl-6-oxo-1,6-dihydropyridine-3-carboxamide, or a pharmaceutically acceptable salt thereof.

6. A combination product according to claim 1, wherein the MEK inhibitor is 4-(4-Bromo-2-fluorophenylamino)-N-(2-hydroxyethoxy)-1,5-dimethyl-6-oxo-1,6-dihydropyridazine-3-carboxamide, or a pharmaceutically acceptable salt thereof.

7. A combination product according to claim 1 wherein the mTOR-selective inhibitor inhibits TORC2.

8. A combination product according to claim 1 wherein the mTOR-selective inhibitor inhibits TORC1 and TORC2.

9. A combination product according to claim 1 wherein the mTOR-selective inhibitor is selected from any one of
5-[2,4-bis[(3S)-3-methylmorpholin-4-yl]pyrido[5,6-e]pyrimidin-7-yl]-2-methoxy-N-methylbenzamide;
4-[2,4-bis[(3S)-3-methylmorpholin-4-yl]pyrido[5,6-e]pyrimidin-7-yl]aniline;
6-[2,4-bis[(3S)-3-methylmorpholin-4-yl]pyrido[5,6-e]pyrimidin-7-yl]-1H-indazol-3-amine;
8-[2,4-bis[(3S)-3-methylmorpholin-4-yl]pyrido[5,6-e]pyrimidin-7-yl]-1,2,3,4-tetrahydro-1,4-benzodiazepin-5-one;
5-[2,4-bis[(3S)-3-methylmorpholin-4-yl]pyrido[5,6-e]pyrimidin-7-yl]-2-methoxy-N-methylbenzamide;
5-[2,4-bis[(3S)-3-methylmorpholin-4-yl]pyrido[5,6-e]pyrimidin-7-yl]pyridin-2-amine;
N-[3-[2,4-bis[(3S)-3-methylmorpholin-4-yl]pyrido[5,6-e]pyrimidin-7-yl]phenyl]methanesulfonamide;
3-[2,4-bis[(3S)-3-methylmorpholin-4-yl]pyrido[5,6-e]pyrimidin-7-yl]aniline;
5-[2,4-bis[(3S)-3-methylmorpholin-4-yl]pyrido[6,5-d]pyrimidin-7-yl]-2-ethoxybenzamide;
5-[2,4-bis[(3S)-3-methylmorpholin-4-yl]pyrido[5,6-e]pyrimidin-7-yl]-2-(difluoromethoxy)-N-methylbenzamide;
5-[2,4-bis[(3S)-3-methylmorpholin-4-yl]pyrido[5,6-e]pyrimidin-7-yl]-1H-indazol-3-amine;
[5-[2,4-bis[(3S)-3-methylmorpholin-4-yl]pyrido[5,6-e]pyrimidin-7-yl]-2-methoxyphenyl]methanol;
N-[[4-[2,4-bis[(3S)-3-methylmorpholin-4-yl]pyrido[5,6-e]pyrimidin-7-yl]phenyl]methyl]methanesulfonamide;
5-[2,4-bis[(3S)-3-methylmorpholin-4-yl]pyrido[5,6-e]pyrimidin-7-yl]-1,3-dihydroindol-2-one;
6-[2,4-bis[(3S)-3-methylmorpholin-4-yl]pyrido[5,6-e]pyrimidin-7-yl]-1,3-dihydroindol-2-one;
3-[2,4-bis[(3S)-3-methylmorpholin-4-yl]pyrido[5,6-e]pyrimidin-7-yl]-N-methylbenzamide;
5-[2,4-bis[(3S)-3-methylmorpholin-4-yl]pyrido[5,6-e]pyrimidin-7-yl]-2-(difluoromethoxy)benzamide;
6-[2,4-bis[(3S)-3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

10. A method of treating cancer, which comprises administration of a combination product according to claim 1 to a patient, having or suspected of having cancer.

11. A method of treating cancer according to claim 10 wherein the cancer is selected from lung cancer, melanoma, colorectal cancer, hepatocellular carcinoma, gastric cancer, breast cancer, ovarian cancer, thyroid cancer, pancreatic cancer, liver cancer, acute myeloid leukaemia or multiple myeloma.

12. A combination product comprising a pharmaceutical composition which comprises a MEK inhibitor, or a pharmaceutically-acceptable salt thereof, and a mTOR-selective inhibitor, or a pharmaceutically-acceptable salt thereof.
